(19) United States
(12) Patent Application Publication
(10) Pub. No.: US 2022/0178944 A1
(43) Pub. Date: Jun. 9, 2022

(54) METHODS FOR IDENTIFYING DIFFERENTIAL HISTONE RETENTION FOR EPIGENETIC ALTERATIONS IN GERMLINE DNA OF ANIMALS

(71) Applicant: Washington State University, Pullman, WA (US)

(72) Inventor: Michael K. Skinner, Pullman, WA (US)

(21) Appl. No.: 17/541,589

(22) Filed: Dec. 3, 2021

Related U.S. Application Data
(60) Provisional application No. 63/121,644, filed on Dec. 4, 2020.

(51) Int. Cl. G01N 33/68; G01N 33/53; G01N 33/574; G01N 33/577

(52) U.S. Cl. CPC G01N 33/6875; G01N 33/5308; G01N 2800/52; G01N 33/577; G01N 33/6893; G01N 33/57488

(57) ABSTRACT

The present disclosure relates to methods of analyzing epigenetic alterations in the germline DNA of animals. More particularly, the present disclosure relates to methods of analyzing histone retention involved in epigenetic alterations as well as the identification and treatment of diseases associated with such epigenetic alterations.

### CROSS REFERENCE TO RELATED APPLICATIONS

This application claims the benefit under 35 USC § 119(e) of U.S. Provisional Application Ser. No. 63/121,644, filed on Dec. 4, 2020, the entire disclosure of which is incorporated herein by reference.

### BACKGROUND

The present disclosure relates to methods of analyzing epigenetic alterations in the germline DNA of animals. More particularly, the present disclosure relates to methods of analyzing histone retention involved in epigenetic alterations as well as the identification and treatment of diseases associated with such epigenetic alterations.

### SUMMARY

Generally, epigenetics refers to molecular factors and processes around DNA that regulate genome activity but are independent of DNA sequences. Various epigenetic processes include DNA methylation, histone modifications, non-coding RNA, chromatin structure, and RNA methylation. Epigenetics is becoming increasingly important for evaluation of diseases that may be induced by exposure to environmental toxicants. For instance, a variety of environmental factors can promote epigenetic transgenerational inheritance of disease or phenotypic alterations through epigenetic changes in the germline (sperm or eggs). Although direct exposure of an animal to a particular toxicant may not result in a disease pathology, it is important to recognize that subsequent offspring of the animal may have an increased propensity to develop a disease as a result of the toxicant exposure to a prior generation of relation.

For example, glyphosate is one of the most commonly used herbicides in agriculture. A low level of glyphosate direct exposure to animals (i.e., below the No Observational Adverse Effect Level (NOAEL) dose of 50 mg/kg per day) has no effect on the pathologies of the directly exposed individuals using a mammalian rat model. When the exposed gestating female F0 generation and F1 generation offspring were examined later in life, both generations were found to have negligible detectable alteration in pathology compared to controls. However, evaluation of the subsequent F2 generation grand offspring and F3 generation great-grand offspring demonstrated a significant increase in the frequency of pathology and disease. In particular, the F2 generation demonstrated an increase in testis and kidney disease, altered pubertal onset, increased obesity, increased ovarian disease, mammary tumors, altered pubertal onset, obesity, premature birth abnormalities, and the presence of multiple disease. The glyphosate induced transgenerational disease in the F3 generation pathology included prostate disease, obesity, weaning weight alterations, ovarian disease, kidney disease, parturition abnormalities, obesity, and increased presence of multiple disease.

However, there exists a need for further mechanisms to evaluate epigenetic alterations in animals, especially following exposure to toxicants. Accordingly, the present disclosure provides evaluation of differential histone retention sites (DHRs) as disease biomarkers. The present disclosure provides methods for evaluating DHRs in sperm as disease biomarkers as well as the use of overlapping differential DNA methylation region (DMR) DMR and DHR biomarkers between different diseases or pathologies.

In illustrative embodiments, a method of inducing an epigenetic alteration in germline DNA of a male progeny animal is provided. For these embodiments, the method comprises administering one or more toxicants to a female parental animal wherein the female parental animal subsequently gives birth to the male progeny animal, and wherein the male progeny animal comprises the epigenetic alteration in germline DNA.

In illustrative embodiments, a method for preparing a DNA fraction from an animal useful for analyzing histone retention involved in an epigenetic alteration is provided. For these embodiments, the method comprises (a) extracting DNA from a germline sample of an animal, (b) producing a fraction of the DNA extracted in (a) by selecting DNA comprising histone retention, and (c) analyzing the histone retention in the fraction of DNA produced in (b).

In illustrative embodiments, a method of identifying a disease or a disease propensity in a male progeny animal is provided. For these embodiments, the method comprises identifying a profile of histone retention DNA in the male progeny animal, wherein the profile is associated with the disease or the disease propensity in the male progeny animal, and wherein the disease or the disease propensity is associated with an epigenetic alteration in germline DNA resulting from contact of the germline DNA of the male progeny animal with one or more toxicants during gestation of the male progeny animal.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIGS. 1A-1F show DMR identification and numbers. The number of DMRs found using different p-value cutoff thresholds. The All Window column shows all DMRs. The Multiple Window column shows the number of DMRs containing at least two significant windows (1 kb each). The number of DMRs with the number of significant windows (1 kb per window) at an edgeR p-value threshold bolded for DMR. (A) Glyphosate versus control DMRs; (B) Prostate disease DMRs; (C) Kidney disease DMRs; (D) Obesity disease DMRs; and (E) Multiple disease DMRs. (F) Venn diagram DMR overlap of each data set with edgeR p-value indicated. The bolded edgeR p-value was used for all subsequent data analysis.

FIG. 2 shows DMR Site List Glyphosate edgeR p<1e−05. DMR name, chromosome, start, stop, length, number signature windows, minimum p-value, max log-fold change, CpG number, CpG density, gene annotation, and gene category are presented.

FIG. 3 shows DMR Site List Prostate edgeR p<1e−04. DMR name, chromosome, start, stop, length, number signature windows, minimum p-value, max log-fold change, CpG number, CpG density, gene annotation, and gene category are presented.

FIG. 4 shows DMR Site List Kidney edgeR p<1e−04. DMR name, chromosome, start, stop, length, number signature windows, minimum p-value, max log-fold change, CpG number, CpG density, gene annotation, and gene category are presented.

FIG. 5 shows DMR Site List Obese edgeR p<1e−04. DMR name, chromosome, start, stop, length, number signature windows, minimum p-value, max log-fold change, CpG number, CpG density, gene annotation, and gene category are presented.

FIG. 6 shows DMR Site List Multiple 2p edgeR p<1e−04. DMR name, chromosome, start, stop, length, number signature windows, minimum p-value, max log-fold change, CpG number, CpG density, gene annotation, and gene category are presented.

FIGS. 7A-7E show DMR chromosomal locations. The DMR locations on the individual chromosomes is represented with an arrowhead and a cluster of DMRs with a black box. All DMRs containing at least one significant window at the select (bold) edgeR p-value threshold are shown. The chromosome number and size of the chromosome (megabase) are presented. (A) Glyphosate versus control DMRs; (B) Prostate disease DMRs; (C) Kidney disease DMRs; (D) Obesity disease DMRs; and (E) Multiple disease DMRs.

FIGS. 8A-8J show DMR genomic features. The number of DMRs at different CpG densities. All DMRs at a p-value threshold of edgeR p<1e−04 for disease specific comparisons and p<1e−05 for exposure comparisons are shown. (A) Glyphosate versus control DMR CpG density; (B) Glyphosate versus control DMR lengths; (C) Prostate disease DMR CpG density; (D) Prostate disease DMR length; (E) Kidney disease DMR CpG density; (F) Kidney disease DMR length; (G) Obesity disease DMR CpG density; (H) Obesity disease DMR length; (I) Multiple disease DMR CpG density; (J) Multiple disease DMR length.

FIGS. 9A-9E show DMR principal component analysis. The first two principal components used. The underlying data is the RPKM read depth for DMR only genomic windows. (A) Glyphosate versus control DMRs PCA; (B) Prostate disease DMRs PCA; (C) Kidney disease DMRs PCA; (D) Obesity disease DMRs PCA; (E) Multiple disease DMRs PCA.

FIGS. 10A-10F show DHR identification and numbers. The number of DHRs found using different edgeR p-value cutoff thresholds. The All Window column shows all DHRs. The Multiple Window column shows the number of DHRs containing at least two significant windows (1 kb each). The number of DHRs with the number of significant windows (1 kb per window) at a bolded p-value threshold is presented. (A) Glyphosate versus control DHRs; (B) Prostate disease DHRs; (C) Kidney disease DHRs; (D) Obesity disease DHRs; and (E) Multiple disease DHRs. (F) Venn diagram DHR overlap for each data set with p-value indicated.

FIG. 11 shows DHR Site List Glyphosate edgeR p<1e−05. DMR name, chromosome, start, stop, length, number signature windows, minimum p-value, max log-fold change, CpG number, CpG density, gene annotation, and gene category are presented.

FIG. 12 shows DHR Site List Prostate edgeR p<1e−04. DMR name, chromosome, start, stop, length, number signature windows, minimum p-value, max log-fold change, CpG number, CpG density, gene annotation, and gene category are presented.

FIG. 13 shows DHR Site List Kidney edgeR p<1e−04. DMR name, chromosome, start, stop, length, number signature windows, minimum p-value, max log-fold change, CpG number, CpG density, gene annotation, and gene category are presented.

FIG. 14 shows DHR Site List Obese edgeR p<1e−04. DMR name, chromosome, start, stop, length, number signature windows, minimum p-value, max log-fold change, CpG number, CpG density, gene annotation, and gene category are presented.

FIG. 15 shows DHR Site List Multiple 2p edgeR p<1e−04. DMR name, chromosome, start, stop, length, number signature windows, minimum p-value, max log-fold change, CpG number, CpG density, gene annotation, and gene category are presented.

FIGS. 16A-16E show DHR chromosomal locations. The DHR locations on the individual chromosomes is represented with an arrowhead and a cluster of DHRs with a black box. All DHRs containing at least one significant window at the edgeR p-value threshold selected (bold) are shown. The chromosome number and size (megabase) are presented. (A) Glyphosate versus control DHRs; (B) Prostate disease DHRs; (C) Kidney disease DHRs; (D) Obesity disease DHRs; and (E) Multiple disease DHRs.

FIGS. 17A-17J show DHR genomic features. The number of DMRs at different CpG densities. All DHRs at a p-value threshold of edgeR p<1e−04 for disease specific comparisons and p<1e−05 for exposure comparisons are shown. (A) Glyphosate versus control DHR CpG density; (B) Glyphosate versus control DHR lengths; (C) Prostate disease DHR CpG density; (D) Prostate disease DHR length; (E) Kidney disease DHR CpG density; (F) Kidney disease DHR length; (G) Obesity disease DHR CpG density; (H) Obesity disease DHR length; (I) Multiple disease DHR CpG density; (J) Multiple disease DHR length.

FIGS. 18A-18E show DHR principal component analysis. The first two principal components used. The underlying data is the RPKM read depth for DHR only genomic windows. (A) Glyphosate versus control DHRs PCA; (B) Prostate disease DHRs PCA; (C) Kidney disease DHRs PCA; (D) Obesity disease DHRs PCA; (E) Multiple disease DHRs PCA.

FIG. 19 shows extended overlap disease DMRs and DHRs. The p-value data set at p<1e−04 for disease specific and p<1e−05 for exposure specific are compared to the p<0.05 data to identify potential overlap between the different pathologies with DMR or DHR number and percentage of the total presented. The gray highlight is the expanded 100% overlap and yellow highlight overlaps >25%.

FIG. 20 shows overlapping common DMRs and DHRs lists between all pathology biomarkers. Common DMRs with all specific pathology biomarkers: (A) Kidney disease DMR, (B) Prostate disease DMR, (C) Obesity DMR, and (D) Multiple disease DMR. Common DHRs with all pathology biomarkers: (E) Kidney disease DHR, (F) Prostate disease DHR, (G) Obesity DHR, and (H) Multiple disease DHR.

FIGS. 21A-21B show overlap of specific disease overlap epimutations. Venn diagram of extended overlap analysis for specific disease states with common epimutations in common with other diseases at a lower statistical threshold. The overlap of each specific disease overlapping DMRs (A), and DHRs (B) are presented.

FIGS. 22A-22B show associated gene categories. (A) DMR associated gene categories. (B) DHR associated gene categories. The different gene categories and number DMR or DHR presented with color index insert. No statistical analysis was performed for over-represented genes, but sample correlations provided for each gene category.

FIGS. 23A-23C show DMR associated genes within the pathology biomarker DMR set for each individual pathology. The physiologic and pathology process is listed with direct gene links. (A) Prostate disease, (B) kidney disease, and (C) obesity.

FIG. 24 shows DMR associated genes within the pathology biomarker DMR set for multiple disease pathology. The physiologic and pathology process is listed with direct gene links.

FIGS. 25A-25E show DMR principal component analysis (PCA). The first two principal components used. The underlying data is the RPKM read depth for DMR associated genomic windows. (A) Lean phenotype DMRs PCA; (B) Kidney disease DMRs PCA; (C) Testis disease DMRs PCA; (D) Late puberty DMRs PCA; and (E) Multiple disease DMRs PCA.

FIGS. 26A-26F show DMR identification and numbers. The number of DMRs found using different p-value cutoff thresholds. The All Window column shows all DMRs. The Multiple Window column shows the number of DMRs containing at least two nearby significant windows (1 kb each). The number of DMRs with the number of significant windows (1 kb per window) at a p-value threshold of p<1e−04 for DMR is bolded. (A) Lean phenotype DMRs; (B) Kidney disease DMRs; (C) Testis disease DMRs; (D) Late puberty DMRs; and (E) Multiple disease DMRs. (F) Venn diagram overlap disease specific DMR at p<1e−04.

FIG. 27 shows DMR Site List Lean p<1e−04. DMR name, chromosome, start, stop, length, number signature windows, minimum p-value, max log-fold change, CpG number, CpG density, gene annotation, and gene category are presented.

FIG. 28 shows DMR Site List Kidney p<1e−04. DMR name, chromosome, start, stop, length, number signature windows, minimum p-value, max log-fold change, CpG number, CpG density, gene annotation, and gene category are presented.

FIG. 29 shows DMR Site List Testis p<1e−04. DMR name, chromosome, start, stop, length, number signature windows, minimum p-value, max log-fold change, CpG number, CpG density, gene annotation, and gene category are presented.

FIG. 30 shows DMR Site List Puberty p<1e−04. DMR name, chromosome, start, stop, length, number signature windows, minimum p-value, max log-fold change, CpG number, CpG density, gene annotation, and gene category are presented.

FIG. 31 shows DMR Site List Multiple p<1e−04. DMR name, chromosome, start, stop, length, number signature windows, minimum p-value, max log-fold change, CpG number, CpG density, gene annotation, and gene category are presented.

FIGS. 32A-32E show DMR chromosomal locations. The DMR locations on the individual chromosomes is represented with an arrowhead and a cluster of DMRs with a black box. All DMRs containing at least one significant window at a p-value threshold of p<1e−04 for DMR are shown. (A) Lean phenotype DMRs; (B) Kidney disease DMRs; (C) Testis disease DMRs; (D) Late puberty DMRs; and (E) Multiple disease DMRs. The chromosome number versus size (megabase) is presented.

FIGS. 33A-33J show DMR genomic features. The number of DMRs at different CpG densities. All DMRs at a p-value threshold of p<1e−04 are shown. (A) Lean phenotype DMR CpG density; (B) Lean phenotype DMR length; (C) Kidney disease DMR CpG density; (D) Kidney disease DMR length; (E) Testis disease DMR CpG density; (F) Testis disease DMR length; (G) Late puberty DMR CpG density; (H) Late puberty DMR length; (I) Multiple disease DMR CpG density; and (J) Multiple disease DMR length.

FIGS. 34A-34F shows DHR identification and numbers. The number of DHRs found using different p-value cutoff thresholds. The All Window column shows all DHRs. The Multiple Window column shows the number of DHRs containing at least two nearby significant windows (1 kb each). The number of DMRs with the number of significant windows (1 kb per window) at a p-value threshold of p<1e−04 for DHR is presented. (A) Lean phenotype DHRs; (B) Kidney disease DHRs; (C) Testis disease DHRs; (D) Late puberty DHRs; and (E) Multiple disease DHRs.

FIG. 35 shows DHR Site List Lean p<1e−05. DHR name, chromosome, start, stop, length, number signature windows, minimum p-value, max log-fold change, CpG number, CpG density, gene annotation, and gene category are presented.

FIG. 36 shows DHR Site List Kidney p<1e−04. DHR name, chromosome, start, stop, length, number signature windows, minimum p-value, max log-fold change, CpG number, CpG density, gene annotation, and gene category are presented.

FIG. 37 shows DHR Site List Testis p<1e−04. DHR name, chromosome, start, stop, length, number signature windows, minimum p-value, max log-fold change, CpG number, CpG density, gene annotation, and gene category are presented.

FIG. 38 shows DHR Site List Puberty p<1e−04. DHR name, chromosome, start, stop, length, number signature windows, minimum p-value, max log-fold change, CpG number, CpG density, gene annotation, and gene category are presented.

FIG. 39 shows DHR Site List Multiple p<1e−04. DHR name, chromosome, start, stop, length, number signature windows, minimum p-value, max log-fold change, CpG number, CpG density, gene annotation, and gene category are presented.

FIGS. 40A-40E show DHR chromosomal locations. The DHR locations on the individual chromosomes is represented with an arrowhead and a cluster of DHRs with a black box. All DHRs containing at least one significant window at a p-value threshold of 1e−04 for DHR are shown. (A) Lean phenotype DHRs (p<1e−5); (B) Kidney disease DHRs; (C) Testis disease DHRs; (D) Late puberty DHRs; and (E) Multiple disease DHRs.

FIGS. 41A-41J show DHR genomic features. The number of DHRs at different CpG densities. All DHRs at a p-value threshold of 1e−04 are shown. (A) Atrazine versus control DHR CpG density; (B) Atrazine versus control DHR lengths; (C) Lean phenotype DHR CpG density; (D) Lean phenotype DHR length; (E) Kidney disease DHR CpG density; (F) Kidney disease DHR length; (G) Testis disease DHR CpG density; (H) Testis disease DHR length; (I) Late puberty DHR CpG density; (J) Late puberty DHR length; (K) Multiple disease DHR CpG density; and (L) Multiple disease DHR length.

FIGS. 42A-42E show DHR principal component analysis (PCA). The first two principal components used. The underlying data is the RPKM read depth for DHR genomic windows. (A) Lean phenotype DHRs PCA; (B) Kidney disease DHRs PCA; (C) Testes disease DHRs PCA; (D) Late puberty DHRs PCA; and (E) Multiple disease DHRs PCA.

FIGS. 43A-43E show transgenerational atrazine versus control lineage DHRs. (A) DHRs identified at various edgeR p-value thresholds for All Window (1 kb) and Multiple Window (≥2 nearby 1 kb) with the DHR numbers presented. The DHRs at p<1e−04 were selected for subsequent analysis. (B) DHR chromosomal locations with red arrowhead indicating location of DHRs and black box DHR clusters and different chromosome numbers versus chromosome size (megabase). (C) DHR CpG density for number of DHRs per number of CpG/100 bp. (D) DHR length with number of DHR versus DHR length (kb). (E) Principal component analysis (PCA) of DHR read depths for principal components 1 and 2 for control and autism DHRs.

FIG. 44 shows atrazine versus control F3 generation lineage sperm DHRs. DHR site list for atrazine versus control sperm DHRs at p<1e−04. DHR name, chromosome, start, stop, length, number signature windows, minimum p-value, max log-fold change, CpG number, CpG density, gene annotation, and gene category are presented.

FIGS. 45A-45B show a Venn diagram of DMR and DHR overlaps. (A) DMR overlaps with disease DMRs at p<1e−04 and all different diseases DMR overlaps at p<0.05 (All). (B) DHR overlaps at p<1e−04 and all different diseases DHR overlaps at p<0.05 (All).

FIGS. 46A-46B show associated gene categories. (A) DMR associated gene categories. (B) DHR associated gene categories. The gene categories and number of associated genes are presented for each disease group.

FIGS. 47A-47B show epimutation associated KEGG gene pathways. (A) DMR associated gene pathways for each disease DMR data set. (B) DHR associated gene pathways for each disease DHR data set. The pathway and number of associated DMR or DHR in brackets indicated.

FIGS. 48A-48C show epimutation associated previously identified disease genes. (A) Epimutation associated kidney disease genes. (B) Epimutation associated lean disease genes. (C) Epimutation associated testis disease genes. The green shading represents DMRs and pink shading DHRs. The gene shapes are identified as follows inset in FIG. 7.

FIG. 49 shows multiple disease associated previously identified disease genes. The disease categories and gene identified shapes are presented. The green shading represents DMRs and pink shading DHRs.

### DETAILED DESCRIPTION

In an illustrative aspect, a method of inducing an epigenetic alteration in germline DNA of a male progeny animal is provided. For these embodiments, the method comprises administering one or more toxicants to a female parental animal wherein the female parental animal subsequently gives birth to the male progeny animal, and wherein the male progeny animal comprises the epigenetic alteration in germline DNA.

Any toxicant capable of inducing an epigenetic alteration is contemplated by the present disclosure. For instance, the examples described herein utilize glyphosate and atrazine as exemplary environmental toxicants. Without being bound by any hypothesis, it is contemplated that other toxicants are capable of inducing an epigenetic alteration according to the methodology of the present disclosure.

In an embodiment, the epigenetic alteration is differential histone retention of DNA in the germline DNA of the male progeny animal. In an embodiment, the epigenetic alteration is differential histone retention of DNA in the germline DNA of the male progeny animal. In an embodiment, the epigenetic alteration in the germline DNA of the male progeny animal is associated with a disease in the male progeny animal. In an embodiment, the disease is selected from the group consisting of kidney disease, prostate disease, male infertility, immune cell activation, cancer, and any combination thereof.

In an embodiment, the epigenetic alteration in germline DNA is associated with in reduced fertility of the male progeny animal or sterility of the male progeny animal. In an embodiment, the reduced fertility or sterility of the male progeny animal is associated with abnormal testicular development, abnormal spermatogenesis, decreased sperm motility, decreased forward sperm movement, or any combination thereof.

In an embodiment, the animal is a human. In an embodiment, the animal is a companion animal. In an embodiment, the companion animal is a dog or a cat.

In an illustrative aspect, a male progeny animal formed according to the method of claim 1. In an embodiment, the male progeny animal is of a generation selected from the group consisting of a F1 generation, a F2 generation, a F3 generation, and a F4 generation.

In an illustrative aspect, a method for preparing a DNA fraction from an animal useful for analyzing histone retention involved in an epigenetic alteration. The method comprises (a) extracting DNA from a germline sample of an animal, (b) producing a fraction of the DNA extracted in (a) by selecting DNA comprising histone retention, and (c) analyzing the histone retention in the fraction of DNA produced in (b).

In an embodiment, the germline sample is sperm. In an embodiment, the sperm comprises purified sperm. In an embodiment, the sperm comprises sonicated sperm.

In an embodiment, analyzing the histone retention comprises histone antibody chromatin immunoprecipitation (ChIP) analysis. Briefly, ChIP analysis is well known to the skilled artisan as a means to analyze histone retention. In an embodiment, analyzing the histone retention comprises PCR. In an embodiment, analyzing the histone retention comprises ChIP and PCR

In an embodiment, the animal is a male progeny animal born from a female parental animal, wherein the female parental animal is administered one or more toxicants. In an embodiment, the epigenetic alteration is associated with in reduced fertility of the male progeny animal or sterility of the male progeny animal. In an embodiment, the reduced fertility or sterility of the male progeny animal is associated with abnormal testicular development, abnormal spermatogenesis, decreased sperm motility, decreased forward sperm movement, or any combination thereof.

In an embodiment, the histone retention comprises an altered histone retention DNA profile associated with a disease. In an embodiment, the disease is selected from the group consisting of kidney disease, prostate disease, male infertility, immune cell activation, cancer, and any combination thereof. In an embodiment, the method further comprises identification of one or more genes associated with the altered histone retention DNA profile. In an embodiment, the histone retention analysis is combined with a differential DNA methylation region analysis.

In an embodiment, the animal is a human. In an embodiment, the animal is a companion animal. In an embodiment, the companion animal is a dog or a cat.

In an illustrative aspect, a method of identifying a disease or a disease propensity in a male progeny animal is provided. The method comprises identifying a profile of histone retention DNA in the male progeny animal. In an embodiment, the profile is associated with the disease or the disease propensity in the male progeny animal, and In an embodiment, the disease or the disease propensity is associated with an epigenetic alteration in germline DNA resulting from contact of the germline DNA of the male progeny animal with one or more toxicants during gestation of the male progeny animal.

In an embodiment, the male progeny animal is administered a pharmaceutical composition to treat the identified disease or the disease propensity. In an embodiment, the disease is selected from the group consisting of kidney disease, prostate disease, male infertility, immune cell activation, cancer, and any combination thereof.

In an embodiment, the profile of histone retention DNA is identified according to a method for preparing a DNA fraction from the male progeny animal comprising (a) extracting DNA from a germline sample of the male progeny animal, (b) producing a fraction of the DNA extracted in (a) by selecting DNA comprising histone retention, and (c) analyzing the histone retention in the fraction of DNA produced in (b).

In an embodiment, the histone retention analysis is combined with a differential DNA methylation region analysis. In an embodiment, the animal is a human. In an embodiment, the animal is a companion animal. In an embodiment, the companion animal is a dog or a cat.

The following numbered embodiments are contemplated and are non-limiting:
1. A method of inducing an epigenetic alteration in germline DNA of a male progeny animal, comprising: administering one or more toxicants to a female parental animal wherein the female parental animal subsequently gives birth to the male progeny animal, and wherein the male progeny animal comprises the epigenetic alteration in germline DNA.
2. The method of clause 1, any other suitable clause, or any combination of suitable clauses, wherein the epigenetic alteration is differential histone retention of DNA in the germline DNA of the male progeny animal.
3. The method of clause 1, any other suitable clause, or any combination of suitable clauses, wherein the epigenetic alteration in the germline DNA of the male progeny animal is associated with a disease in the male progeny animal.
4. The method of clause 4, any other suitable clause, or any combination of suitable clauses, wherein the disease is selected from the group consisting of kidney disease, prostate disease, male infertility, immune cell activation, cancer, and any combination thereof.
5. The method of clause 1, any other suitable clause, or any combination of suitable clauses, wherein the epigenetic alteration in germline DNA is associated with in reduced fertility of the male progeny animal or sterility of the male progeny animal.
6. The method of clause 5, any other suitable clause, or any combination of suitable clauses, wherein the reduced fertility or sterility of the male progeny animal is associated with abnormal testicular development, abnormal spermatogenesis, decreased sperm motility, decreased forward sperm movement, or any combination thereof.
7. The method of clause 1, any other suitable clause, or any combination of suitable clauses, wherein the animal is a human.
8. The method of clause 1, any other suitable clause, or any combination of suitable clauses, wherein the animal is a companion animal.
9. The method of clause 8, any other suitable clause, or any combination of suitable clauses, wherein the companion animal is a dog or a cat.
10. A male progeny animal formed according to the method of claim 1.
11. The male progeny animal of clause 10, any other suitable clause, or any combination of suitable clauses, wherein the male progeny animal is of a generation selected from the group consisting of a F1 generation, a F2 generation, a F3 generation, and a F4 generation.
12. A method for preparing a DNA fraction from an animal useful for analyzing histone retention involved in an epigenetic alteration, comprising
   (a) extracting DNA from a germline sample of an animal,
   (b) producing a fraction of the DNA extracted in (a) by selecting DNA comprising histone retention, and
   (c) analyzing the histone retention in the fraction of DNA produced in (b).
13. The method of clause 12, any other suitable clause, or any combination of suitable clauses, wherein the germline sample is sperm.
14. The method of clause 13, any other suitable clause, or any combination of suitable clauses, wherein the sperm comprises purified sperm.
15. The method of clause 13, any other suitable clause, or any combination of suitable clauses, wherein the sperm comprises sonicated sperm.
16. The method of clause 12, any other suitable clause, or any combination of suitable clauses, wherein analyzing the histone retention comprises histone antibody chromatin immunoprecipitation (ChIP) analysis.
17. The method of clause 12, any other suitable clause, or any combination of suitable clauses, wherein analyzing the histone retention comprises PCR.
18. The method of clause 12, any other suitable clause, or any combination of suitable clauses, wherein analyzing the histone retention comprises ChIP and PCR.
19. The method of clause 12, any other suitable clause, or any combination of suitable clauses, wherein the animal is a male progeny animal born from a female parental animal, wherein the female parental animal is administered one or more toxicants.
20. The method of clause 19, any other suitable clause, or any combination of suitable clauses, wherein the epigenetic alteration is associated with in reduced fertility of the male progeny animal or sterility of the male progeny animal.
21. The method of clause 20, any other suitable clause, or any combination of suitable clauses, wherein the reduced fertility or sterility of the male progeny animal is associated with abnormal testicular development, abnormal spermatogenesis, decreased sperm motility, decreased forward sperm movement, or any combination thereof.
22. The method of clause 12, any other suitable clause, or any combination of suitable clauses, wherein the histone retention comprises an altered histone retention DNA profile associated with a disease.
23. The method of clause 22, any other suitable clause, or any combination of suitable clauses, wherein the disease is selected from the group consisting of kidney disease, prostate disease, male infertility, immune cell activation, cancer, and any combination thereof.
24. The method of clause 22, any other suitable clause, or any combination of suitable clauses, wherein the method further comprises identification of one or more genes associated with the altered histone retention DNA profile.
25. The method of clause 12, any other suitable clause, or any combination of suitable clauses, wherein the histone retention analysis is combined with a differential DNA methylation region analysis.
26. The method of clause 12, any other suitable clause, or any combination of suitable clauses, wherein the animal is a human.
27. The method of clause 12, any other suitable clause, or any combination of suitable clauses, wherein the animal is a companion animal.
28. The method of clause 27, any other suitable clause, or any combination of suitable clauses, wherein the companion animal is a dog or a cat.
29. A method of identifying a disease or a disease propensity in a male progeny animal, comprising: identifying a profile of histone retention DNA in the male progeny animal, wherein the profile is associated with the disease or the disease propensity in the male progeny animal, and wherein the disease or the disease propensity is associated with an epigenetic alteration in germline DNA resulting from contact of the germline DNA of the male progeny animal with one or more toxicants during gestation of the male progeny animal.
30. The method of clause 29, any other suitable clause, or any combination of suitable clauses, wherein the male progeny animal is administered a pharmaceutical composition to treat the identified disease or the disease propensity.
31. The method of clause 29, any other suitable clause, or any combination of suitable clauses, wherein the disease is selected from the group consisting of kidney disease, prostate disease, male infertility, immune cell activation, cancer, and any combination thereof.
32. The method of clause 29, any other suitable clause, or any combination of suitable clauses, wherein the profile of histone retention DNA is identified according to a method for preparing a DNA fraction from the male progeny animal comprising
   (a) extracting DNA from a germline sample of the male progeny animal,
   (b) producing a fraction of the DNA extracted in (a) by selecting DNA comprising histone retention, and
   (c) analyzing the histone retention in the fraction of DNA produced in (b).
33. The method of clause 29, any other suitable clause, or any combination of suitable clauses, wherein the histone retention analysis is combined with a differential DNA methylation region analysis.
34. The method of clause 29, any other suitable clause, or any combination of suitable clauses, wherein the animal is a human.
35. The method of clause 29, any other suitable clause, or any combination of suitable clauses, wherein the animal is a companion animal.
36. The method of clause 35, any other suitable clause, or any combination of suitable clauses, wherein the companion animal is a dog or a cat.

### EXAMPLES

### Example 1

Evaluation of Epigenetic Alterations Induced by Glyphosate

In the instant example, glyphosate is utilized as an exemplary toxicant. Rats were used as the exemplary animal.

### Animal Studies and Breeding

Outbred Sprague Dawley SD male and female rats were fed a standard diet with water ad lib and mated. Gestating female rats were exposed to glyphosate and offspring bred for three generations in the absence of exposure. Female and male rats of an outbred strain Hsd:Sprague Dawley SD (Harlan) at 70 to 100 days of age were fed ad lib with a standard rat diet and ad lib tap water. Timed-pregnant females on days 8 through 14 of gestation were administered daily intraperitoneal injections of glyphosate (25 mg/kg BW/day dissolved in PBS) (Chem Service, Westchester Pa.) or dimethyl sulfoxide (DMSO) or Phosphate Buffered Saline (PBS).

The instant example is designed to examine glyphosate induced transgenerational disease. To control dose and not cause stress from gavage oral administration, an IP exposure was utilized. Twenty-five mg/kg for glyphosate is 0.4% of rat oral LD50 and 50% of the NOAEL and considering glyphosate rapid metabolism approximately twice the occupational exposure 3-5 mg/kg per daily exposure A portion of original control colony was excluded from the study due to the obesity founder effects as previously identified. These animals were replaced with offspring from DMSO-treated controls from a concurrent study. Disease phenotypes were compared from both DMSO lineage and PBS lineage controls, with no significant differences observed with histopathology evaluations between the two populations.

The gestating female rats treated were designated as the F0 generation. Breeding for each generation occurred at 70-90 days and 5 different litters were generated with no cousin or sibling breeding to avoid any inbreeding artifacts. Litters were culled to 10 pups (5 males and 5 females) between days 3-5 of birth to avoid litter effects. F1-F3 generation control and glyphosate lineages were housed and aged in the same room and racks with lighting, food and water. The F3 generation males were aged to 1 year of age for pathology analysis.

The F3 generation male rats were euthanized at 12 months of age by CO₂ inhalation and cervical dislocation for tissue harvest. Testis, prostate, ovary, kidney, and gonadal fat pads were fixed in Bouin's solution (Sigma) for less than 6 hours, followed by 70% ethanol, then processed for paraffin embedding and hematoxylin, and eosin (H & E) staining by standard procedures for histopathological examination. Paraffin five micron sections were processed and stained (Nationwide Histology, Spokane Wash., USA).

### Histopathology Examination and Disease Classification

Full necropsies as required on animals that died prior to the time of scheduled sacrifice at one year were undertaken and tumor classifications were also performed. Upon dissection, a brief examination of abdominal and thoracic organs was performed to look for obvious abnormalities. The current study found no significant gross pathology of heart, lung, liver, gastro-intestinal track, or spleen. The tissues evaluated histologically were selected from previous literature showing them to have pathology in transgenerational models, with an emphasis on reproductive organs. Histopathology readers were trained to recognize the specific abnormalities evaluated for this study in rat testis, ventral prostate, ovary and kidney. Three different pathology readers were used for each tissue that were blinded to the treatment groups. A set of quality control (QC) slides was generated for each tissue and was read by each reader prior to evaluating any set of experimental slides. These QC slide results are monitored for reader accuracy and concordance.

Testis histopathology criteria included the presence of vacuoles in the seminiferous tubules, azoospermic atretic seminiferous tubules, and ‘other’ abnormalities including sloughed spermatogenic cells in center of the tubule and a lack of a tubule lumen. Prostate histopathology criteria included the presence of vacuoles in the glandular epithelium, atrophic glandular epithelium and hyperplasia of prostatic gland epithelium. Kidney histopathology criteria included reduced size of glomerulus, thickened Bowman's capsule, and the presence of proteinaceous fluid-filled cysts >50 μm in diameter. A cut-off was established to declare a tissue ‘diseased’ based on the mean number of histopathological abnormalities plus two standard deviations from the mean of control group tissues, as assessed by each of the three individual observers blinded to the treatment groups. This number (i.e. greater than two standard deviations) was used to classify rats into those with and without testis, prostate, or kidney disease in each lineage. A rat tissue section was finally declared ‘diseased’ only when at least two of the three observers marked the same tissue section ‘diseased’.

Obesity was assessed with an increase in adipocyte size (area), body mass index (BMI) and abdominal adiposity. BMI was calculated with weight (g)/length (cm)² with the length of the animal measured from the nose to the base of the tail. Gonadal fat pad slides were imaged using a Nikon Eclipse E800 microscope (10×) with an AVT Prosilica GE1050C Color GigE camera. Five field of view image captures were taken per slide in varying parts of the fat pad. Adipocyte size was measured converting pixels into microns using Adiposoft. Measurements of the 20 largest cells from each image for a total of 100 were averaged as hypertrophic cells are the most metabolically relevant and susceptible to cell death. Obesity and lean phenotypes were determined utilizing the mean of the control population males and females, and a cut off of 1.5 standard deviations above and below the mean.

### Epididymal Sperm Collection and DNA Isolation

Briefly, the epididymis was dissected free of fat and connective tissue, then, after cutting open the cauda, placed into 6 ml of phosphate buffer saline (PBS) for 20 minutes at room temperature. Further incubation at 4° C. will immobilize the sperm. The tissue was then minced, the released sperm pelleted at 4° C. 3,000×g for 10 min, then resuspended in NIM buffer and stored at −80° C. for further processing.

An appropriate amount of rat sperm suspension was used for DNA extraction. Previous studies have shown mammalian sperm heads are resistant to sonication unlike somatic cells. Somatic cells and debris were therefore removed by brief sonication (Fisher Sonic Dismembrator, model 300, power 25), then centrifugation and washing 1-2 times in 1×PBS. The resulting pellet was resuspended in 820 μL DNA extraction buffer and 80 μl 0.1M DTT added, then incubated at 65° C. for 15 minutes. Then, 80 μl proteinase K (20 mg/ml) was added and the sample was incubated at 55° C. for 2-4 hours under constant rotation. Protein was removed by addition of protein precipitation solution (300 μl, Promega A795A), incubation for 15 min on ice, then centrifugation at 13,500 rpm for 30 minutes at 4° C. One ml of the supernatant was precipitated with 2 μl of glycoblue (Invitrogen, AM9516) and 1 ml of cold 100% isopropanol. After incubation, the sample was spun at 13,500×g for 30 min at 4° C., then washed with 70% cold ethanol. The pellet was air-dried for about 5 minutes then resuspended in 100 μl of nuclease free water. For all generations, equal amounts of DNA from each individual's sample was used for methylated DNA immunoprecipitation (MeDIP) and chromatin immunoprecipitation (ChIP).

### Methylated DNA Immunoprecipitation (MeDIP)

Genomic DNA was sonicated and run on 1.5% agarose gel for fragment size verification. The sonicated DNA was then diluted with TE buffer to 400 μl, then heat-denatured for 10 minutes at 95° C., and immediately cooled on ice for 10 min to create single-stranded DNA fragments. Then 100 μl of 5×IP buffer and 5 μg of antibody (monoclonal mouse anti 5-methyl cytidine; Diagenode #C15200006) were added, and the mixture was incubated overnight on a rotator at 4° C. The following day magnetic beads (Dynabeads M-280 Sheep anti-Mouse IgG; Life Technologies 11201D) were pre-washed per manufacturer's instructions, and 50 μl of beads were added to the 500 μl of DNA-antibody mixture from the overnight incubation, then incubated for 2 h on a rotator at 4° C. After this incubation, the samples were washed three times with 1×IP buffer using a magnetic rack. The washed samples were then resuspended in 250 μl digestion buffer (5 mM Tris PH 8, 10. mM EDTA, 0.5% SDS) with 3.5 μl Proteinase K (20 mg/ml), and incubated for 2-3 hours on a rotator at 55° C. DNA clean-up was performed using a Phenol-Chloroform-Isoamyalcohol extraction, and the supernatant precipitated with 2 μl of glycoblue (20 mg/ml), 20 μl of 5M NaCl and 500 μl ethanol in −20° C. freezer for one to several hours. The DNA precipitate was pelleted, washed with 70% ethanol, then dried and resuspended in 20 μl H₂O or TE. DNA concentration was measured in Qubit (Life Technologies) with the ssDNA kit (Molecular Probes Q10212).

### DHR H3 Histone Chromatin Immunoprecipitation (ChIP)

Histone chromatin immunoprecipitation with genomic DNA was performed. Individual rat sperm were collected, and the sperm counts were determined for each individual. A sonication of 10 seconds was performed for each sperm sample to remove any somatic cell contamination using a Sonic Dismembrator Model 300 (Thermo Scientific Fisher, USA) then centrifuged 1800×g for 5 minutes at 4° C. then resuspended and counted individually on a Neubauer counting chamber (Propper manufacturing Co., Inc., New York, USA). The sperm samples were reconstituted up to 1 ml with PBS (phosphate buffered saline). To reduce disulfide bonds, 50 μl of 1 M DTT was added to each sample and an incubation of 2 hours at room temperature under constant rotation followed. To quench any residual DTT (dithiothreitol, Fisher Scientific, NY USA) in the reaction, 120 μl of fresh 1 M NEM (N-Ethylmaleimide, Thermo Scientific, Rockford, USA) was then added and the samples were incubated for 30 min at room temperature under constant rotation. After 5 minutes of centrifugation at 450×g at room temperature, the sperm samples were pelleted and the supernatant discarded. Pellets were resuspended in PBS and then spun again at 450×g for 5 min at room temperature. The supernatant was discarded, and the pellets were resuspended in 130 μl of complete buffer supplemented with tergitol 0.5% and DOC 1%. The samples were then sonicated using the Covaris M220. Each tube in the experiment went through the “Chromatin shearing” program for 10 minutes.

After the Covaris sonication, the fragmentation was checked by running 10 μl of each sample on a 1.5% agarose gel. Samples were then centrifuged at 12,500×g for 10 min at room temperature. The supernatant was transferred to a fresh microfuge tube and 65 μl of protease inhibitor cocktail (1 tablet dissolved in 500 μl, 20× concentrated) (Roche, cat. no. 11 873 580 001) were added in each sample as well as 3 μl of antibody (anti-histone H3 pan-monoclonal antibody, cat no. 05-928, with broad spectrum species specifically form Millipore Corp, Temecula Calif. USA). The DNA-antibody mixture was incubated overnight on a rotator at 4° C. The following day, magnetic beads (ChIP-Grade protein G magnetic beads, Cell Signaling 9006) were pre-washed as follows: the beads were resuspended in the vial, then 30 μl per sample was transferred to a microfuge tube. The same volume of Washing Buffer (at least 1 ml) was added and the bead sample was resuspended. The tube was then placed into a magnetic rack for 1-2 min and the supernatant was discarded. The tube was removed from the magnetic rack and the beads were washed once. The washed beads were resuspended in the same volume of 1×IP buffer as the initial volume of beads. 30 μl of beads were added to each DNA-antibody mixture from the overnight incubation, then incubated for 2 h on a rotator at 4° C. After the incubation, the bead-antibody-DNA complex was washed three times with 1×IP buffer as follows: the tube was placed into a magnetic rack for 1-2 min and the supernatant was discarded, then washed with 1×IP buffer 3 times. The washed bead-antibody-DNA solution was then resuspended in 300 μl of digestion buffer (1 M Tris HCl, pH 8.0, 0.5 M EDTA, 10% SDS) and 3 μl proteinase K (20 mg/ml). The sample was incubated for 3 h on a rotator at 56° C. After incubation the samples were extracted with Phenol-Chloroform-Isoamylalcohol and precipitated with 2 μl of Glycoblue (20 mg/ml), a one-tenth volume of 3 M sodium acetate and two volumes of ethanol overnight at −20° C. After a centrifugation at 18,000×g for 30 min at 4° C., the supernatant was removed without disturbing the pellet. The pellet was washed with 500 μl cold 70% ethanol, then centrifuged again at 18,000×g for 10 min at 4° C. and the supernatant was discarded. The tube was spun briefly to collect residual ethanol to bottom of tube and as much liquid as possible was removed with a gel loading tip. Pellet was air-dried at room temperature until it looked dry (about 5 min), then resuspended in 20 μl H₂O. DNA concentration was measured in the Qubit (Life Technologies) with the BR dsDNA kit (Molecular Probes Q32853).

### MeDIP-Seq and H3 Histone ChIP-Seq Analysis

MeDIP DNA and H3 ChIP DNA was used to create libraries for next generation sequencing (NGS) using the NEBNext Ultra RNA Library Prep Kit for Illumina (San Diego, Calif.) starting at step 1.4 of the manufacturer's protocol to generate double stranded DNA from the single-stranded DNA resulting from MeDIP. After this step, the manufacturer's protocol was followed indexing each sample individually with NEBNext Multiplex Oligos for Illumina. The library quality control used an Agilent 2100 Bio analyzer analysis for size, amount and quality. The samples were sequenced on the Illumina HiSeq 2500 at PESO, with a read size of approximately 50 bp and approximately 20 million reads per pool. Twelve libraries were run in one lane.

### Statistics and Bioinformatics

Sequencing data quality was assessed using the FastQC program. Sequencing reads were cleaned and filtered to remove adapters and low quality bases using Trimmomatic. The basic read quality was verified using summaries produced by the FastQC program. The reads for each MeDIP and H3 histone ChIP samples were mapped to the Rnor 6.0 rat genome using Bowtie2 with default parameter options. The mapped read files were then converted to sorted BAM files using SAMtools. The MEDIPS R package was used to calculate differential coverage between control and exposure sample groups. The edgeR p-value was used to determine the relative difference between the two groups for each genomic window. Windows with an edgeR p-value less than the selected stringent threshold were considered DMR or DHR sites. The site edges were extended until no genomic window with an edgeR p-value less than 0.1 remained within 1000 bp of the DMR or DHR. The edgeR p-value is optimal for individual epimutation identification and used to assess the significance of the DMR or DHR identified. A false discovery rate (FDR) analysis for the exposure versus control was performed and provided value less than 0.05. Due to the lower number of individuals with one specific disease type, an FDR analysis was generally not useful, nor permutation analysis for the specific disease biomarkers.

Differential epimutation sites were annotated using the biomaRt R package to access the Ensembl database. The DMR associated genes were then automatically sorted into functional groups using information provided by the publicly available DAVID and Panther databases incorporated with an internal curated database (www.skinner.wsu.edu under genomic data). The DAVID and Panther databases have a greater accuracy for experimental gene functional links than literature based searches such as GO categories. A Pathway Studio, Elsevier, database and network tool was used to assess physiological and disease process gene correlations (Pathway Studio software (Elsevier, Inc. 2020), Pathway Studio 12.3.0.16). All molecular data has been deposited into the public database at NCBI (GEO #GSE118557 and GSE152678) and R code computational tools.

### Animal Breeding

Sprague Dawley gestating female rats (F0 generation) at 90 days of age were exposed in order to study the transgenerational effects of glyphosate. The pregnant rats were transiently exposed (25 mg/kg body weight glyphosate daily) between days 8-14 of gestation during fetal gonadal sex determination when germ cell epigenetic programming occurs. Twenty-five mg/kg/day is half the No Observable Adverse Effect Level (NOAEL) exposure of 50 mg/kg/day. Glyphosate has a rapid metabolism turnover of 5-10 hour half-life, such that the concentration will decrease approximately 75-90% daily during the transient exposure period. The 2-4 half-lives that occur each 24-hour period indicates that after 7 days of exposure less than 50 mg/kg/daily, the exposures NOAEL, would be present. The offspring F1 generation rats (directly exposed in utero) were aged to 90 days of age and bred within the lineage to generate the grand-offspring F2 generation (directly exposed through the F1 generation germline), which were then bred at 90 days of age to generate the F3 generation (not directly exposed so transgenerational). The control lineage used F0 gestating rats exposed to the vehicle control dimethyl sulfoxide (DMSO) or phosphate buffered saline (PBS). All the lineages were aged to 1 year and euthanized for pathology and sperm epigenetic analysis. At each generation five litters were obtained with no sibling or cousin breeding to prevent any inbreeding artifacts in the control or glyphosate lineages. Due to the lack of any inbreeding, the potential frequency of genetic segregation is minimal in the F3 generation and was not observed with sibling comparisons. As previously described and to prevent litter bias, at each generation between 6 and 8 unrelated founder gestating females from different litters were bred, and 5 litters were obtained and culled early in postnatal development to 10 pups per litter with animals of each sex from each litter used to generate 25-50 individuals of each sex for each generation for analysis. Similar numbers of males and females were used from each litter to avoid any litter bias.

### Pathology Analysis

Pathology calls were made by assessing histology sections of testis, kidney, prostate, and gonadal adipocytes. For the testis pathology, the abnormalities quantified were atrophy of seminiferous tubules, vacuoles within seminiferous tubules, and sloughing of cellular debris into the tubular lumen (maturation arrest). For prostate pathology, the abnormalities quantified were atrophy of prostatic epithelium, vacuoles within the prostatic epithelium, and prostatic epithelial hyperplasia. For kidney pathology, the abnormalities quantified were reduced glomerular size, thickening of the Bowman's capsule, and renal cysts. The age of puberty onset was determined. Obesity was assessed with an increase in adipocyte size (area), body mass index (BMI) and abdominal adiposity. In all cases the number of abnormalities in an animal's tissue was compared with the mean number of abnormalities in the control group to determine if that tissue was diseased. For the F3 generation glyphosate lineage male pathology, the individual animals are listed and a (+) indicates presence of disease and (−) absence of disease (Table 1). Table 1 shows the F3 generation glyphosate lineage male pathology. The individual animals for the glyphosate lineage males are listed and a (+) indicates presence of disease and (−) absence of disease. The animals with shaded (+) were used for the epigenetic analysis due to the presence of only one disease, except the multiple (≥2) disease.

The F3 generation control lineage male pathology, the individuals are listed similarly in Table 2. As shown in Table 2, individual animals for the control lineage males are listed and a (+) indicates presence of disease and (−) absence of disease.

TABLE 2
F3 Generation Control Males Pathology

| | | | | | | | | Multiple | Total |
|---|---|---|---|---|---|---|---|---|---|
| ID | Puberty | Testis | Prostate | Kidney | Tumor | Lean | Obese | Disease | Disease |
| Gly-60 | — | — | + | — | — | — | — | — | 1 |
| Gly-57 | + | — | — | — | + | + | — | + | 3 |
| Gly-58 | + | — | + | — | — | — | — | + | 2 |
| Gly-59 | — | — | — | — | — | — | — | — | 0 |
| Gly-46 | — | — | — | — | — | — | — | — | 0 |
| Gly-47 | — | — | — | + | — | + | — | + | 2 |
| Gly-48 | — | + | — | — | — | — | + | + | 2 |
| Gly-49 | — | — | + | — | — | — | — | — | 1 |
| Gly-50 | — | — | — | — | — | — | — | — | 0 |
| Gly-51 | — | + | — | — | — | — | + | + | 2 |
| Gly-52 | — | — | — | — | — | — | — | — | 0 |
| Gly-53 | — | — | — | + | — | — | + | + | 2 |
| Gly-54 | — | — | — | — | — | — | — | — | 0 |
| Gly-55 | | — | — | + | — | — | + | + | 2 |
| Gly-56 | — | — | — | — | — | — | + | — | 1 |
| Gly-61 | — | — | — | — | — | — | — | . | 0 |
| Gly-62 | — | | | | | — | — | — | 0 |
| Gly-63 | — | — | — | — | — | — | — | — | 0 |
| Gly-64 | — | — | — | — | — | — | — | — | 0 |
| Gly-65 | — | — | — | — | — | — | — | — | 0 |
| Gly-66 | — | — | — | — | — | — | — | — | 0 |
| Gly-67 | — | — | — | + | — | — | — | — | 1 |
| Gly-68 | — | — | — | — | — | — | — | — | 0 |
| Gly-69 | — | — | — | — | — | — | — | — | 0 |
| Gly-70 | | — | — | — | — | — | — | — | 0 |
| Gly-71 | — | — | — | — | — | — | + | — | 1 |
| Gly-72 | — | — | — | — | — | — | — | — | 0 |
| Gly-73 | — | — | — | — | — | — | — | — | 0 |
| Gly-74 | — | — | — | — | — | — | — | — | 0 |
| Gly-75 | — | — | — | — | — | — | — | — | 0 |
| Gly-76 | — | — | — | — | — | — | — | — | 0 |
| Gly-77 | — | — | + | — | + | — | + | 2 | |
| Gly-78 | — | — | — | — | — | — | — | 0 | |
| Disease | 2 | 2 | 3 | 5 | 1 | 3 | 6 | 8 | |
| Population | 31 | 30 | 32 | 32 | 32 | 33 | 33 | 33 | |

Only the individuals with a single disease for a specific pathology were used for that pathology, except in the case of the multiple category disease when animals with two of more diseases were used. This allows a more accurate association with disease and eliminates the confounding presence of other disease or co-morbidities. The control lineage did not have sufficient numbers of animals with a specific disease (see Table 2), so were not analyzed further. Although the F3 generation males had a testis disease group, two individuals had only testis disease, so too few for further analysis. The other disease had n=4 individuals for kidney disease, n=7 with prostate disease, n=13 for obesity, and n=10 for multiple 2) disease, see Table 1. The disease animals were compared to animals with no disease n=8, see Table 1. The limited number of individuals needs to be considered in data interpretation and statistical analysis. In contrast to previous analyses using DDT or vinclozolin sperm biomarkers for disease, when all animals with a specific disease were analyzed independent of co-morbidities, the instant example sought to optimize disease specific biomarker assessment.

### Sperm DNA Methylation Analysis

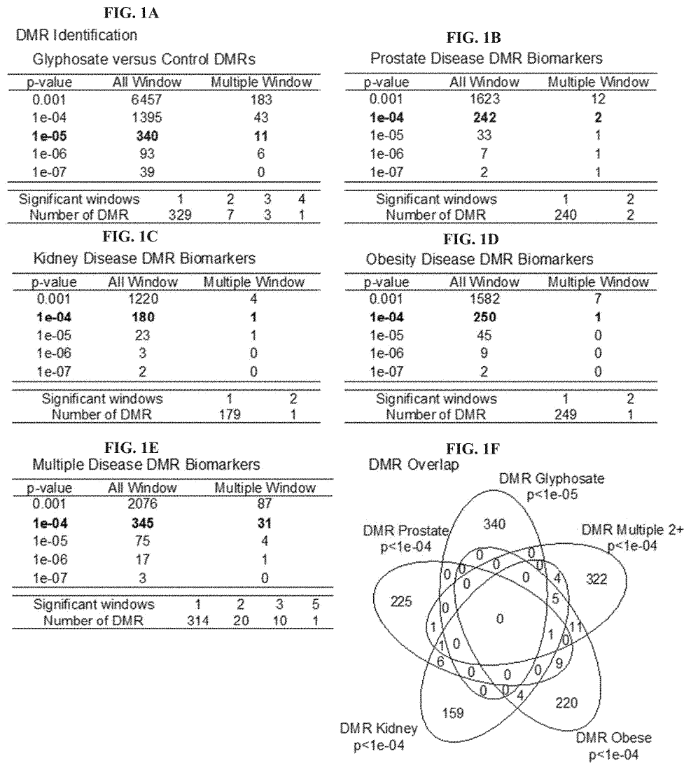

Transgenerational inheritance of pathology and disease requires the germline (sperm or egg) to transmit epigenetic information between generations. Purified sperm were collected from the control and glyphosate lineage F3 generation males for epigenetic analysis. Potential differential DNA methylation regions (DMRs) in the sperm were identified using a comparison between the control and the glyphosate lineage (FIG. 1A). Within the glyphosate lineage, individuals with a given disease (prostate, kidney, obesity, multiple disease 2)) were compared to non-diseased animals from the glyphosate lineage to define disease specific DMRs (FIG. 1B-E).

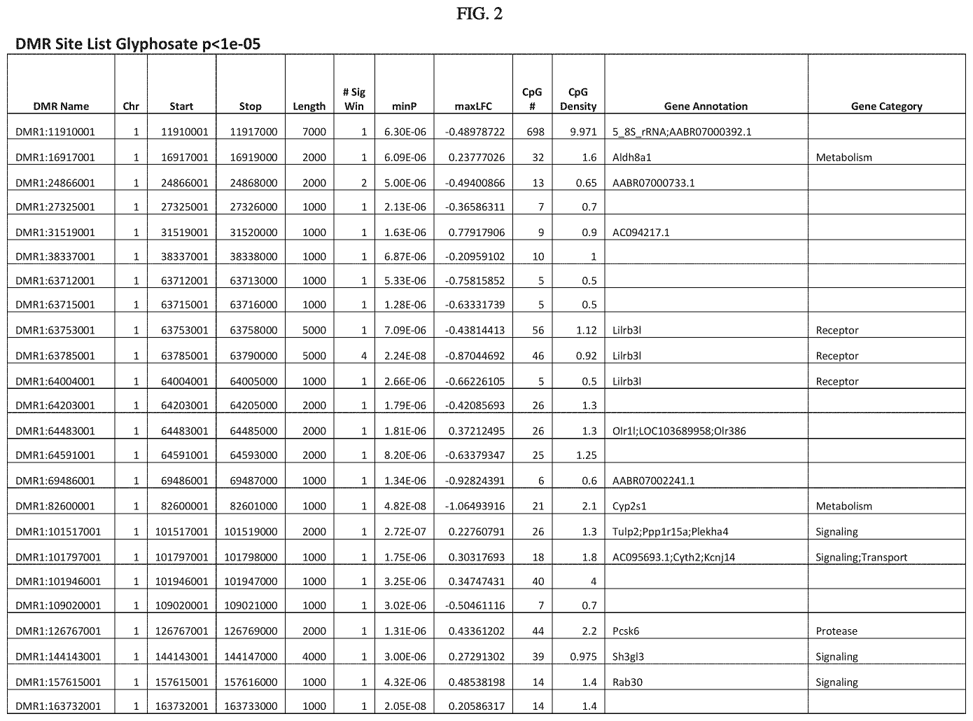

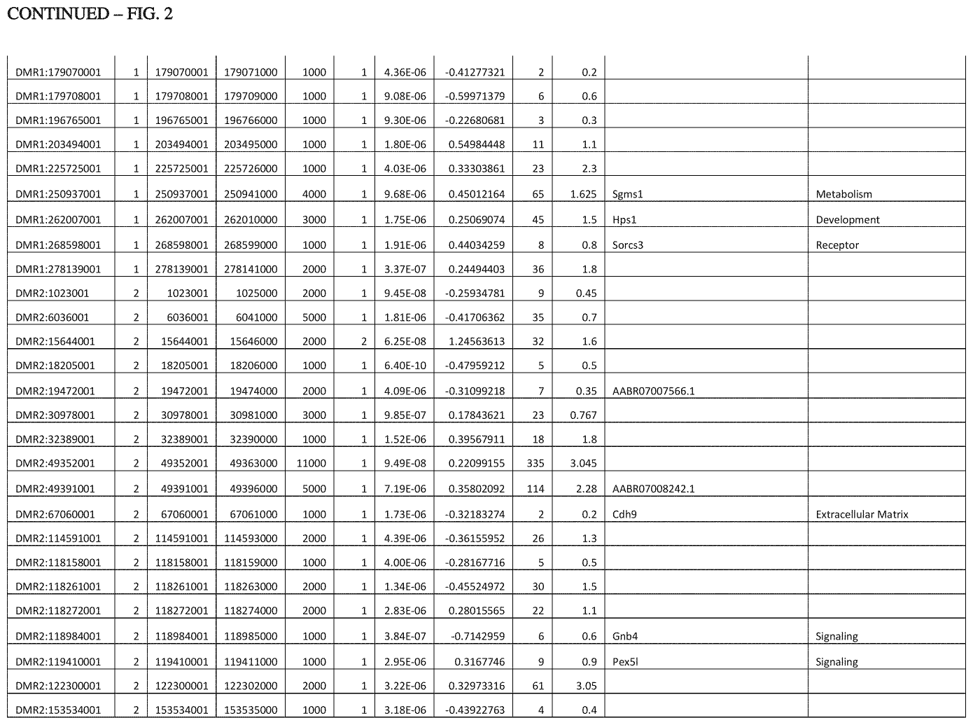

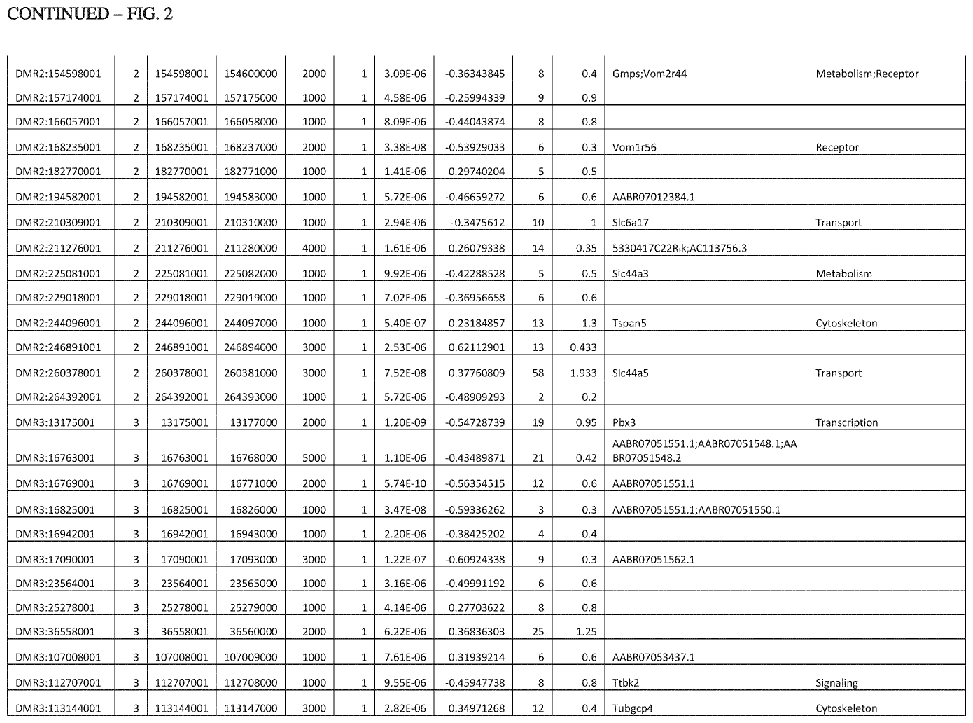

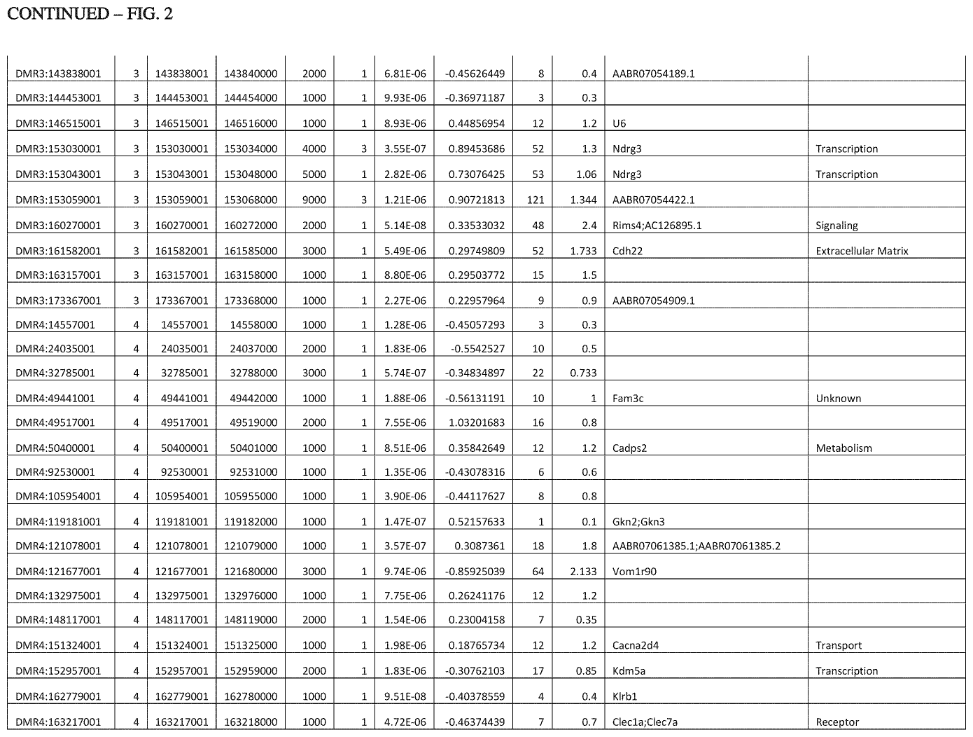

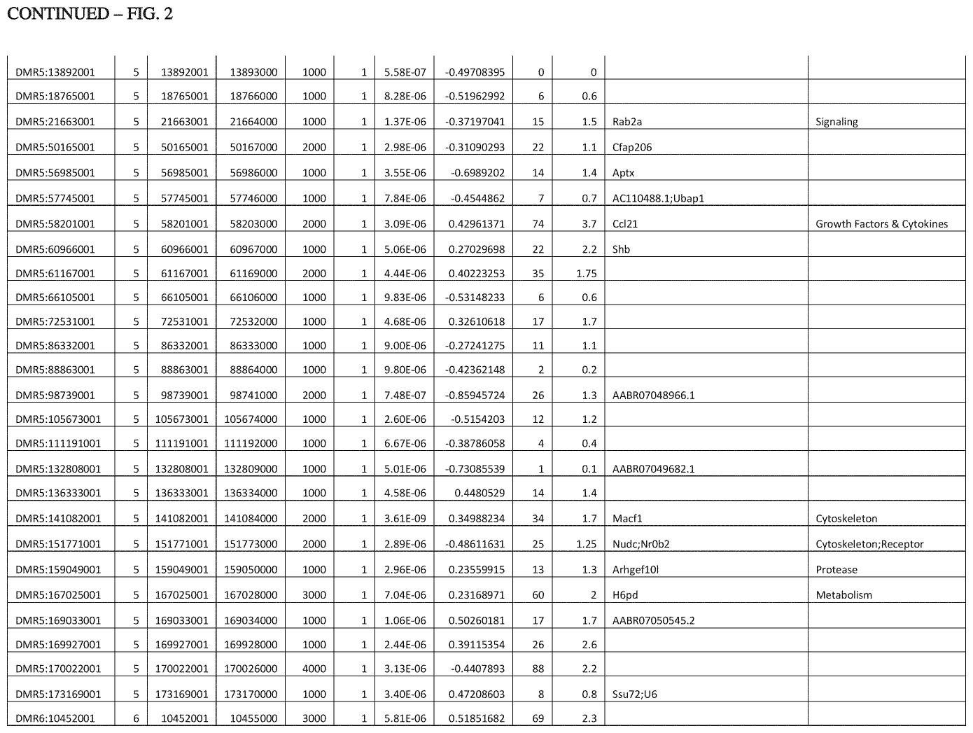

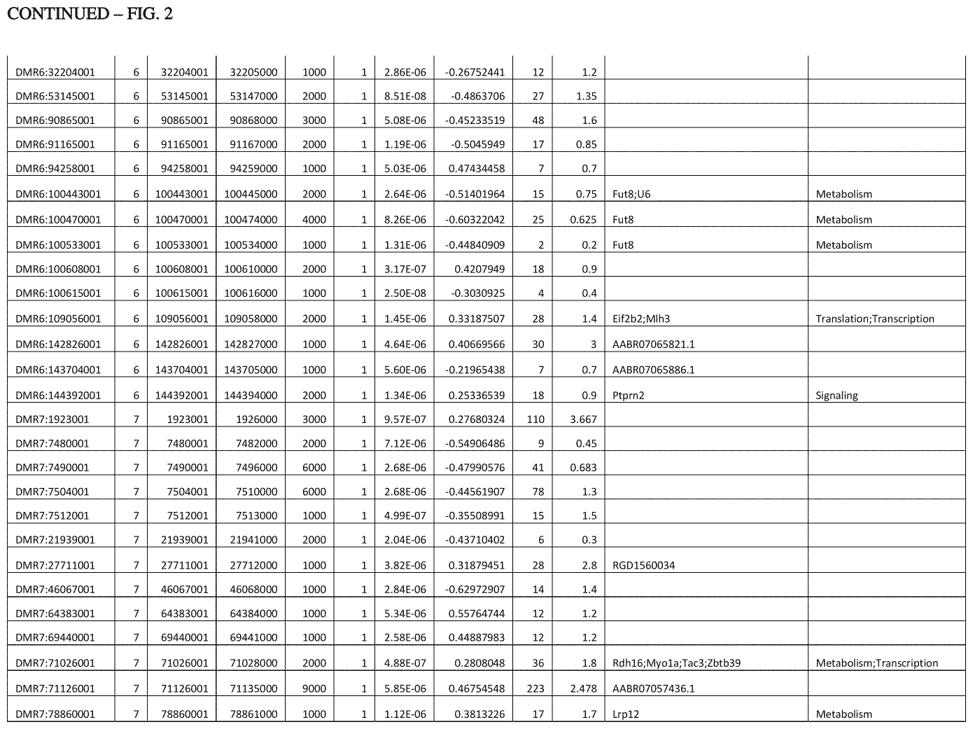

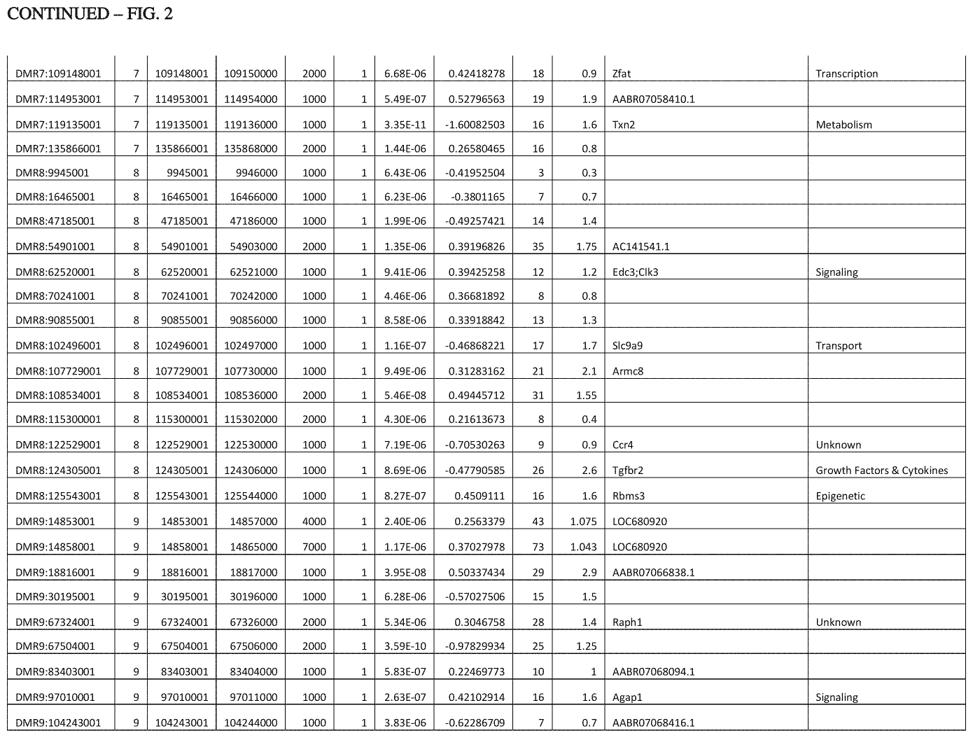

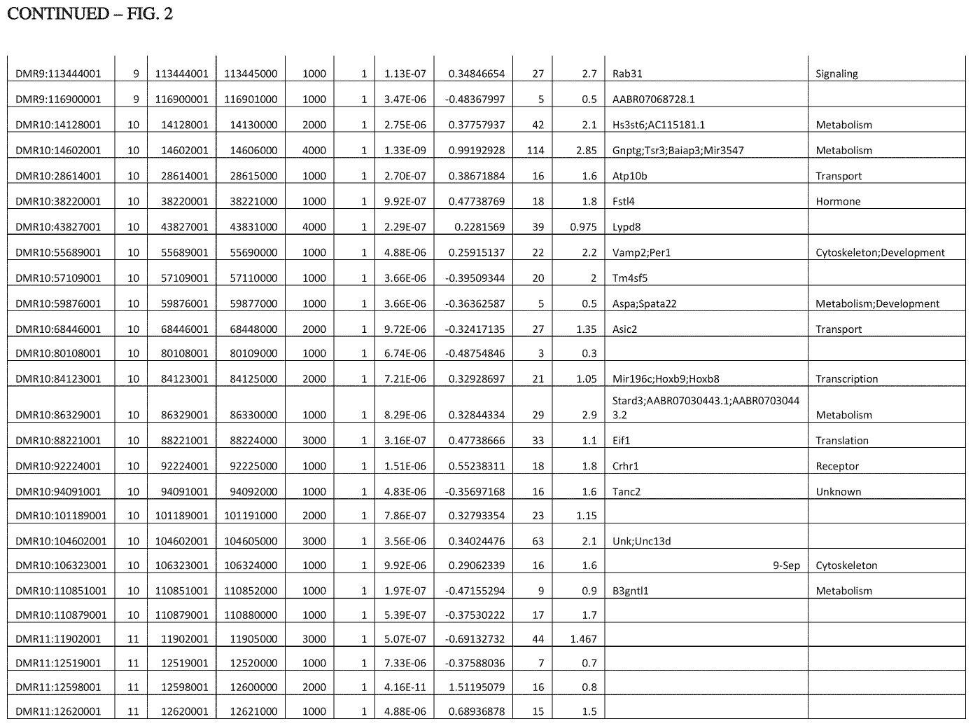

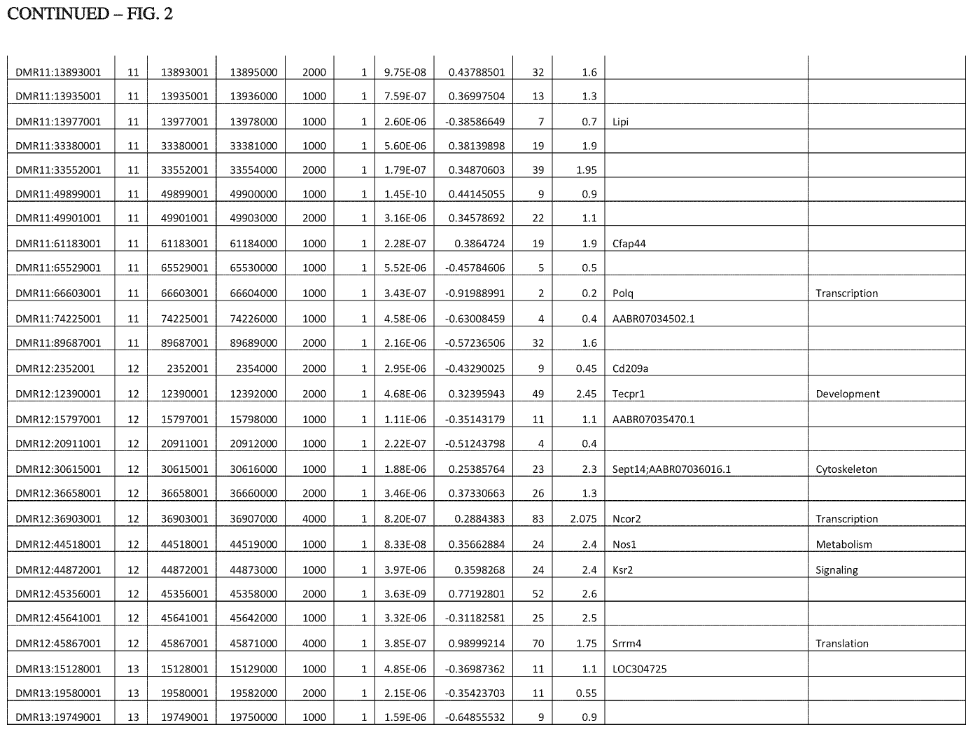

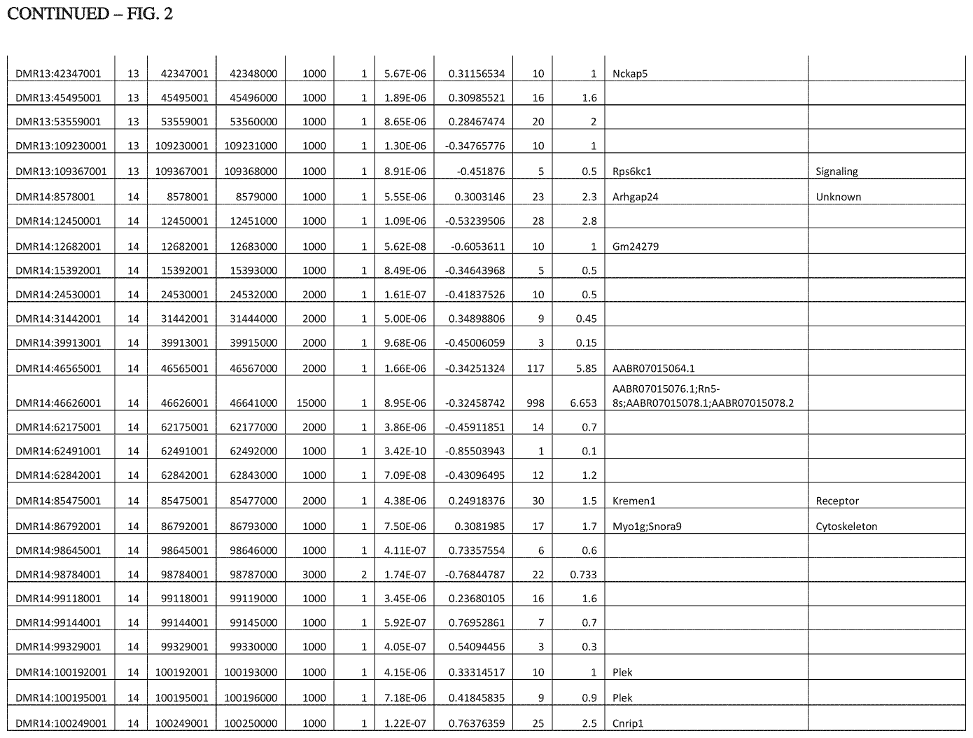

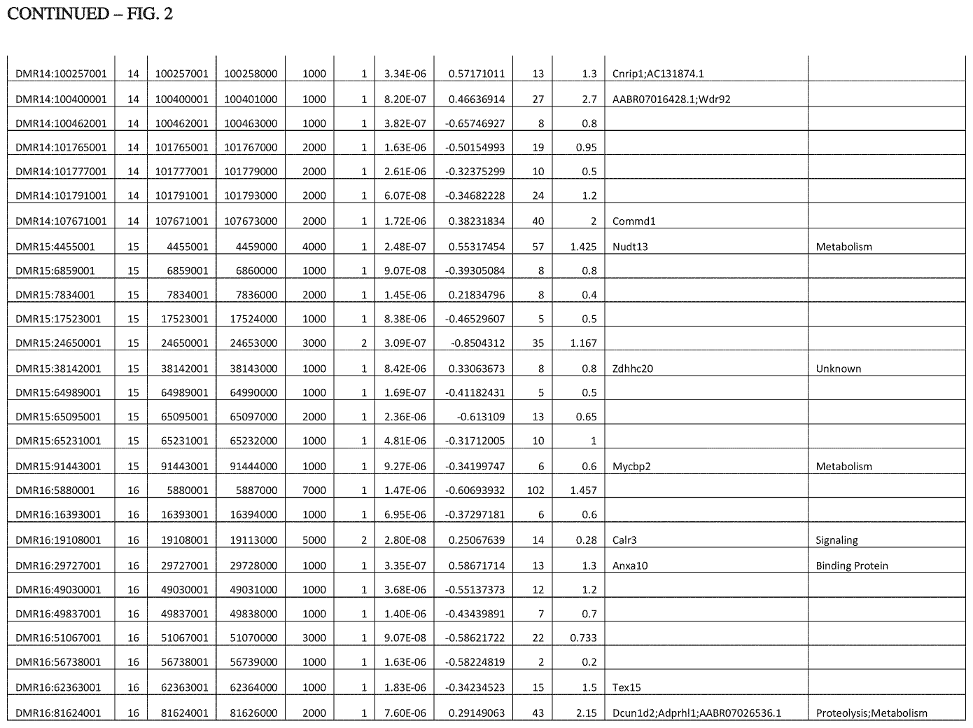

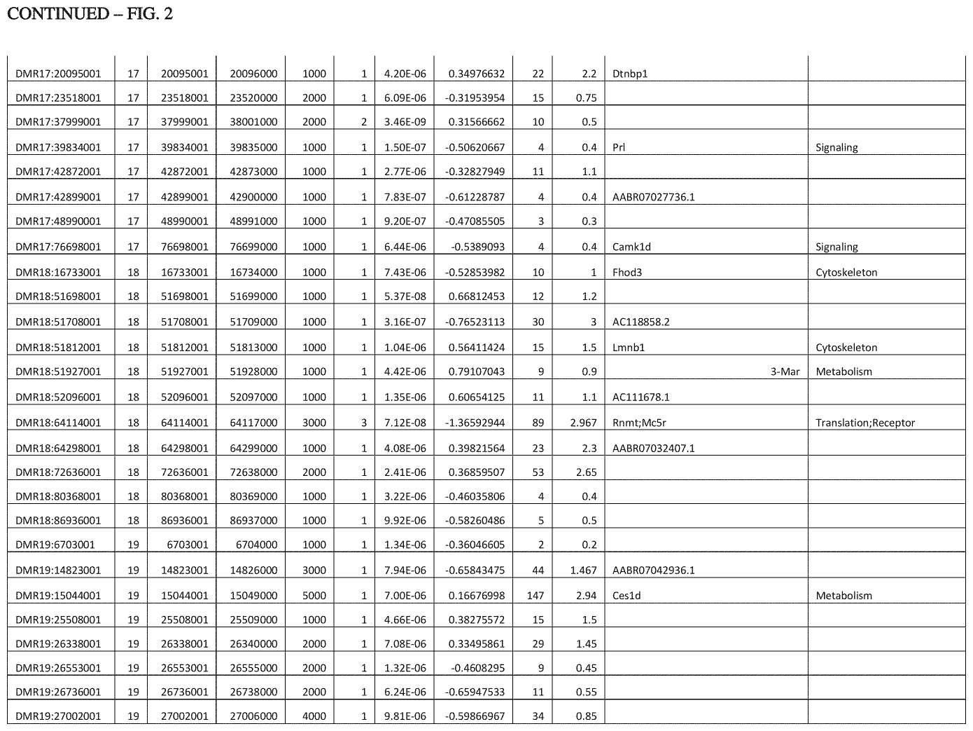

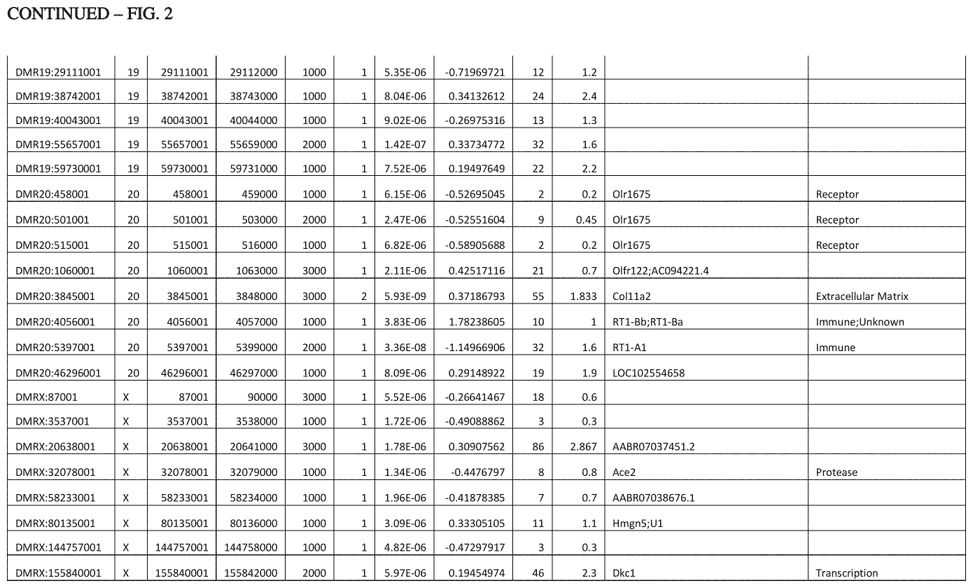

DNA from the sonicated purified sperm was isolated, fragmented and the methylated DNA immunoprecipitated using a methyl-cytosine antibody (MeDIP). The MeDIP DNA fragments were then sequenced for an MeDIP-Seq analysis. The MeDIP-Seq analysis was used since >90% of the genome has low CpG density regions, so can assess >90% of the genome-wide DNA methylation. The sperm DMR numbers are presented in FIG. 1 for various edgeR statistical p-value cutoff threshold values, and a stringent p-value of p<1e−05 (control versus glyphosate) or p<1e−04 (diseased versus non-diseased) were selected as the threshold for subsequent analyses. The total number of DMRs for the control versus glyphosate is 340 with 11 of them having multiple neighboring 1000 bp windows, (FIG. 1A and FIG. 2).

The previously reported transgenerational F3 generation sperm glyphosate versus control lineage DMRs used three pools of different animals to identify the glyphosate induced sperm DNA alterations. The current study used individual animal sperm analysis to identify the glyphosate induced transgenerational F3 generation DMRs (FIG. 1A). A comparison of these two studies demonstrates the 340 DMRs with a p<1e−05 has an overlap of 129 DMRs with the previous DMRs at p<0.05. Therefore, a 38% overlap was observed at this reduced statistical threshold.

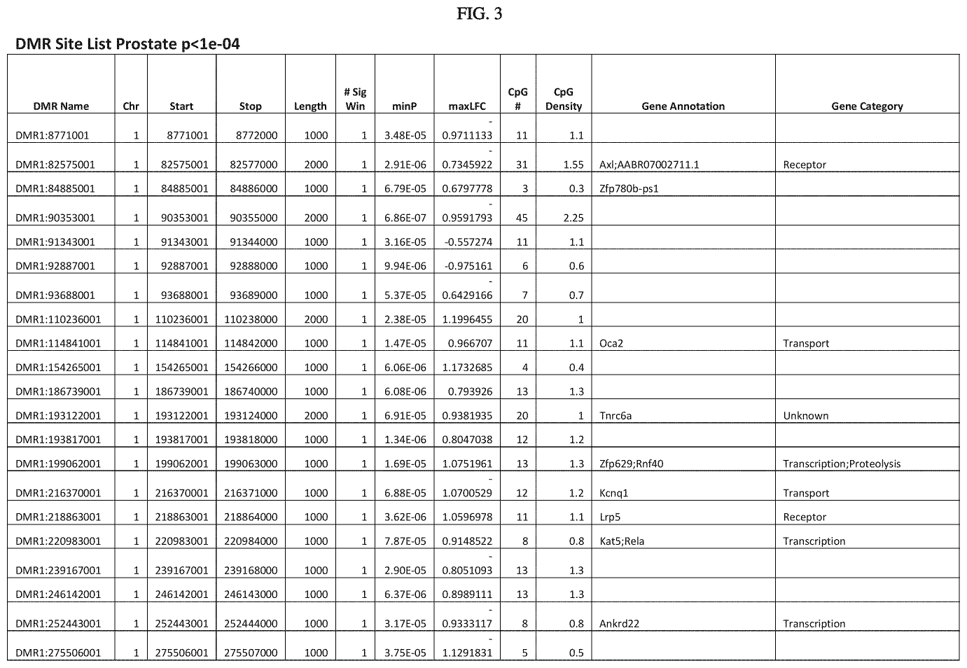

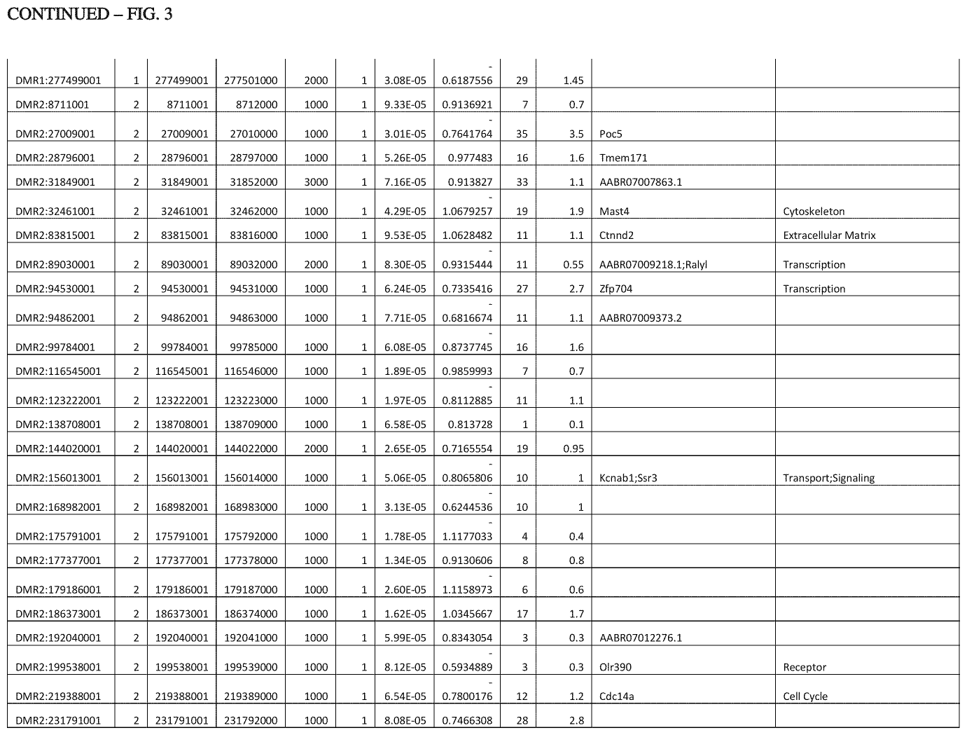

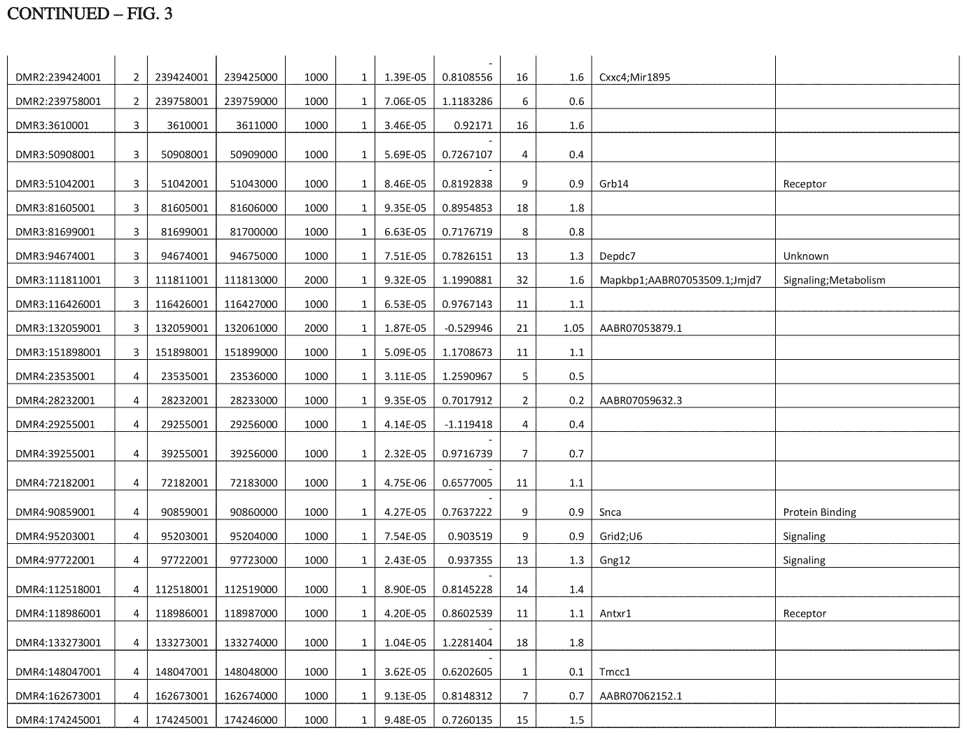

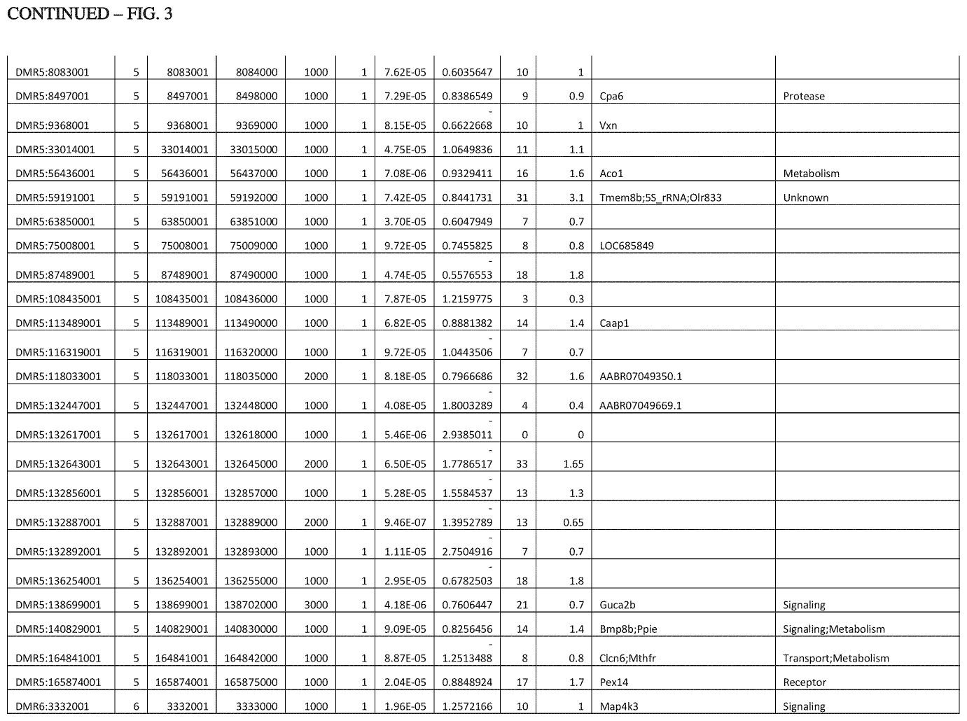

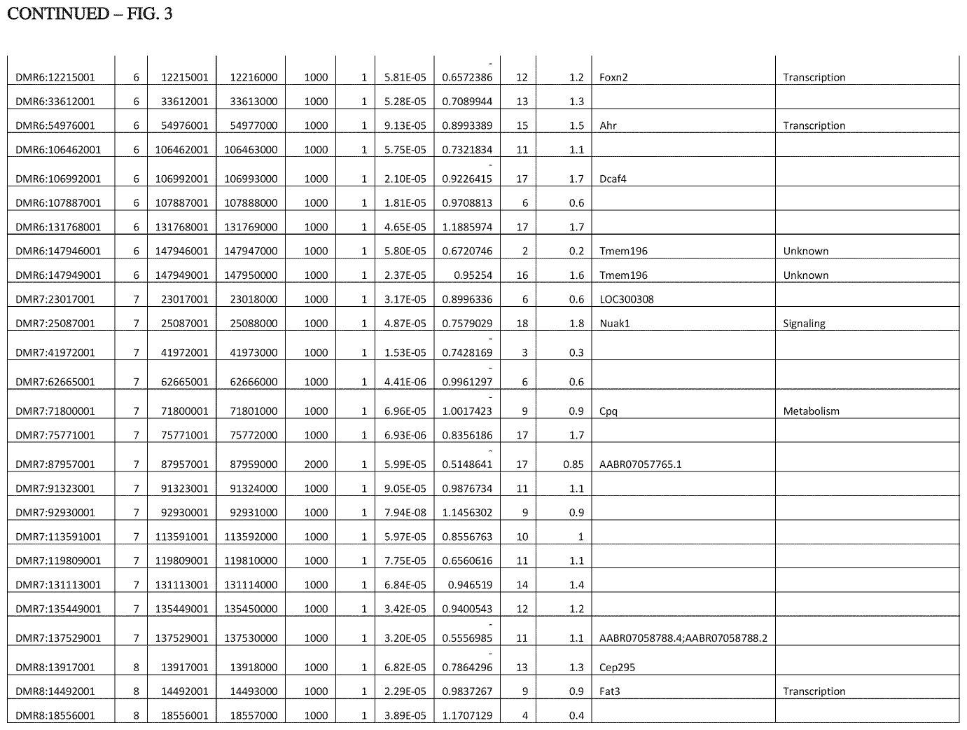

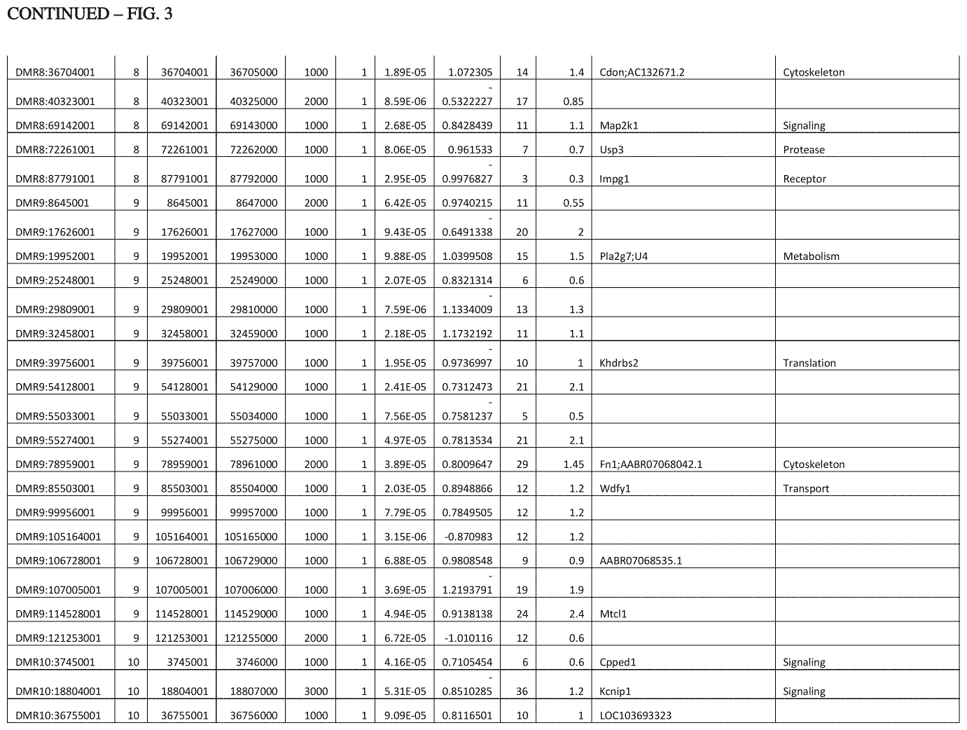

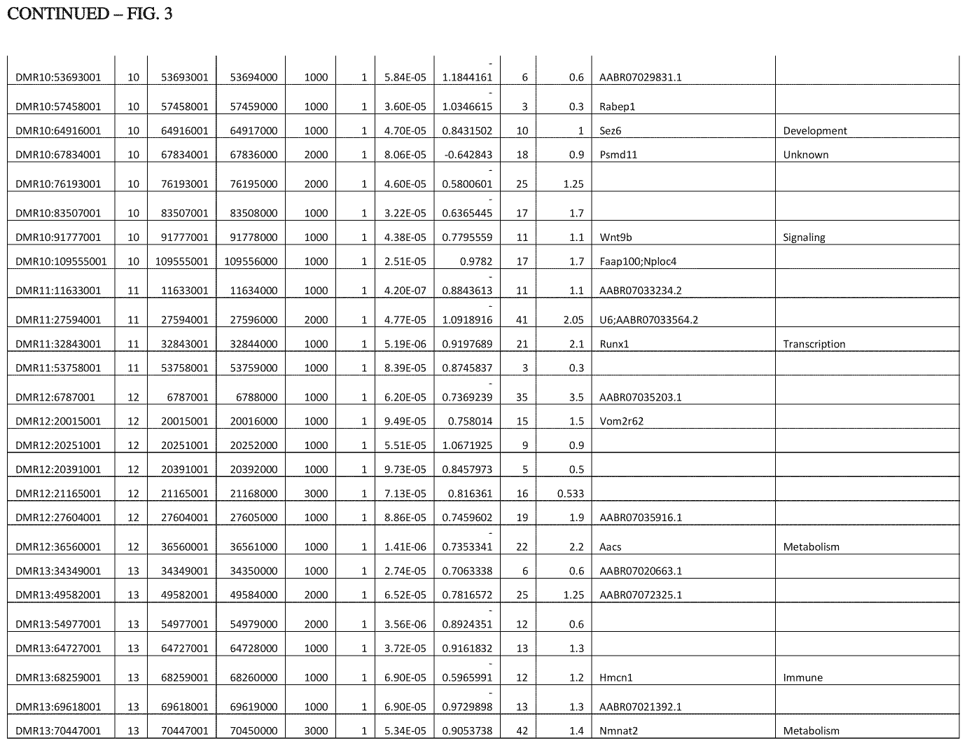

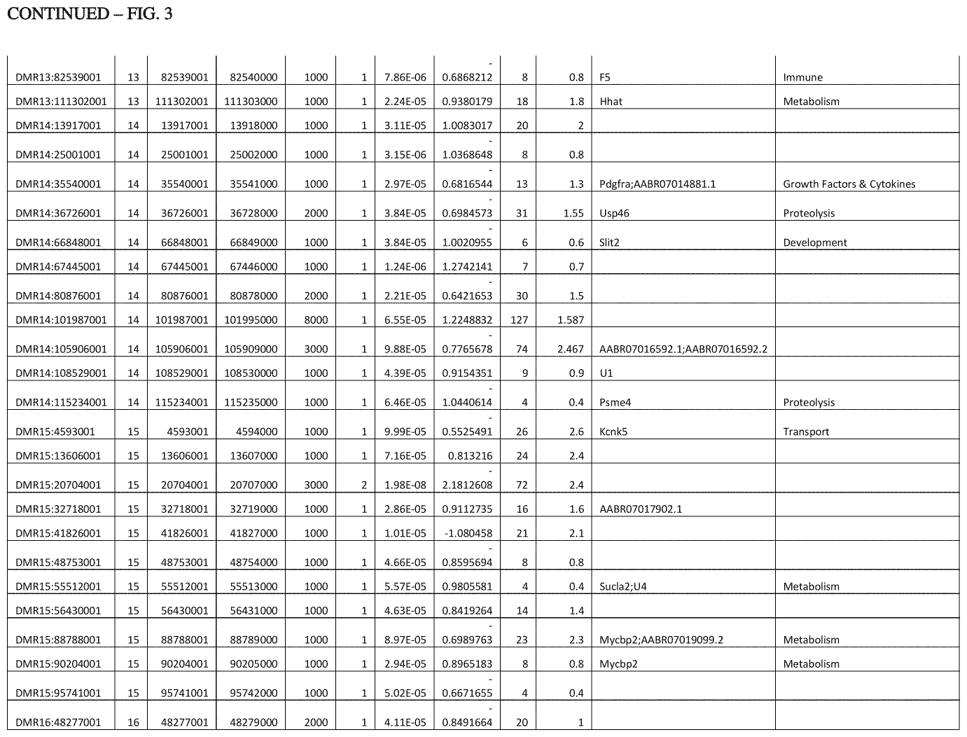

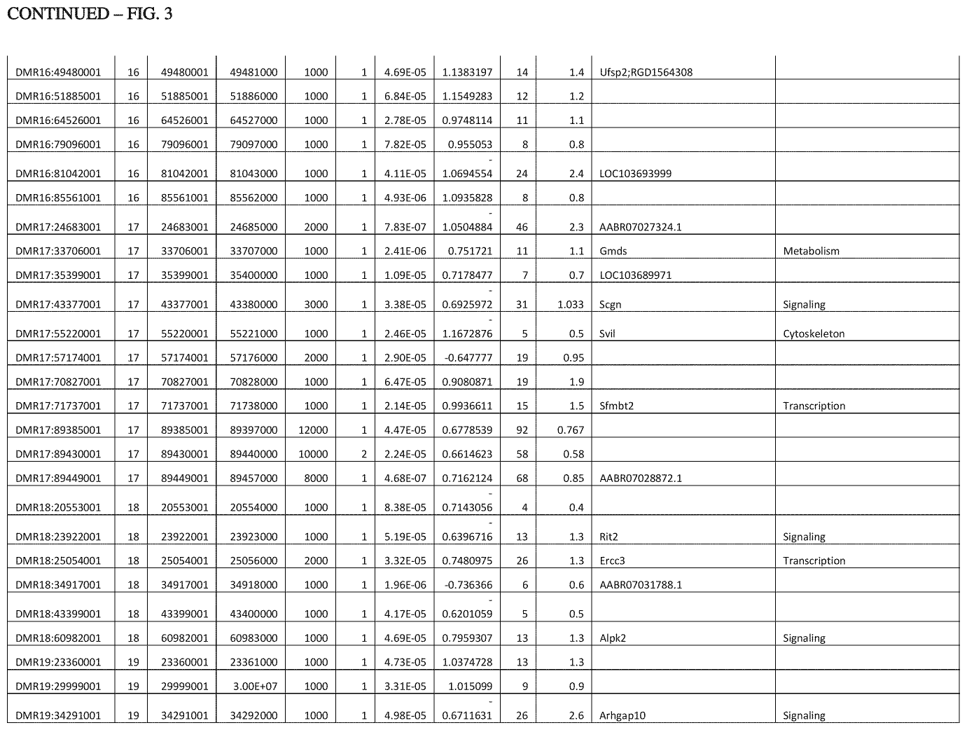

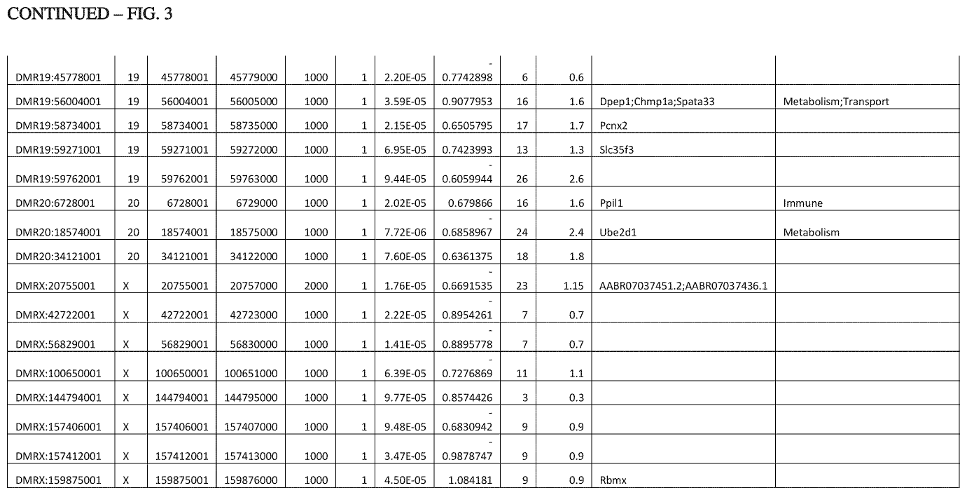

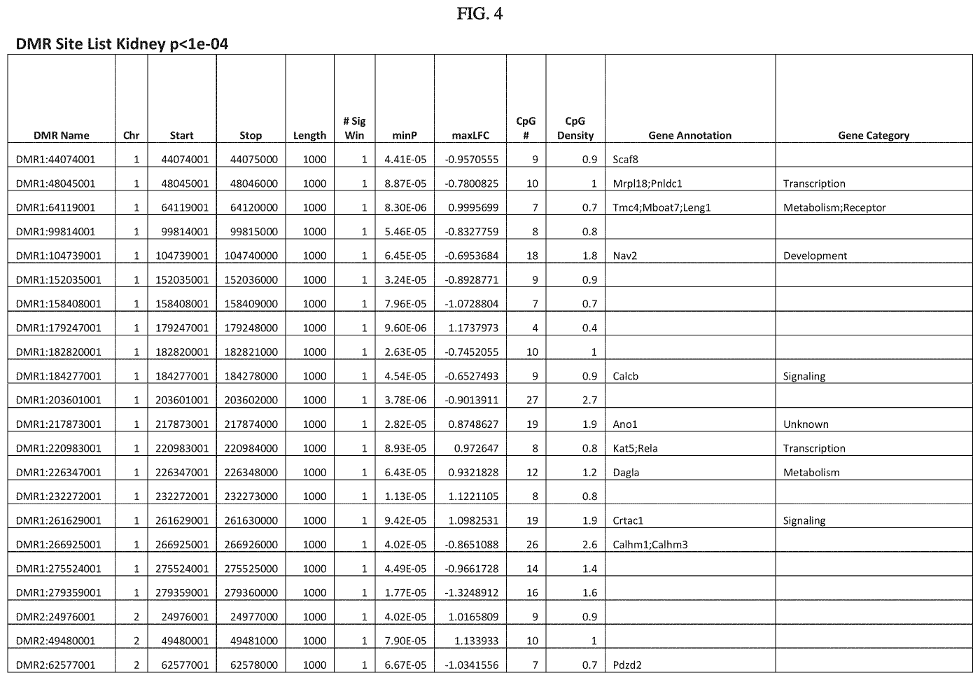

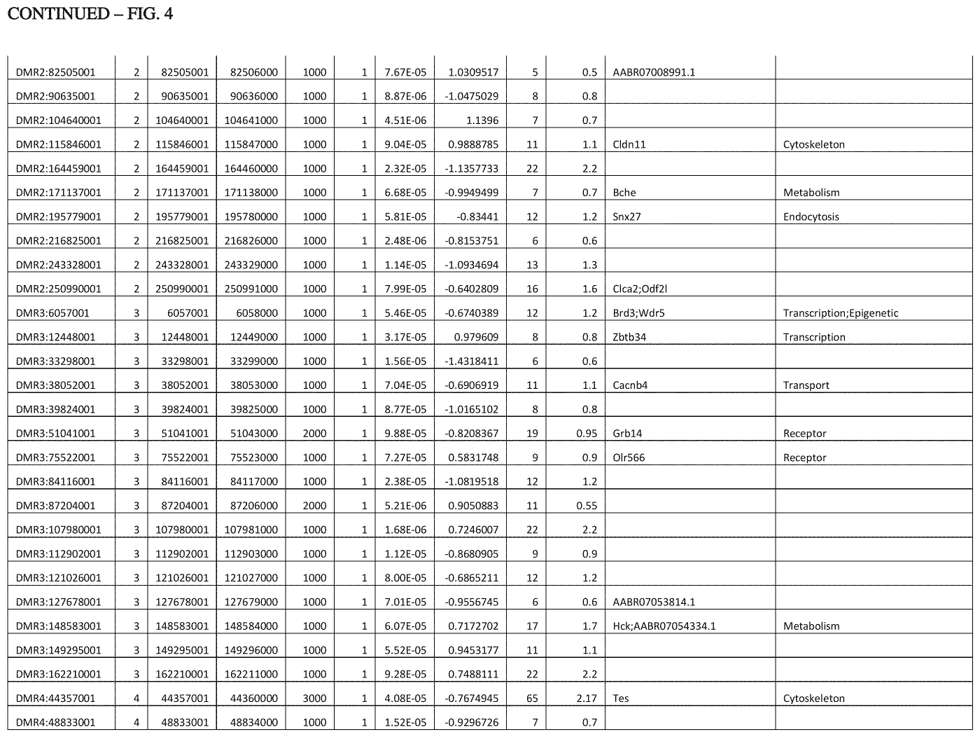

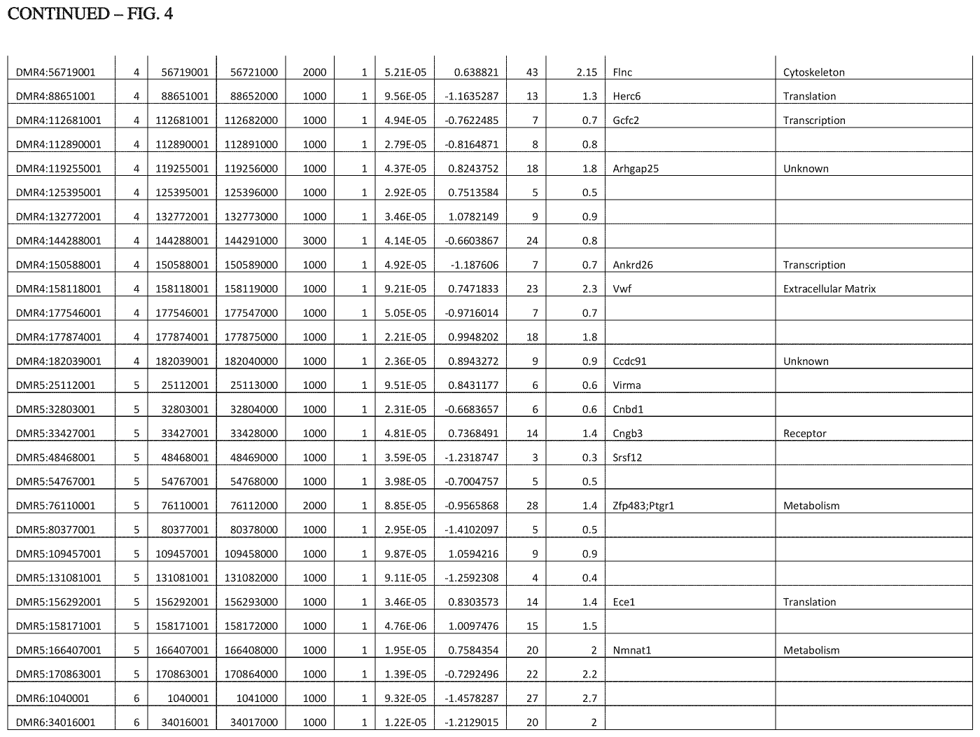

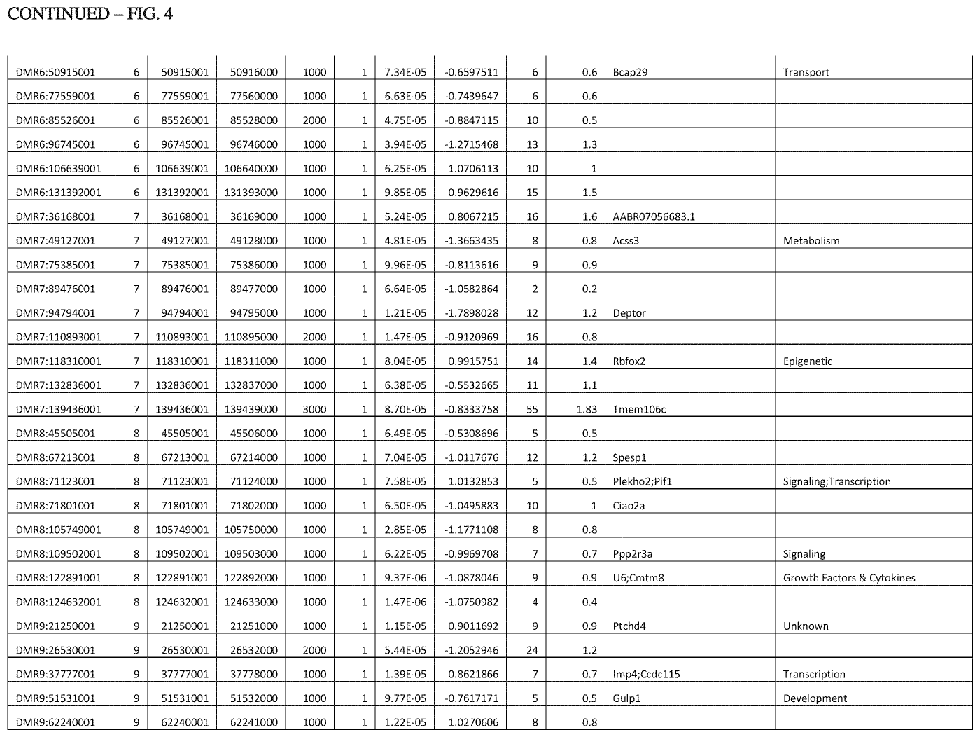

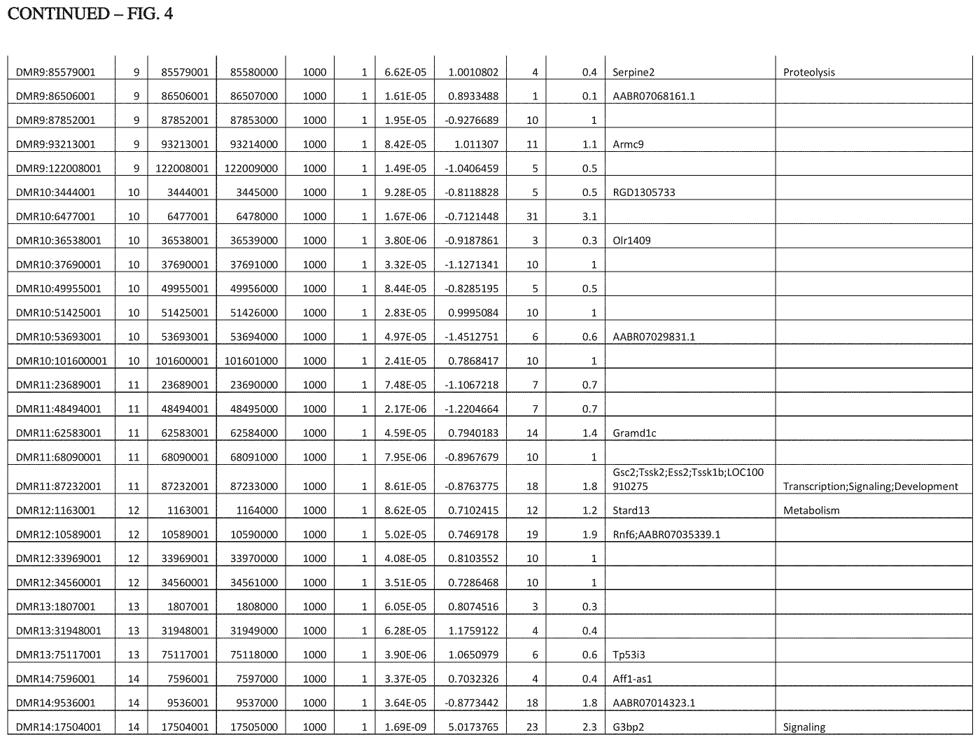

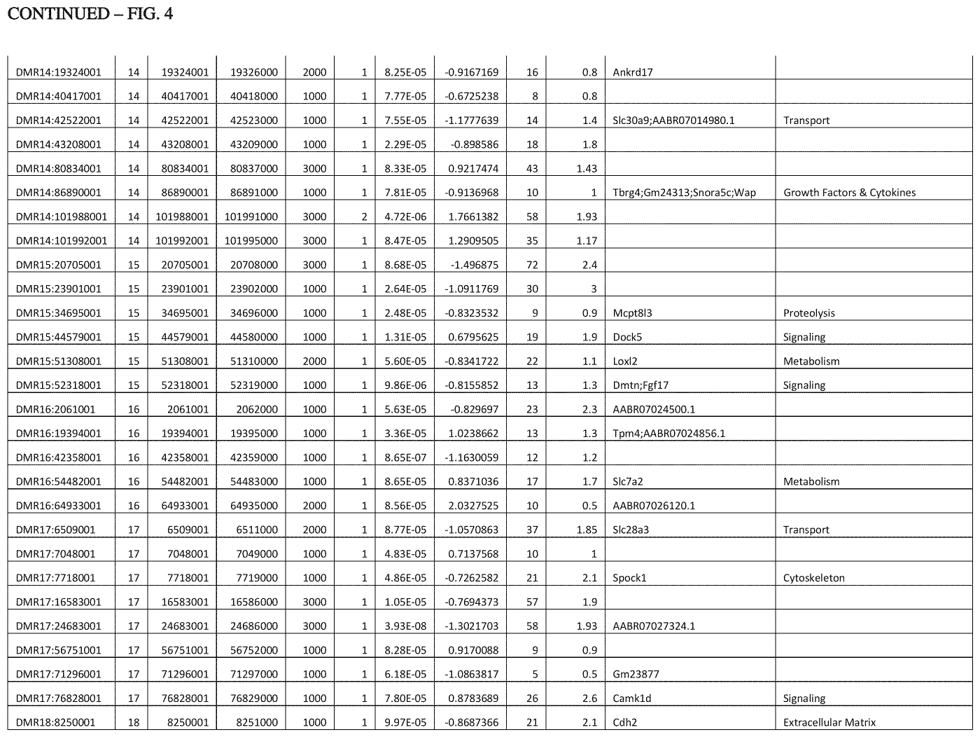

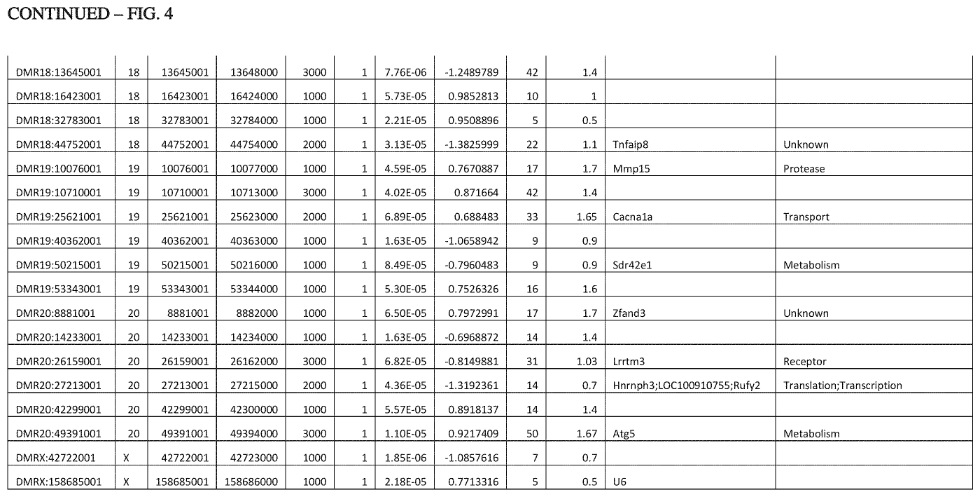

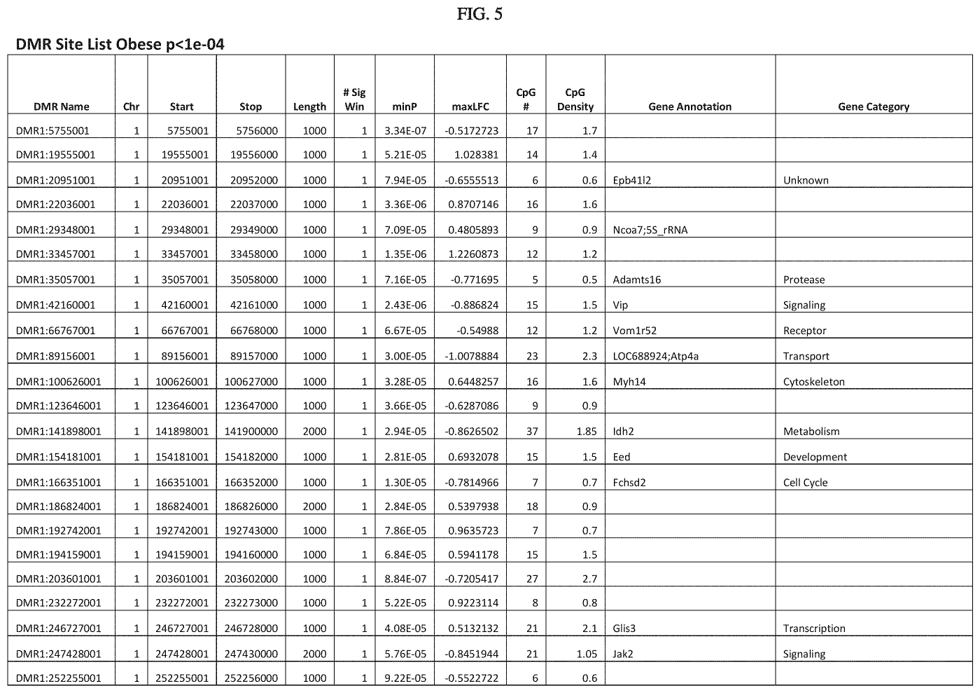

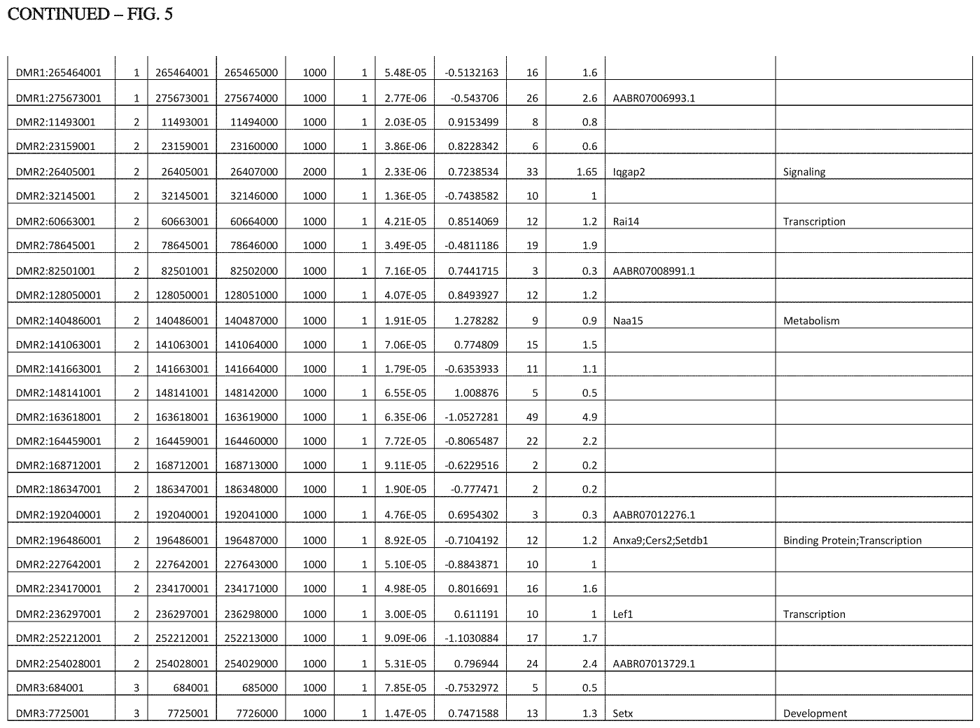

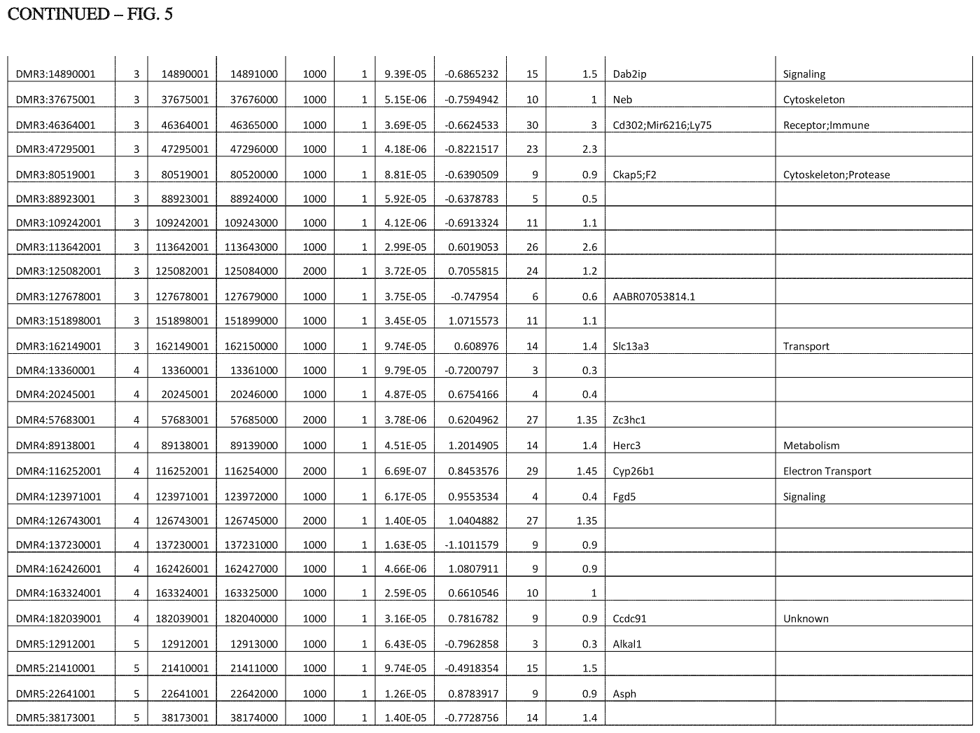

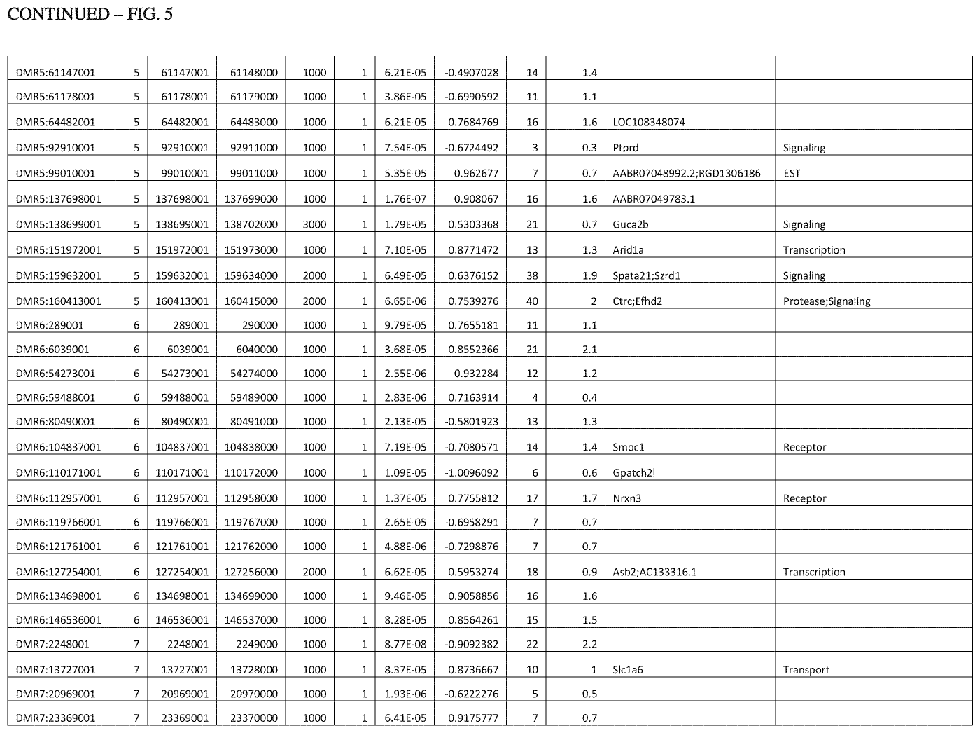

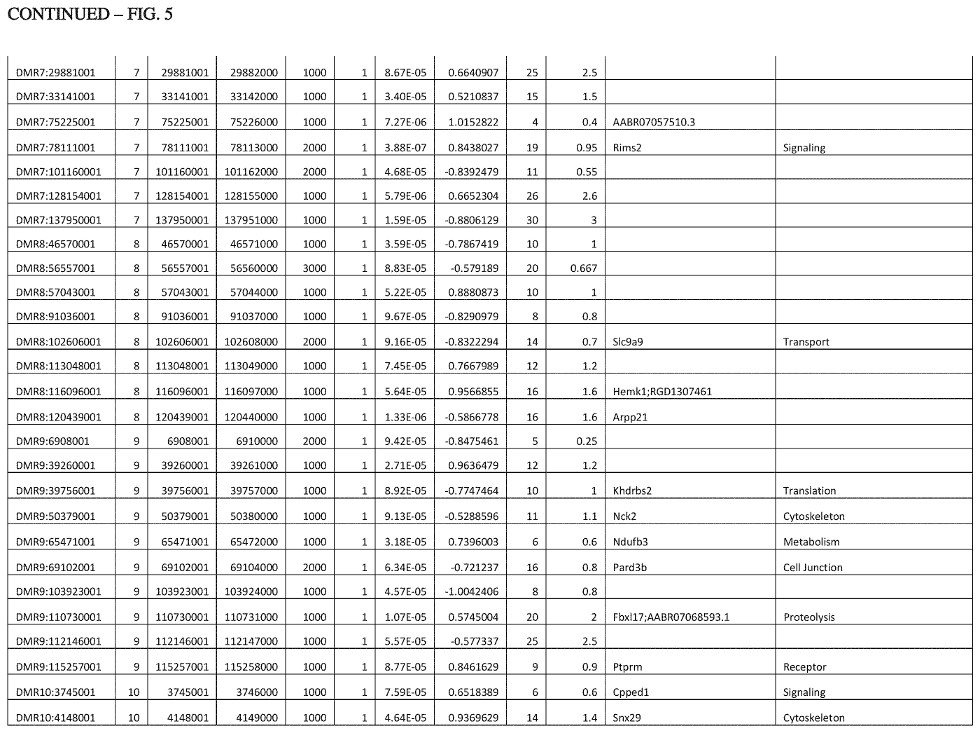

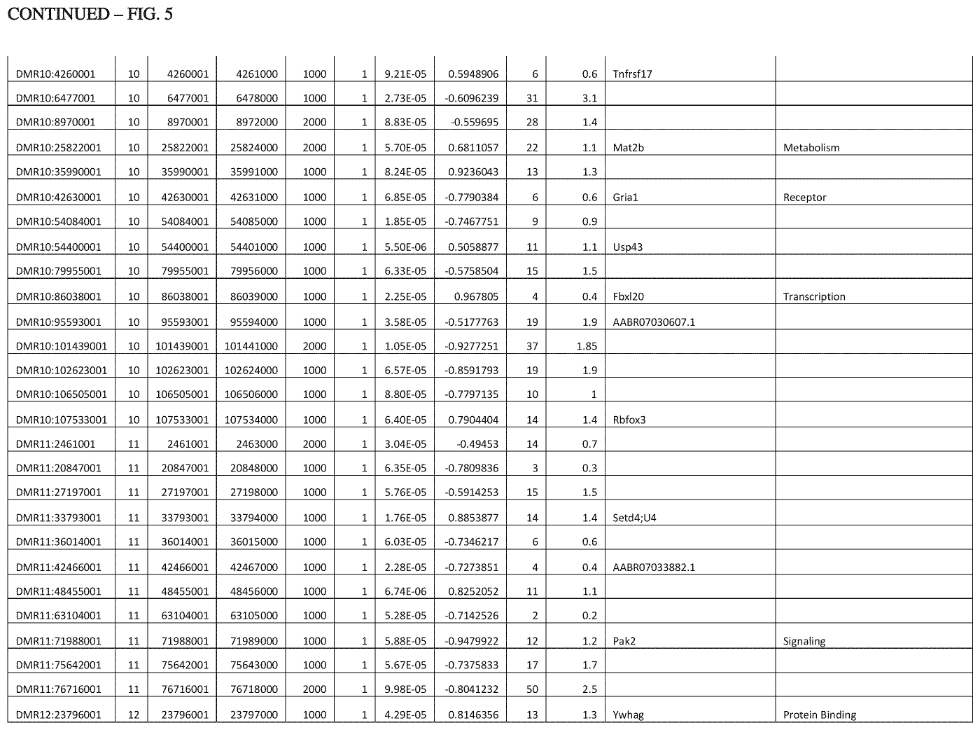

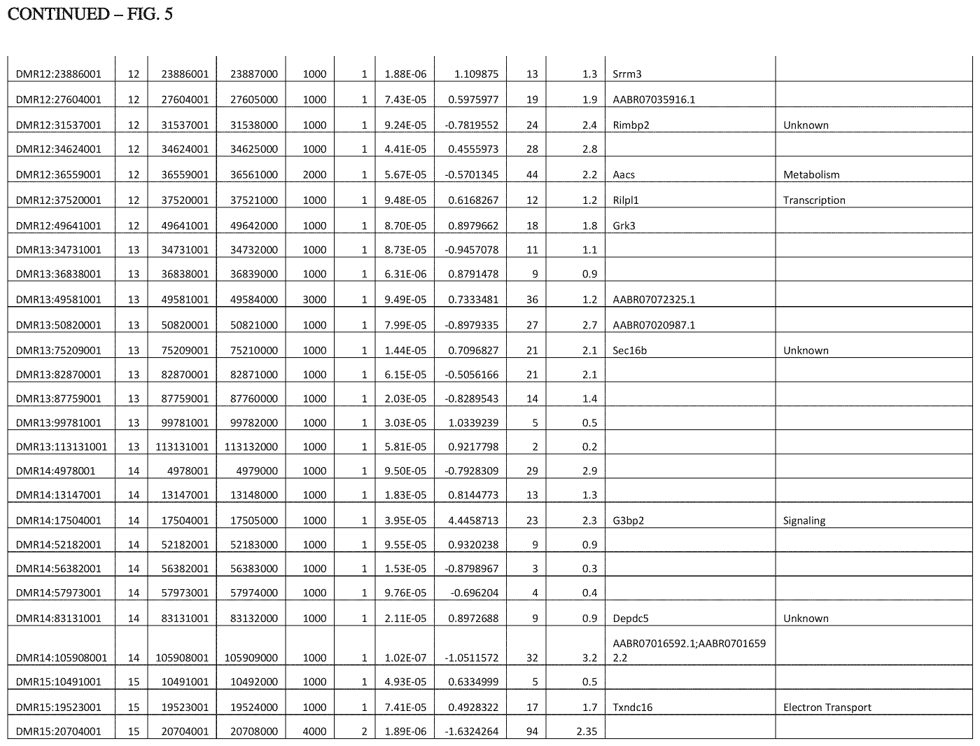

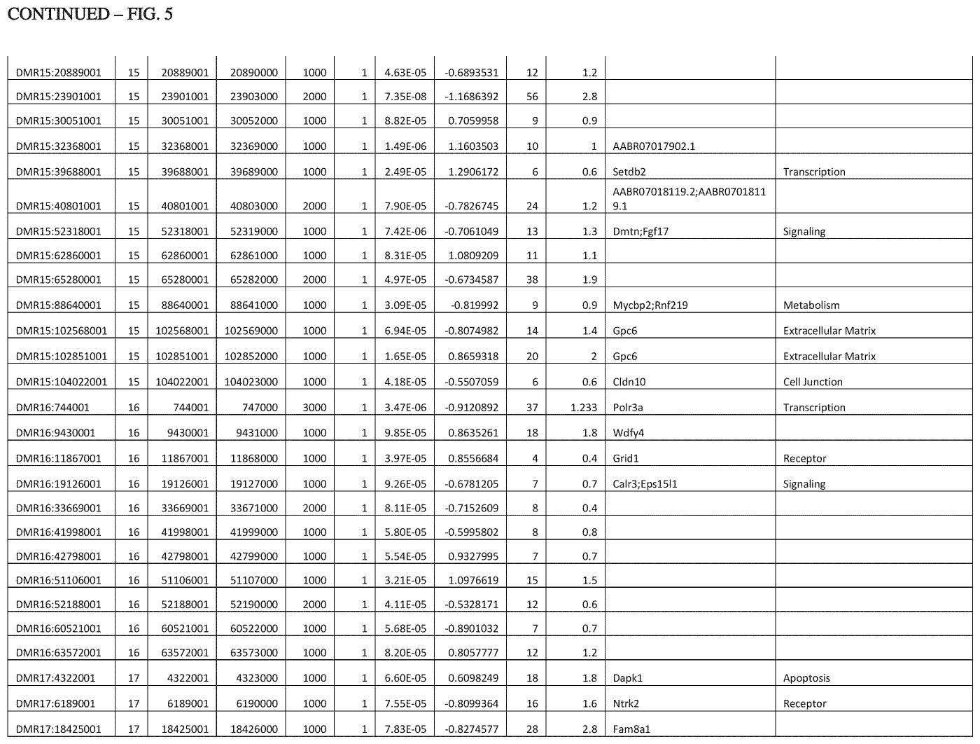

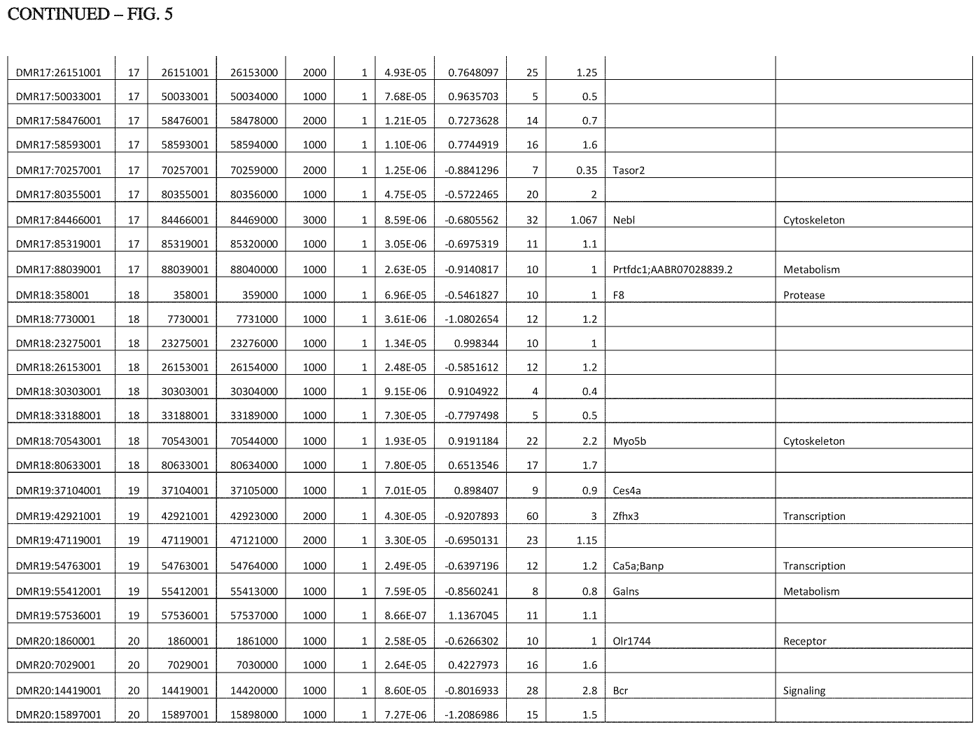

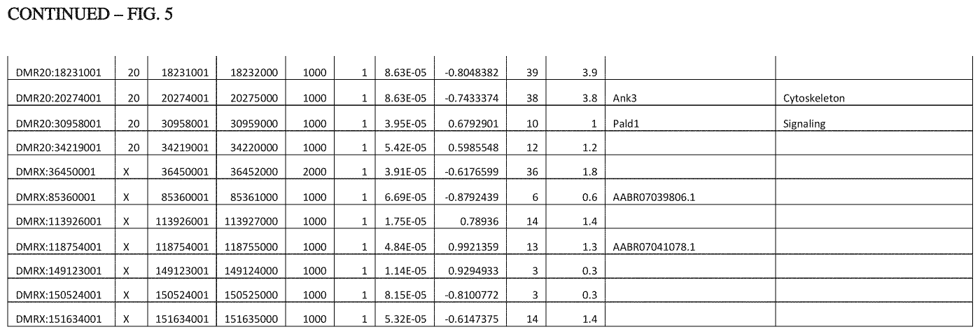

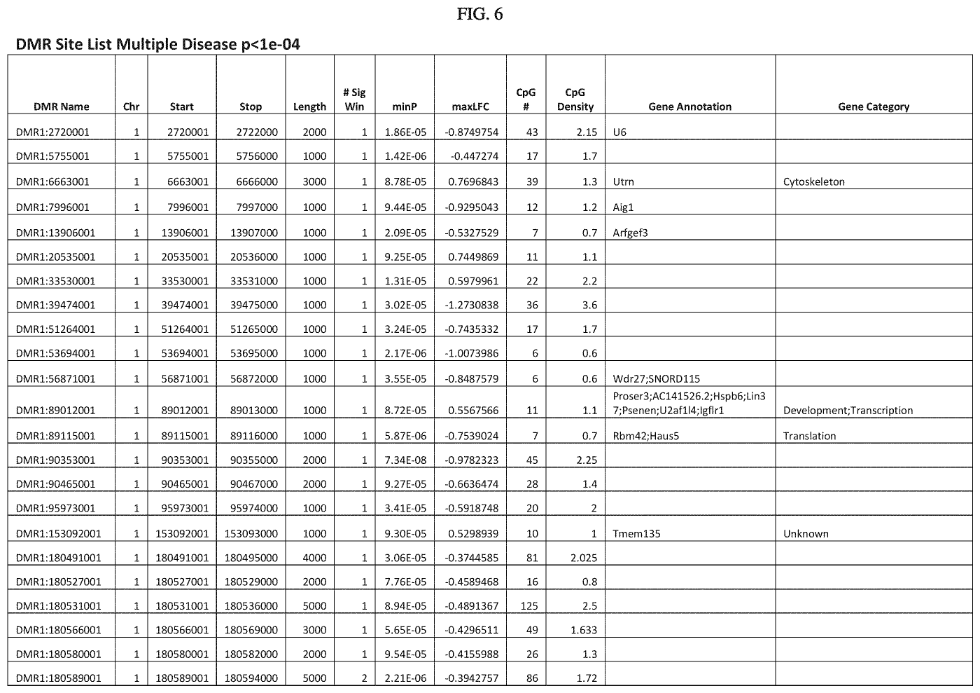

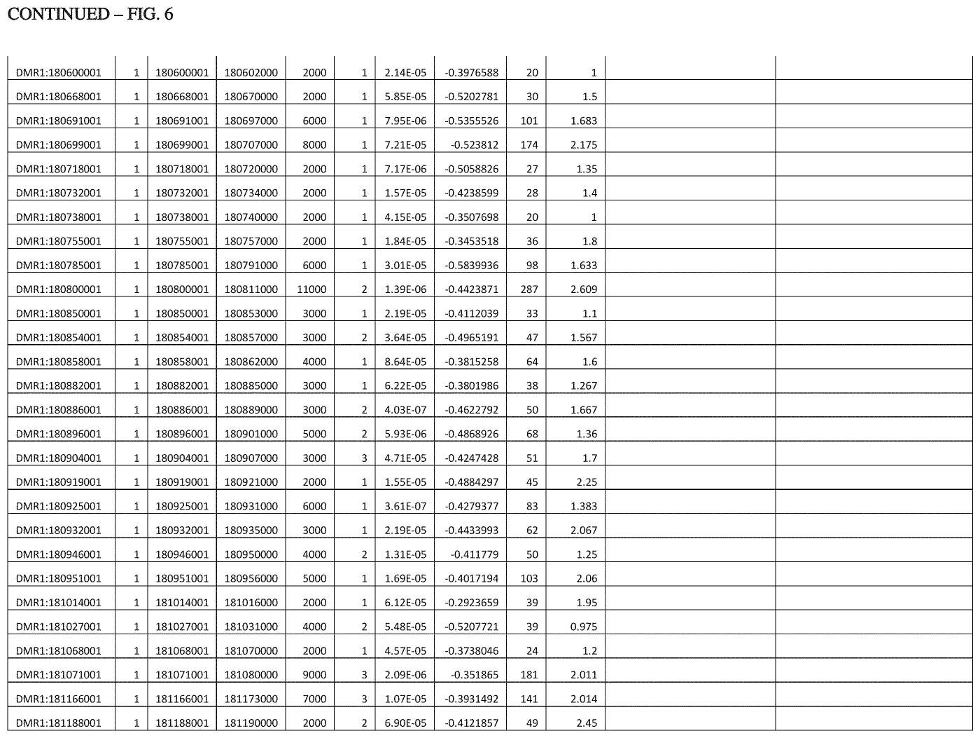

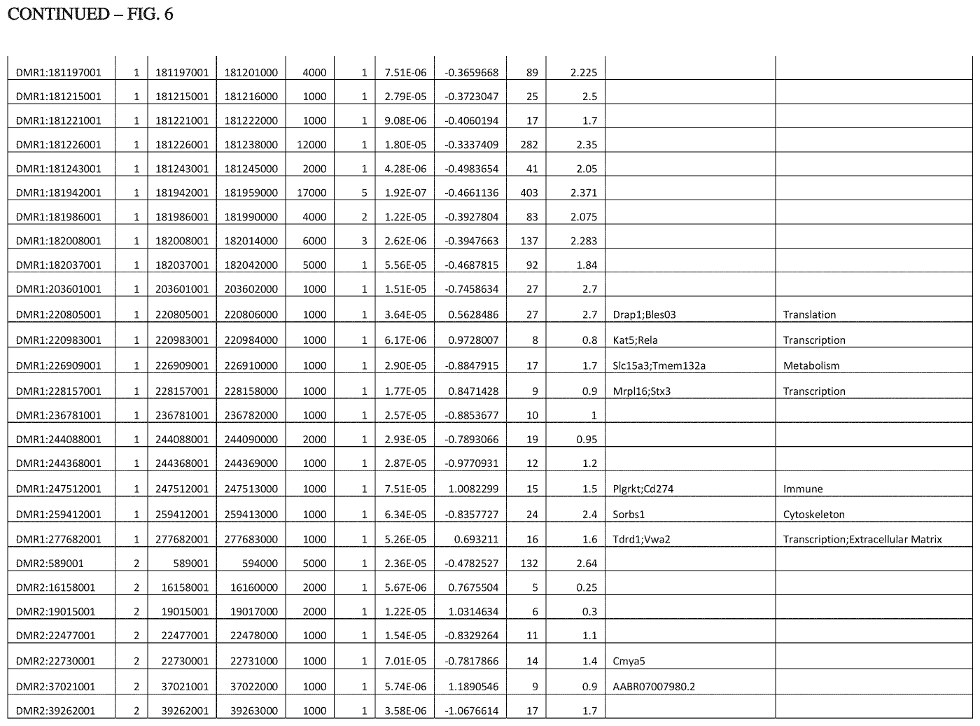

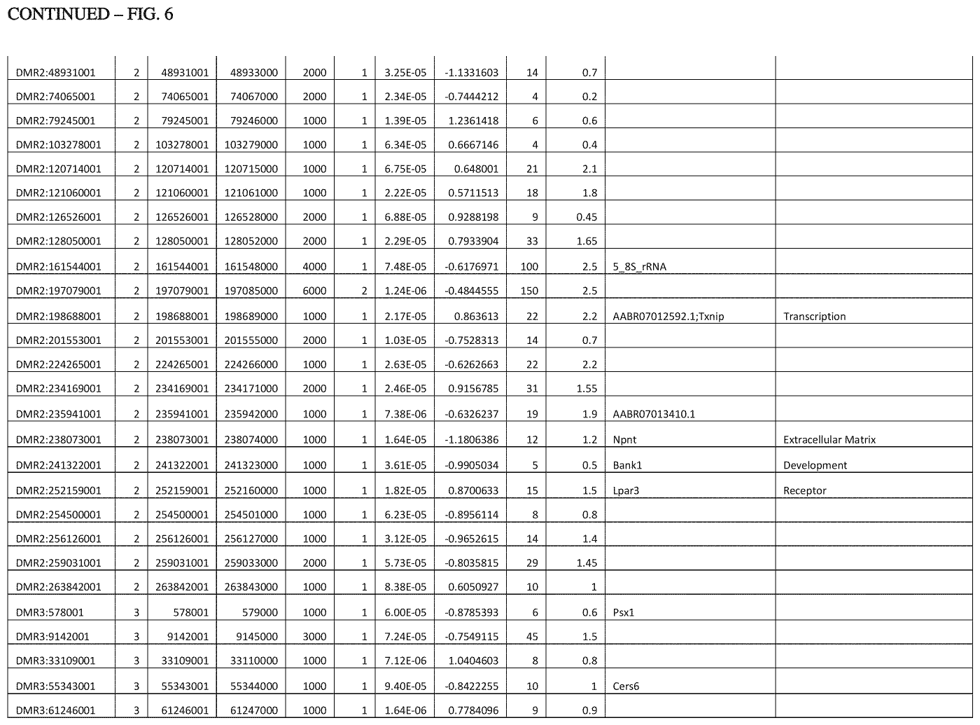

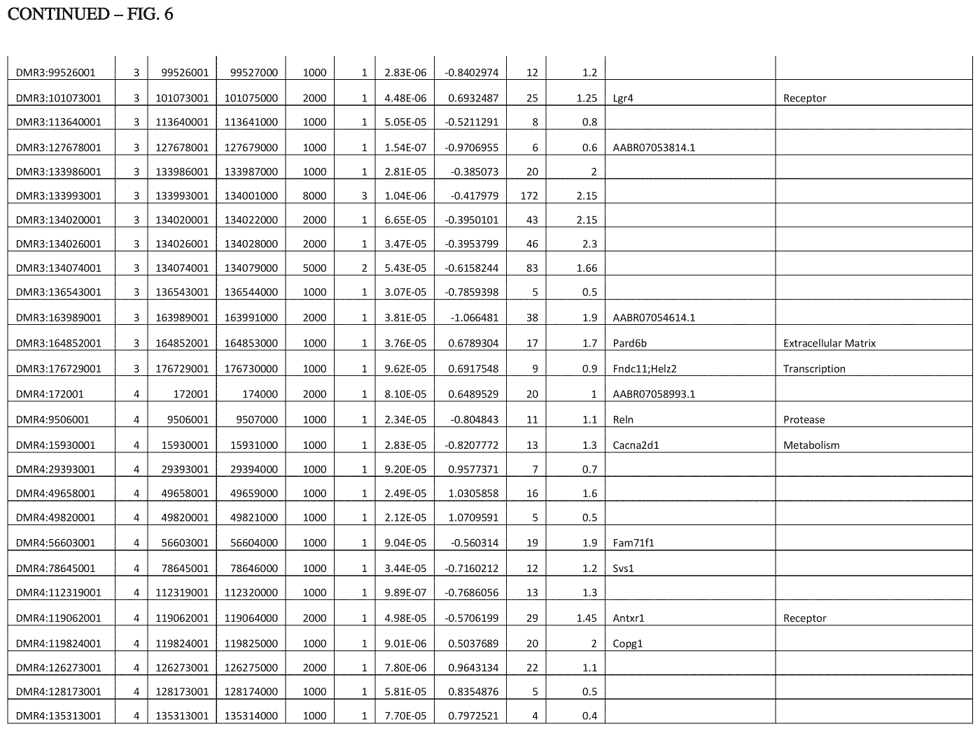

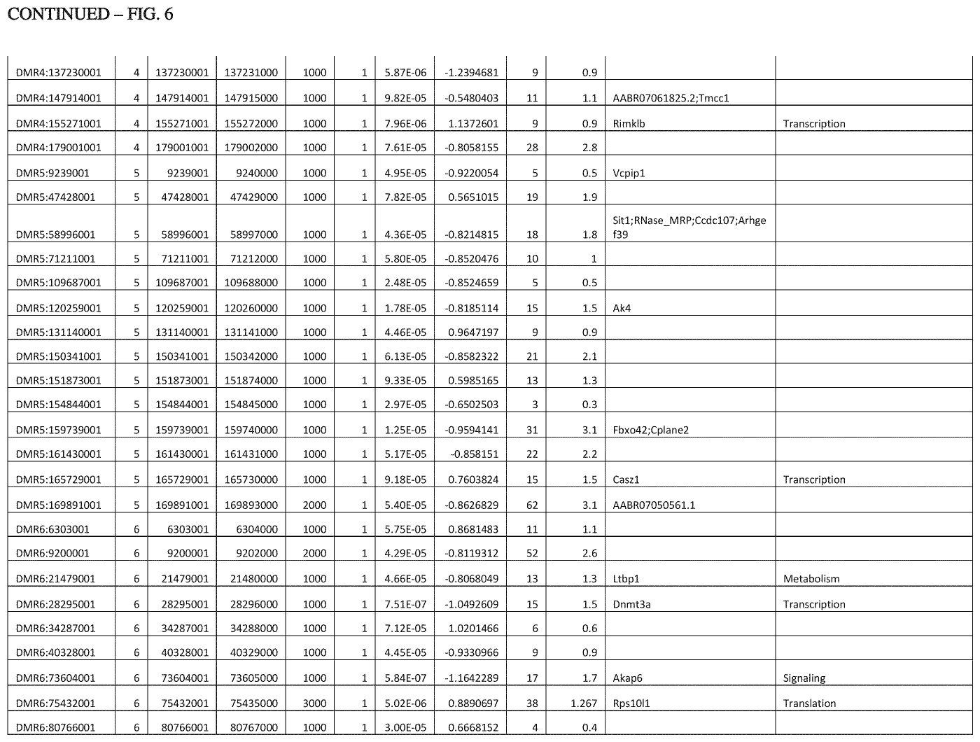

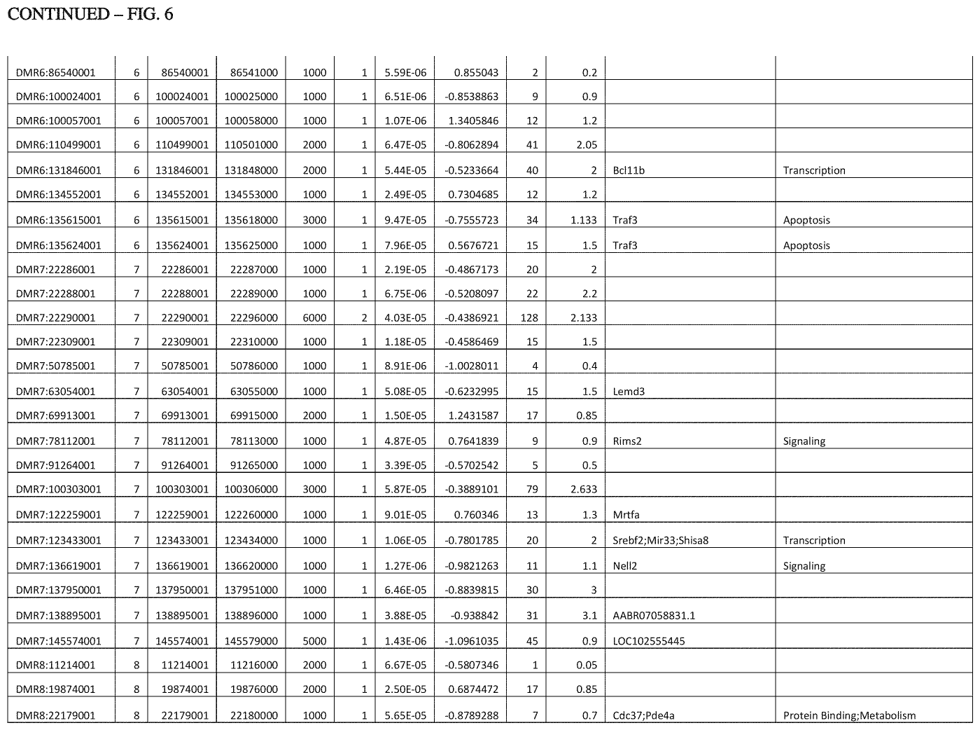

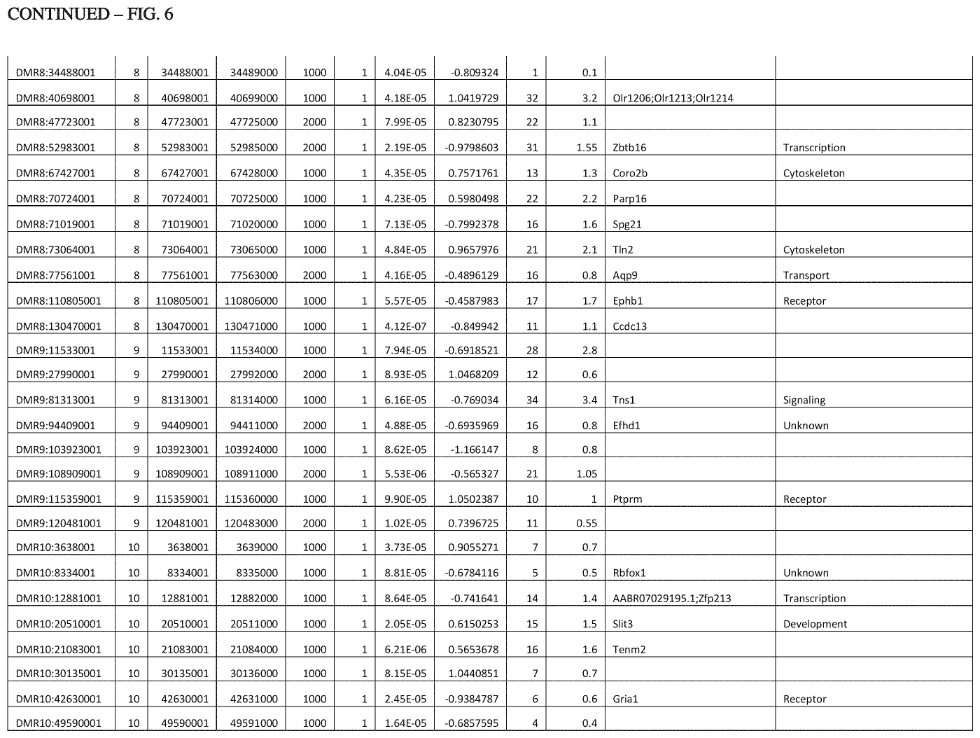

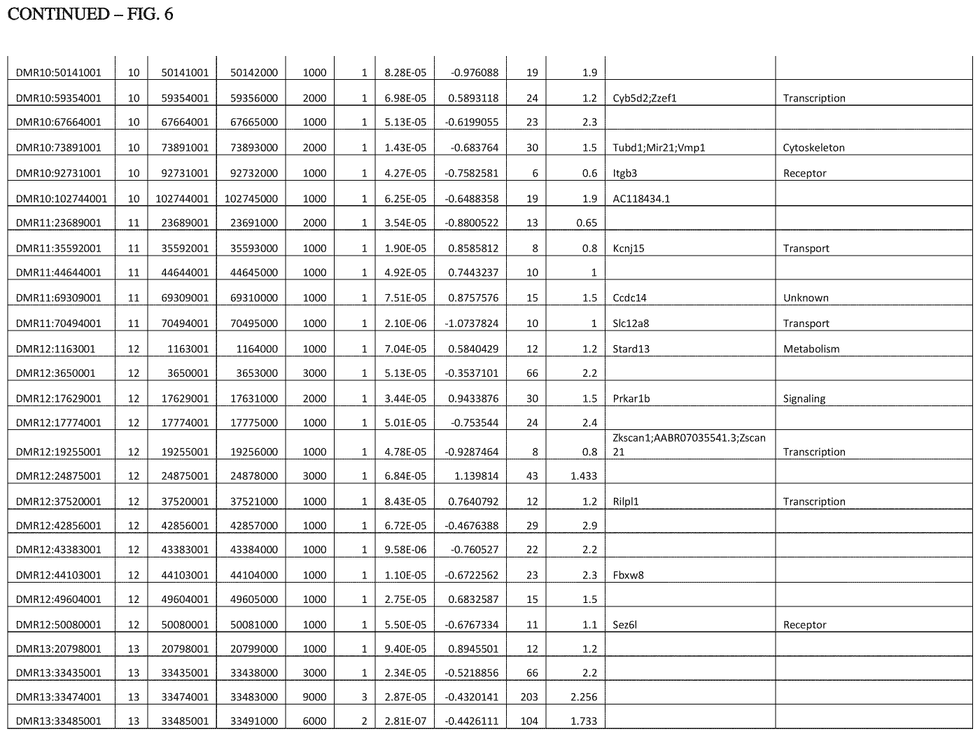

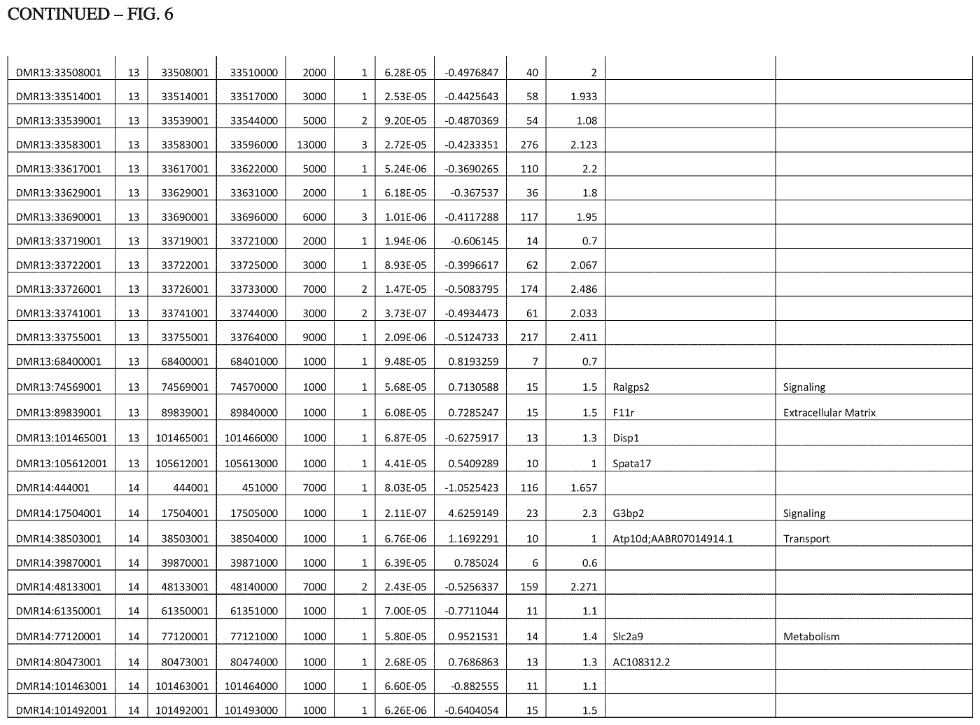

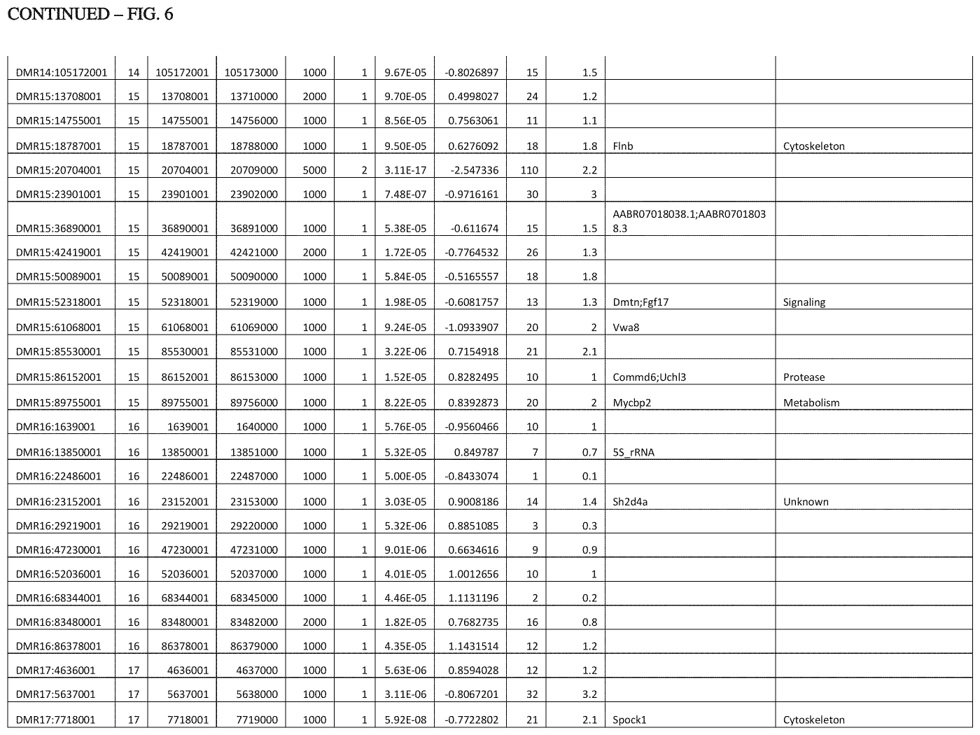

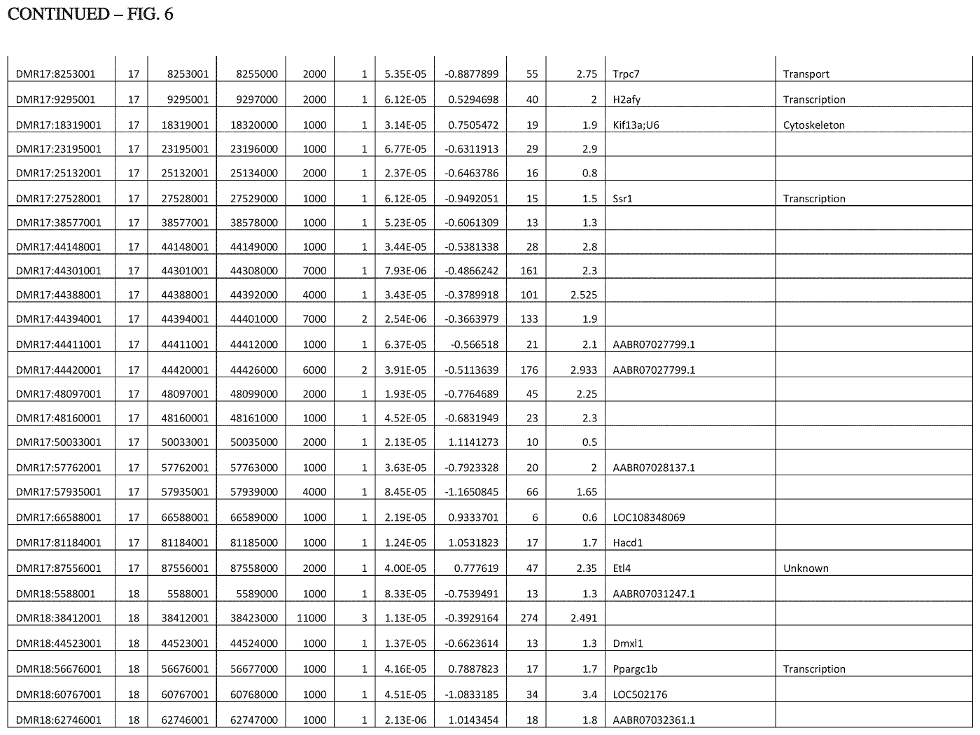

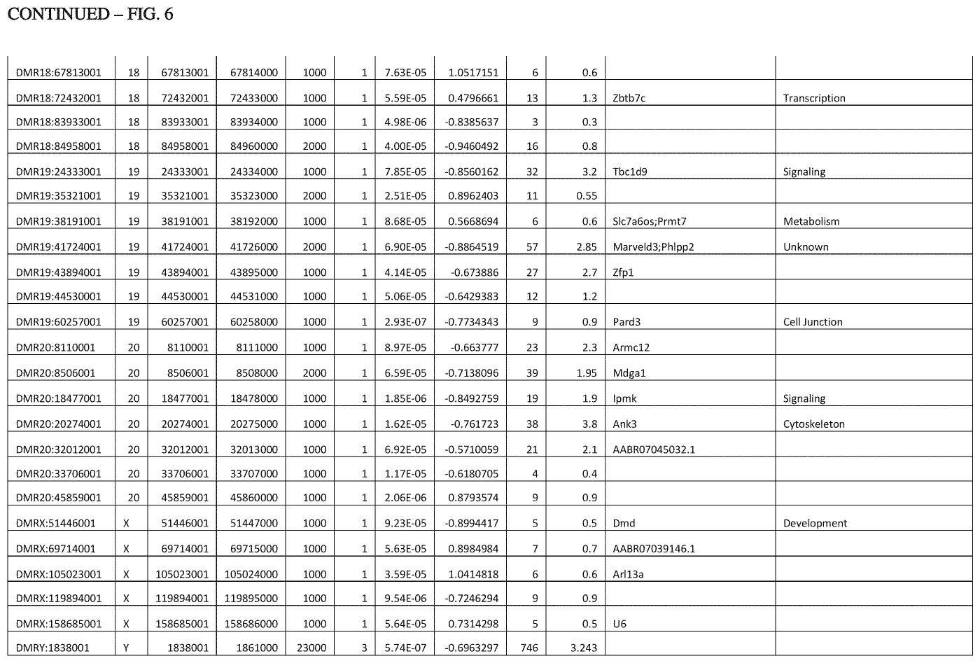

The animals with a specific disease were compared to non-disease animals to identify the disease specific sperm DMRs. The group of animals with prostate disease had 242 DMRs at p<1e−04 with two multiple windows (i.e., 1 kb each) detected, (FIG. 1B). The kidney disease group was found to have 180 total DMRs with 1 of these having multiple neighboring windows, (FIG. 1C). The obesity disease group had 250 DMRs at p<1e−04 with 1 of these having multiple neighboring windows, (FIG. 1D). The multiple disease group had 345 DMRs at p<1e−04 with 31 of them having multiple neighboring 1000 bp windows, FIG. 1E. Using a log-fold-change analysis of individual DMRs, approximately 50% had an increase in DNA methylation with the rest a decrease in DNA methylation (see FIGS. 2-6).

Therefore, the different diseases were found to have altered DNA methylation in the F3 generation sperm. Interestingly, negligible overlap was observed between these different DMRs at a statistical threshold of p<1e−04, (FIG. 1F). Observations indicate glyphosate can promote germline epigenetic alterations in DNA methylation with predominantly disease specific DMRs with an edgeR p<1e−04 threshold (see Tables 3-7).

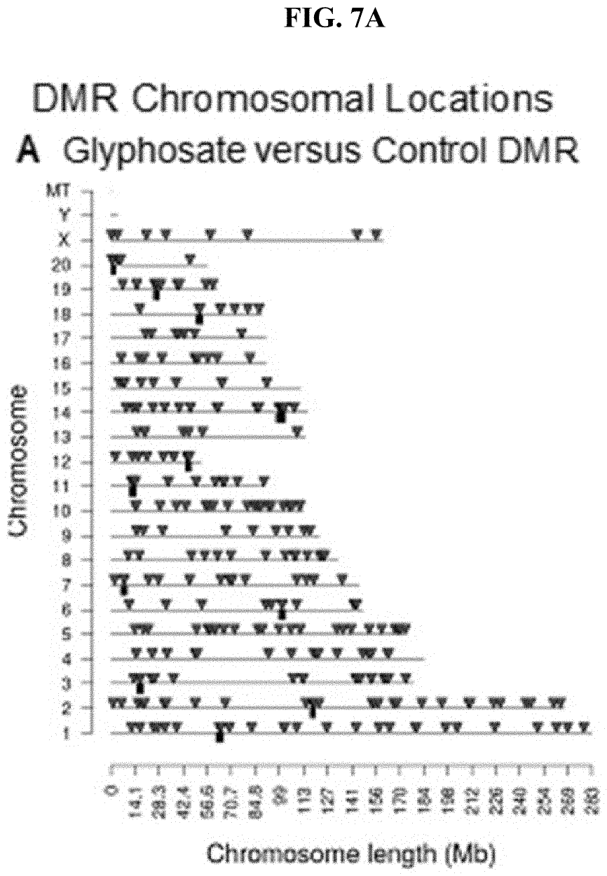

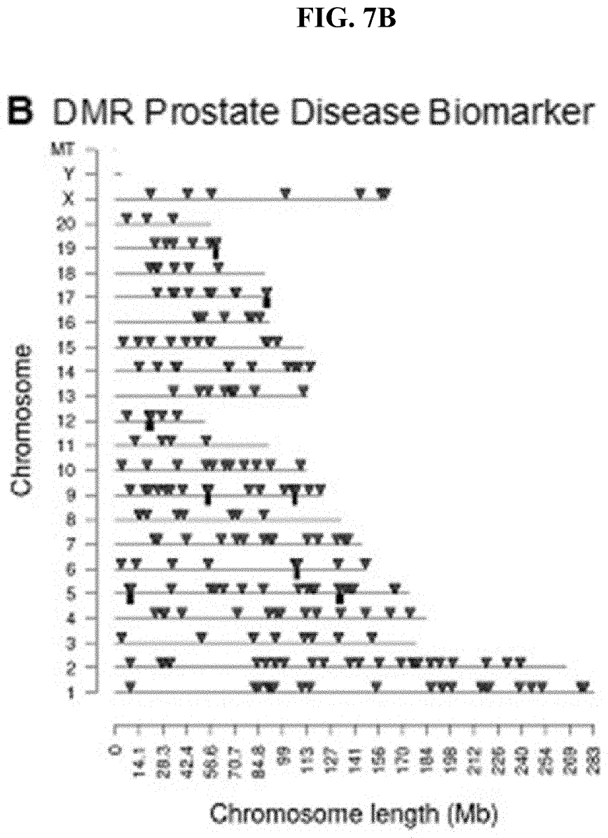

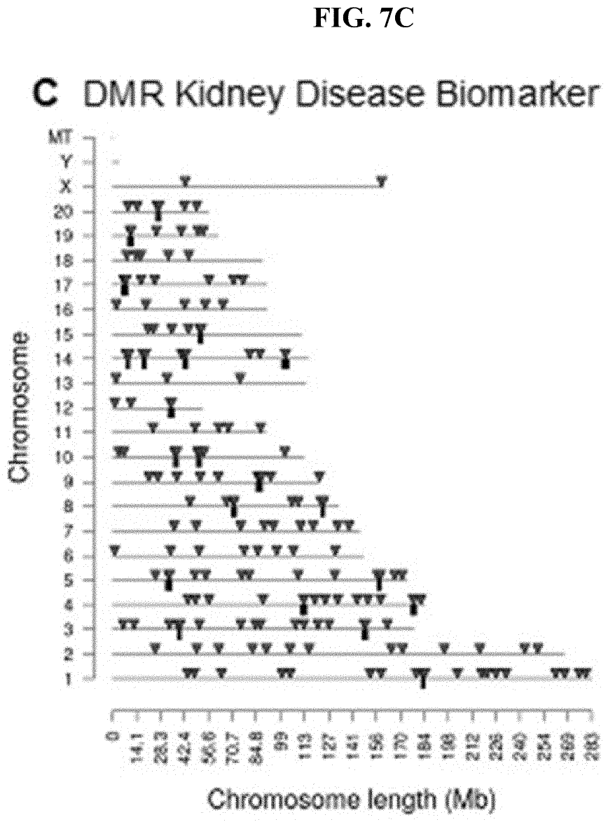

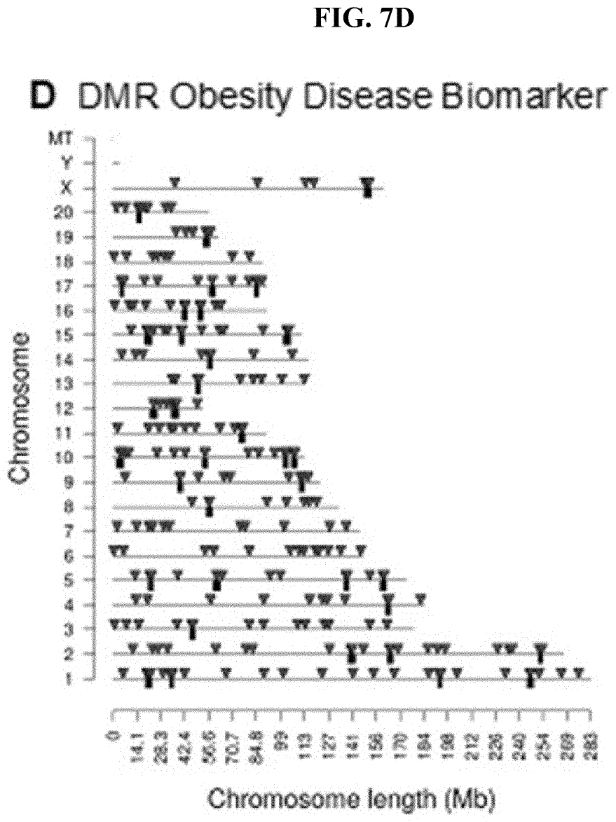

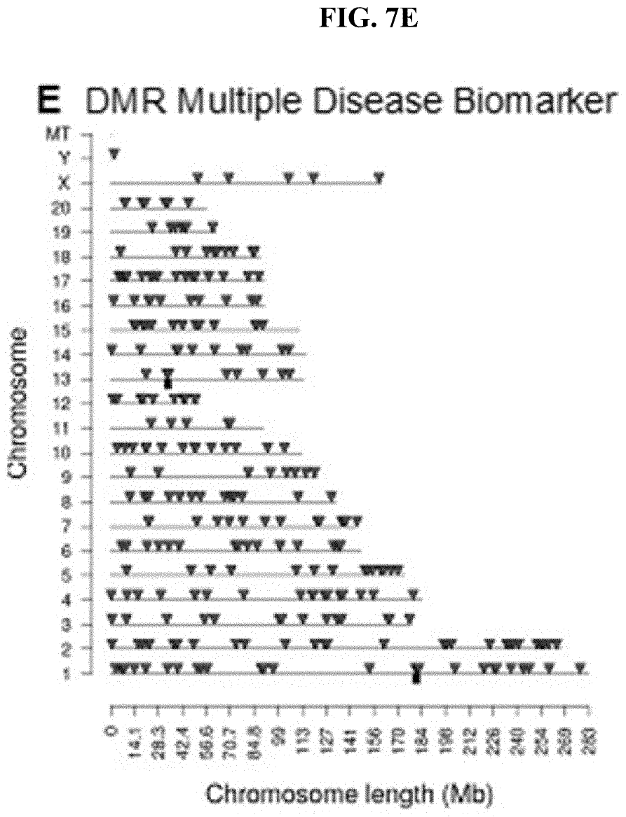

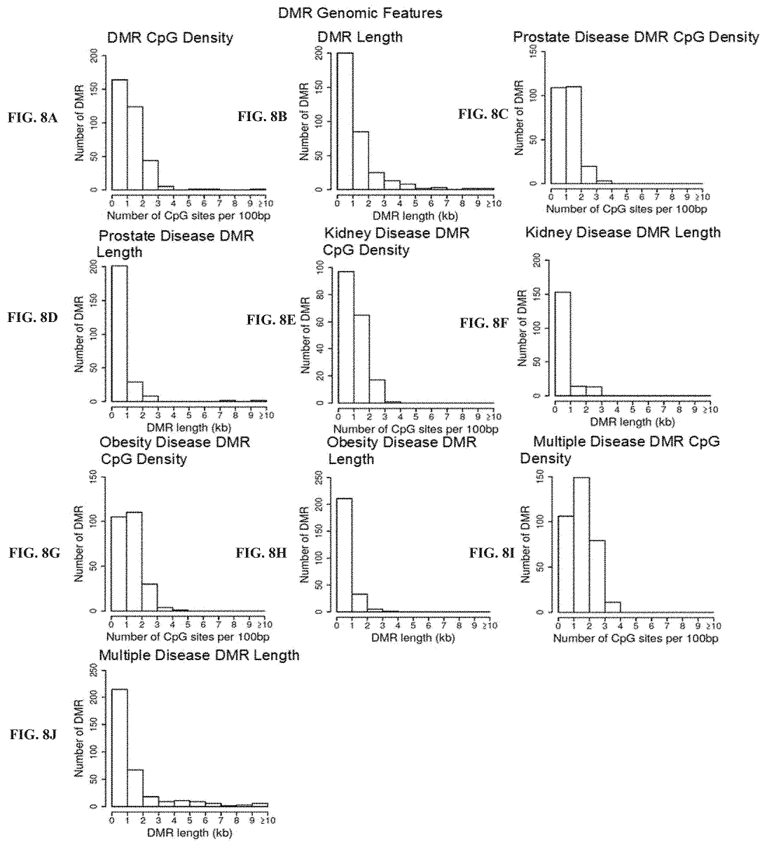

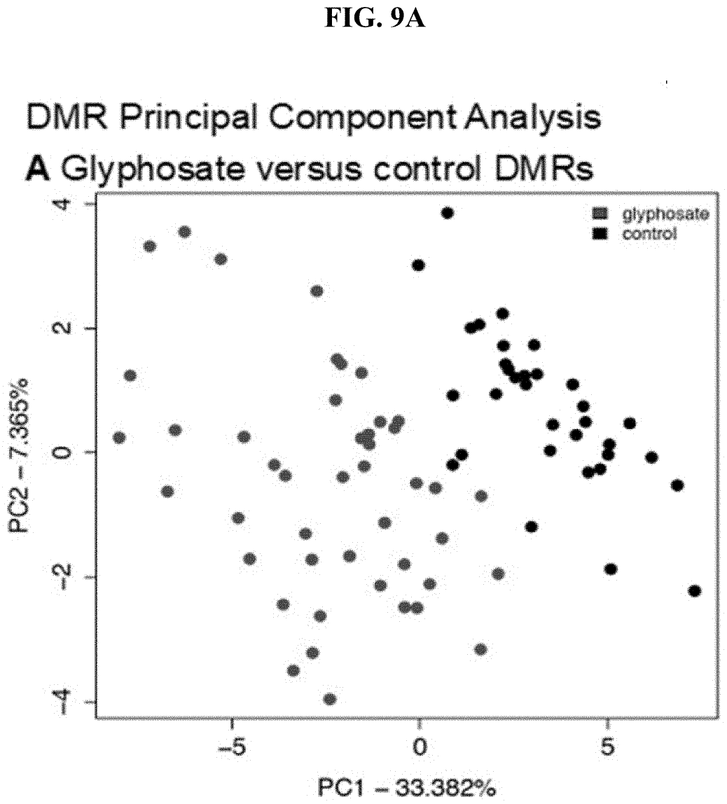

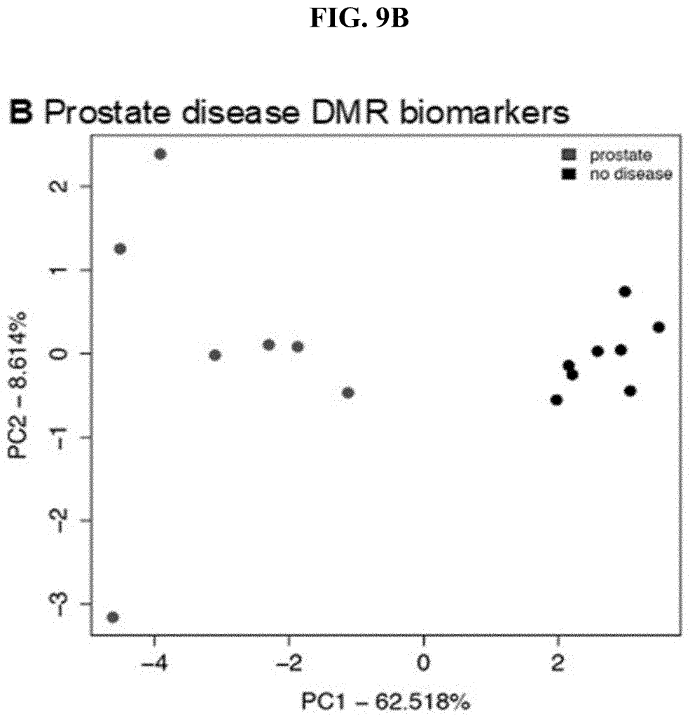

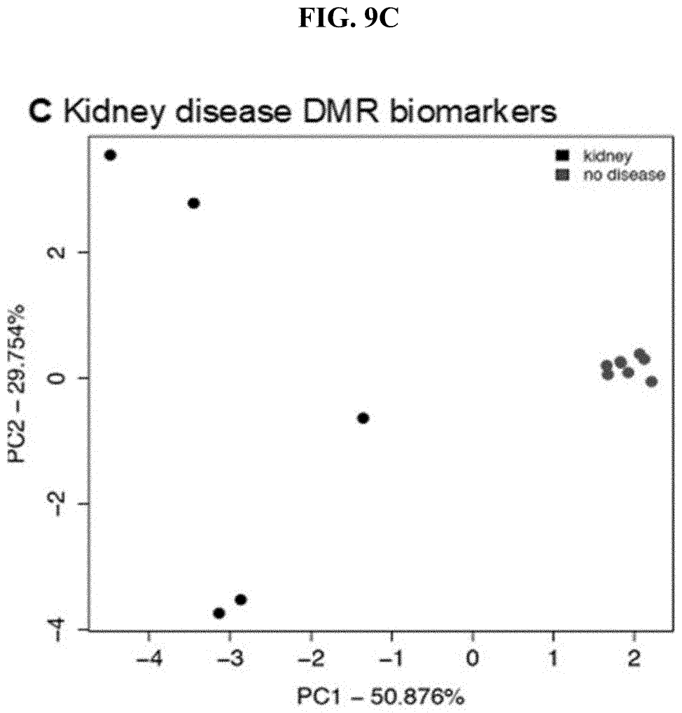

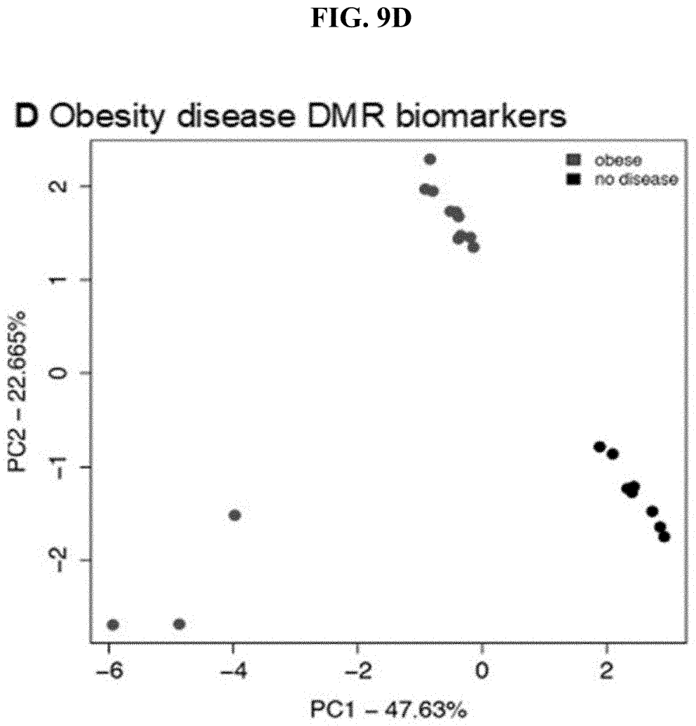

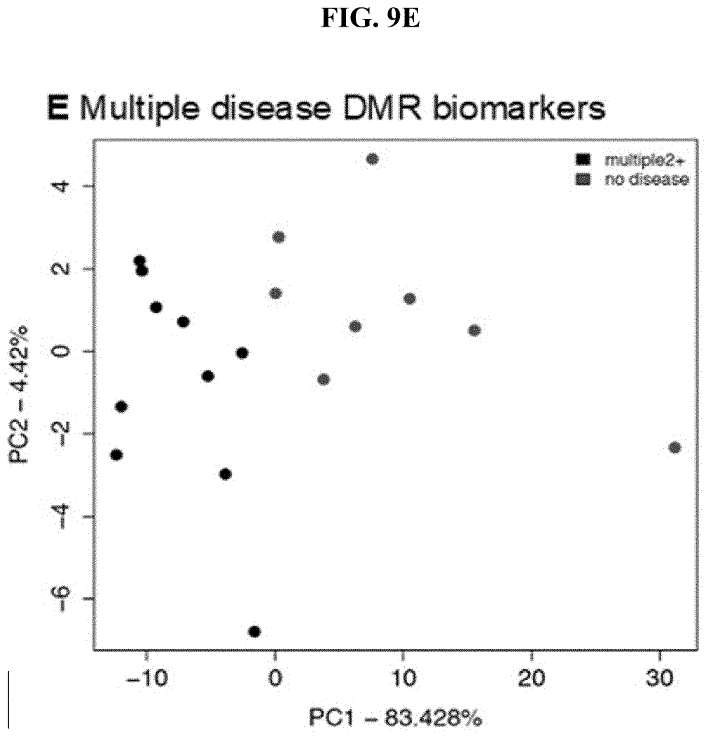

The DMRs chromosomal locations are presented in FIG. 7 where arrowheads indicate DMR locations, and black boxes the DMR clusters. All chromosomes had DMRs for glyphosate versus control, but the prostate, kidney, obesity and multiple disease DMR biomarkers were not on the Y or mitochondrial DNA (MT). Therefore, the DMRs were genome-wide and identified on nearly all chromosomes. These DMR chromosomal signatures are potential sperm biomarkers for disease. The CpG density of the DMRs and the DMRs length are shown in FIG. 8. The CpG density of the DMRs for all the comparisons was 1-5 CpG per 100 bp being predominant (see FIG. 8). This is characteristic of a low-density CpG deserts which was previously reported with other transgenerational DMRs. The length of the DMRs for each disease biomarker and glyphosate versus control were 1-4 kb with 1 kb length being predominant (see FIG. 8). Generally, the DMRs are 1 kb in size with around 10 CpGs. A principal component analysis (PCA) for the different DMR genomic site comparisons (glyphosate versus control, prostate disease biomarker, kidney disease biomarker, obesity disease biomarker and multiple disease biomarker) demonstrated clustered DMR principal component separation of the control versus glyphosate, and the prostate, kidney, obesity and multiple diseased versus non-diseased (see FIG. 9).

### Sperm Histone Retention Analysis

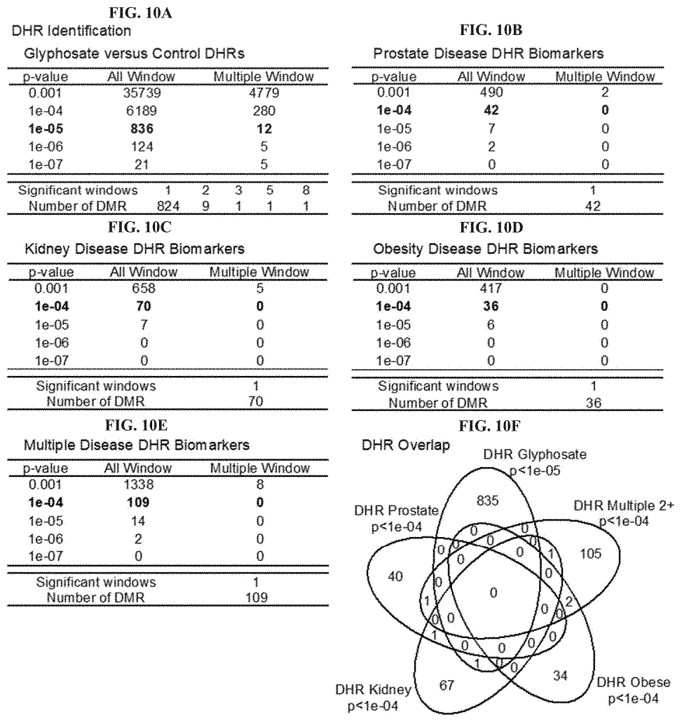

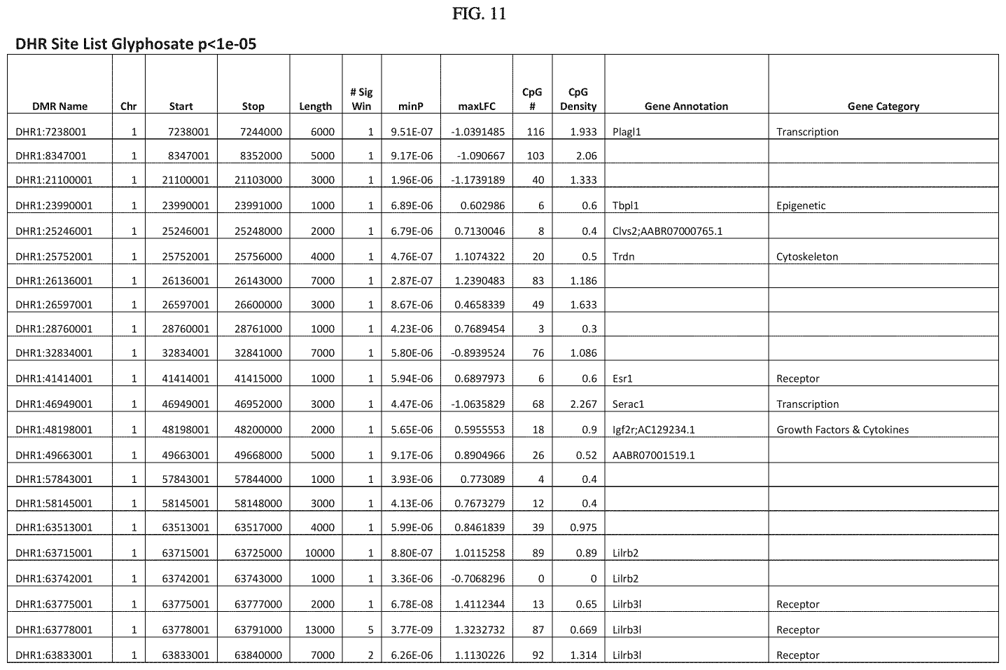

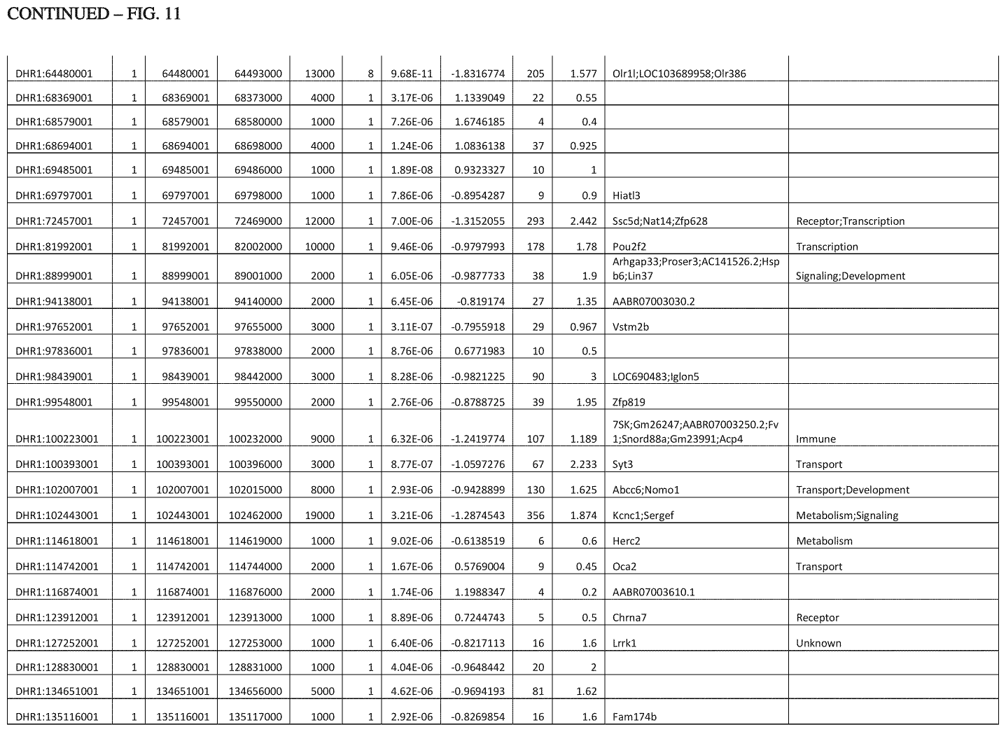

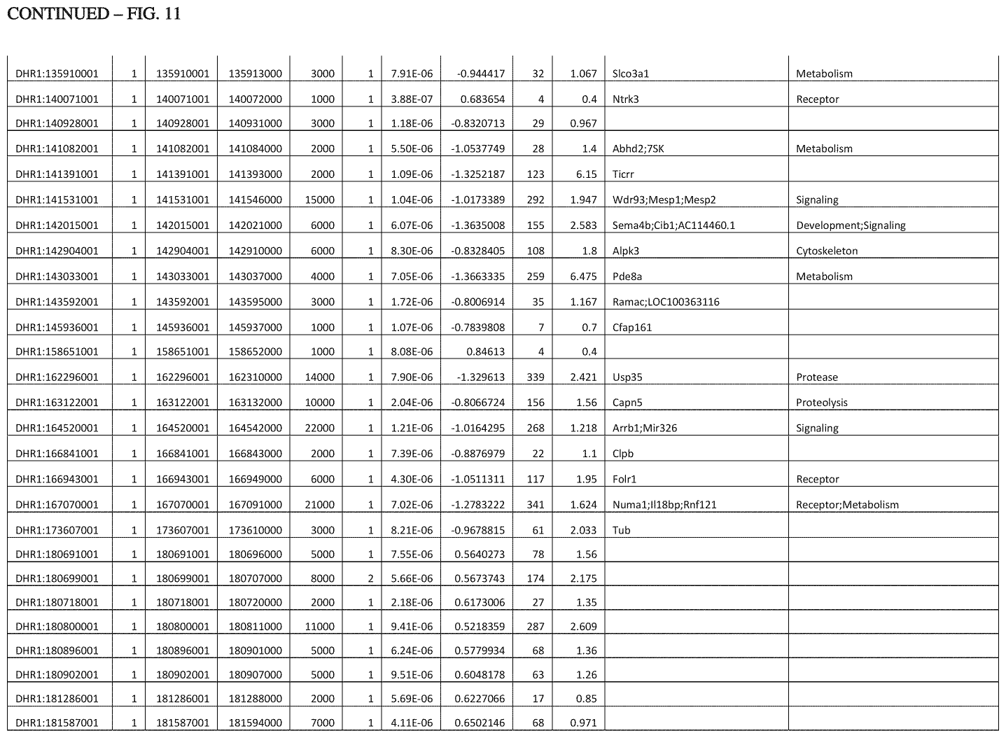

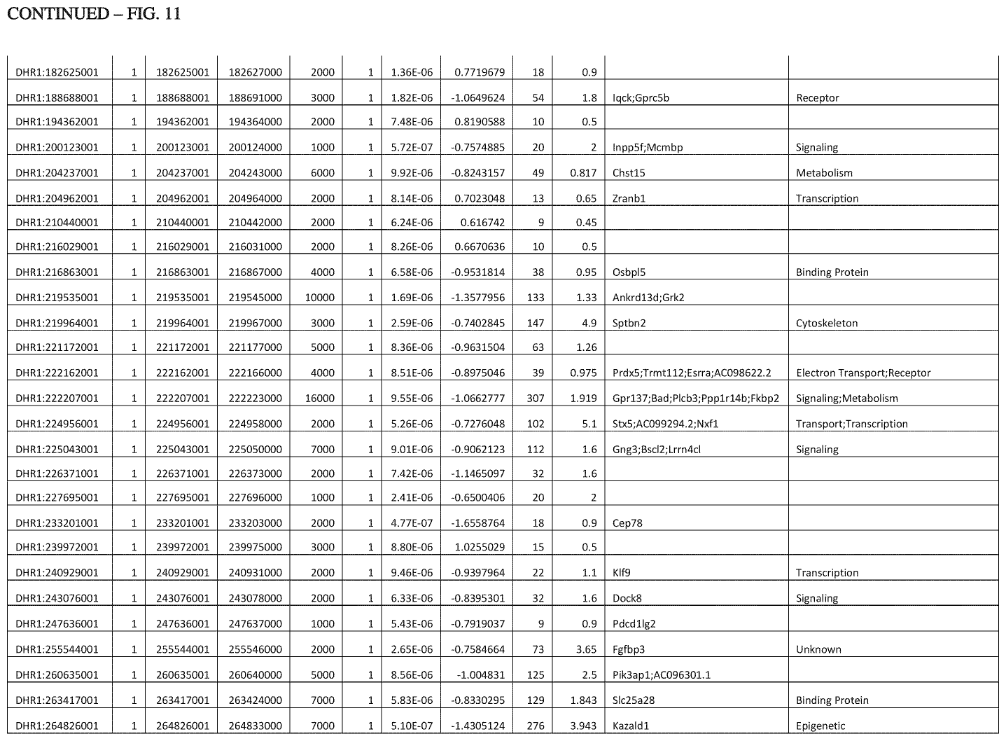

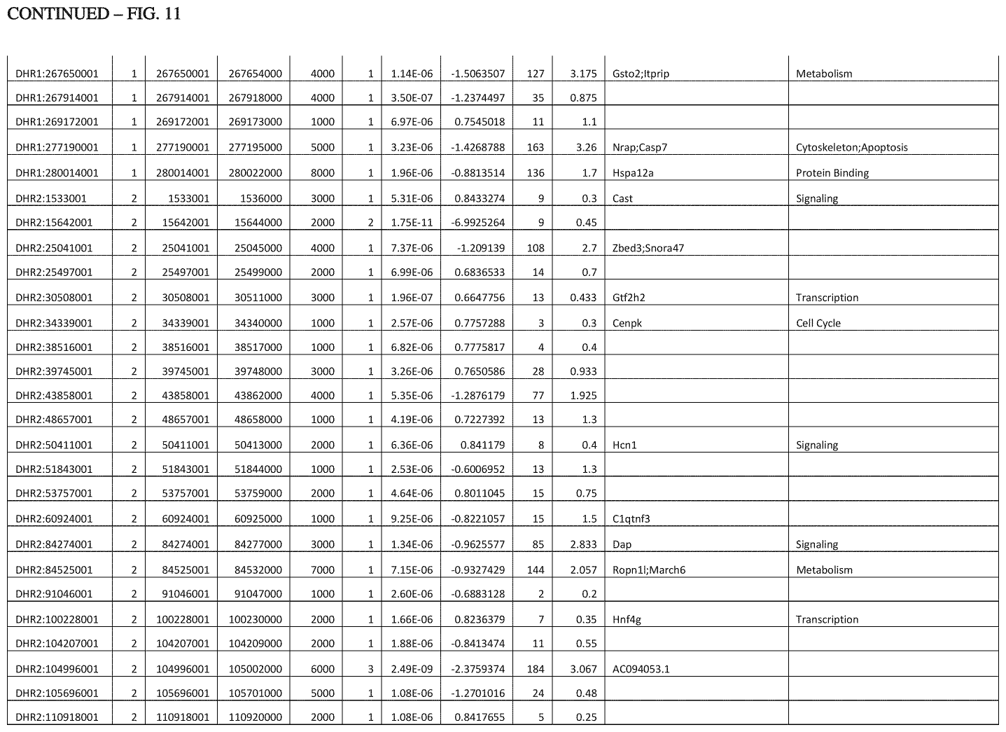

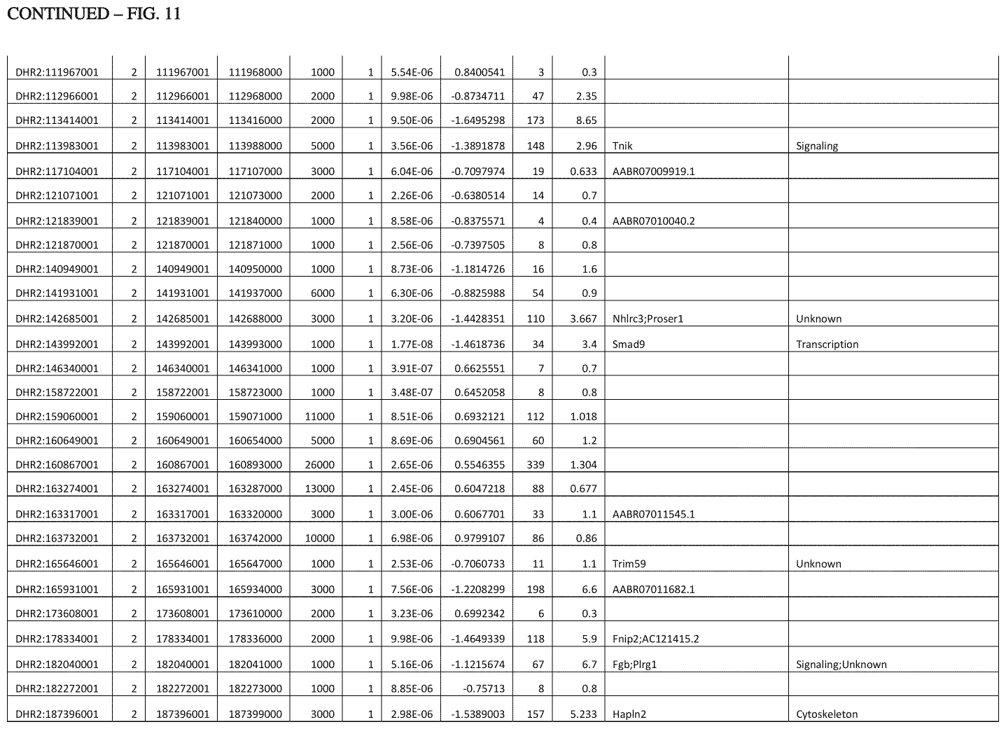

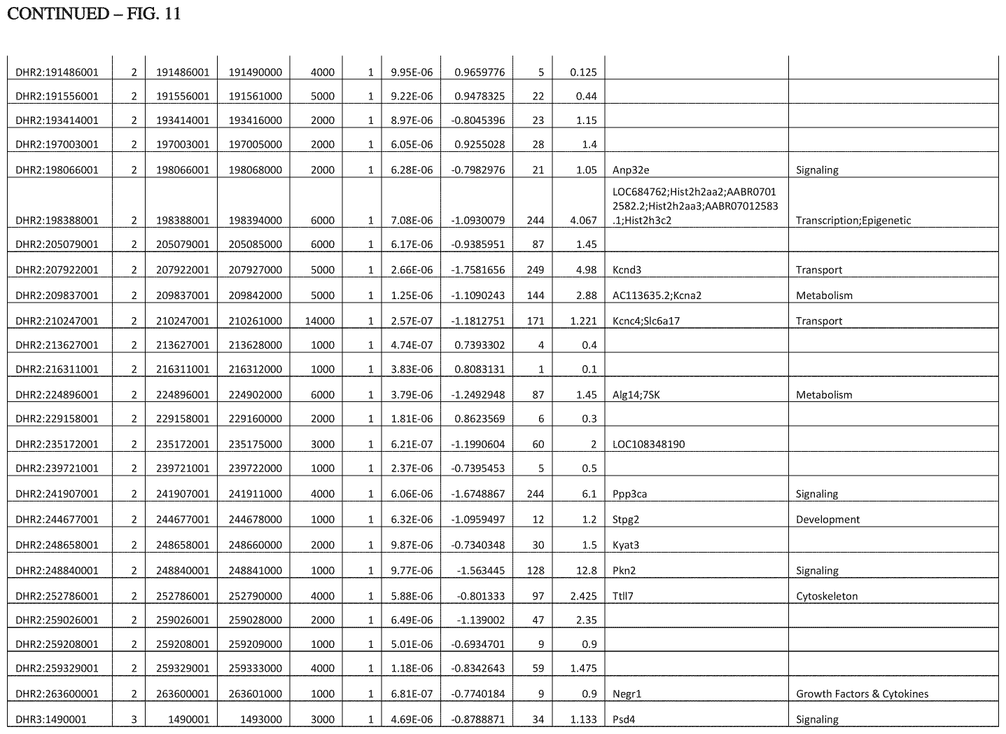

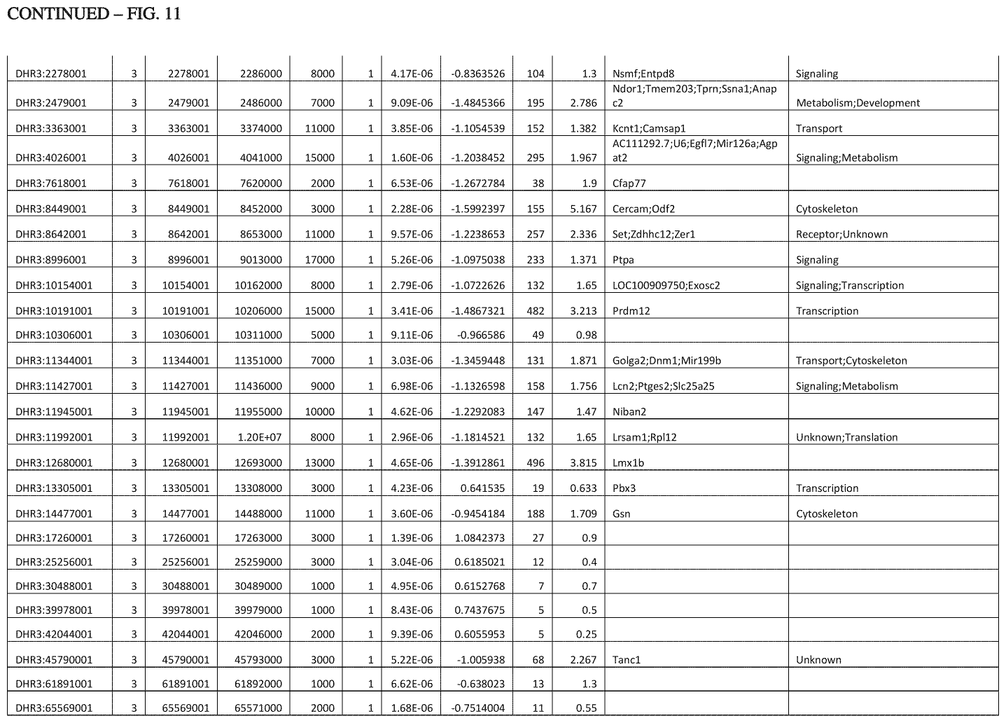

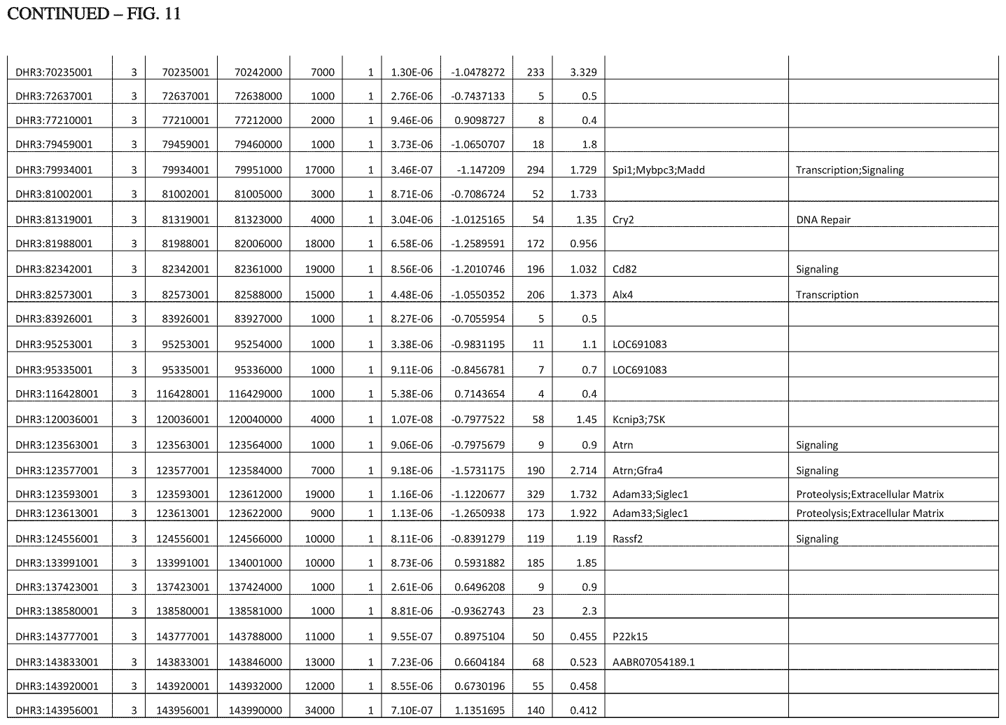

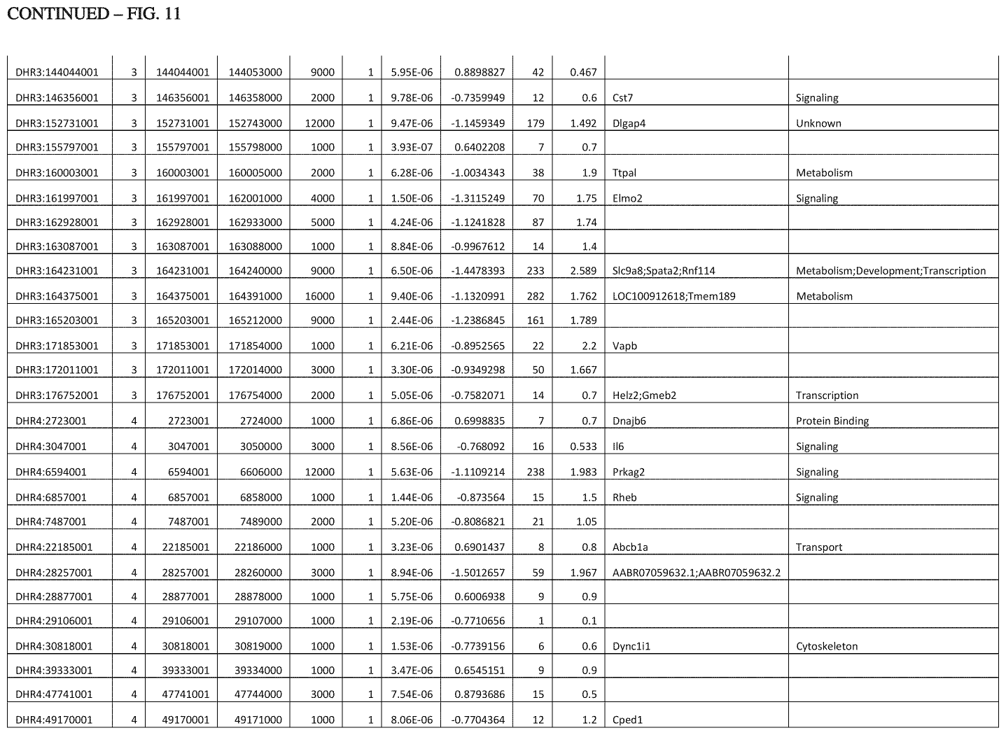

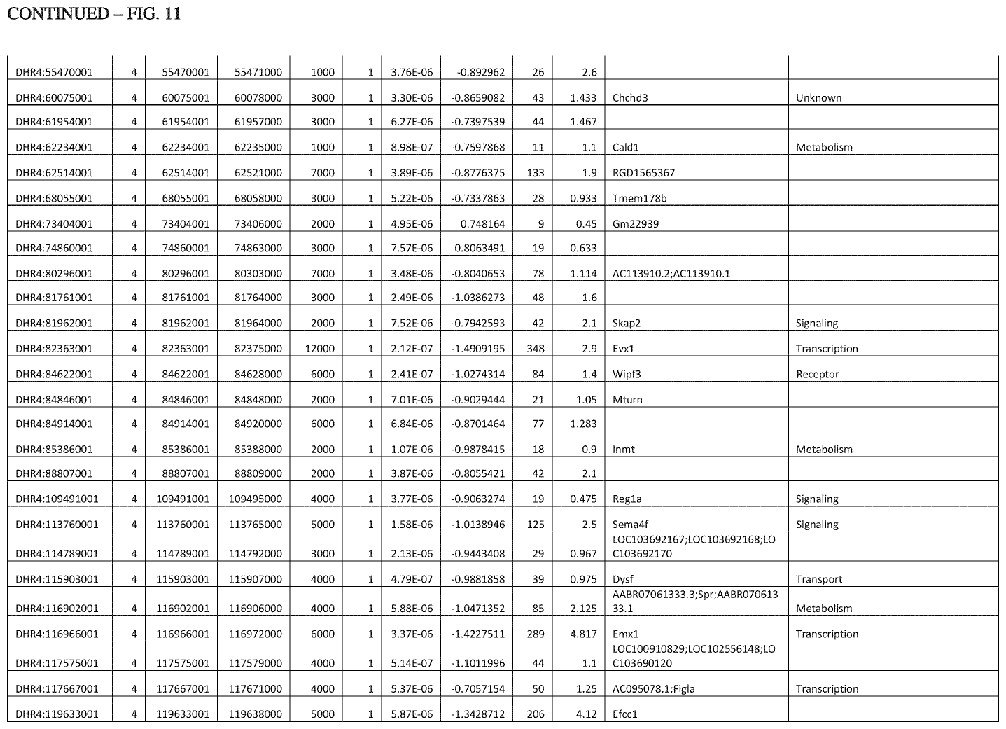

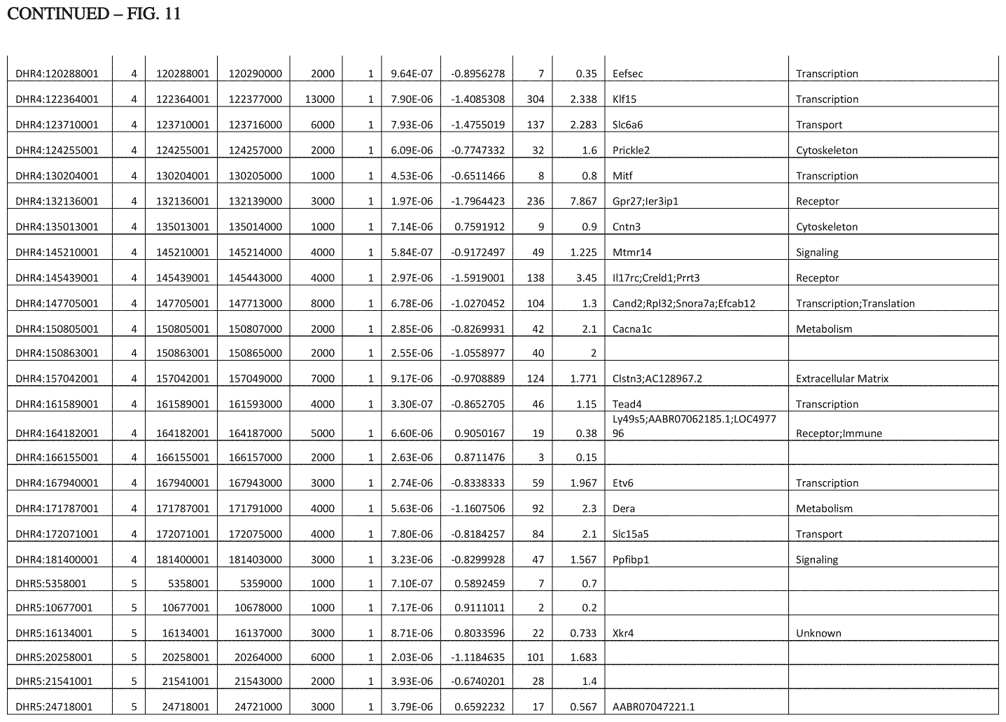

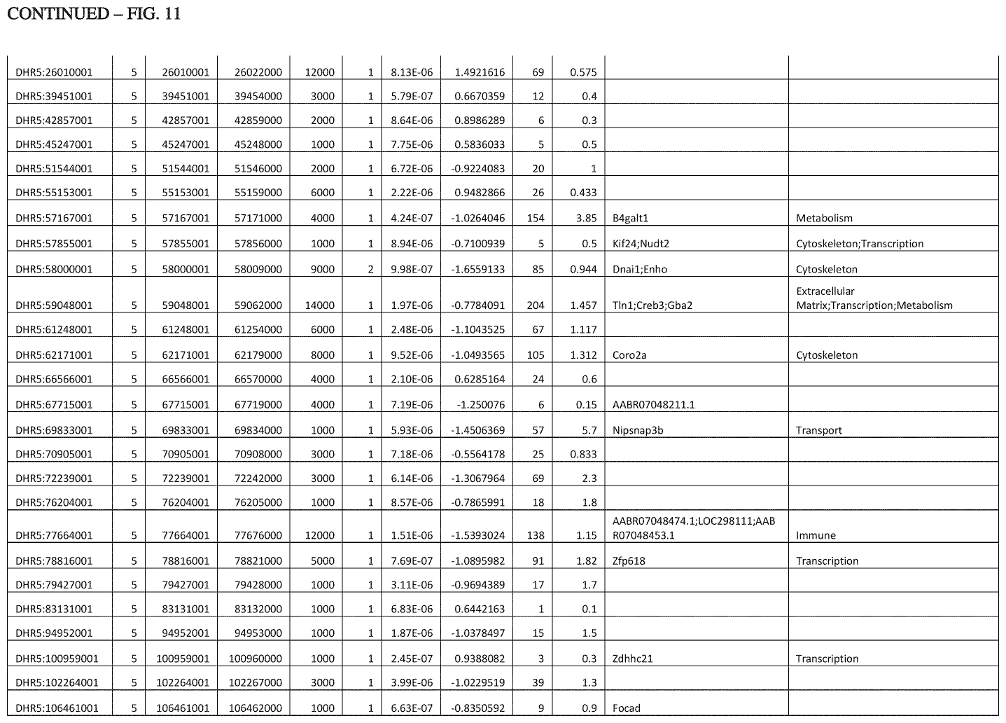

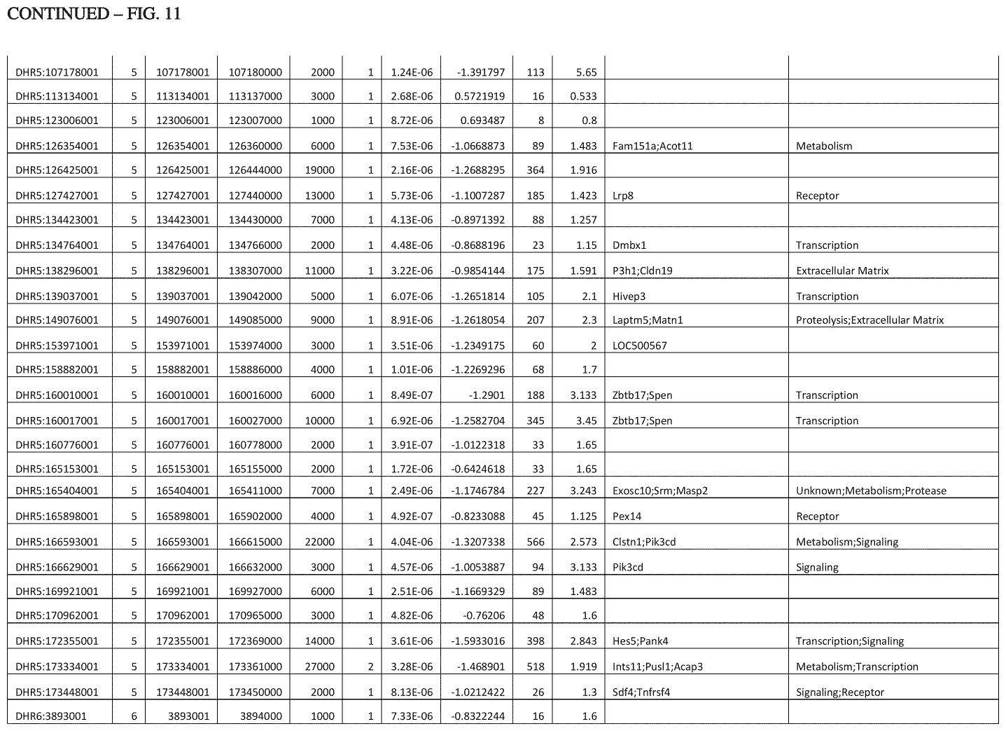

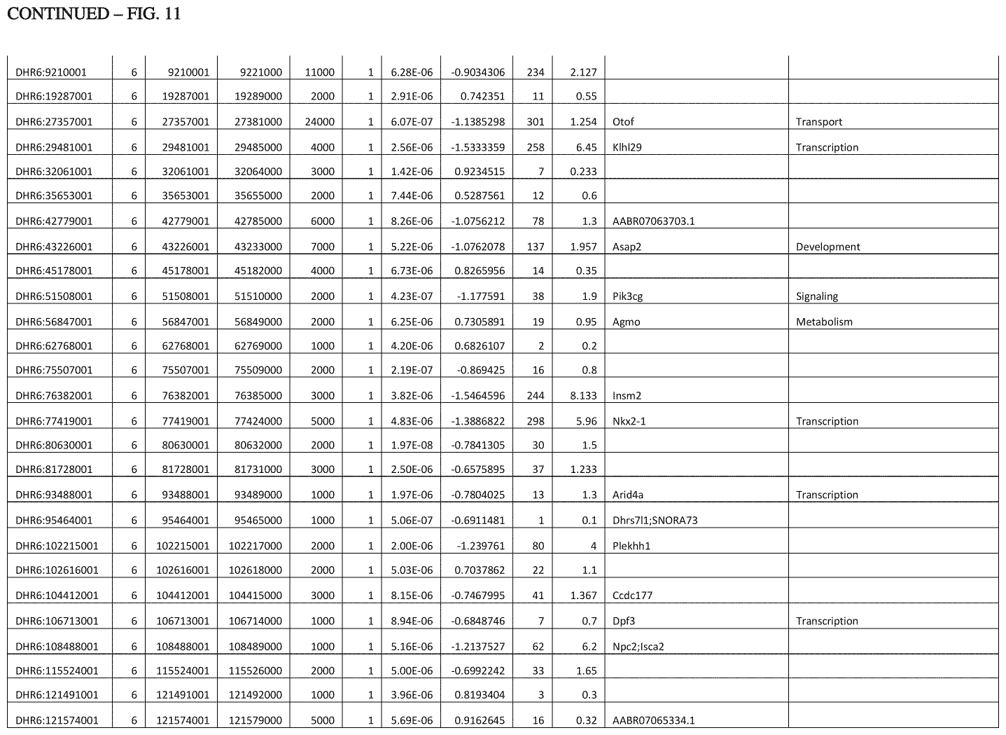

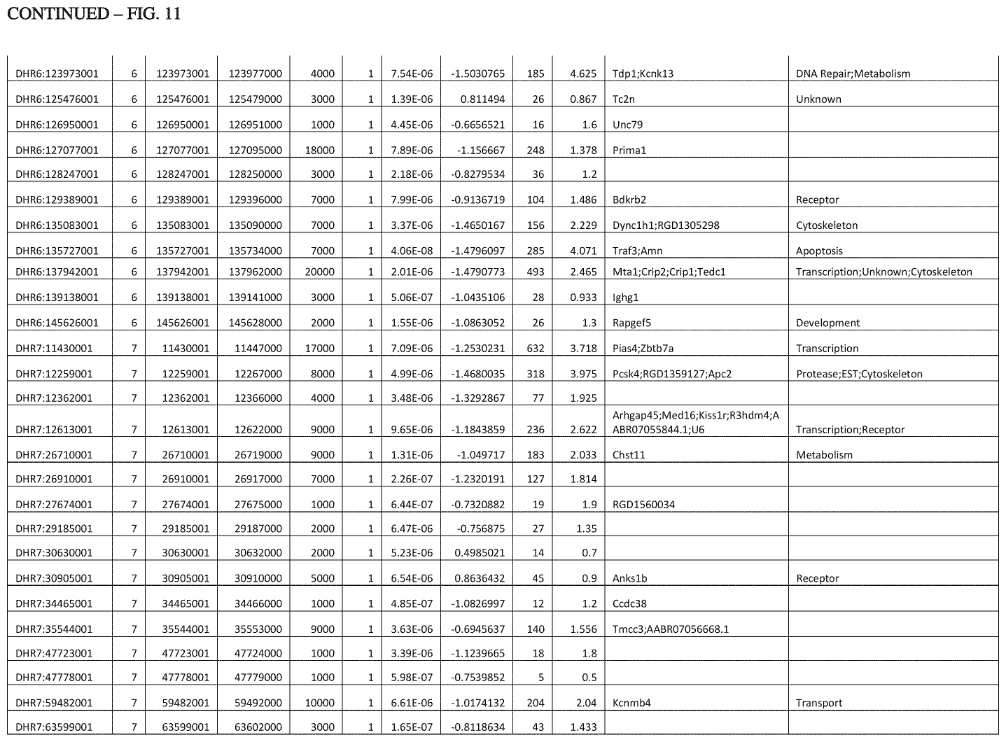

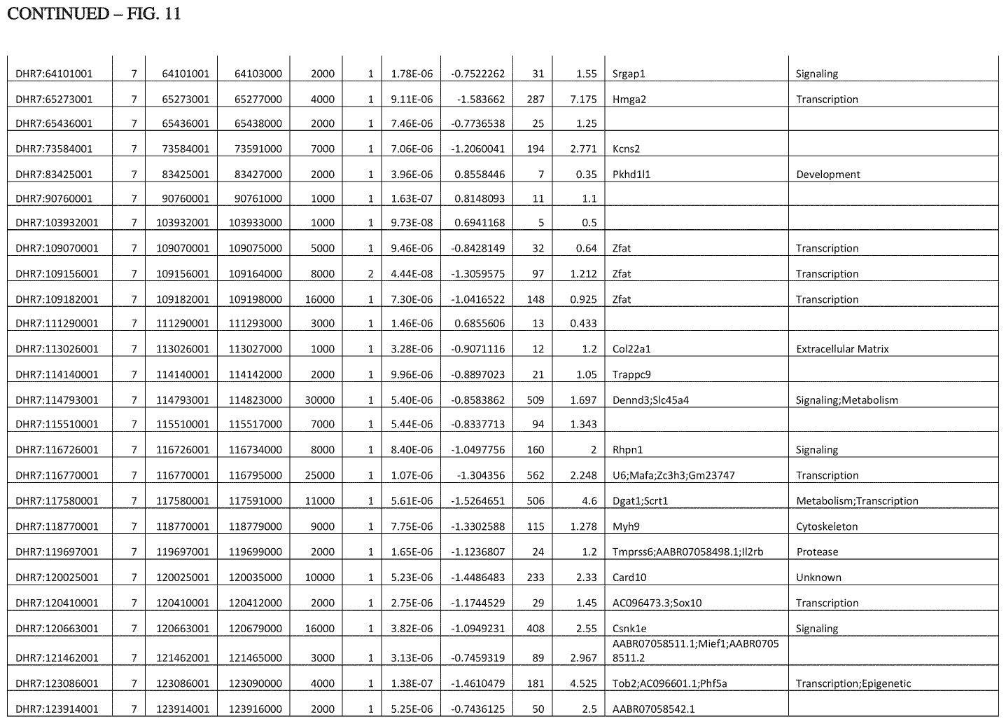

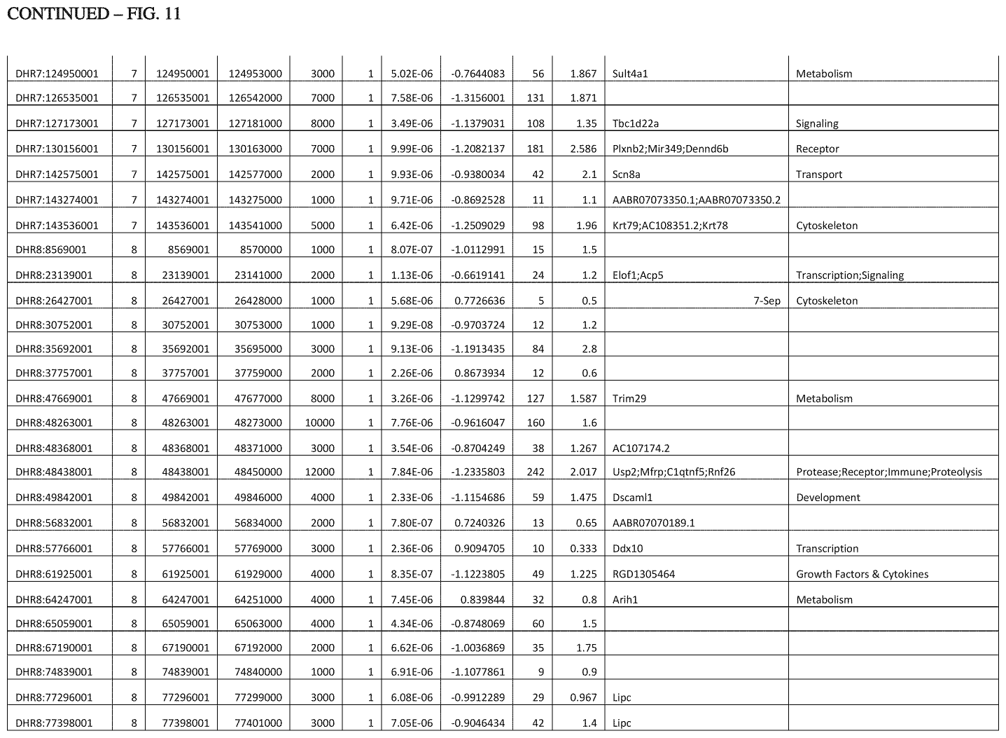

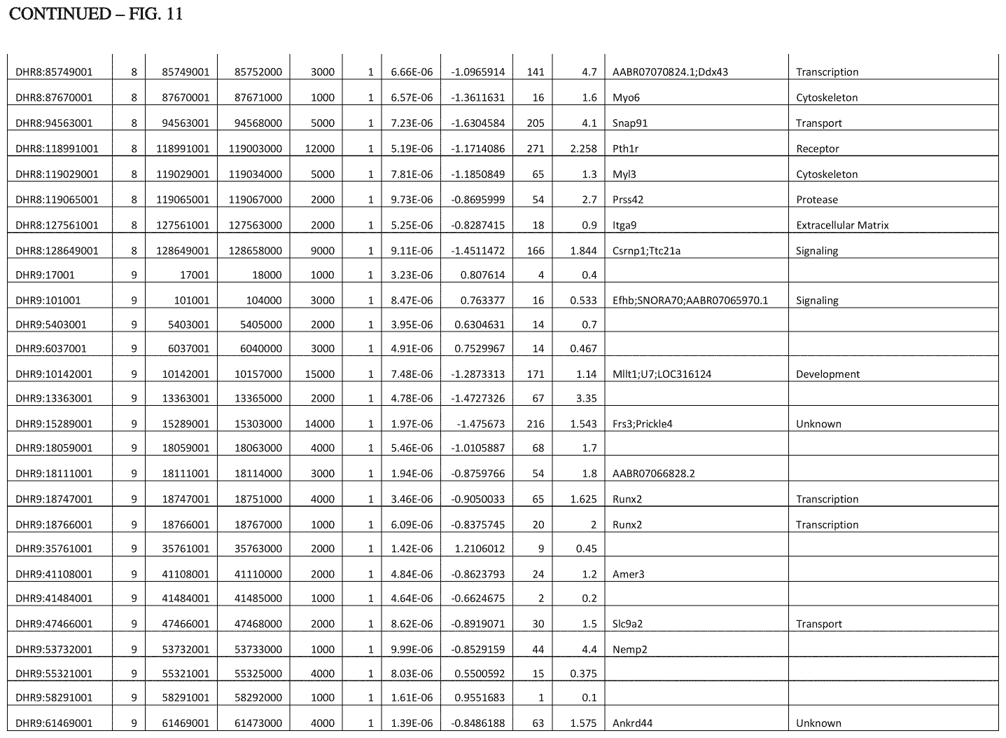

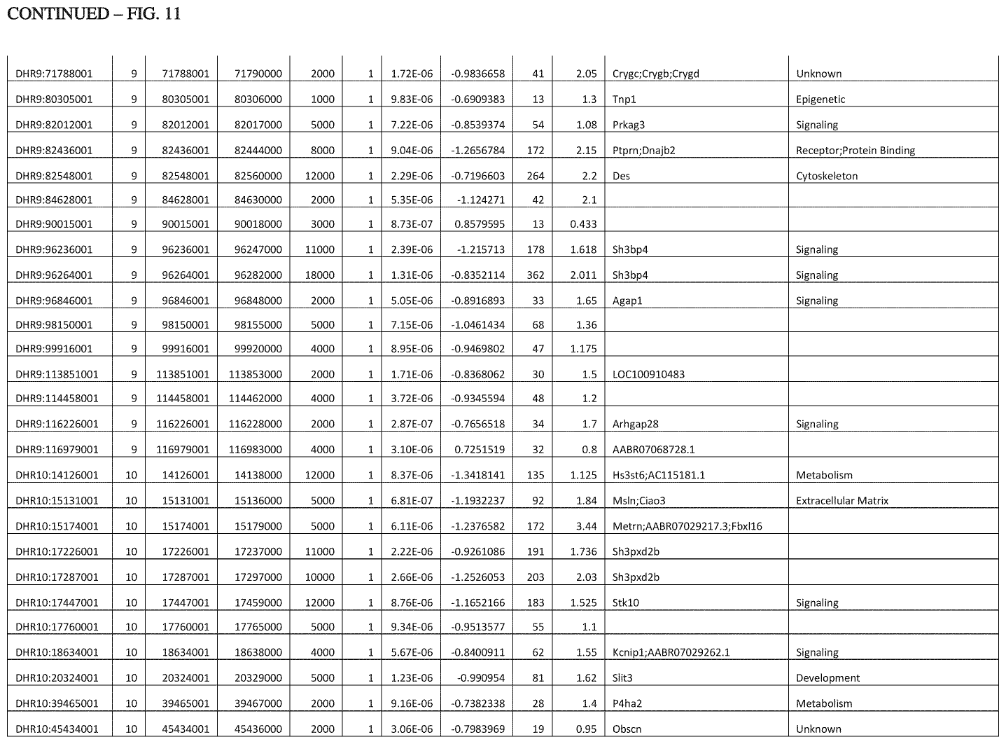

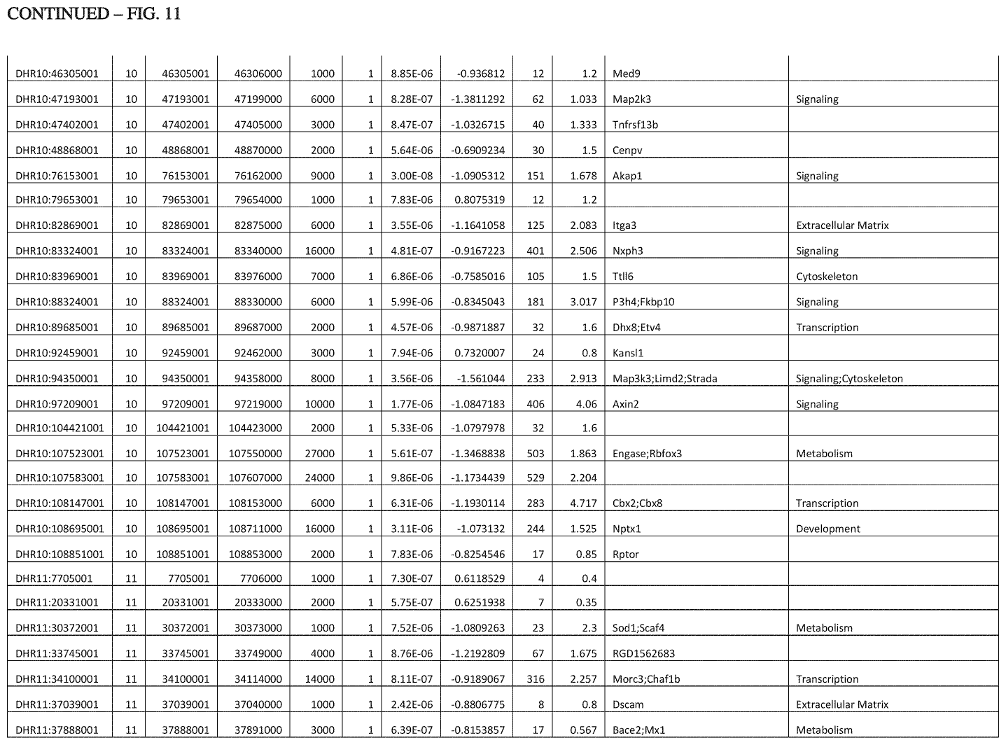

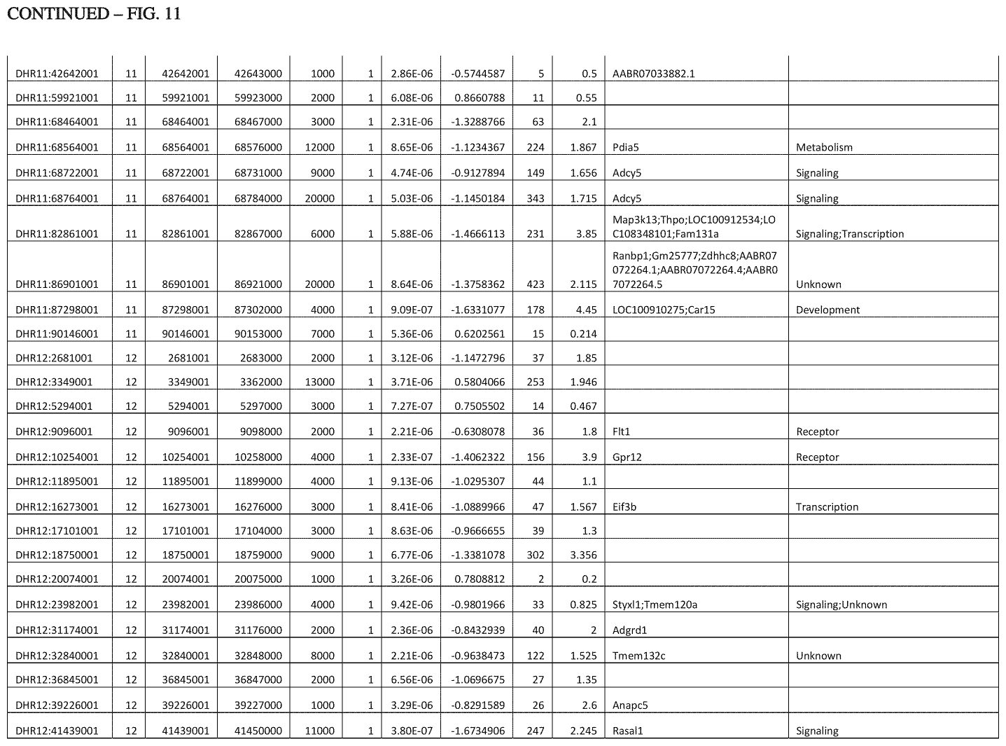

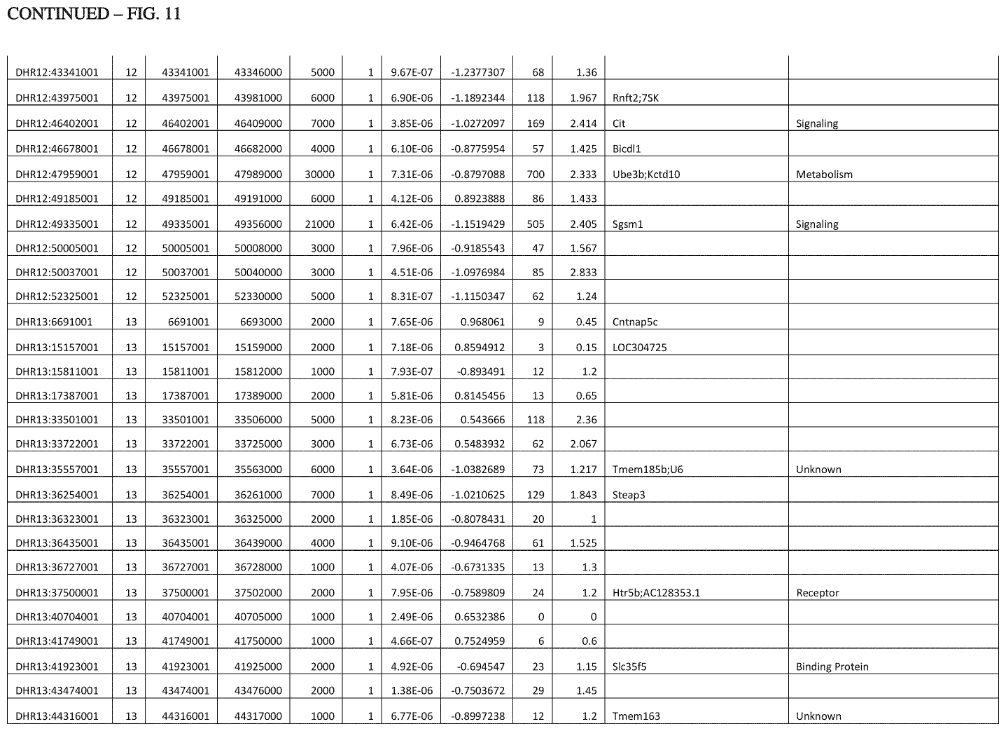

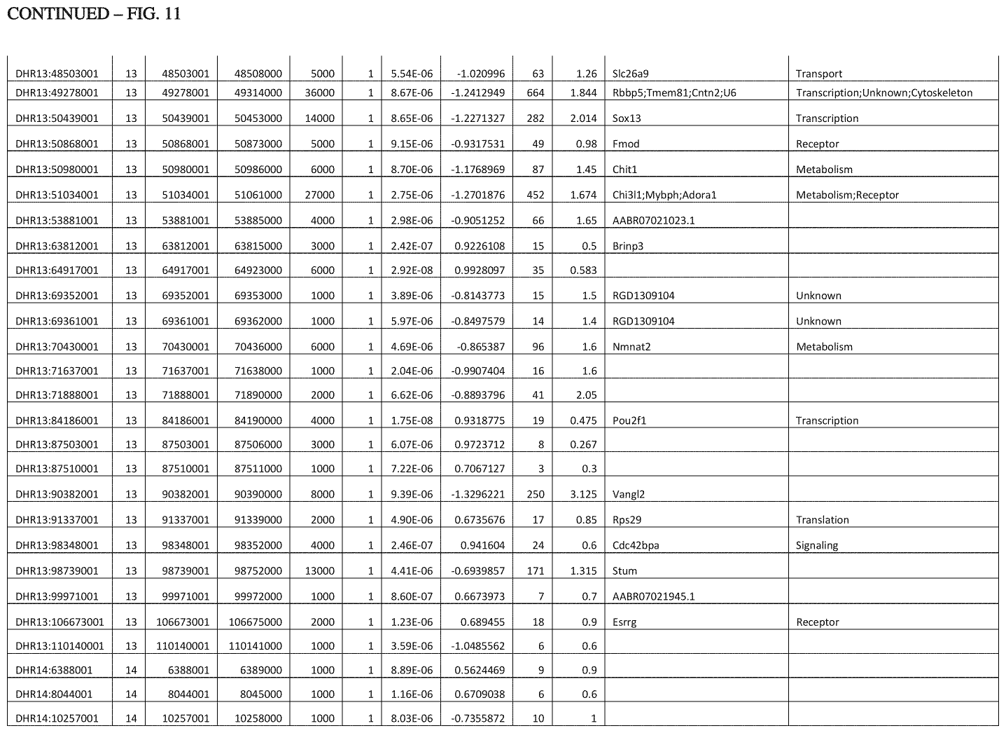

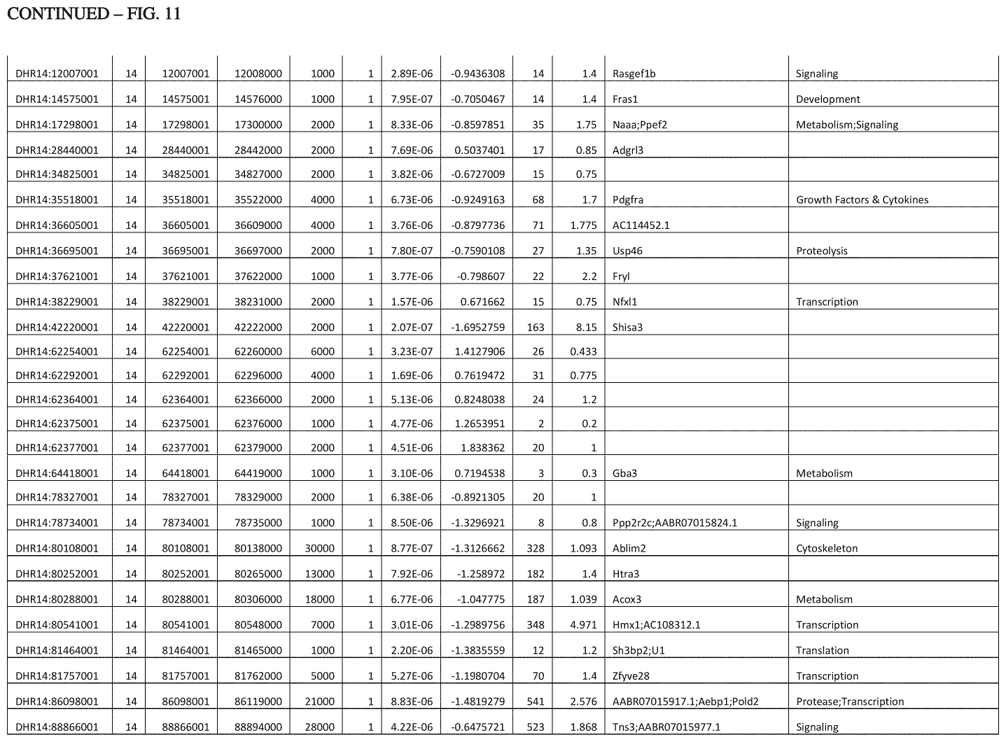

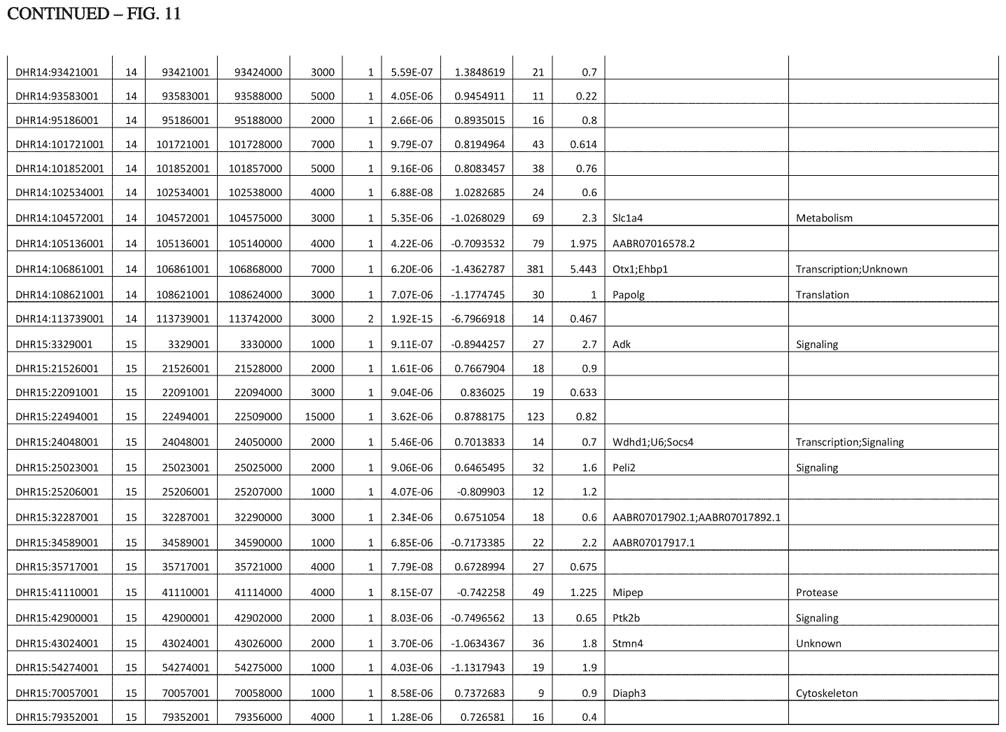

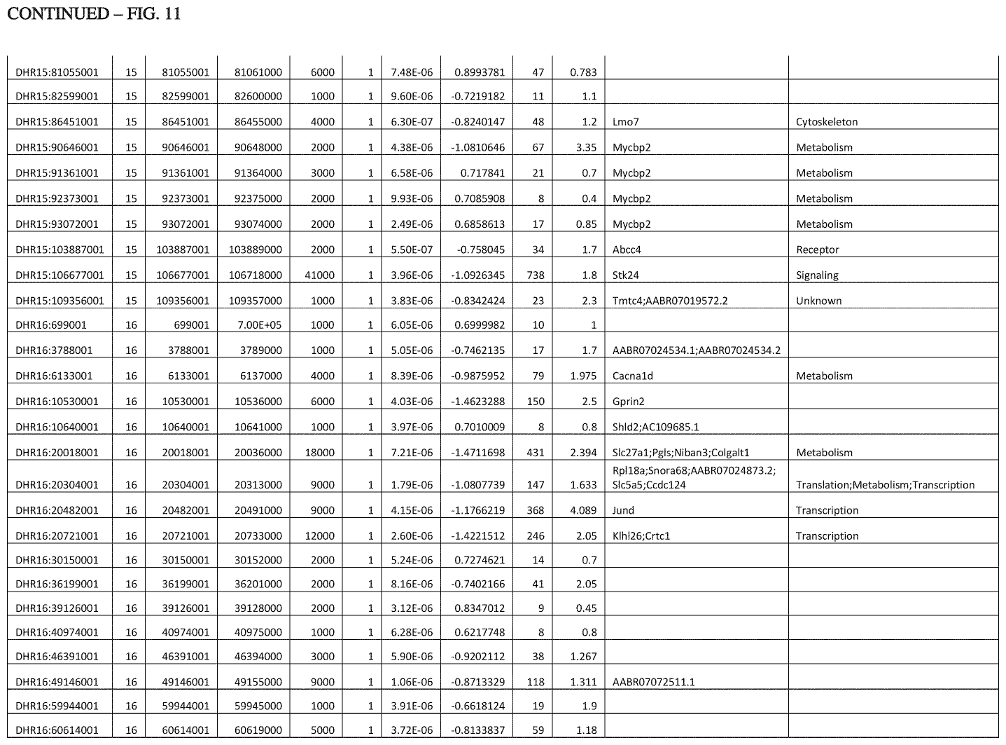

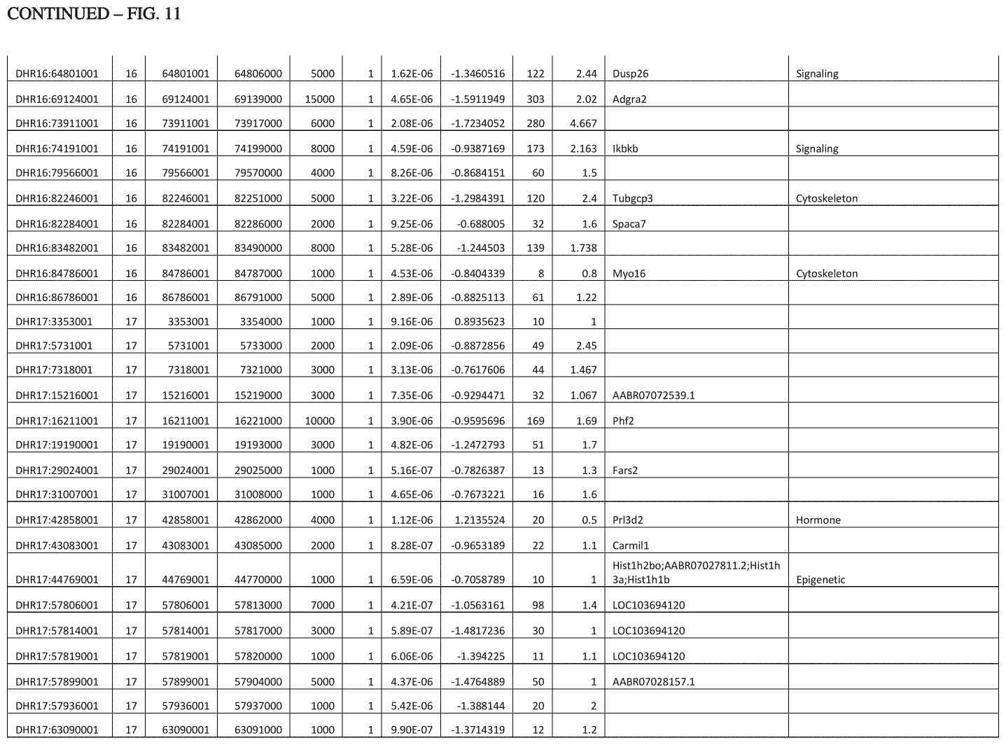

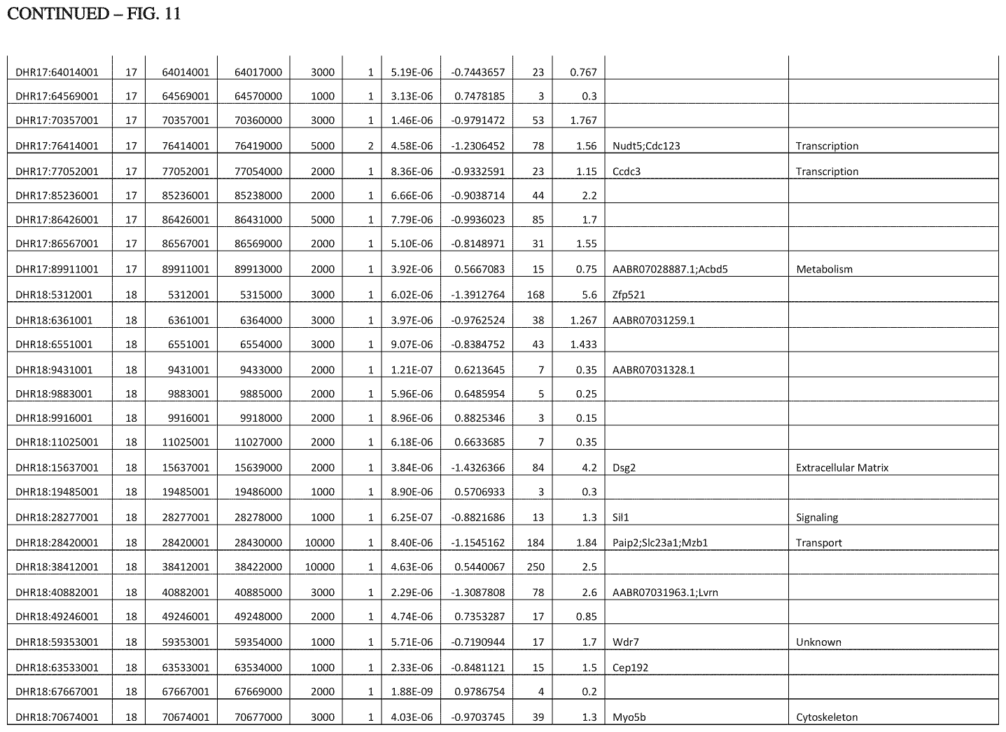

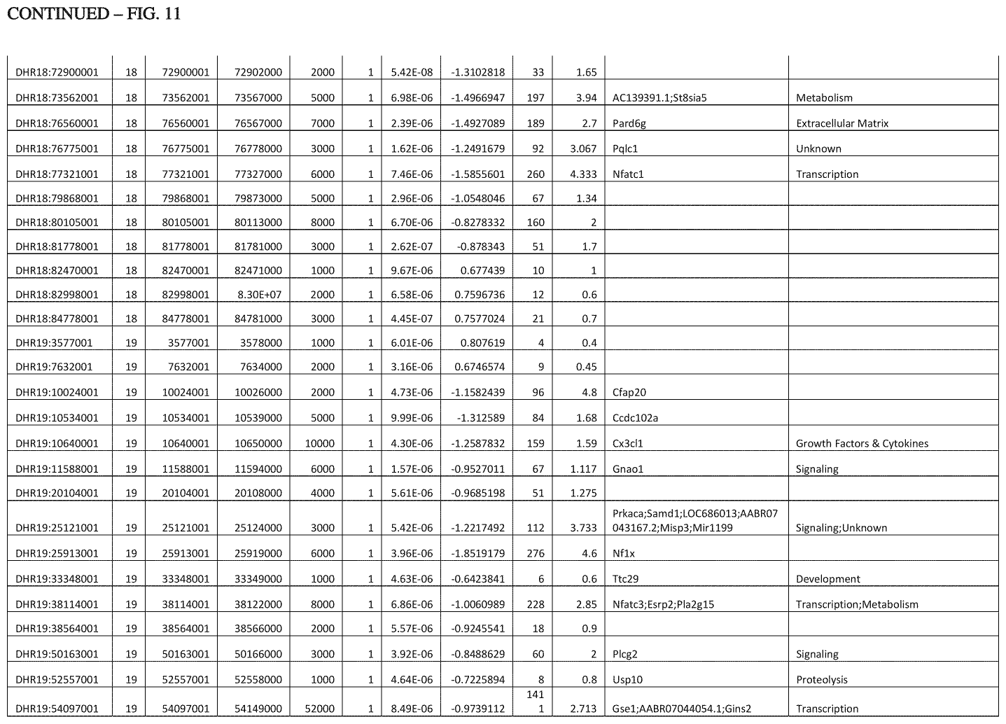

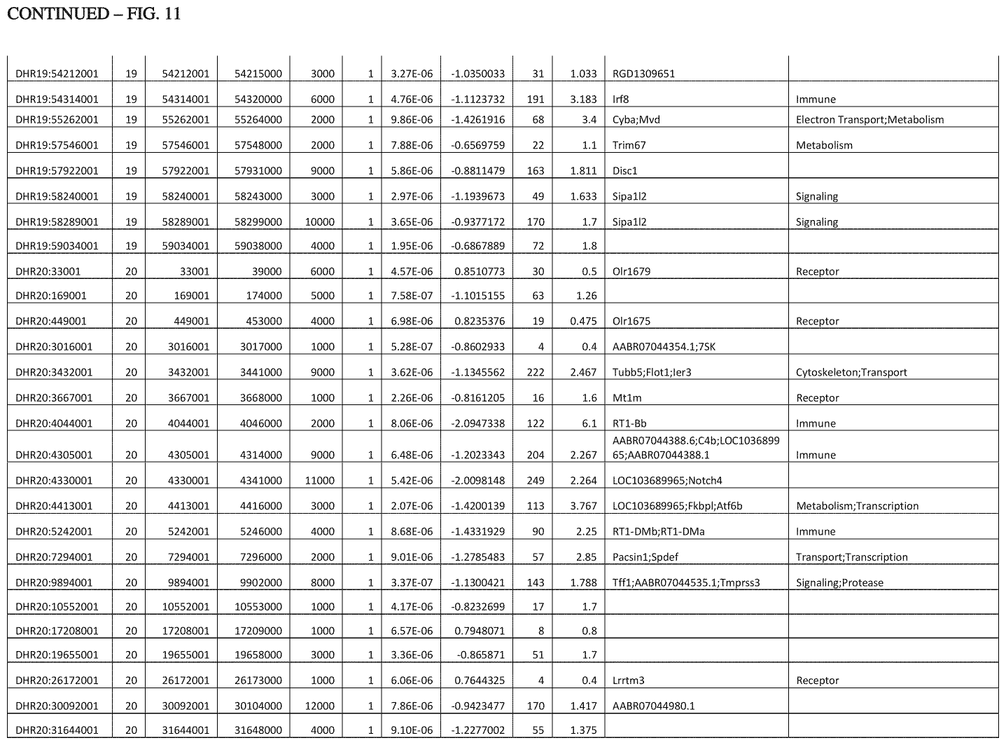

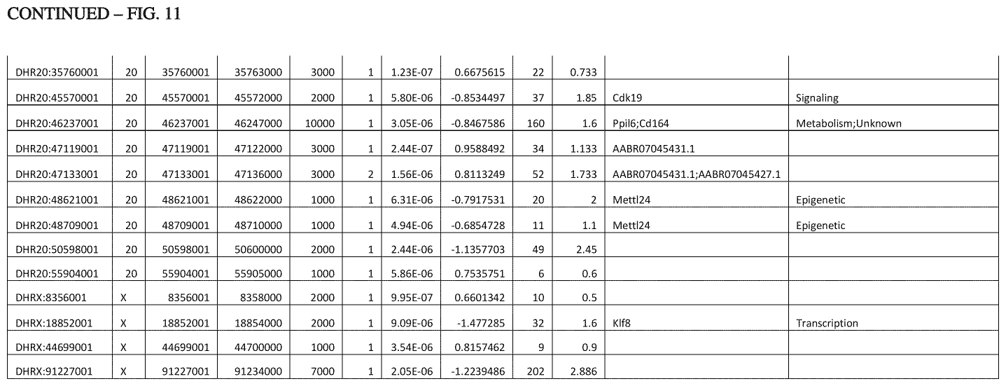

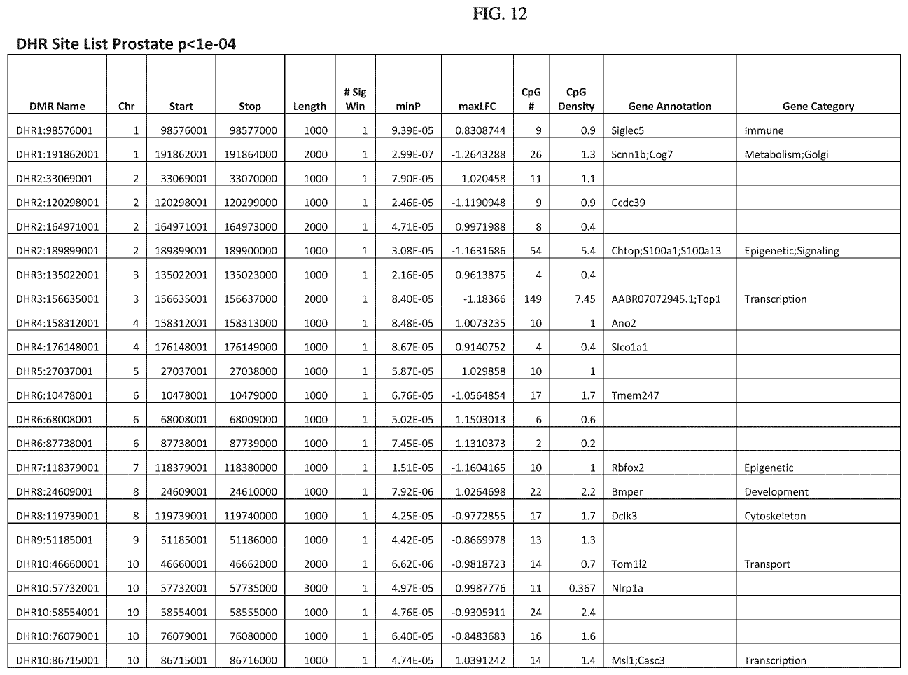

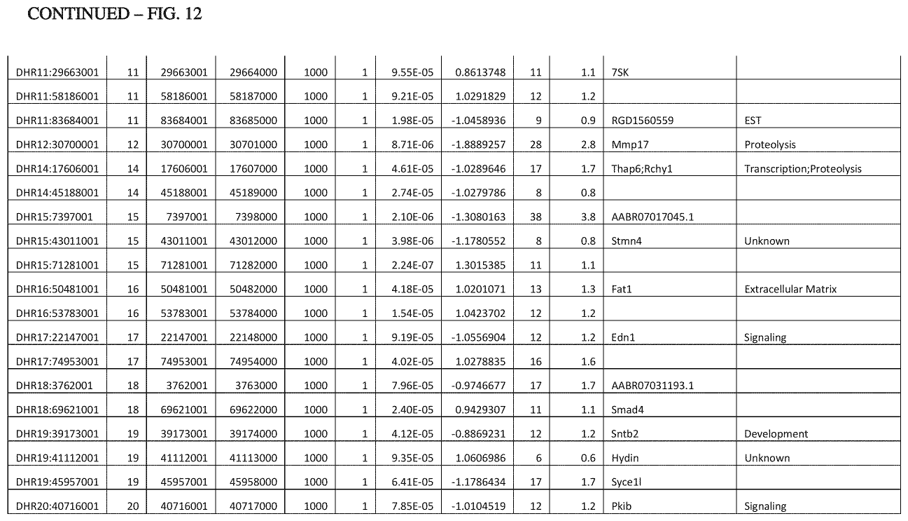

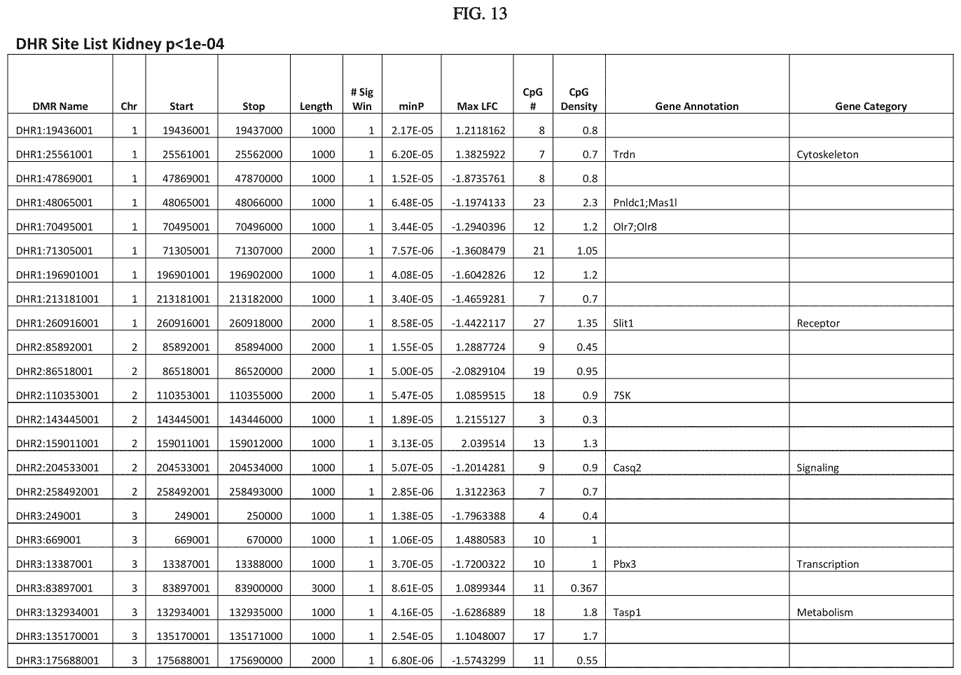

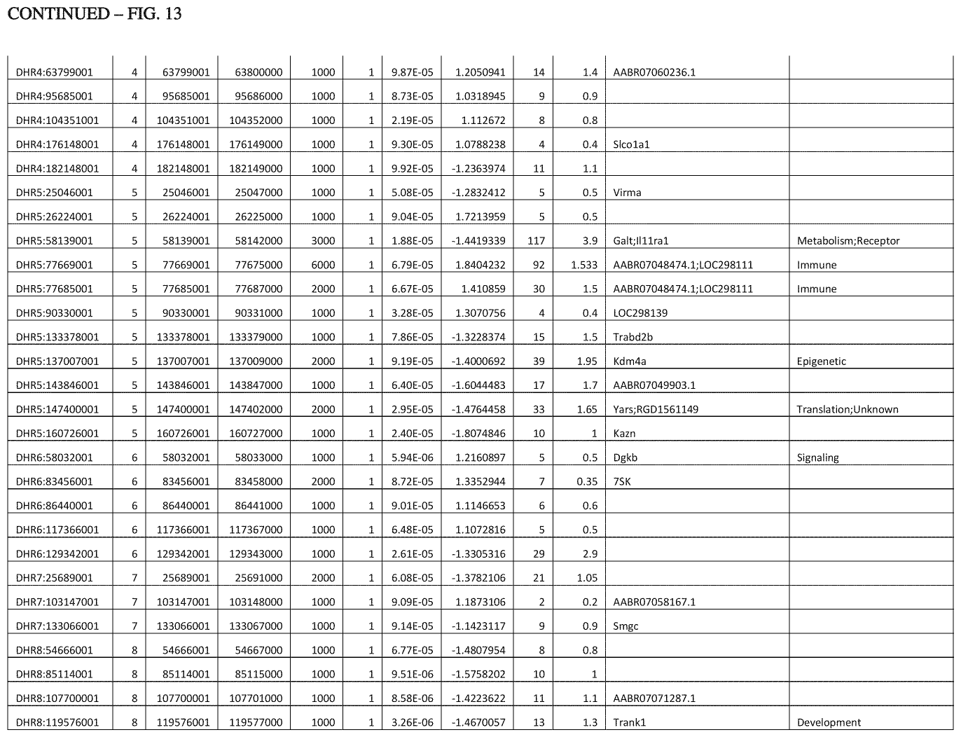

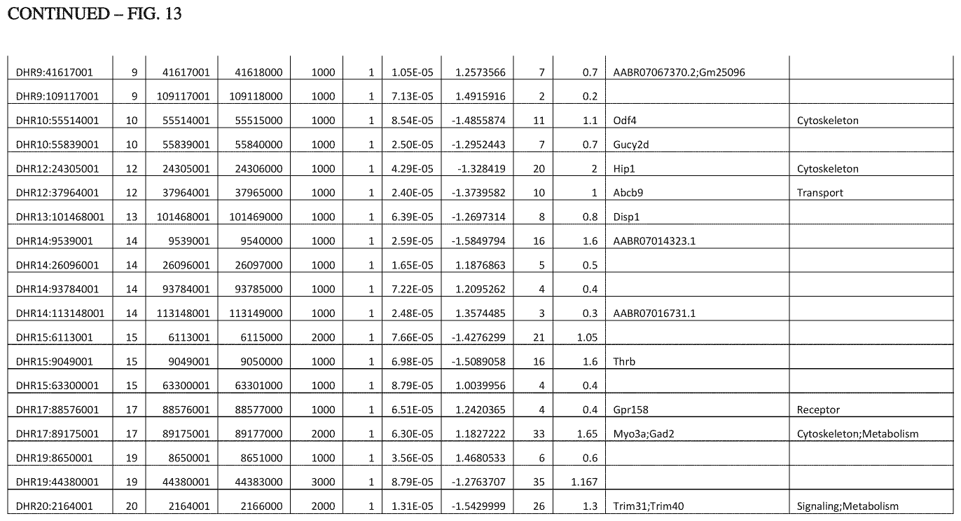

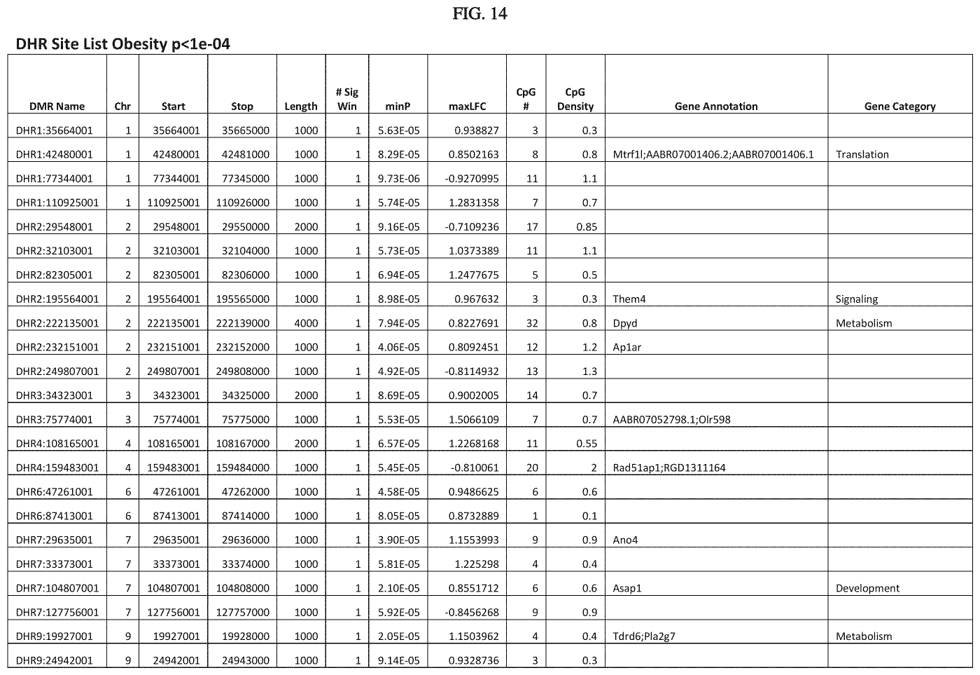

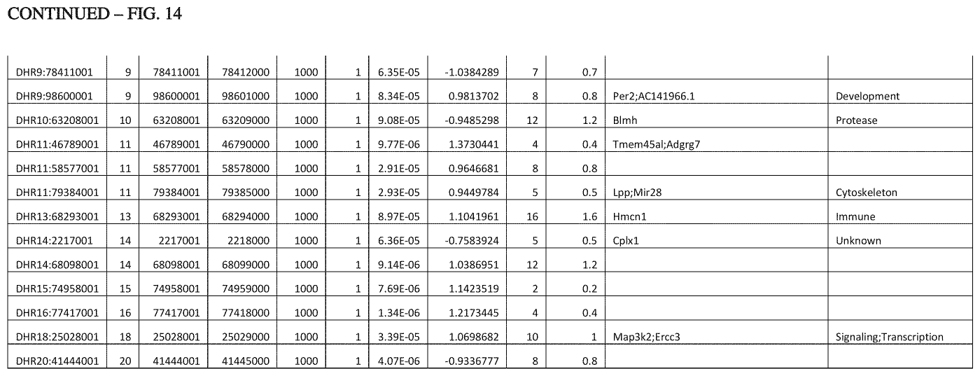

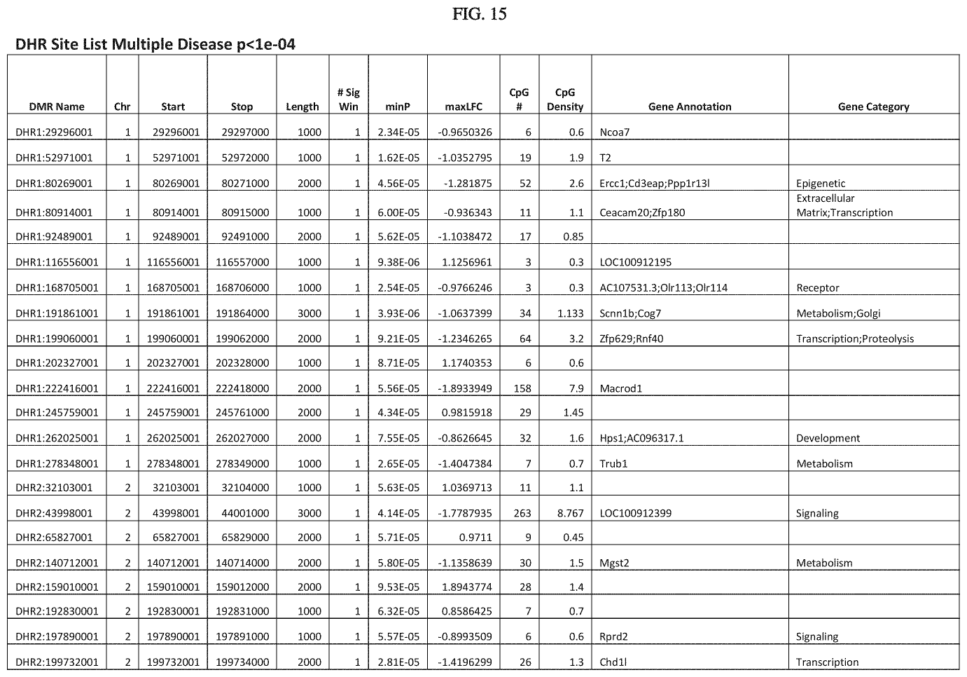

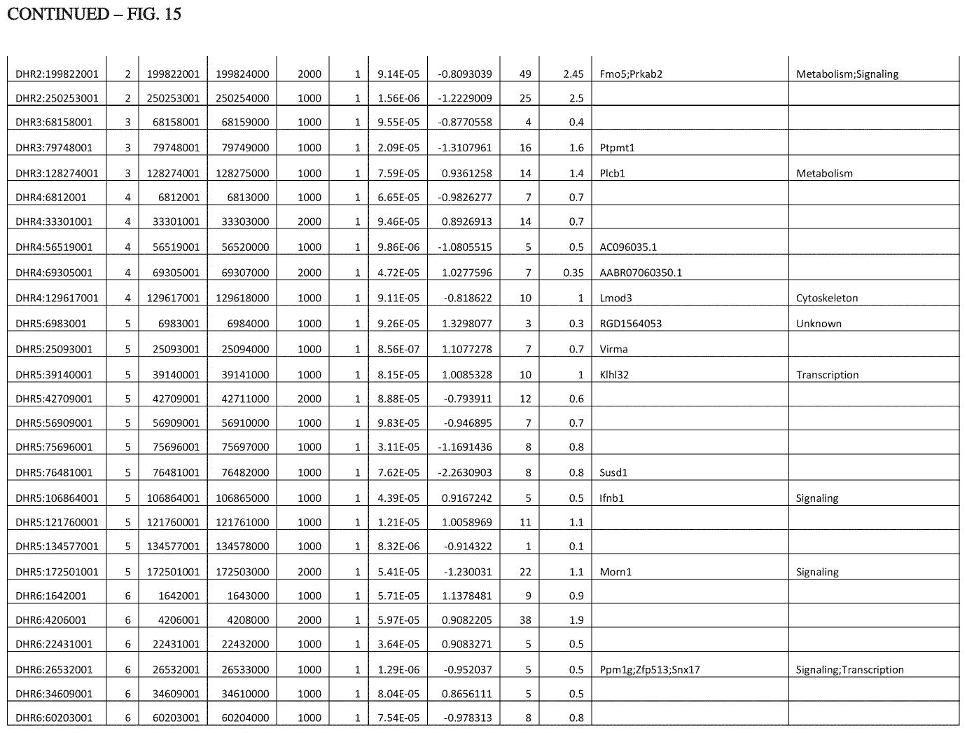

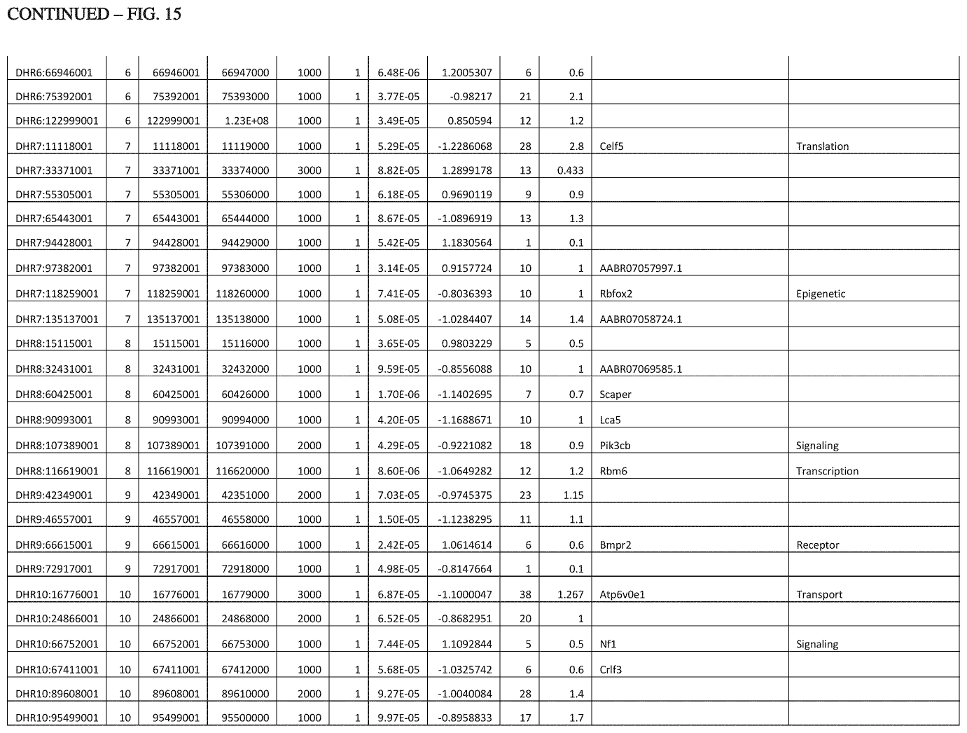

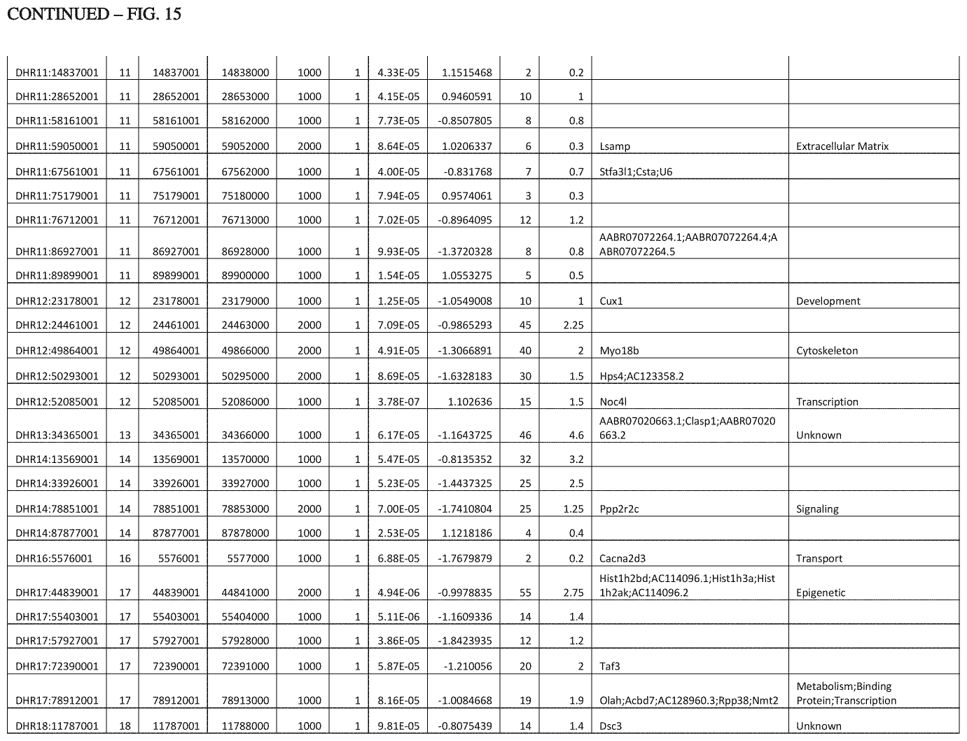

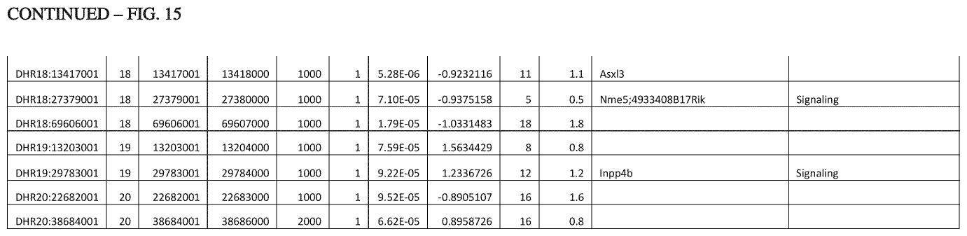

Differential histone retention in sperm also appears to have a role in epigenetic transgenerational inheritance. Similarly to the DMRs, the differential histone retention regions (DHRs) in the sperm were identified using a comparison between the control and the glyphosate lineage (FIG. 10A and FIG. 11). Within the same glyphosate lineage animals with a given disease (prostate, kidney, obesity, multiple disease 2)) versus non-diseased animals were assessed to identify DHRs (FIG. 10B-10E and FIGS. 11-15).

Unexpectedly, a high number of DHRs (836) was found at edgeR p<1e−05 in the glyphosate versus control comparison (see FIG. 10A). A smaller number of DHRs were detected at p<1e−04 in the disease biomarkers (prostate, kidney, obesity and multiple diseases), (see FIGS. 10B-10E). Similar to the DMR analysis, an overlap of these disease specific DHRs at edgeR p<1e−04 revealed minimal overlap, (see FIG. 10F).

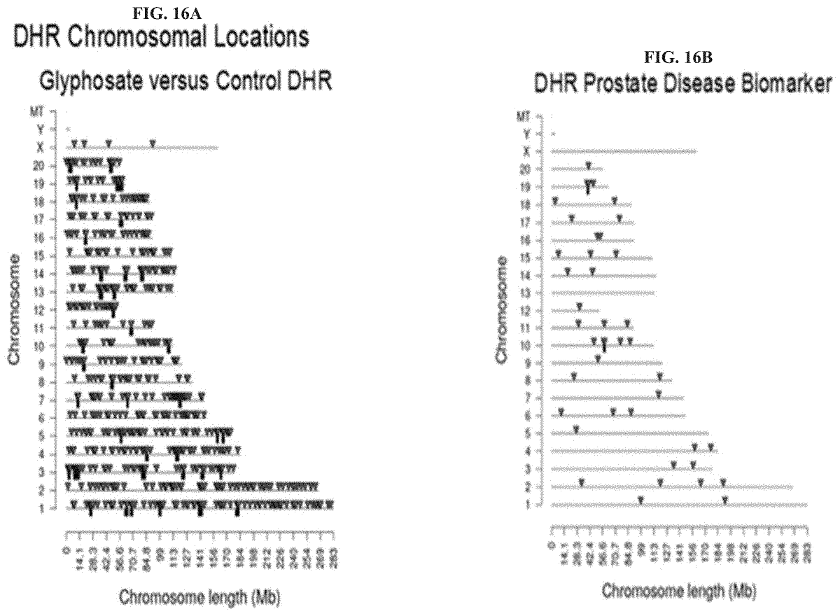

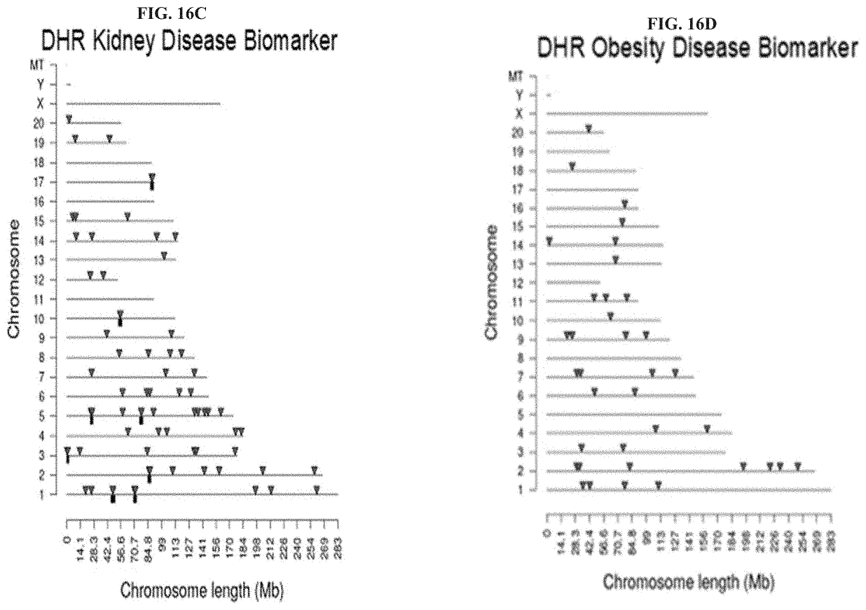

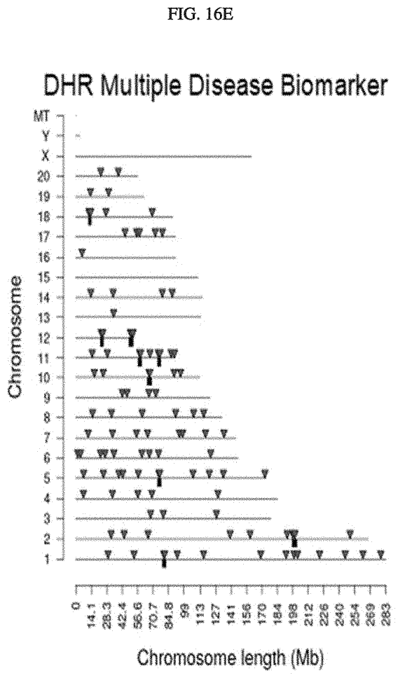

The chromosomal locations of these DHRs are presented in FIG. 16. The different DHRs appear to be genome-wide for the glyphosate versus control comparison, but are more specific over the genome for prostate, kidney, obesity and multiple disease.

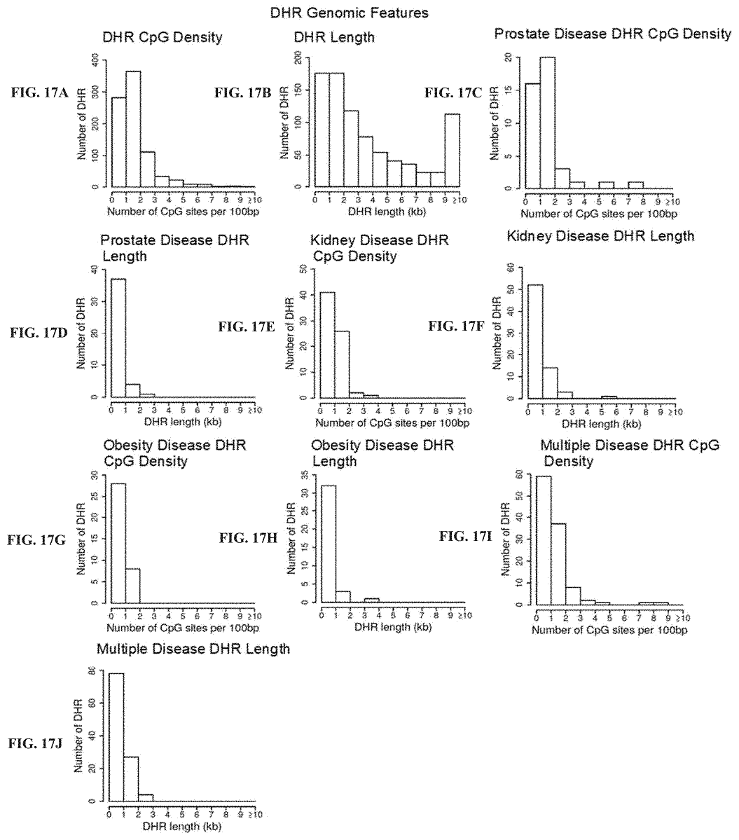

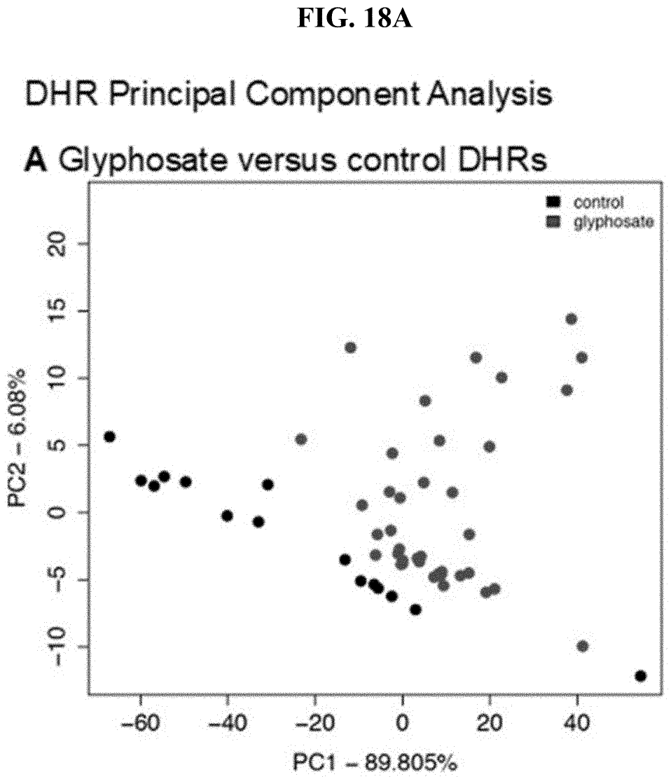

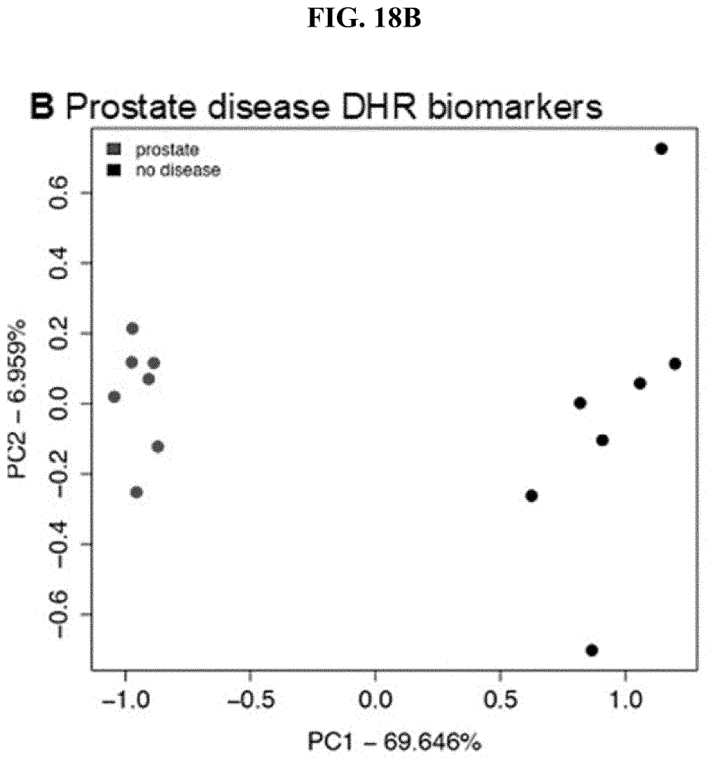

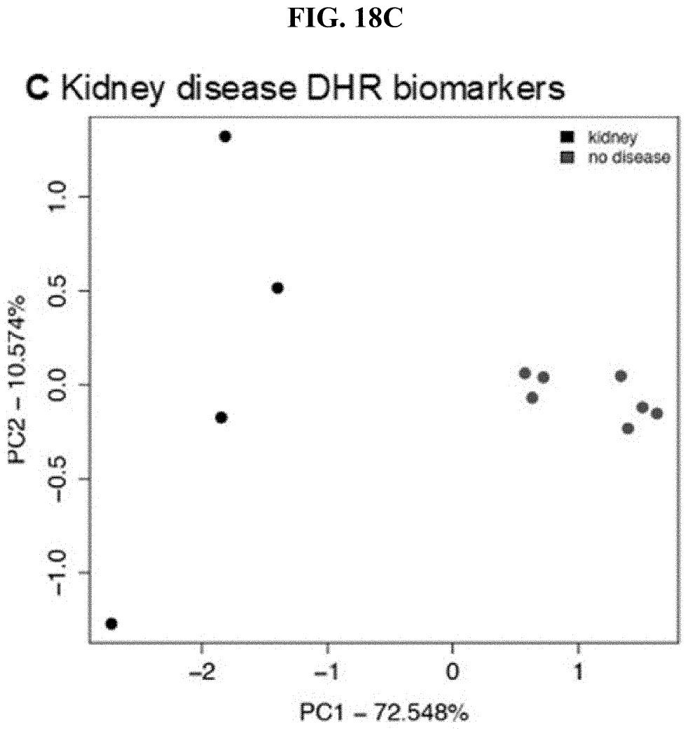

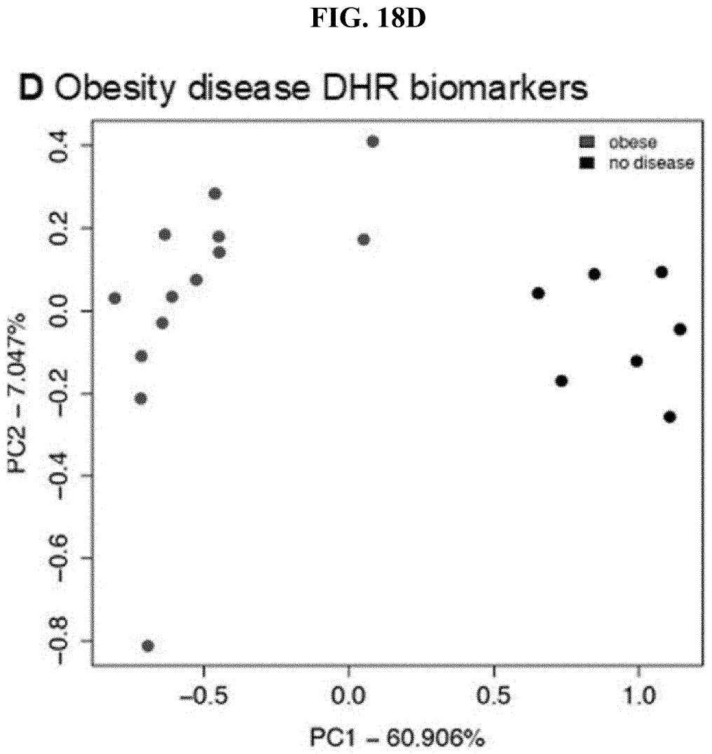

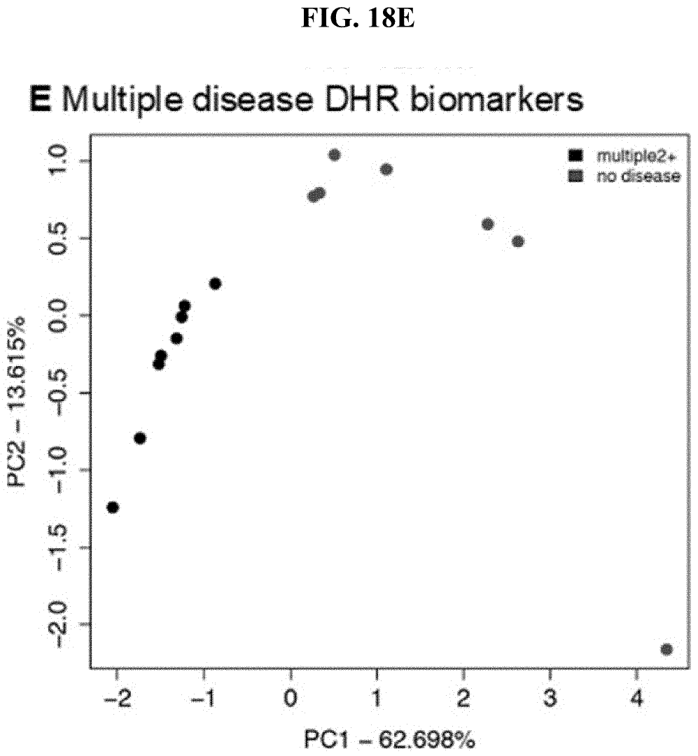

The DHRs CpG density and length of DHRs are presented in FIG. 17. The CpG density of the DHRs for all the comparisons was 1-5 CpG per 1000 bp being predominant (see FIG. 17). The length of the DHRs for each disease biomarker and glyphosate versus control were 1-3 kb with 1 kb length being predominant except for the glyphosate versus control showing DHRs length from 1 to 10 kb with 1 and 2 kb length being predominant (see FIG. 17). Generally, the DHRs are 1 kb in size with around 10 CpGs as previously reported. A PCA of the different DMR genomic site comparisons (glyphosate versus control, prostate disease biomarker, kidney disease biomarker, obesity disease biomarker and multiple disease biomarker) for the DHRs revealed a clustered separation of the diseases versus non-disease, (see FIG. 18). This helps confirm the edgeR analysis is identifying differential disease sites.

### Epimutation Comparison Analysis

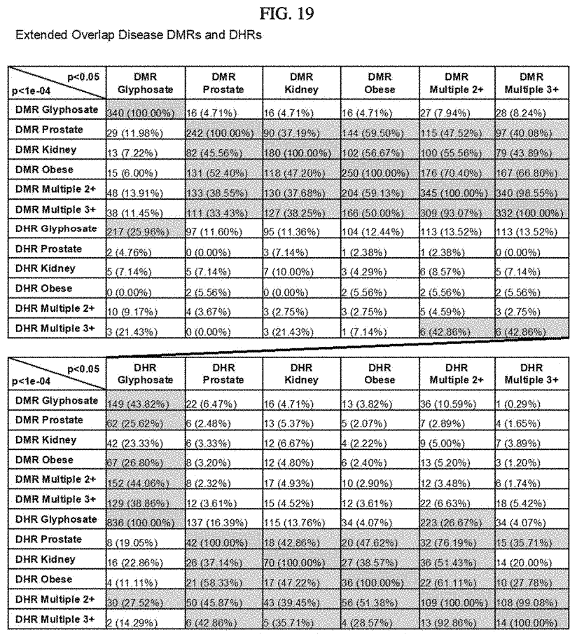

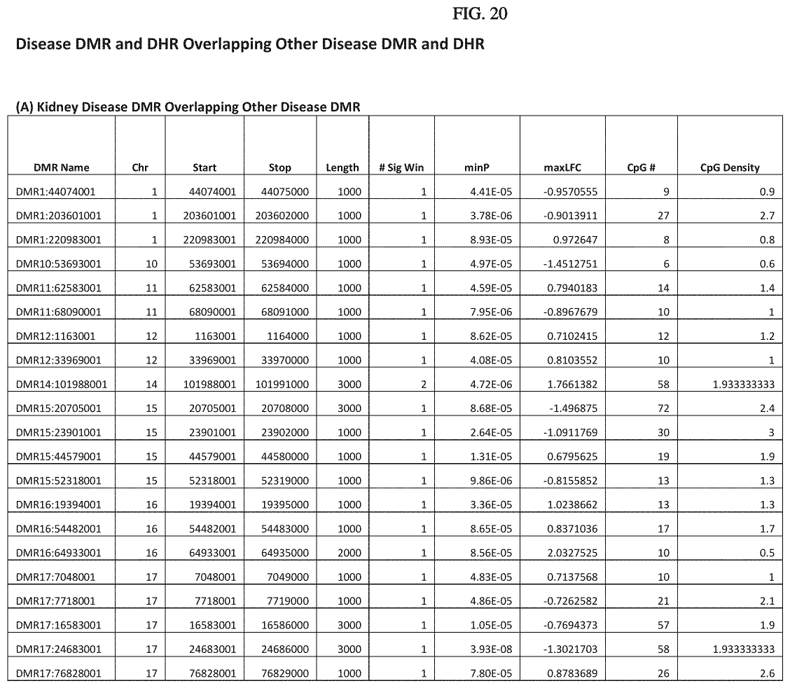

A relatively stringent edgeR p-value for the DMRs or DHRs is used for the identification of these disease specific epimutations. A reduced statistical threshold of p<0.05 was used to compare and further evaluate the potential overlap of the DMRs or DHRs between the glyphosate versus control and the different disease biomarkers when compared to the higher edgeR p<1e−04 statistical threshold DMRs. By lowering the stringency to a p-value of <0.05 for the comparison (i.e. extended overlap) the procedure allows for the identification of more potential overlaps between the glyphosate versus control, and the prostate, kidney, obesity and multiple disease comparisons. The relatively high statistical threshold is used as the epigenetic site definition. A comparison of the p<1e−04 for the DMRs, with each potential comparison at p<0.05 (see FIG. 19) demonstrates a much higher overlap between the prostate DMRs and the kidney DMRs (37.2%) and obesity DMRs (59.5%) (FIG. 19A). The extended overlap between the DMRs for the different pathologies generally was minimally 30% and as high as 70%, as indicated with highlighted horizontal row comparisons. Interestingly, minimal overlap was observed between the DMRs and DHRs (<10%), except for the glyphosate versus control comparisons with >25% overlap, (FIG. 19). An additional comparison was made between individuals with ≥2 or ≥3 different pathologies. The overlap between these ≥2 and ≥3 multiple disease was greater than 90%, (FIG. 19). Therefore, there was not an increase of DMR or DHR with ≥3 pathologies, and similar DMR and DHR sites were identified. Observations suggest having an increased amount of disease/pathology does not appear to correlate with an increased number of epigenetic alterations. No further analysis of the ≥3 pathology DMR data was performed.

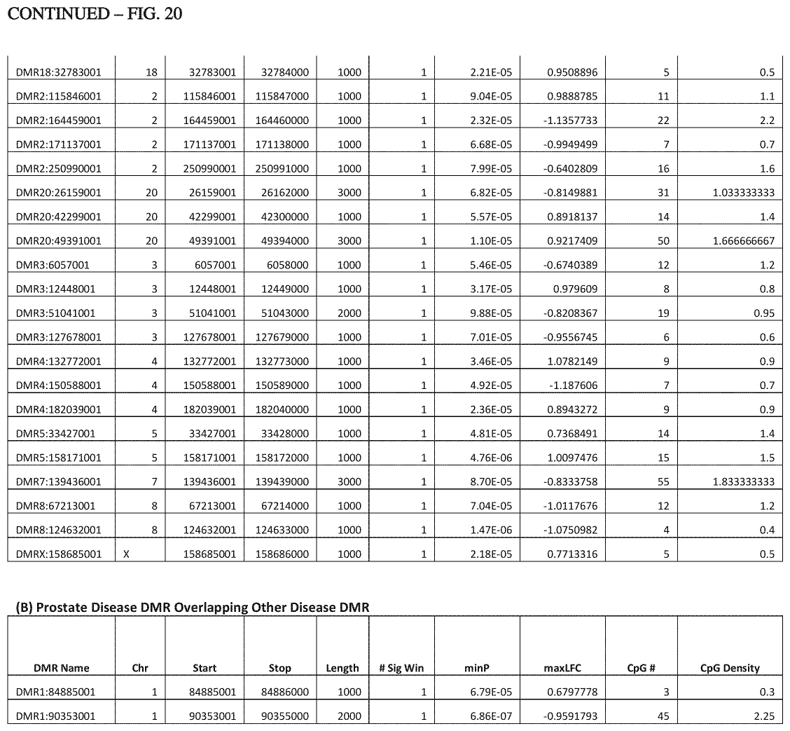

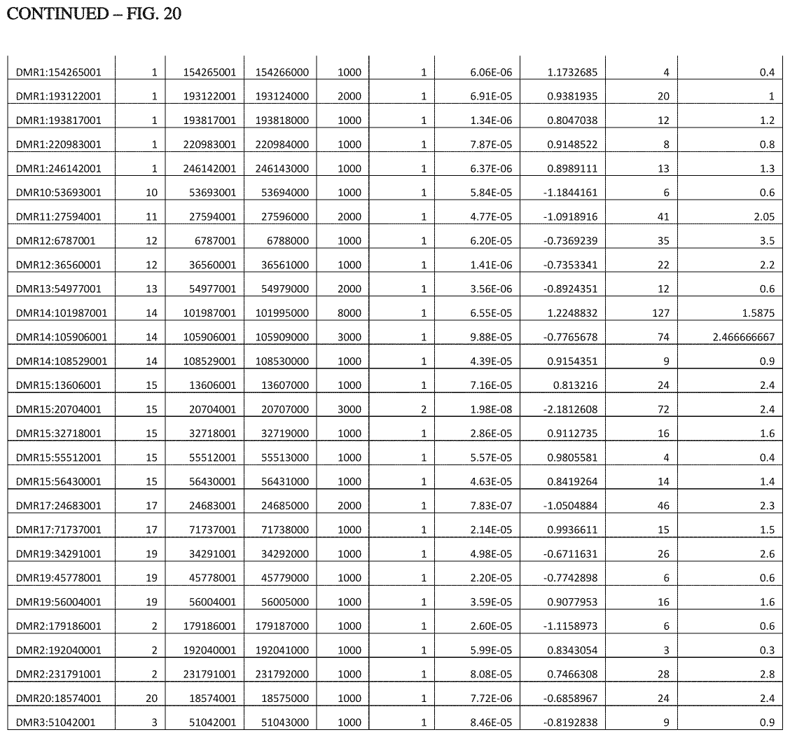

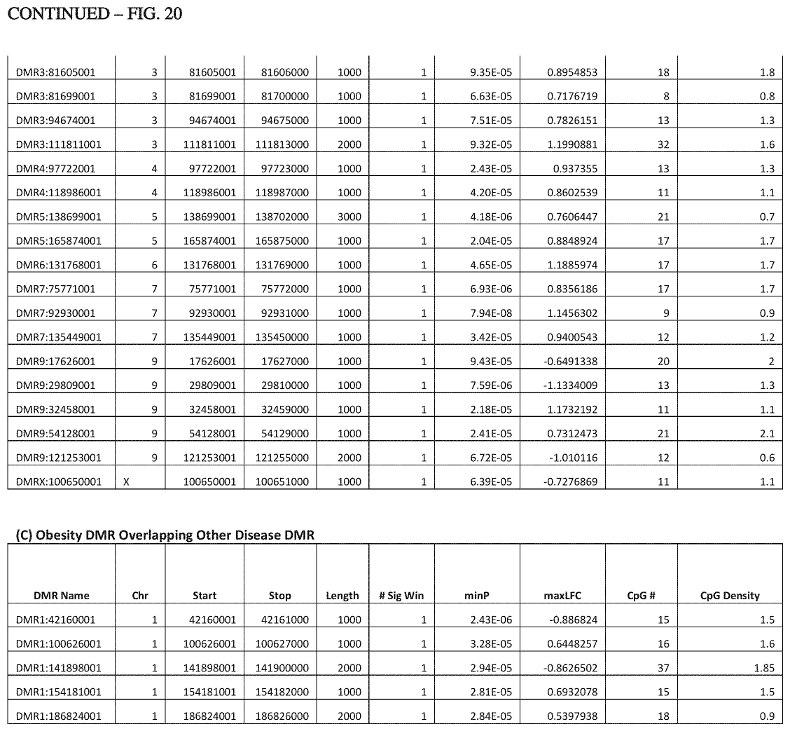

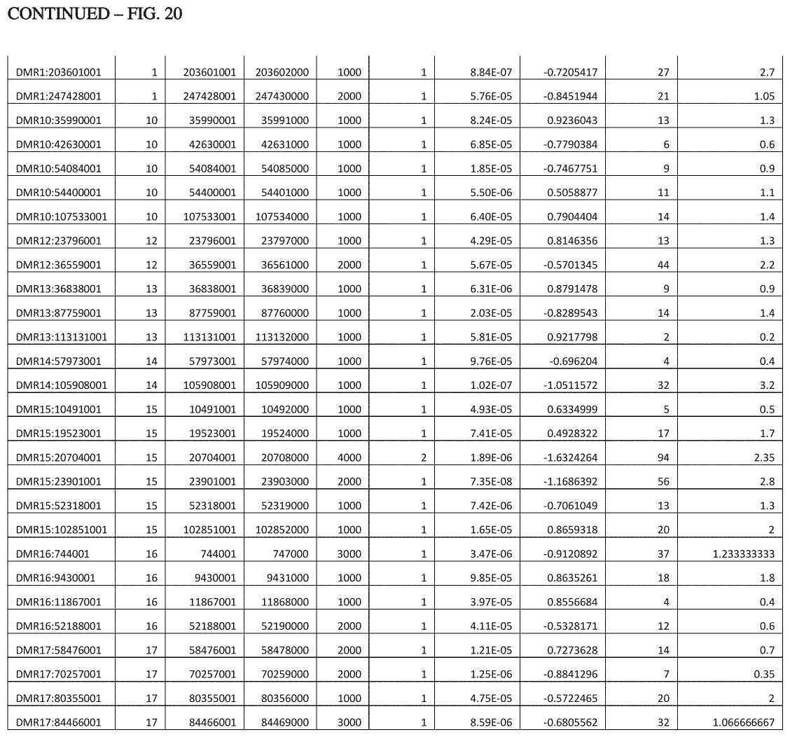

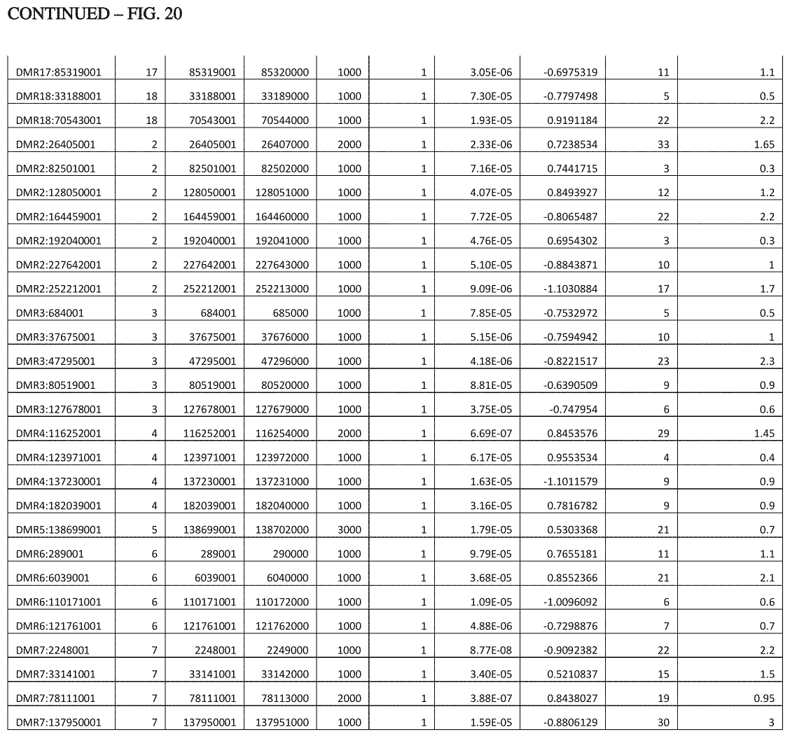

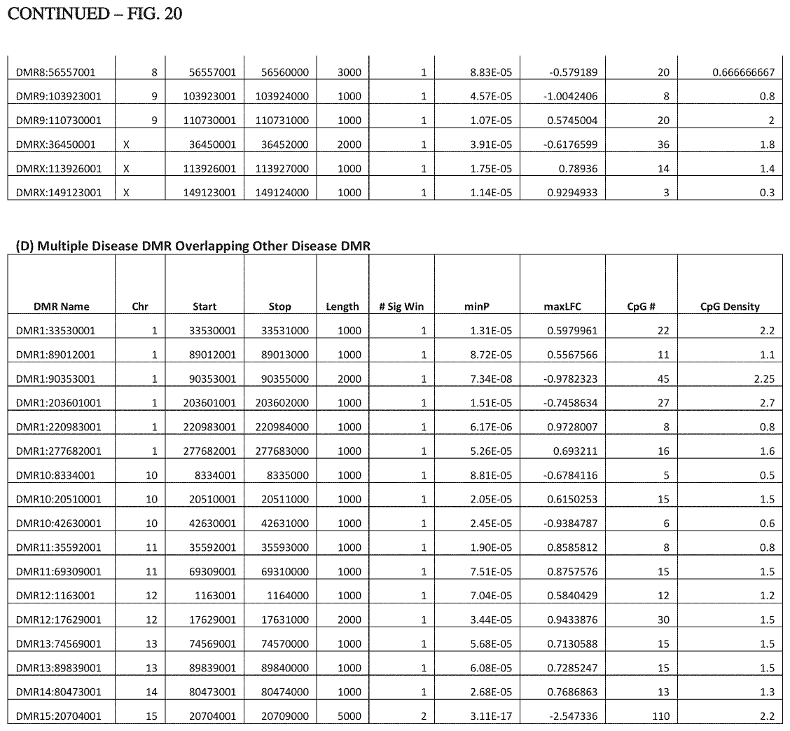

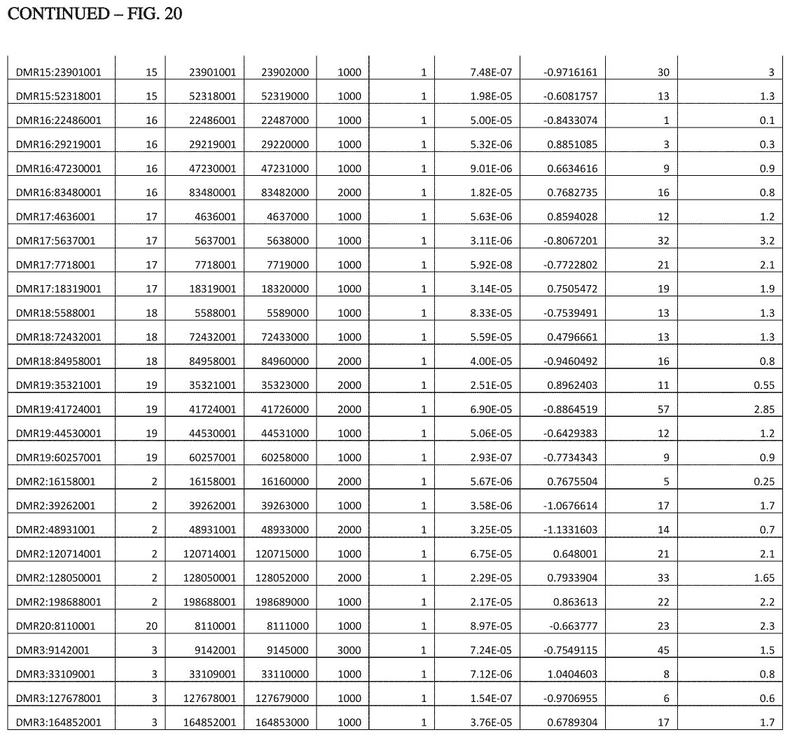

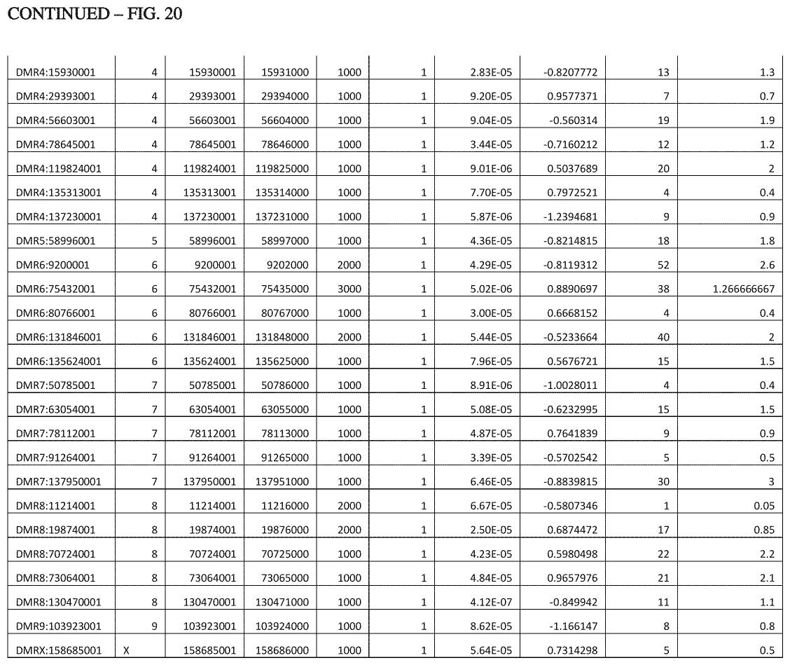

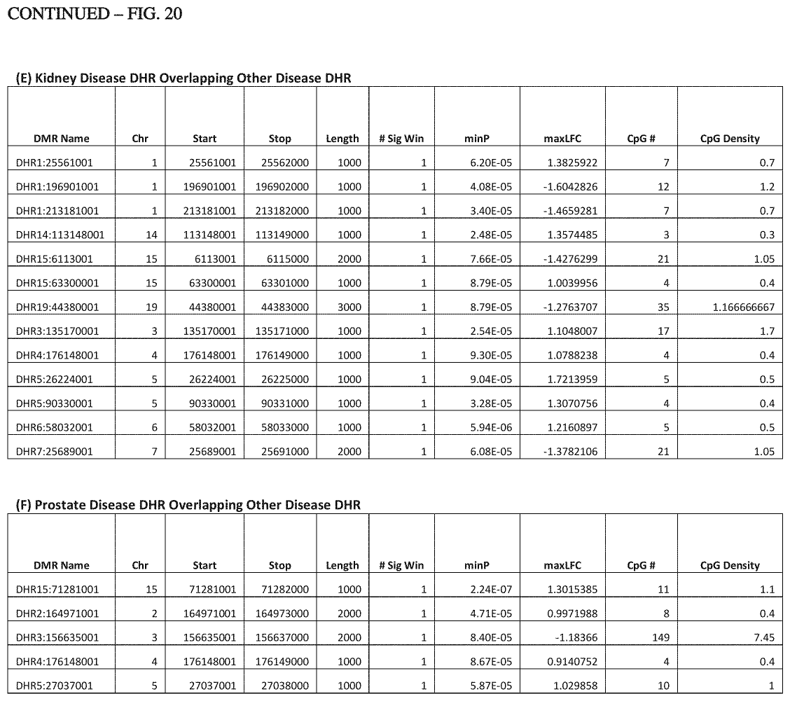

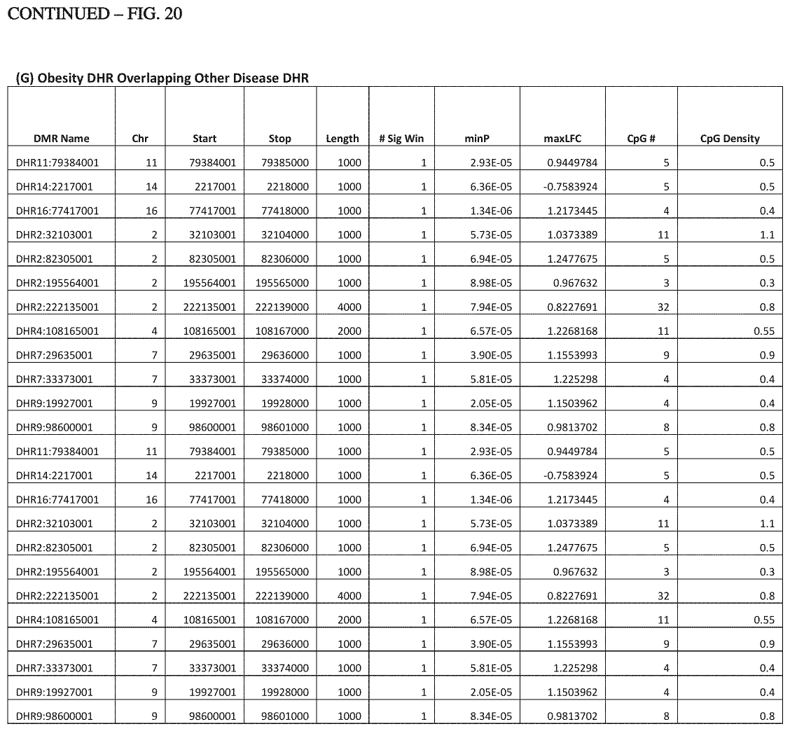

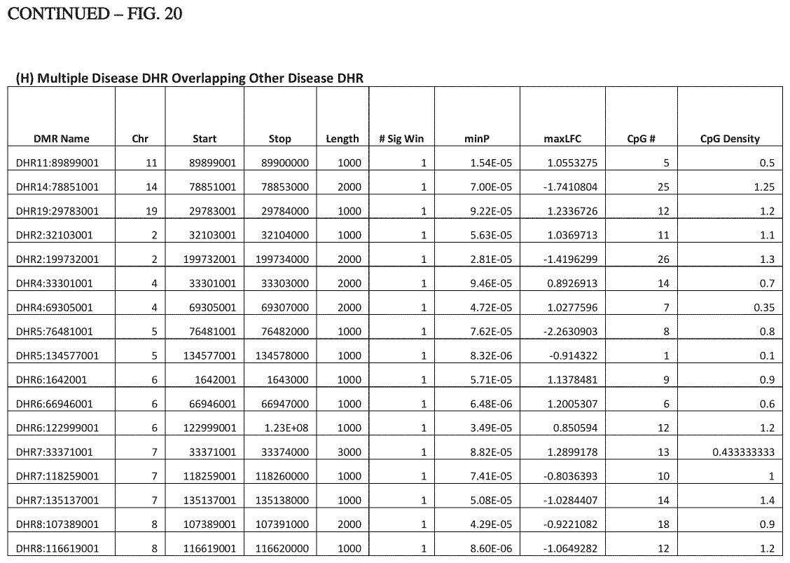

The same reduced statistical edgeR threshold extended overlap was used for the DHRs in FIG. 19B. Similarly to the DMRs, a comparison of the p<1e−04 for the DHRs, with each potential comparison at p<0.05, (FIG. 19B), shows a much higher overlap between the prostate DHRs and the kidney DHRs (42.9%) and obesity DHRs (47.6%) and multiple disease DHRs (76.2%) (FIG. 19B). Although very few overlaps are observed between the DMRs and DHRs (FIG. 19B), observations indicate that a subset of DMRs and DHRs appear to be common among diseases for a specific disease comparison. Analysis of the DMR or DHR overlaps for sites that are common between the different pathologies identified lists for both (see FIG. 20).

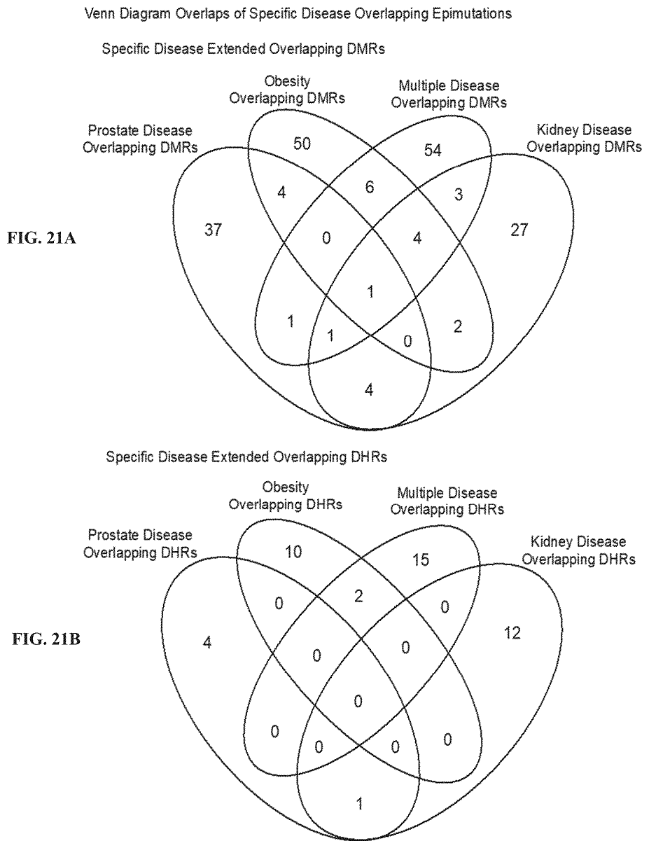

The lists of DMR and DHR in common with all pathologies identify those with associated genes. Interestingly, when the common DMR and DHR sites for a specific disease comparison were identified and then compared between all the diseases, negligible overlap was observed (FIGS. 1F and 10F). Therefore, there are common DMRs within a specific disease comparisons, but these common DMR sets are primarily disease specific (see FIG. 21).

### Epimutation Gene Associations

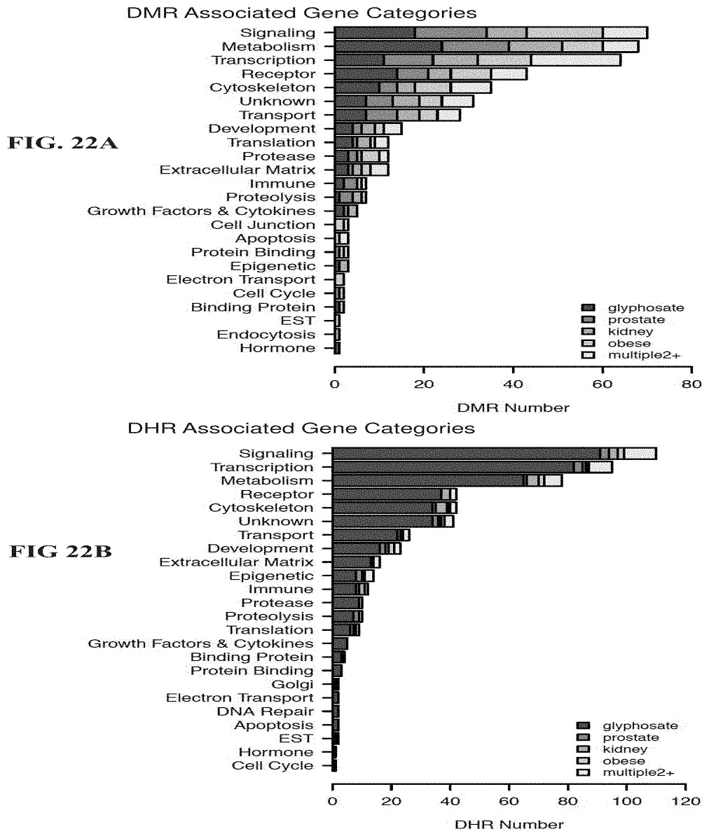

The list of DMRs and DHRs for all the epigenetic alterations identified are presented in Tables 3-12. Epimutation gene associations used DMR or DHR identified within 10 kb of a gene so as to include proximal and distal promoter elements. The minority of DMR or DHR, less than 20%, have epimutations associated with genes. Therefore, the majority are intergenic and not within 10 kb of a gene. The DMR and DHR associated genes found were categorized into relevant functional categories for the glyphosate versus control, and for each set of disease biomarkers (FIG. 22). The associated gene categories listed for the Tables 3-12 used DAVID and Panther public databases with direct experimental gene functional links. The top ten gene categories containing multiple genes are presented for DMRs (FIG. 22A) and DHRs (FIG. 22B). Epimutations were found predominantly in the signaling, metabolism, transcription, receptor and cytoskeleton categories for both DMRs and DHRs, (FIG. 22). The number of epimutations was higher for the DMRs compared to the DHRs. The highest represented gene categories typically involve the gene categories with the highest number of associated genes, such as metabolism.

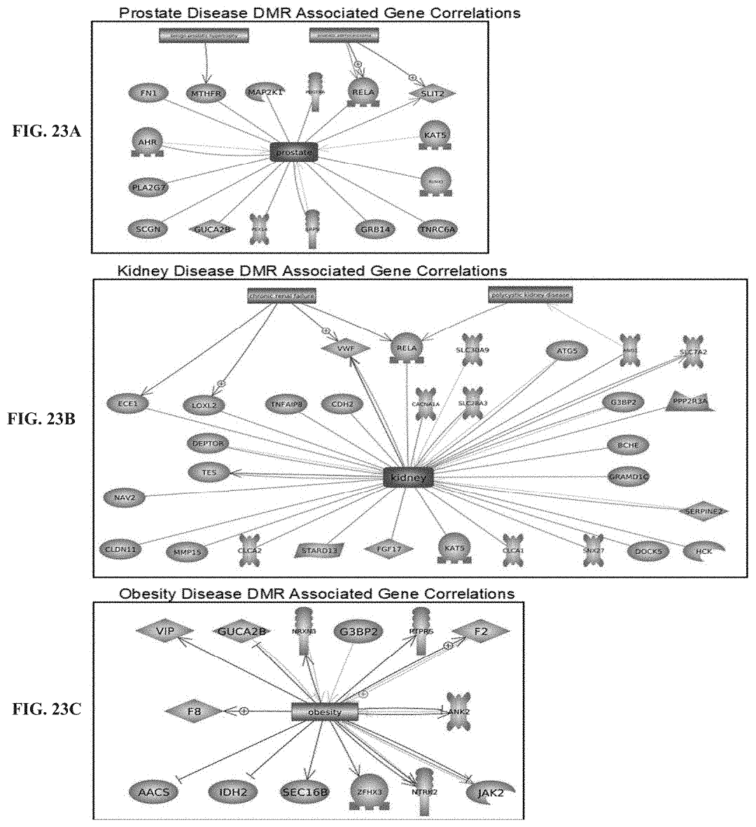

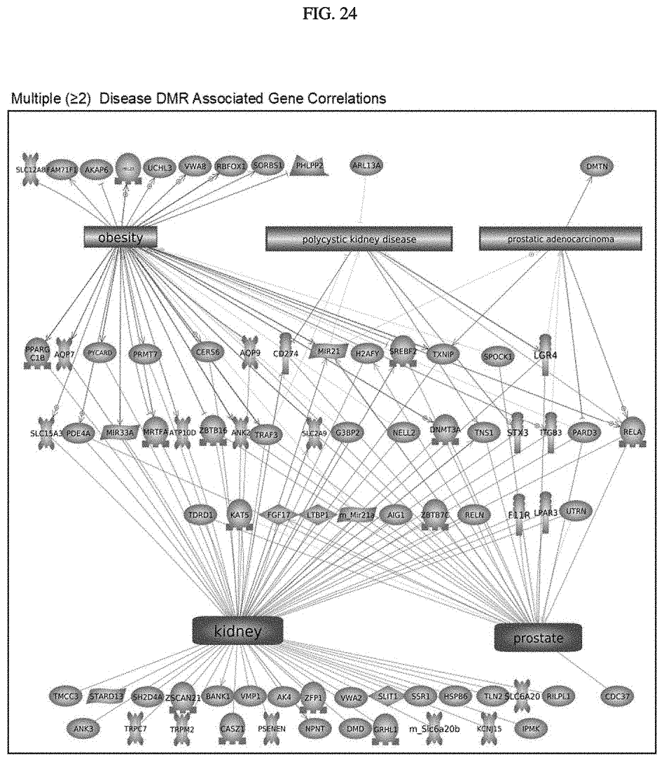

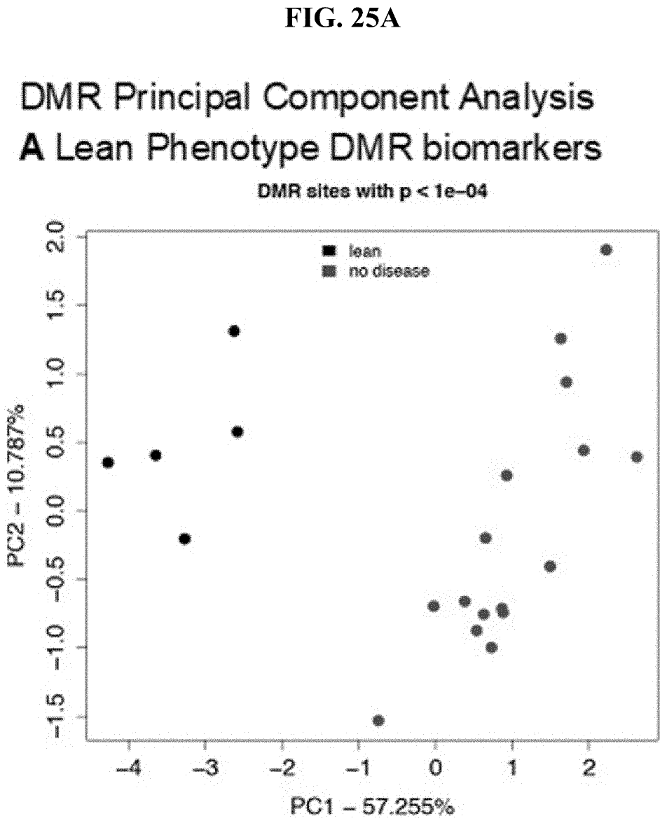

The disease specific DMR associated genes were analyzed using a Pathway Studio gene database and network tool to identify associated gene processes, (FIGS. 23 and 24). The disease specific DMR associated genes predominantly corresponded with the associated disease for prostate disease, kidney disease, and obesity, (FIG. 23). Some additional associated disease groups were also identified. Interestingly, the multiple (≥2) disease epimutation biomarker DMR associated genes were found to be correlated with all the major prostate, kidney, and obesity processes, (FIG. 24). The individual gene processes and shared gene processes are identified in FIG. 24. The epimutation gene associations with previously identified disease-linked genes helps validate the observations and biomarkers. Statistical analysis of over-representation of DMR-associated genes with diseases as performed by Pathway Studio software (Elsevier, Inc. 2020) revealed that for prostate disease DMR-associated genes (FIG. 23A) the disease term Benign Prostatic Hypertrophy was enriched (p=0.048). For obesity disease DMR-associated genes (FIG. 23C) the term Obesity was enriched (p=0.046). For the multiple disease DMR-associated genes (FIG. 24) the disease terms Obesity (p=0.004), Polycystic Kidney Disease (p=0.0008), and Prostatic Adenocarcinoma (p=0.008) were significantly enriched.

### Conclusions

Surprisingly, sperm differential histone retention regions (DHRs) were observed with different pathologies. The number of DHRs were less than the number of DMRs and the DHRs were also found to have disease specificity (FIG. 10). The histones in sperm are replaced by protamines to compact DNA into the head of the sperm at the later stage of spermatogenesis in the testis following meiosis. However, specific histone retention sites were observed and found to be conserved. As shown herein, the toxicant glyphosate appears to promote the epigenetic transgenerational inheritance of DHRs in sperm. These sperm DHRs also appear to provide epigenetic biomarkers for disease suggesting that DHRs are biomarkers for disease. An overlap at edgeR p<1e−04 demonstrated limited overlap of DHRs between the different pathologies, but at a reduced threshold comparison overlap demonstrated a 25-75% overlap between the pathologies (see FIG. 19). Negligible overlap was observed between the DMRs and DHRs at each of the edgeR statistical thresholds. Observations demonstrate the sperm DHRs also appear to provide potential epigenetic biomarkers for disease.

### Example 2

### Evaluation of Epigenetic Alterations Induced by Atrazine

In the instant example, atrazine is utilized as an exemplary toxicant. Rats were used as the exemplary animal.

### Animal Studies and Breeding

Female and male rats of an outbred strain Hsd:Sprague Dawley SD (Harlan) at 70 to 100 days of age were fed ad lib with a standard rat diet and ad lib tap water. Timed-pregnant females on days 8 through 14 of gestation were administered daily intraperitoneal injections of atrazine (25 mg/kg BW/day dissolved in PBS) (Chem Service, Westchester Pa.) or dimethyl sulfoxide (DMSO) or as previously described. Twenty-five mg/kg for atrazine is 4% of rat oral LD50 and 50% of NOAEL.

The gestating female rats treated were designated as the F0 generation. F1-F3 generation control and atrazine lineages were housed in the same room and racks with lighting, food and water.

### Tissue Harvest and Histology Processing

Rats were euthanized at 12 months of age by CO₂ inhalation and cervical dislocation for tissue harvest. Testis, prostate, ovary, kidney, and gonadal fat pads were fixed in Bouin's solution (Sigma) followed by 70% ethanol, then processed for paraffin embedding and hematoxylin, and eosin (H & E) staining by standard procedures for histopathological examination. Paraffin five micron sections were processed and stained (Nationwide Histology, Spokane Wash., USA).

### Histopathology Examination and Disease Classification

Full necropsies were performed as required on animals that died prior to the time of scheduled sacrifice at one year and tumor classifications were performed.

Upon dissection a brief examination of abdominal and thoracic organs was performed to look for obvious abnormalities. The tissues evaluated histologically were selected from previous literature showing them to have pathology in transgenerational models, with an emphasis on reproductive organs. Histopathology readers were trained to recognize the specific abnormalities evaluated for this study in rat testis, ventral prostate and kidney (see below). Two different readers initially evaluated the tissues. If there was disagreement on whether an animal's tissue showed disease, then a third pathology reader was used. Readers were blinded to the exposure groups. A set of quality control (QC) slides was generated for each tissue and was read by each reader prior to evaluating any set of experimental slides. These QC slide results are monitored for reader accuracy and concordance.

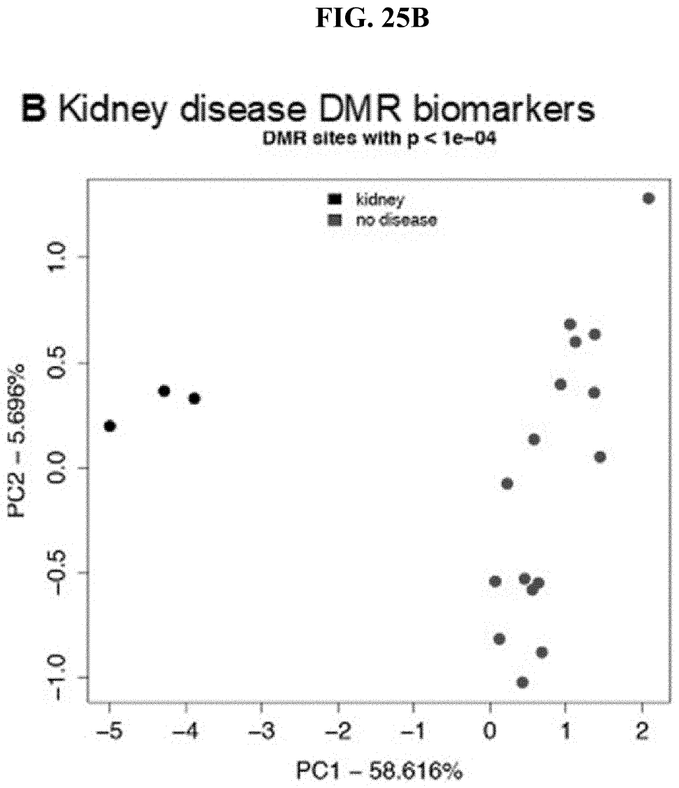

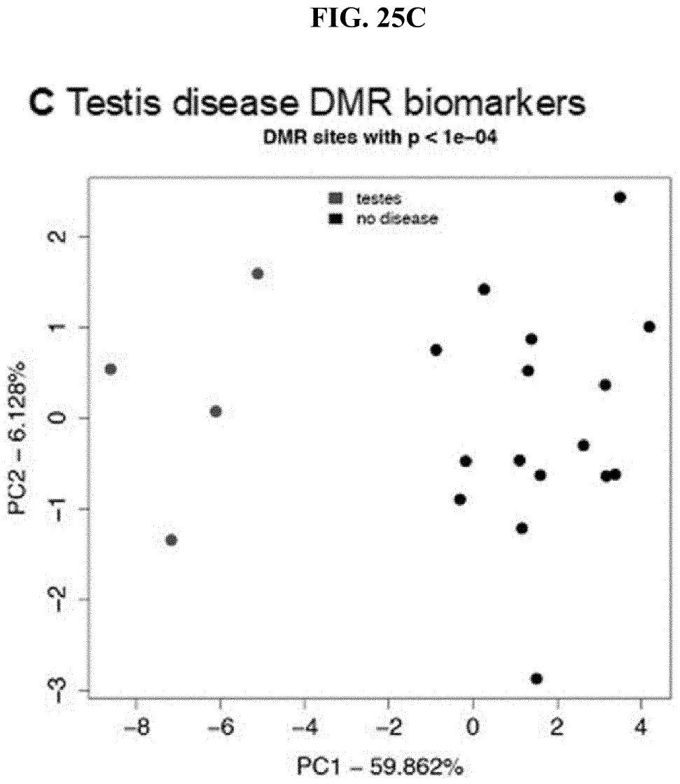

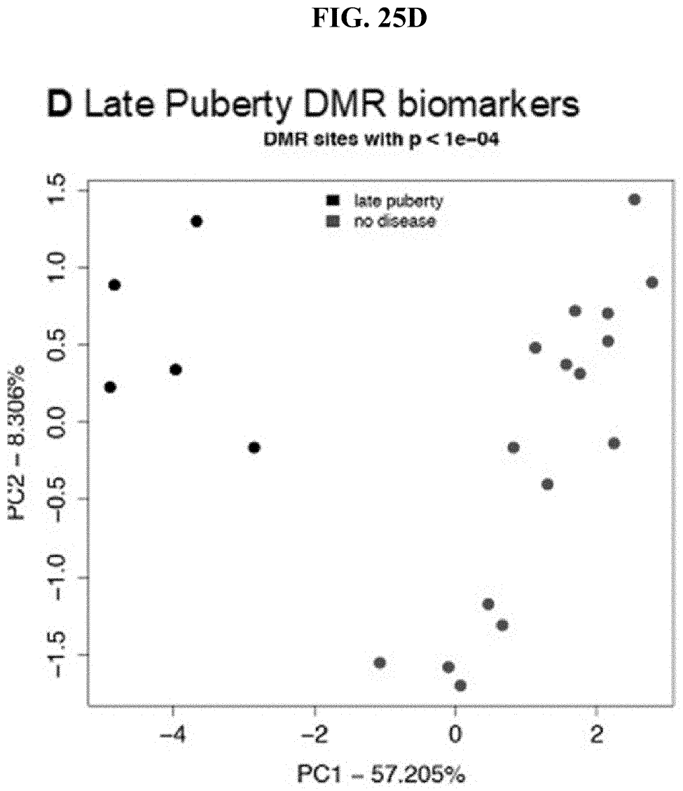

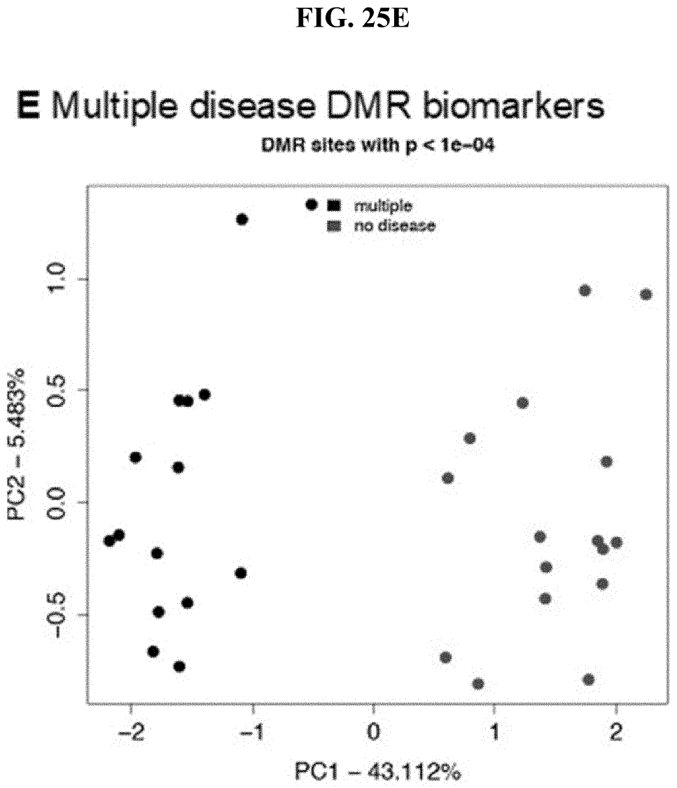

Testis histopathology criteria included the presence of vacuoles in the seminiferous tubules, azoospermic atretic seminiferous tubules, and ‘other’ abnormalities including sloughed spermatogenic cells in the center of the tubule and a lack of a tubule lumen. Prostate histopathology criteria included the presence of vacuoles in the glandular epithelium, atrophic glandular epithelium and hyperplasia of prostatic gland epithelium (FIG. 25). Kidney histopathology criteria included reduced size of glomerulus, thickened Bowman's capsule, and the presence of proteinaceous fluid-filled cysts >50 μm in diameter (FIG. 25). A cut-off was established to declare a tissue ‘diseased’ based on the mean number of histopathological abnormalities plus two standard deviations from the mean of control group tissues, as assessed by each of the individual readers. This number (i.e. greater than two standard deviations) was used to classify rats into those with and without testis, prostate, or kidney disease in the F3 generation lineage. A rat tissue section was finally declared ‘diseased’ only when at least two of the three readers marked the same tissue section ‘diseased’.

Lean phenotype was assessed with a decrease in adipocyte size (area), body mass index (BMI) and abdominal adiposity. BMI was calculated with weight (g)/length (cm)² with the length of the animal measured from the nose to the base of the tail. Gonadal fat pad slides were imaged using a Nikon Eclipse E800 microscope (10×) with an AVT Prosilica GE1050C Color GigE camera. Five field of view image captures were taken per slide in varying parts of the fat pad. Adipocyte size was measured converting pixels into microns using Adiposoft. Measurements of the 20 largest cells from each image for a total of 100 were averaged as hypertrophic cells are the most metabolically relevant and susceptible to cell death. Obesity and lean phenotypes were determined utilizing the mean of the control population males and females, and a cut-off of 1.5 standard deviations above and below the mean.

### Disease Groups for Biomarker Analysis

The individual animals are listed and a (+) indicates presence of disease and (−) absence of disease for the current F3 generation atrazine lineage male pathology (Table 3). Table 3 shows F3 generation atrazine lineage males pathology. The individual animals for the atrazine lineage males are listed and a (+) indicates presence of disease and (−) absence of disease. The boxes marked with an (*) represent animals with a single disease (+) or no disease (0) that were used for the molecular analysis. The number of disease animals/total animals is presented.

TABLE 3
Individual Animal Pathology F3 Generation Atrazine Lineage Males

| Animal | Puberty | | | | | | | | Multiple | Total |
|---|---|---|---|---|---|---|---|---|---|---|
| ID | Early | Late | Testis | Prostate | Kidney | Lean | Obese | Tumor | Disease | Disease |
| AM1 | — | — | — | — | +* | — | — | — | | 1 |
| AM2 | — | — | — | + | + | — | — | — | +* | 2 |
| AM3 | — | — | — | — | — | — | — | — | — | 0 |
| AM4 | — | — | — | — | + | + | — | — | +* | 2 |
| AM5 | — | — | — | — | +* | — | — | — | — | 1 |
| AM6 | — | — | + | — | — | + | — | — | +* | 2 |
| AM7 | — | — | — | — | — | — | — | — | — | 0 |
| AM8 | — | — | — | — | — | — | — | — | — | 0 |
| AM9 | — | — | — | — | — | — | — | — | — | 0 |
| AM10 | — | — | +* | — | — | — | — | — | — | 1 |
| AM11 | — | — | — | — | — | +* | — | — | — | 1 |
| AM12 | — | — | — | — | — | — | — | — | — | 0 |
| AM13 | — | — | — | — | — | — | — | — | — | 0 |
| AM14 | — | — | + | — | — | + | — | — | +* | 2 |
| AM15 | — | — | — | — | — | — | — | — | — | 0 |
| AM16 | — | — | — | — | +* | — | — | — | — | 1 |
| AM17 | — | — | — | — | — | — | — | — | — | 0 |
| AM18 | — | — | — | — | — | — | — | — | — | 0 |
| AM19 | — | — | + | — | — | — | + | — | +* | 2 |
| AM20 | — | — | — | + | — | — | + | — | +* | 2 |
| AM21 | — | — | + | + | — | + | — | — | +* | 3 |
| AM22 | — | — | — | — | — | — | — | — | — | 0 |
| AM23 | — | — | — | — | — | +* | — | — | — | 1 |
| AM24 | — | + | + | — | — | — | — | — | +* | 2 |
| AM25 | — | +* | — | — | — | — | — | — | — | 1 |
| AM26 | — | +* | — | — | — | — | — | — | — | 1 |
| AM27 | — | + | + | — | — | + | — | — | +* | 3 |
| AM28 | — | + | — | — | — | — | — | — | — | 1 |
| AM29 | — | +* | — | — | — | — | — | — | — | 1 |
| AM30 | — | — | — | — | — | — | + | + | +* | 2 |
| AM31 | — | — | — | — | — | — | — | — | — | 0 |
| AM32 | — | — | — | — | — | — | — | — | — | 0 |
| AM33 | — | — | — | — | — | — | — | — | — | 0 |
| A1V134 | — | — | — | — | — | — | — | — | — | 0 |
| AM35 | — | — | — | — | — | — | — | — | — | 0 |
| AM36 | — | — | +* | — | — | — | — | — | — | 1 |
| AM37 | — | — | + | — | — | + | — | — | + | 2 |
| AM38 | — | — | — | — | — | — | — | — | — | 0 |
| AM39 | — | — | +* | — | — | — | — | — | — | 1 |
| AM40 | — | — | +* | — | — | — | — | — | — | 1 |
| AM41 | — | — | — | — | — | — | — | — | — | 0 |
| AM42 | — | — | — | — | — | +* | — | — | — | 1 |
| AM43 | — | — | — | — | — | — | — | — | — | 0 |
| AM44 | — | — | — | — | — | — | + | — | — | 1 |
| AM45 | — | — | — | — | — | — | — | — | — | 0 |
| AM46 | — | + | + | — | + | + | — | — | +* | 4 |
| AM47 | — | + | — | + | — | + | — | — | +* | 3 |
| AM48 | — | +* | — | — | — | — | — | — | — | 1 |
| AM49 | — | +* | — | — | — | — | — | — | — | 1 |
| AM50 | — | — | — | — | — | +* | — | — | — | 1 |
| AM51 | — | — | — | — | — | — | — | — | — | 0 |
| AM52 | — | — | — | — | — | +* | — | — | — | 1 |
| AM53 | — | — | — | — | + | + | — | — | +* | 2 |
| AM54 | — | — | — | — | + | + | — | — | +* | 2 |
| # affected | 0 | 10 | 12 | 4 | 8 | 15 | 4 | 1 | | |
| # evaluated | 55 | 55 | 55 | 55 | 55 | 55 | 55 | 55 | | |

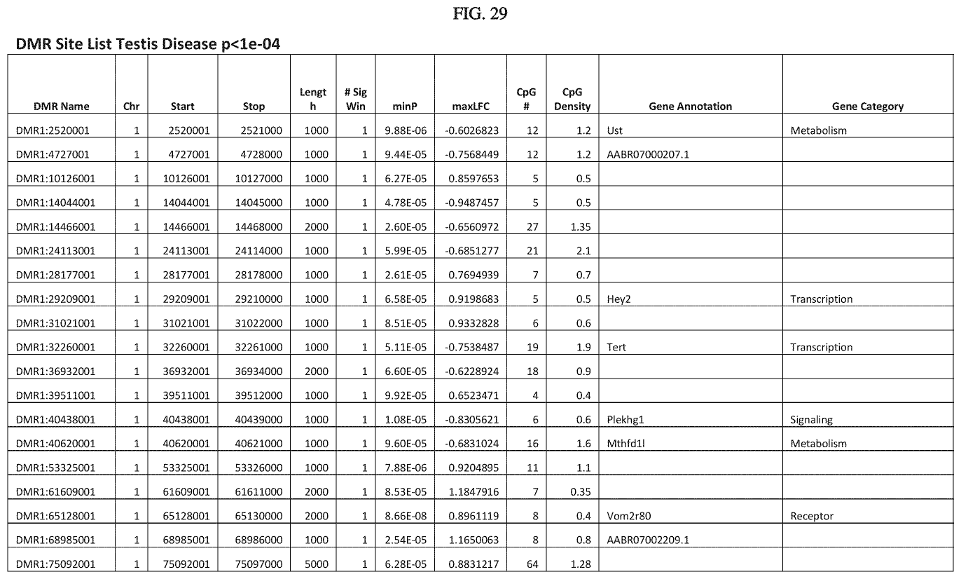

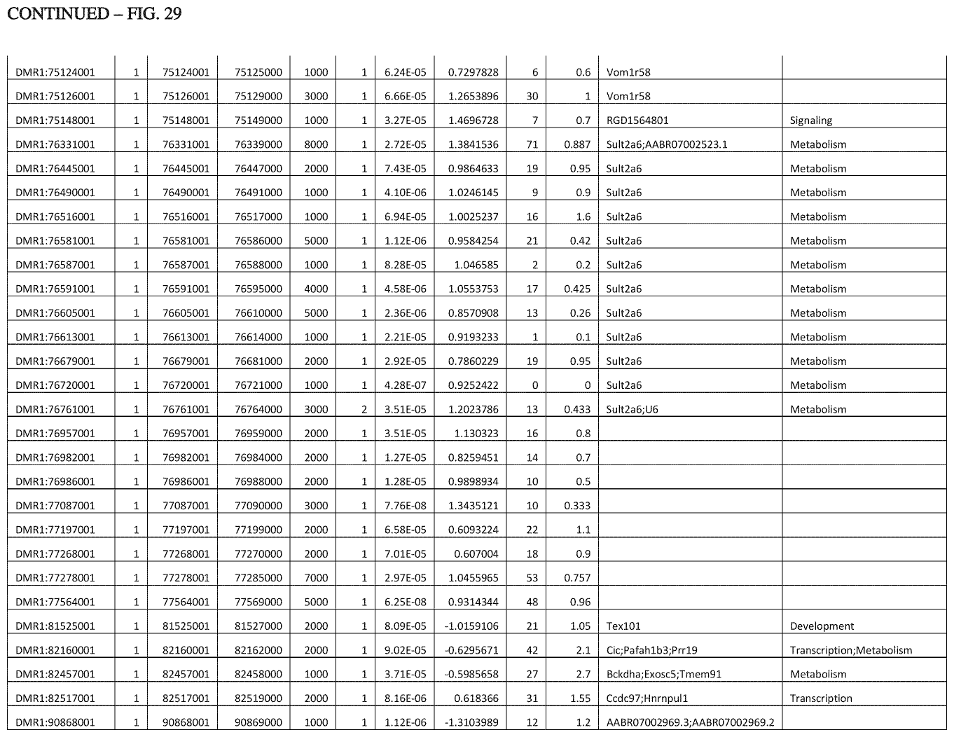

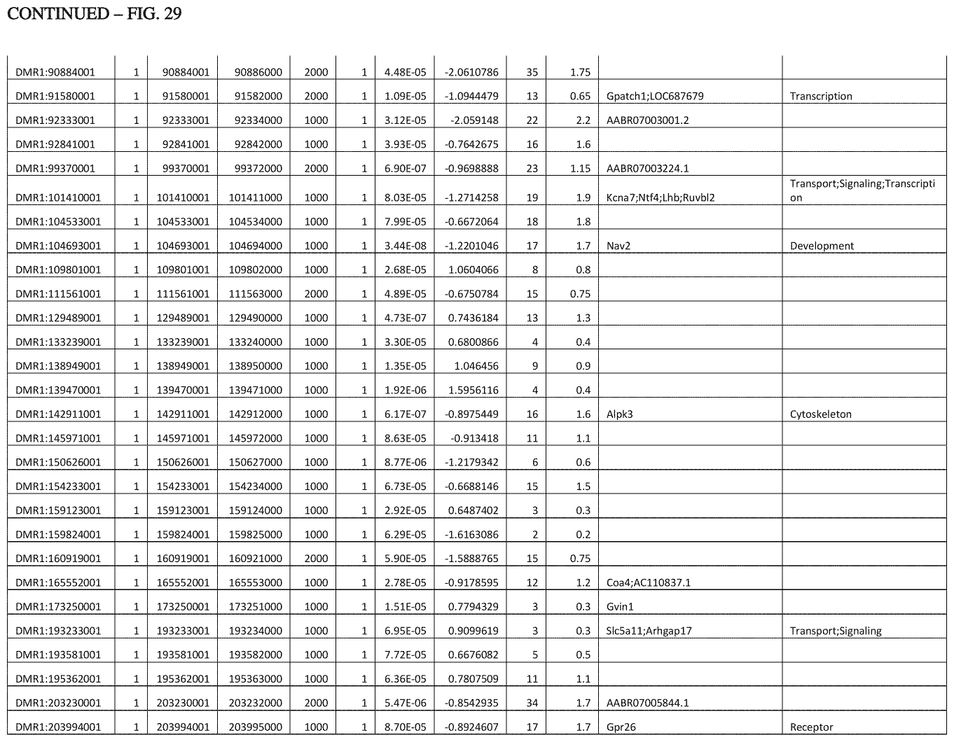

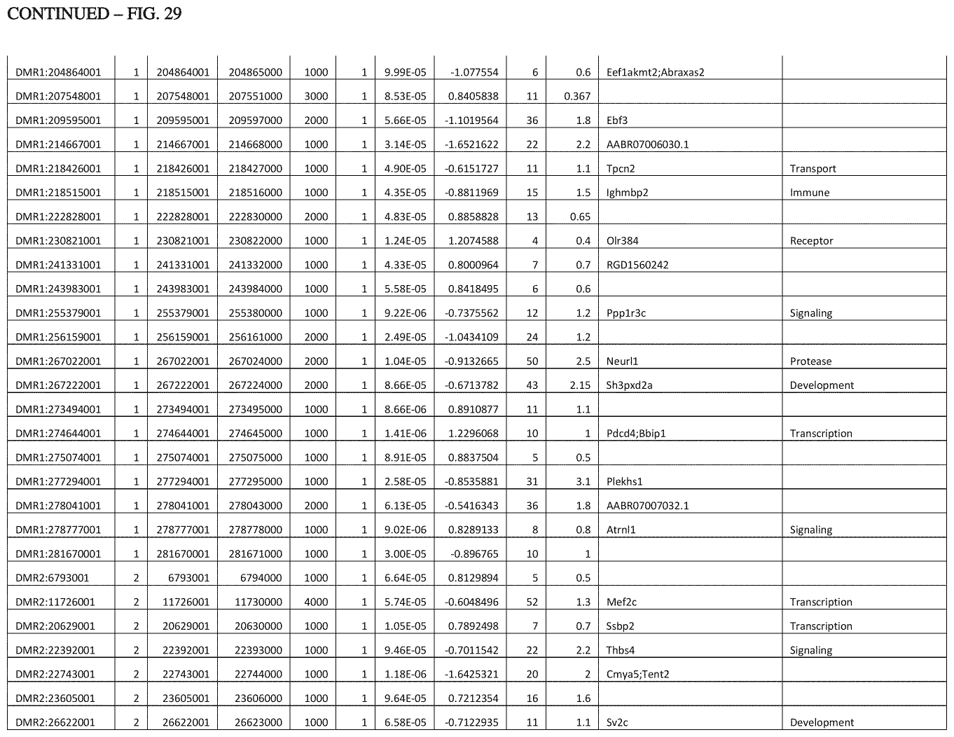

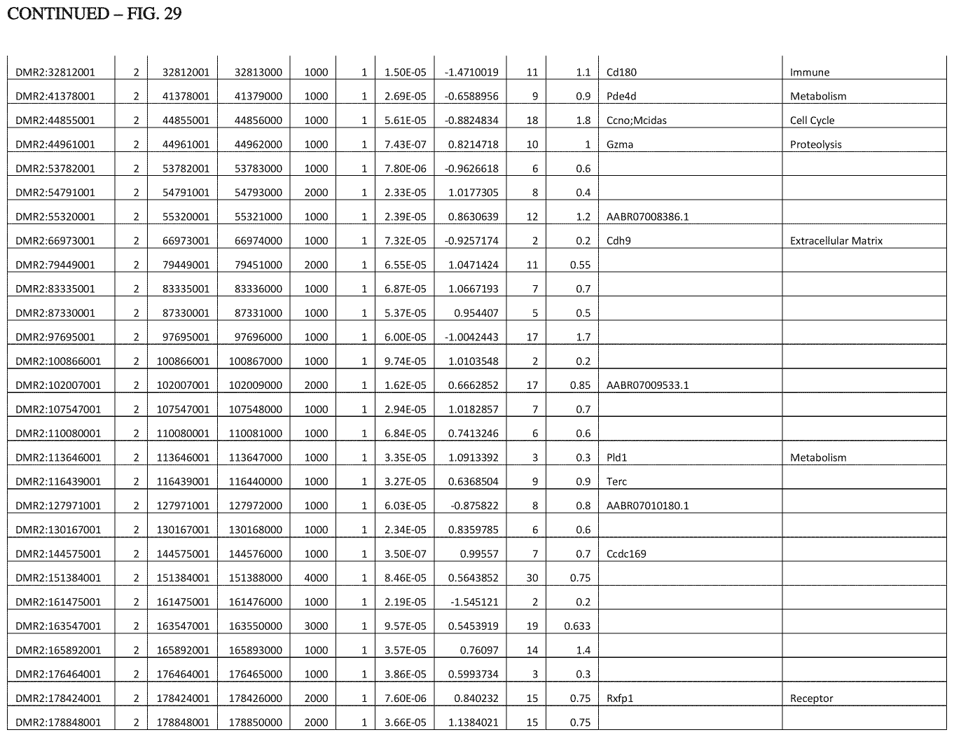

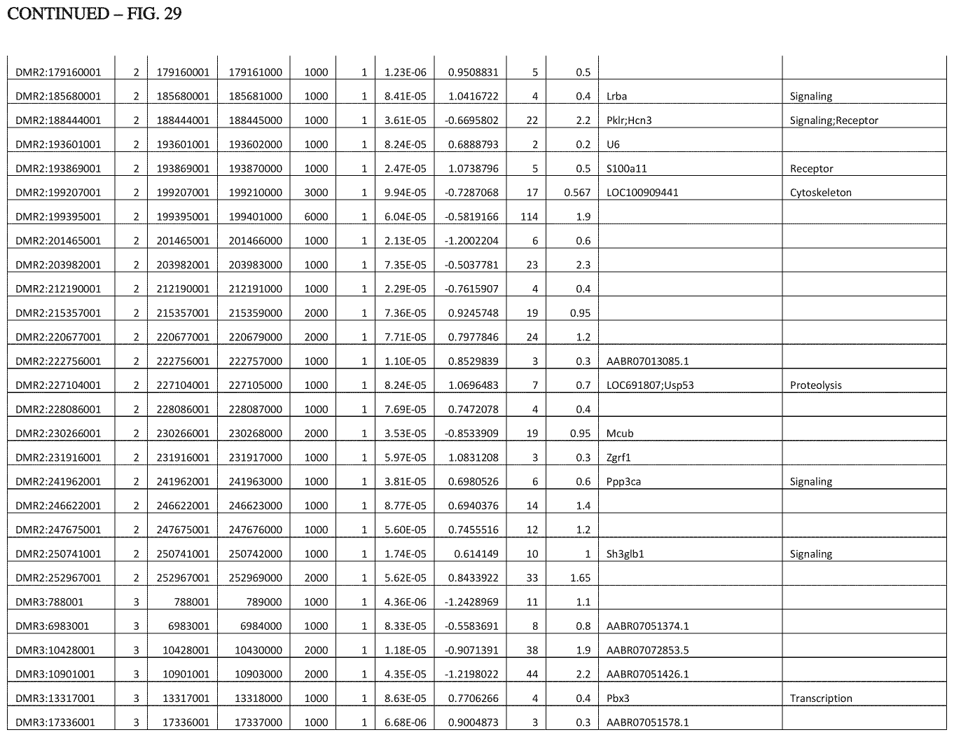

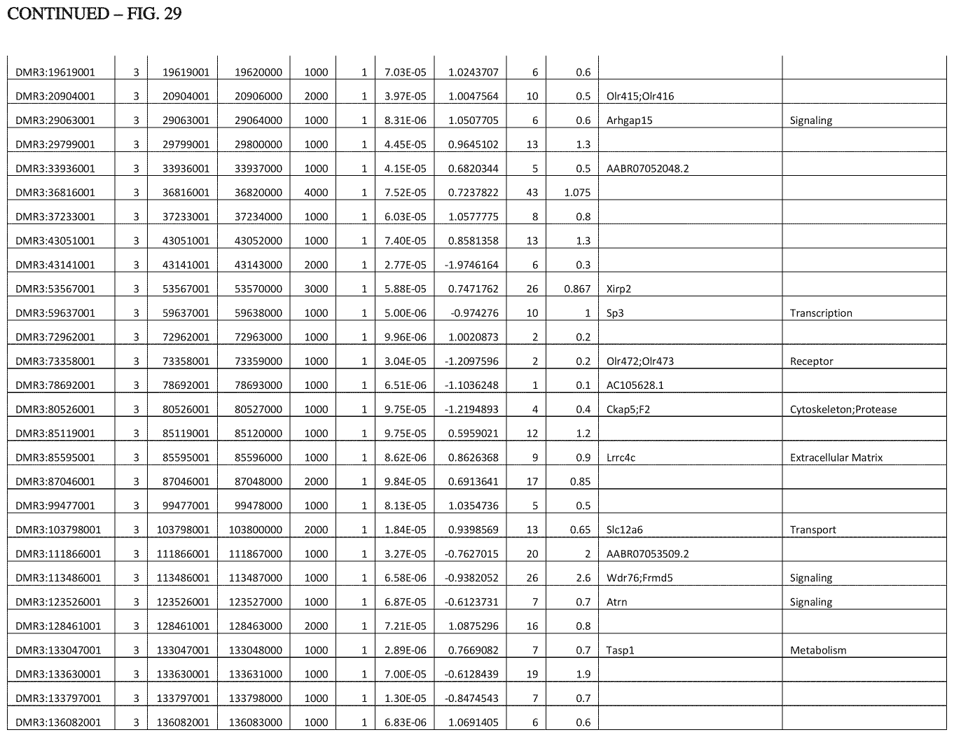

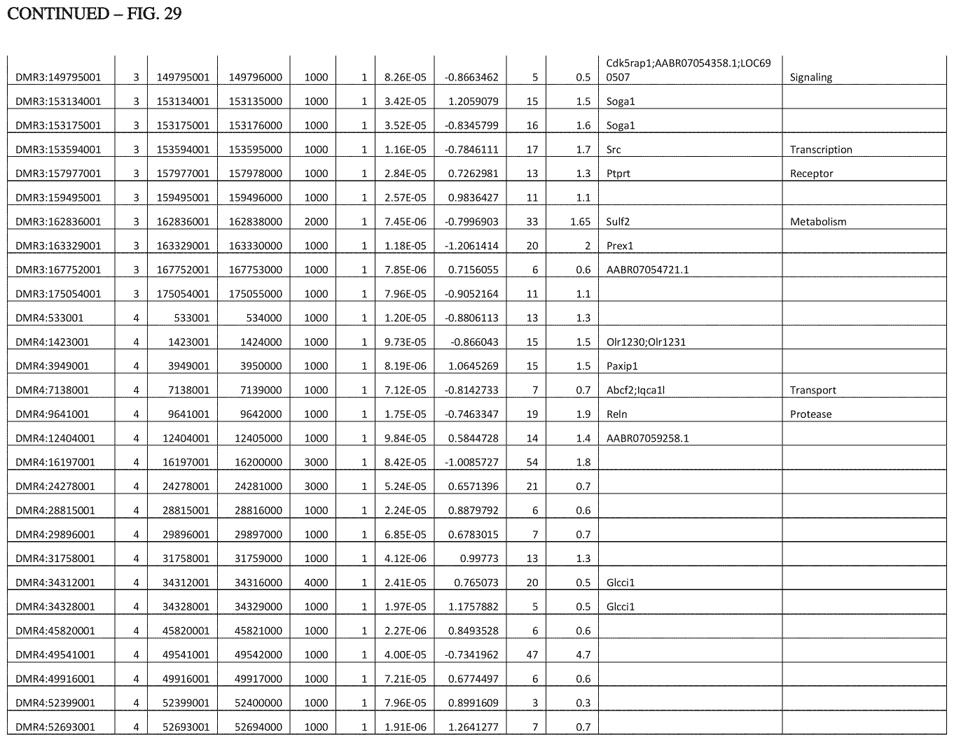

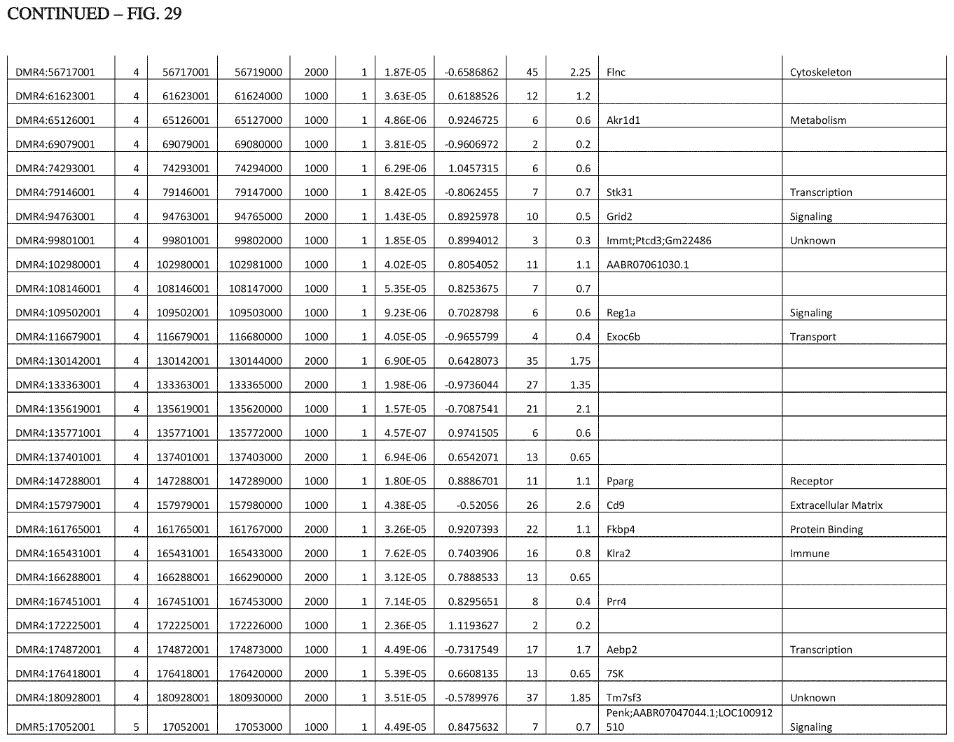

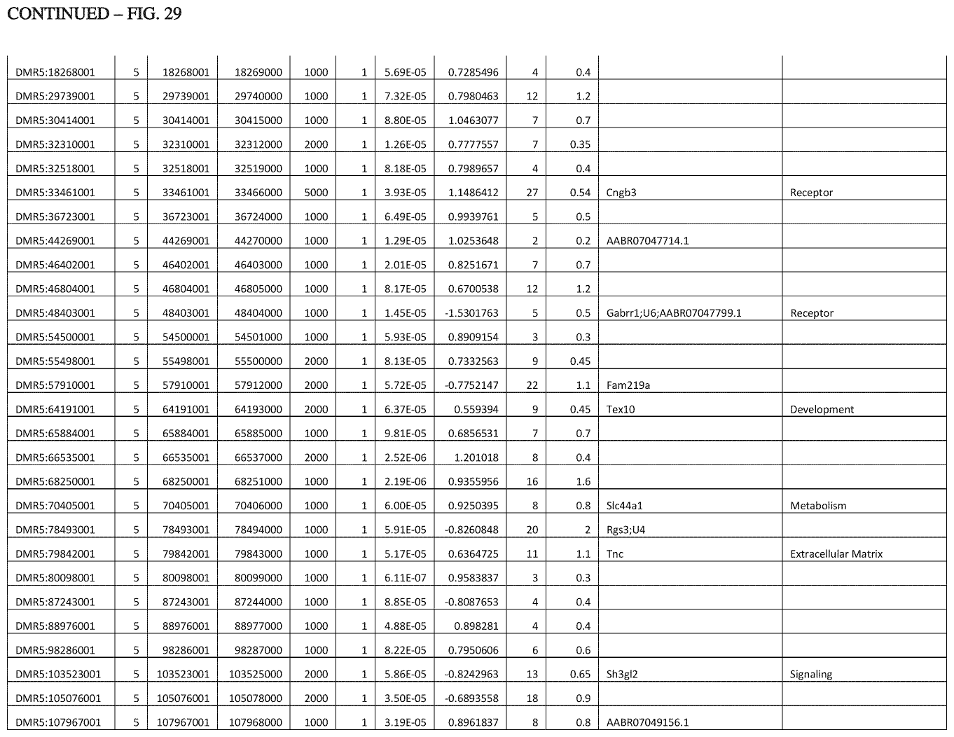

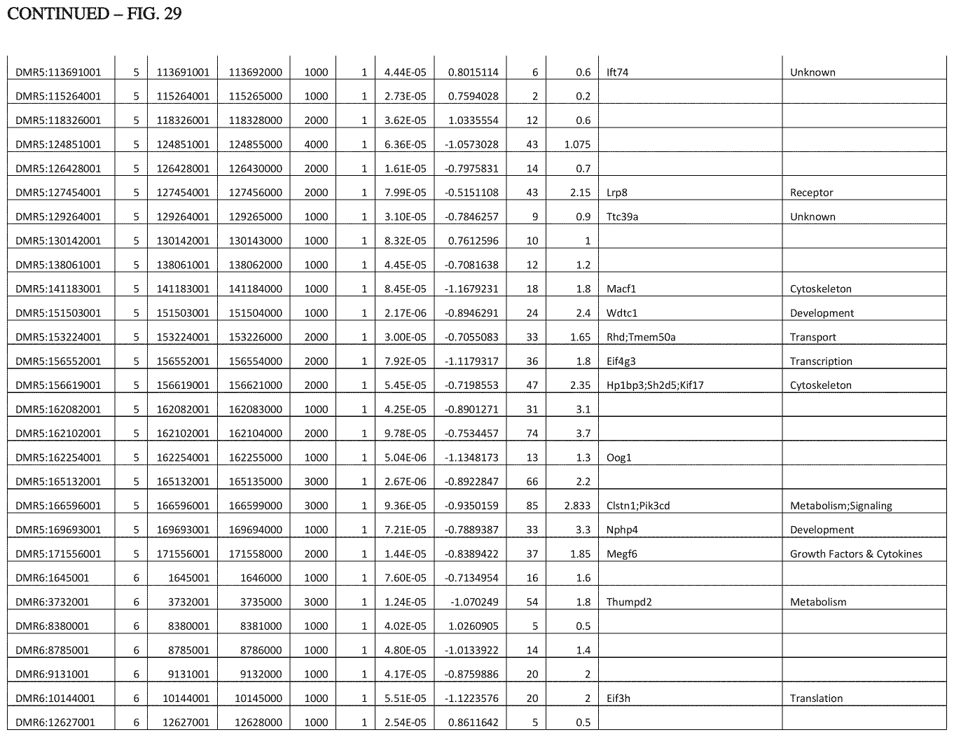

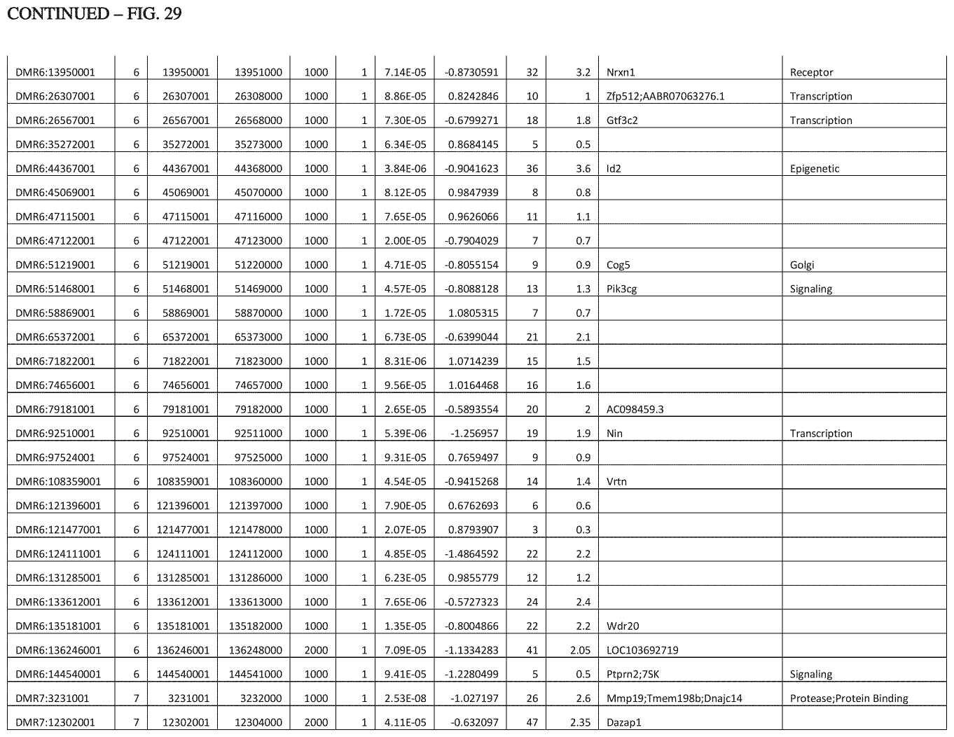

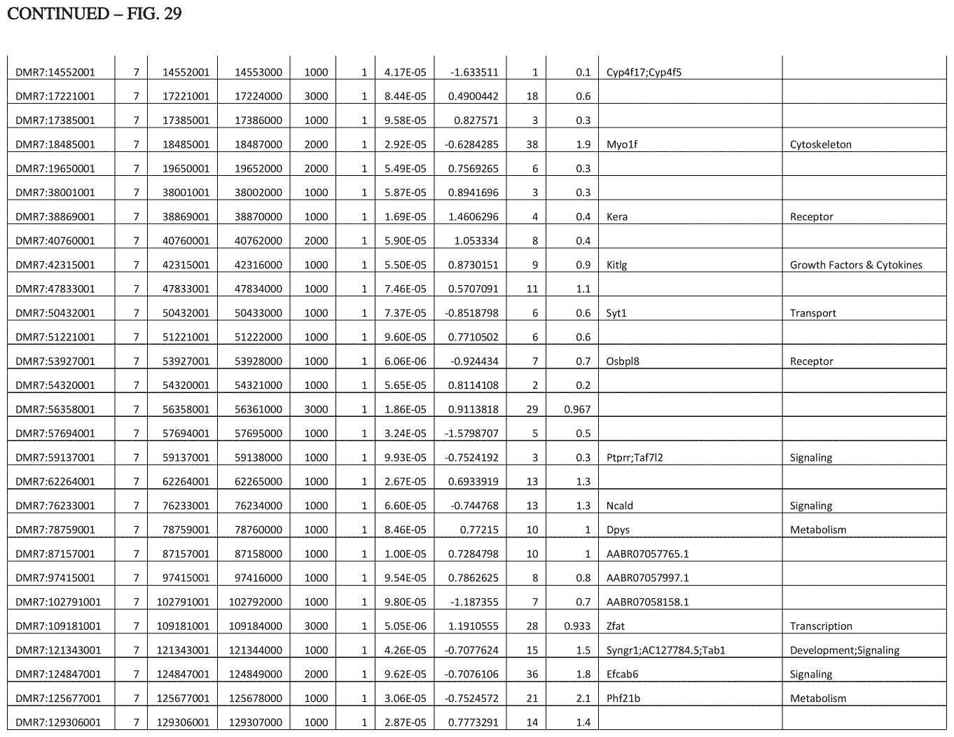

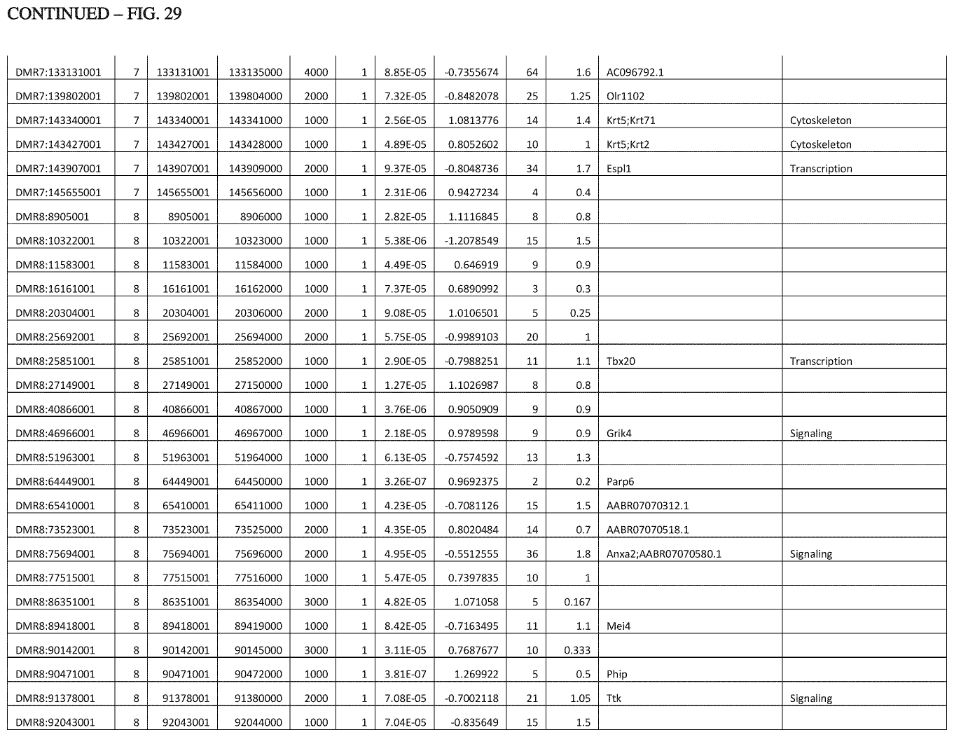

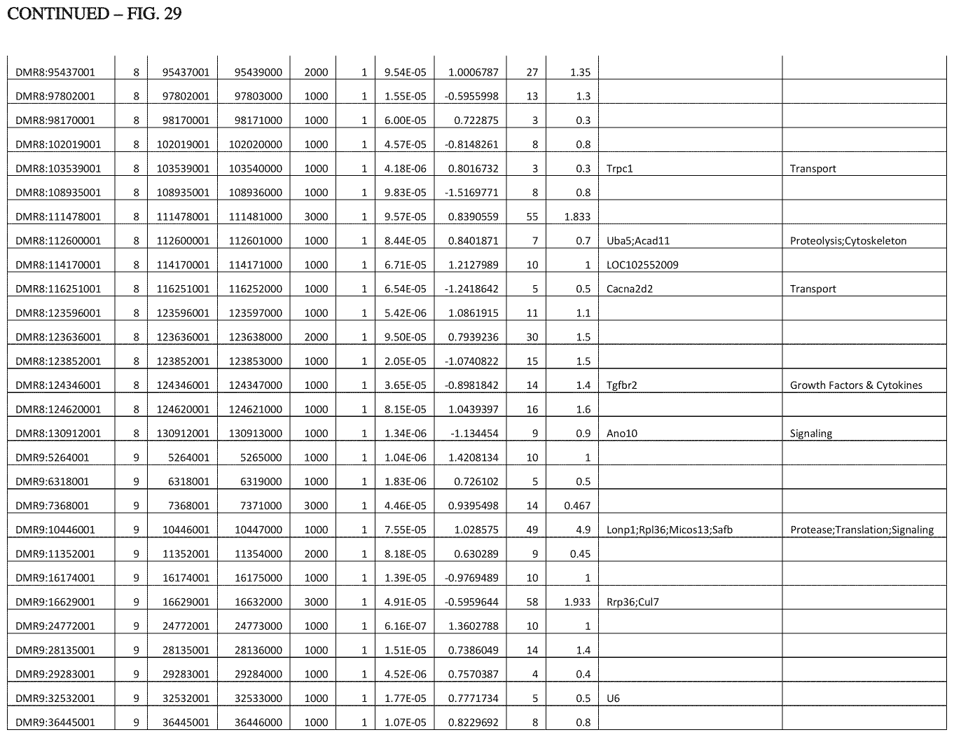

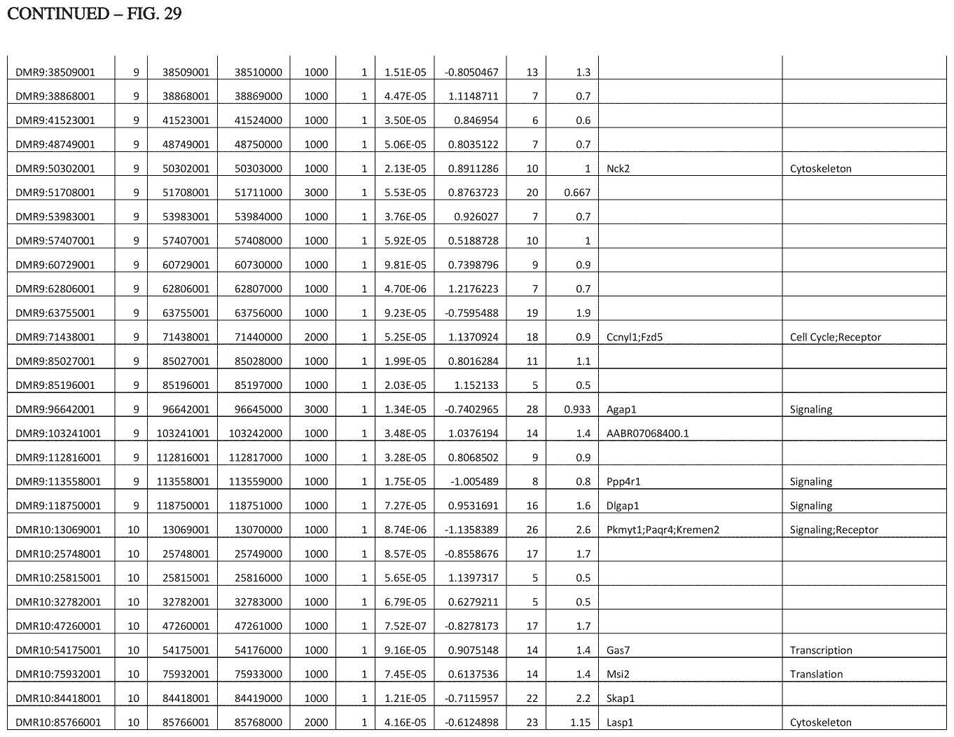

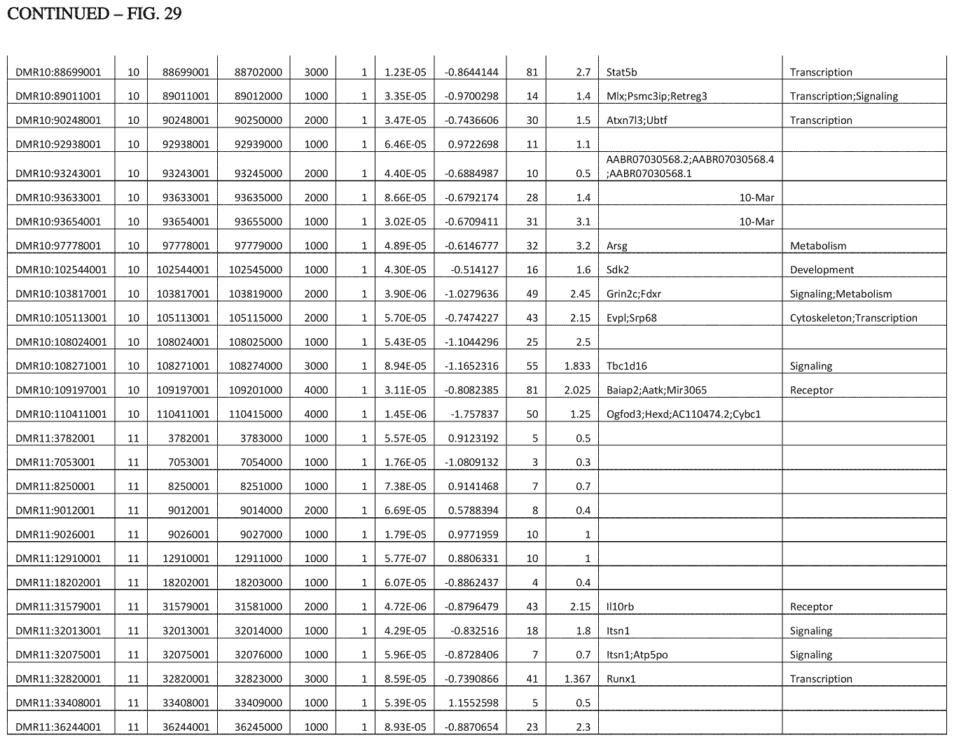

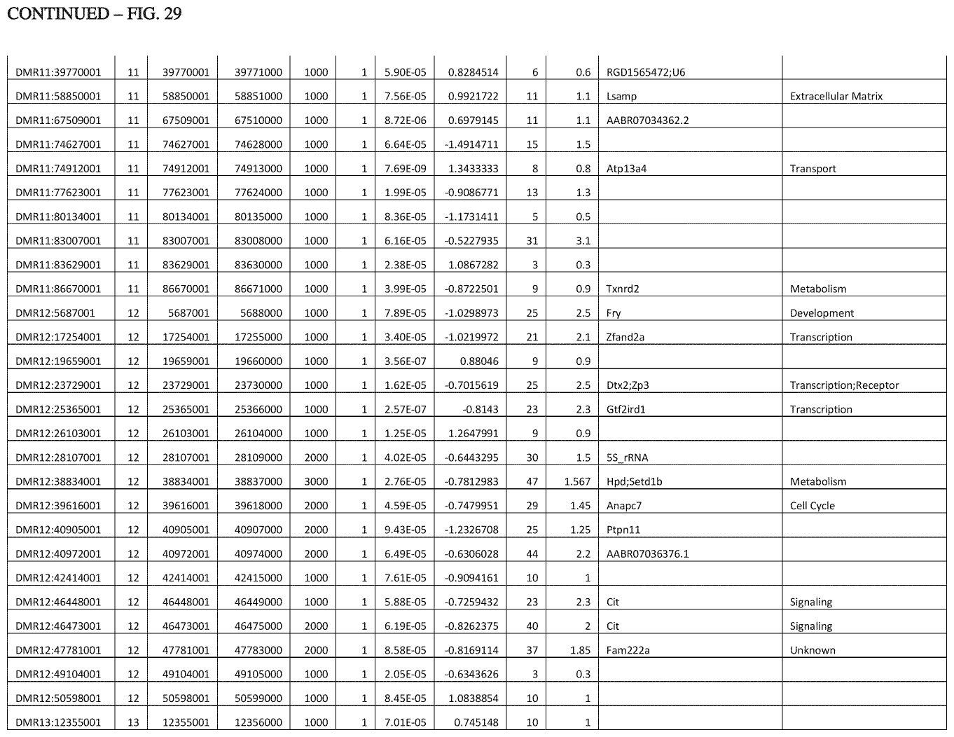

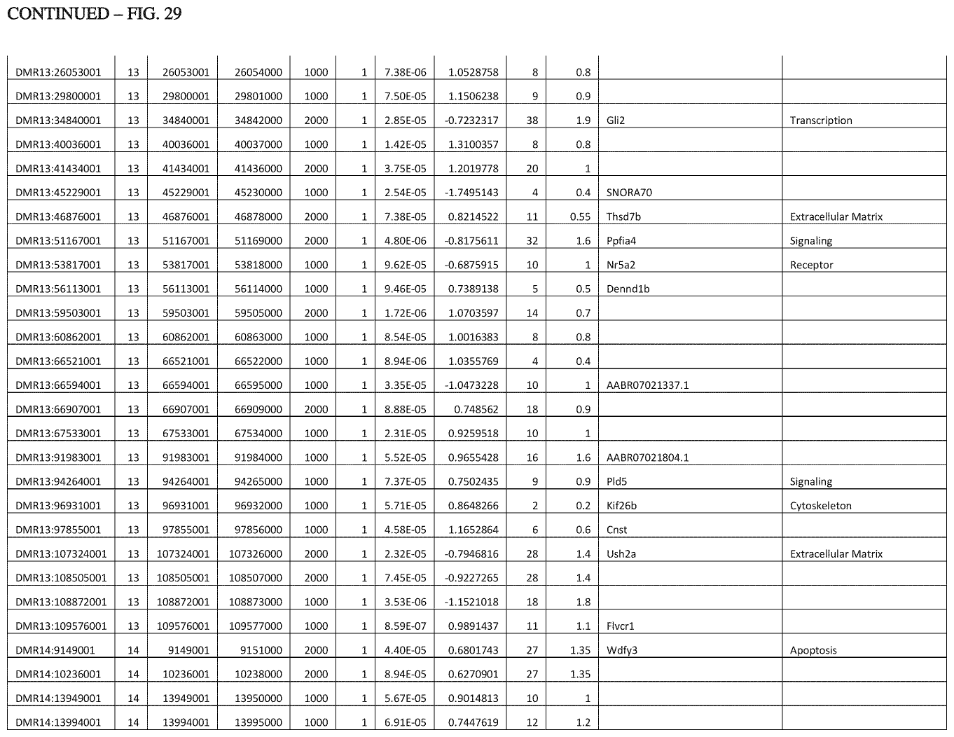

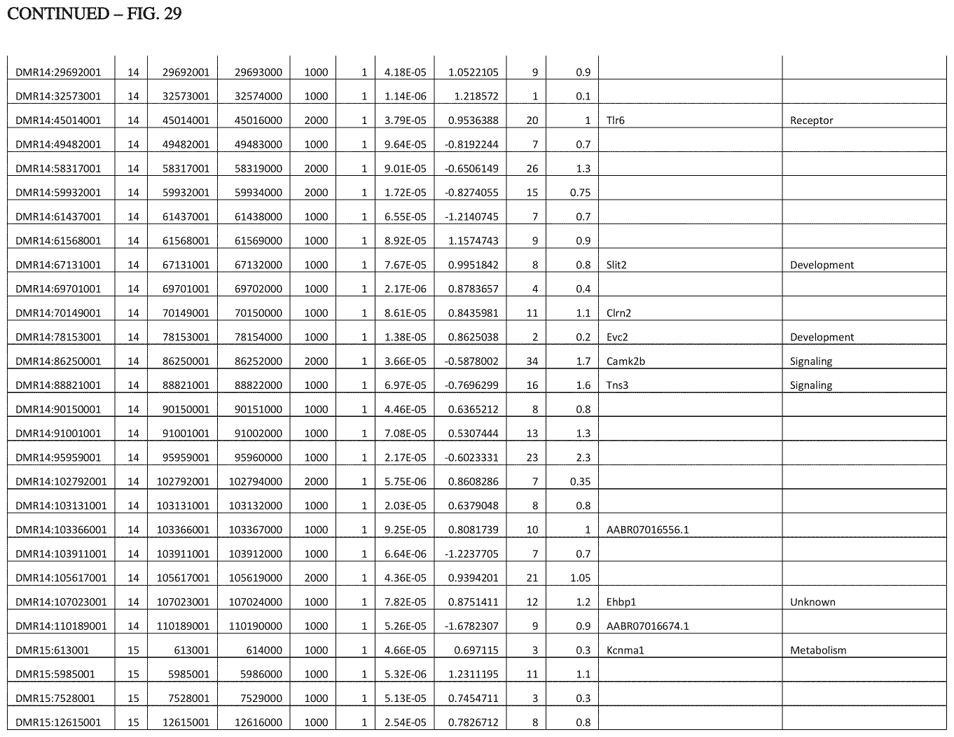

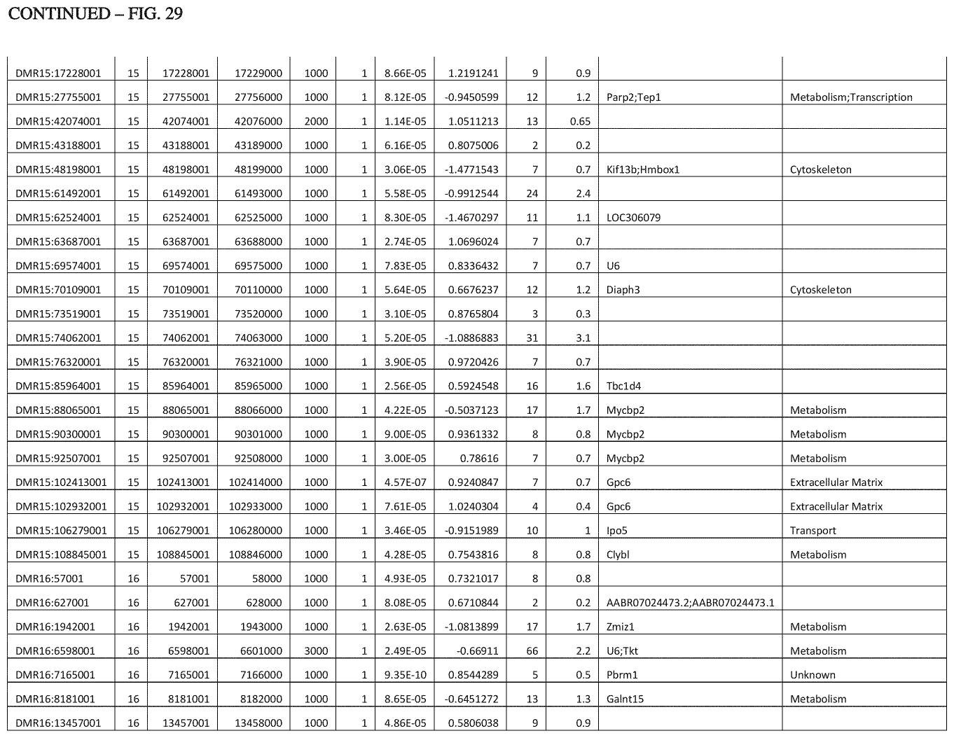

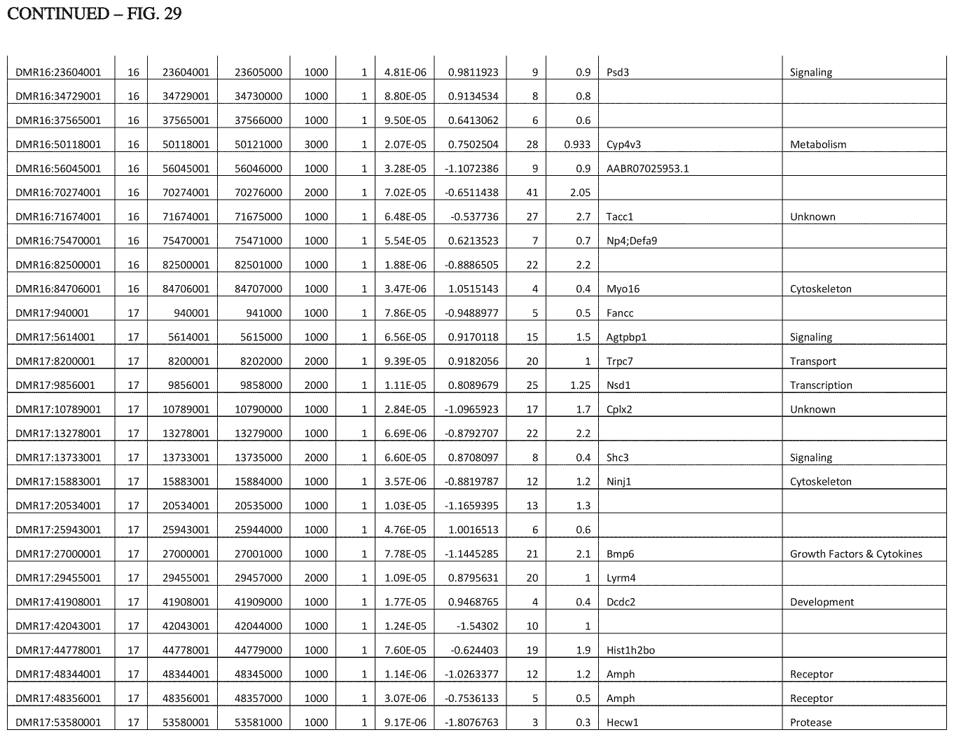

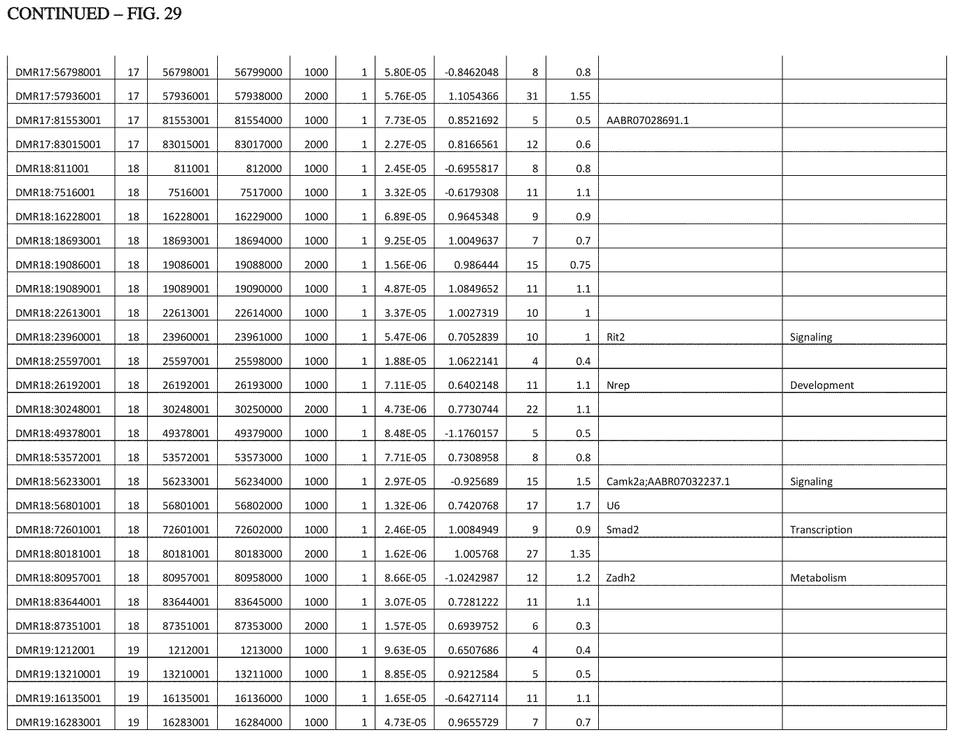

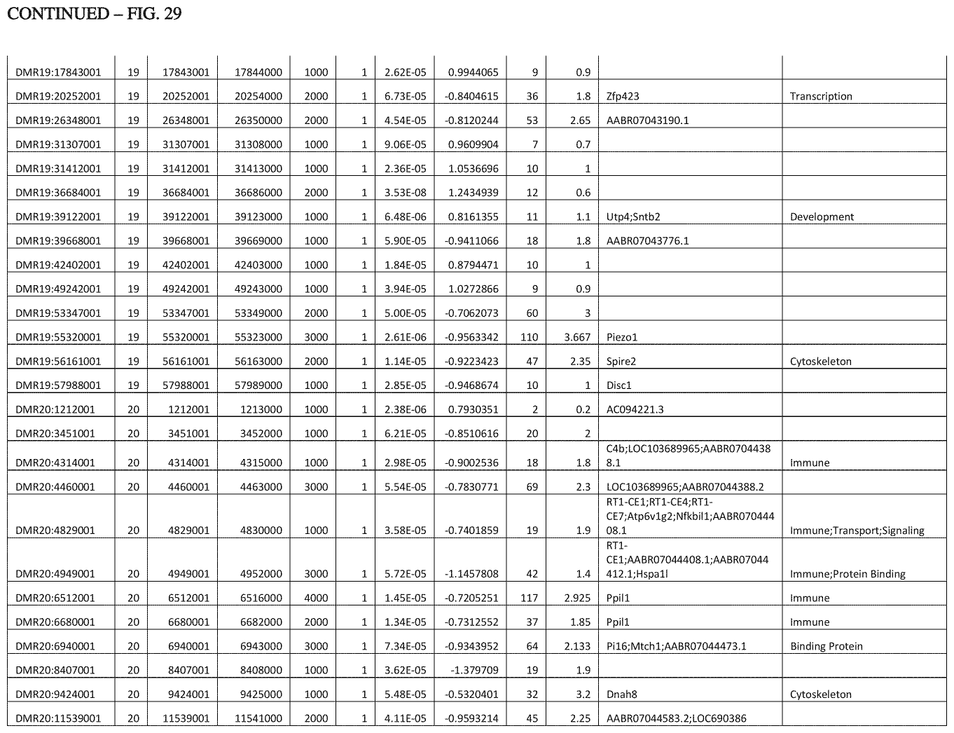

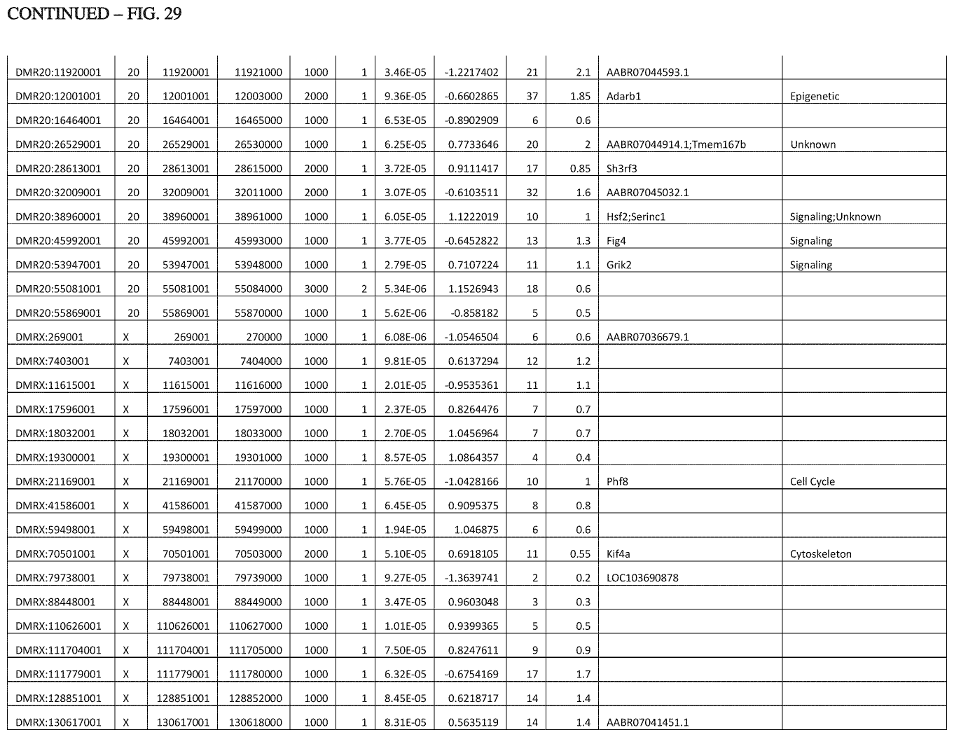

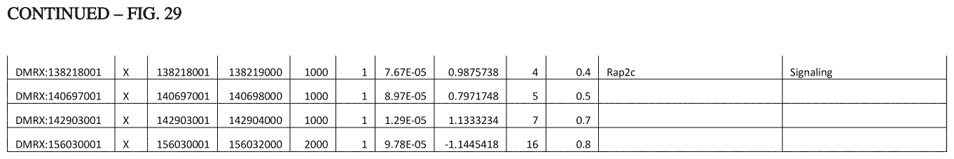

The F3 generation control lineage male pathology is listed in FIG. 29. Table 3 shows which individual animals were used in each disease group. The animals within the treated lineage which exhibited no disease served as the “no disease” or control set in the biomarker analysis. These are the highlighted “0”s in the “Total Disease” column. For each disease, only animals exhibiting a single disease were placed in that disease group for the biomarker analysis, indicated by a “+*” in the specific disease column. Any individual animals showing multiple diseases were included in the “multiple” disease category, indicated by a “+*” in the “Multiple Disease” column. Due to low prevalence of disease in the control animal groups, these animals were not used in the identification of epigenetic biomarkers.

TABLE 4
Individual Animal Pathology - F3 Generation Control Lineage Males

| Animal ID | Puberty | | | | | | | | Multiple | Total |
|---|---|---|---|---|---|---|---|---|---|---|
| (Sample ID) | Early | Late | Testis | Prostate | Kidney | Lean | Obese | Tumor | Disease | Disease |
| CM | — | — | — | — | — | — | + | — | — | 1 |
| (M15) | | | | | | | | | | |
| CM2 | — | — | — | — | — | — | — | — | — | |
| (M13) | | | | | | | | | | |
| CM3 | — | — | — | — | — | — | — | — | — | |
| (M14) | | | | | | | | | | |
| CM4 | — | — | | — | — | — | — | — | — | |
| (M18) | | | | | | | | | | |
| CM5 | — | — | — | — | — | — | — | — | — | |
| (M16) | | | | | | | | | | |
| CM6 | — | — | | — | + | + | — | — | + | 2 |
| (M17) | | | | | | | | | | |
| CM7 | — | — | — | — | — | + | — | — | — | 1 |
| CM8 | — | — | — | — | — | — | — | — | — | |
| CM9 | — | — | — | — | — | + | — | — | — | 1 |
| CM10 | — | — | — | — | — | + | — | — | — | 1 |
| CM11 | — | — | — | — | — | — | — | — | | |
| CM12 | — | — | + | + | — | — | — | — | + | 2 |
| CM13 | — | — | — | — | — | — | — | — | — | |
| CM14 | — | — | — | — | — | — | — | — | — | |
| CM16 | — | — | — | — | — | — | — | — | — | |
| (M1) | | | | | | | | | | |
| GCM17 | — | — | | | | | — | — | — | |
| CM17 | | | | | | | | | | |
| (M2) | | | | | | | | | | |
| CM17 | — | + | — | — | — | — | + | — | + | 2 |
| (M3) | | | | | | | | | | |
| CM18 | — | + | — | — | — | — | — | — | — | 1 |
| (M4) | | | | | | | | | | |
| CM19 | — | — | — | — | — | — | + | — | — | 1 |
| (M5) | | | | | | | | | | |
| CM20 | — | — | — | — | — | — | — | — | — | |
| CM21 | — | — | — | — | — | — | — | — | — | |
| (M6) | | | | | | | | | | |
| CM22 | — | — | — | — | + | — | — | — | — | 1 |
| (M7) | | | | | | | | | | |
| CM23 | — | — | — | — | — | — | — | — | — | |
| (M10) | | | | | | | | | | |
| CM24 | — | — | — | — | — | — | — | — | — | |
| (M8) | | | | | | | | | | |
| CM25 | — | — | — | — | — | — | — | — | — | |
| (M9) | | | | | | | | | | |
| CM26 | — | — | — | — | — | — | + | — | — | 1 |
| (M11) | | | | | | | | | | |
| CM27 | — | — | — | — | — | — | — | — | 0 | |
| (M12) | | | | | | | | | | |
| # affected | 0 | 2 | 1 | 1 | 2 | 4 | 4 | 0 | 3 | |
| # evaluated | 27 | 27 | 24 | 26 | 26 | 25 | 26 | 27 | | |

### Sperm Epigenetic Analysis

### Epididymal Sperm Collection and DNA Isolation

Briefly, the epididymis was dissected free of fat and connective tissue, then, after cutting open the cauda, placed into 6 ml of phosphate buffer saline (PBS) for 20 minutes at room temperature. Further incubation at 4° C. will immobilize the sperm. The tissue was then minced, the released sperm pelleted at 4° C. 3,000×g for 10 min, then resuspended in NIM buffer and stored at −80° C. for further processing.

An appropriate amount of rat sperm suspension was used for DNA extraction. Mammalian sperm heads may be resistant to sonication unlike somatic cells. Somatic cells and debris were therefore removed by brief sonication (Fisher Sonic Dismembrator, model 300, power 25), then centrifugation and washing 1-2 times in 1×PBS. The resulting pellet was resuspended in 820 μL DNA extraction buffer and 80 μl 0.1M DTT added, then incubated at 65° C. for 15 minutes. Then, 80 μl proteinase K (20 mg/ml) was added and the sample was incubated at 55° C. for 2-4 hours under constant rotation. Protein was removed by addition of protein precipitation solution (300 μl, Promega A795A), incubation for 15 minutes on ice, then centrifugation at 13,500 rpm for 30 minutes at 4° C. One ml of the supernatant was precipitated with 2 μl of Glycoblue (Invitrogen, AM9516) and 1 ml of cold 100% isopropanol. After incubation, the sample was spun at 13,500×g for 30 min at 4° C., then washed with 70% cold ethanol. The pellet was air-dried for about 5 minutes then resuspended in 100 μl of nuclease free water. For all generations, equal amounts of DNA from each individual's sample was used to produce 6 different DNA pools per lineage and the pooled DNA used for methylated DNA immunoprecipitation (MeDIP).

### Methylated DNA Immunoprecipitation (MeDIP)

Genomic DNA was sonicated and run on 1.5% agarose gel for fragment size verification. The sonicated DNA was then diluted with TE buffer to 400 μl, then heat-denatured for 10 min at 95° C., and immediately cooled on ice for 10 min to create single-stranded DNA fragments. Then 100 μl of 5×IP buffer and 5 μg of antibody (monoclonal mouse anti 5-methyl cytidine; Diagenode #C15200006) were added, and the mixture was incubated overnight on a rotator at 4° C. The following day magnetic beads (Dynabeads M-280 Sheep anti-Mouse IgG; Life Technologies 11201D) were pre-washed per manufacturer's instructions, and 50 μl of beads were added to the 500 μl of DNA-antibody mixture from the overnight incubation, then incubated for 2 h on a rotator at 4° C. After this incubation, the samples were washed three times with 1×IP buffer using a magnetic rack. The washed samples were then resuspended in 250 μl digestion buffer (5 mM Tris PH 8, 10 mM EDTA, 0.5% SDS) with 3.5 μl Proteinase K (20 mg/ml), and incubated for 2-3 hours on a rotator at 55°. DNA clean-up was performed using a Phenol-Chloroform-Isoamyalcohol extraction, and the supernatant precipitated with 2 μl of Glycoblue (20 mg/ml), 20 μl of 5M NaCl and 500 μl ethanol in −20° C. freezer for one to several hours. The DNA precipitate was pelleted, washed with 70% ethanol, then dried and resuspended in 20 μl H₂O or TE. DNA concentration was measured in Qubit (Life Technologies) with the ssDNA kit (Molecular Probes Q10212).

### Chromatin Immunoprecipitation (ChIP)

Histone chromatin immunoprecipitation with genomic DNA was performed. Individual rat sperm collections were generated, and the sperm counts were determined for each individual. Equal numbers of sperm were added from each individual for a total of 1.5 million sperm. To remove any somatic cell contamination sperm samples from each animal were sonicated for 10 seconds using a Sonic Dismembrator Model 300 (Thermo Scientific Fisher, USA) then centrifuged 1800×g for 5 min at 4° C. then resuspended and counted individually on a Neubauer counting chamber (Propper manufacturing Co., Inc., New York, USA) prior to pooling. The sperm pools were reconstituted up to 1 ml with PBS (phosphate buffered saline). To reduce disulfide bonds, 50 μl of 1 M DTT was added to each pool and the pools were then incubated for 2 hours at room temperature under constant rotation. To quench any residual DTT (dithiothreitol, Fisher Scientific, NY USA) in the reaction, 120 μl of fresh 1 M NEM (N-Ethylmaleimide, Thermo Scientific, Rockford, USA) was then added and the samples were incubated for 30 min at room temperature under constant rotation. The sperm cells were pelleted at 450×g for 5 min at room temperature and the supernatant was discarded. Pellets were resuspended in PBS and then spun again at 450×g for 5 min at room temperature. The supernatant was discarded and resuspended in 130 μl of complete buffer supplemented with tergitol 0.5% and DOC 1%. The samples were then sonicated using the Covaris M220. Covaris was set to a 10 min “Chromatin shearing” program and the program was run for each tube in the experiment.

After the Covaris sonication, 10 μl of each sample was run on a 1.5% agarose gel to verify fragment size. Samples were then centrifuged at 12,500×g for 10 min at room temperature. The supernatant was transferred to a fresh microfuge tube. 65 μl of protease inhibitor cocktail (1 tablet dissolved in 500 μl, 20× concentrated) (Roche, cat. no. 11 873 580 001) were added in each sample as well as 3 μl of antibody (anti-histone H3 pan-monoclonal antibody, cat no. 05-928, or anti-trimethyl-histone H3 (Lys27) polyclonal antibody, cat no. 07-449, both with broad spectrum species specifically form Millipore Corp, Temecula Calif. USA). The DNA-antibody mixture was incubated overnight on a rotator at 4° C. The following day, magnetic beads (ChIP-Grade protein G magnetic beads, Cell Signaling 9006) were pre-washed as follows: the beads were resuspended in the vial, then 30 μl per sample was transferred to a microfuge tube. The same volume of Washing Buffer (at least 1 ml) was added and the bead sample was resuspended. The tube was then placed into a magnetic rack for 1-2 min and the supernatant was discarded. The tube was removed from the magnetic rack and the beads were washed once. The washed beads were resuspended in the same volume of IP buffer as the initial volume of beads. 30 μl of beads were added to each DNA-antibody mixture from the overnight incubation, then incubated for 2 h on a rotator at 4° C. After the incubation, the bead-antibody-DNA complex was washed three times with IP buffer as follows: the tube was placed into a magnetic rack for 1-2 min and the supernatant was discarded, then washed with IP buffer 3 times. The washed bead-antibody-DNA solution was then resuspended in 300 μl of digestion buffer (1 M Tris HCl, pH 8.0, 0.5 M EDTA, 10% SDS) and 3 μl proteinase K (20 mg/ml). The sample was incubated for 3 h on a rotator at 56° C. After incubation the samples were extracted with Phenol-Chloroform-Isoamylalcohol and precipitated with 2 μl of Glycoblue (20 mg/ml), a one-tenth volume of 3 M sodium acetate and two volumes of ethanol overnight at −20° C.

The precipitate was centrifuged at 18,000×g for 30 min at 4° C. and the supernatant was removed, while not disturbing the pellet. The pellet was washed with 500 μl cold 70% ethanol, then centrifuged again at 18,000×g for 10 min at 4° C. and the supernatant was discarded. The tube was spun briefly to collect residual ethanol to bottom of tube and as much liquid as possible was removed with a gel loading tip. Pellet was air-dried at RT until it looked dry (about 5 min), then resuspended in 20 μl H2O. DNA concentration was measured in the Qubit (Life Technologies) with the BR dsDNA kit (Molecular Probes Q32853).

### MeDIP-Seq/ChIP-Seq Analysis

MeDIP DNA was used to create libraries for next generation sequencing (NGS) using the NEBNext Ultra RNA Library Prep Kit for Illumina (San Diego, Calif.) starting at step 1.4 of the manufacturer's protocol to generate double stranded DNA from the single-stranded DNA resulting from MeDIP. After this step the MeDIP DNA, and starting with the ChIP DNA, the manufacturer's protocol was followed indexing each sample individually with NEBNext Multiplex Oligos for Illumina. The WSU Spokane Genomics Core sequenced the samples on the Illumina HiSeq 2500 at PESO, with a read size of approximately 50 bp and approximately 20 million reads per pool. Ten libraries were run in one lane.

### Statistics and Bioinformatics

Data quality was assessed using the FastQC program (http://www.bioinformatics.babraham.ac.uk/projects/fastqc/), and reads were cleaned and filtered to remove adapters and low quality bases using Trimmomatic. The reads for each MeDIP and ChIP sample were mapped to the Rnor 6.0 rat genome using Bowtie2 with default parameter options. The mapped read files were then converted to sorted BAM files using SAMtools. The MEDIPS R package was used to calculate differential coverage between control and exposure sample groups. The reference genome was broken into 1000 bp windows. Only genomic windows with at least an average of 10 reads per sample were kept for subsequent analysis. The edgeR p-value was used to determine the relative difference between the two groups for each genomic window. Windows with an edgeR p-value less than an arbitrarily selected threshold were considered DMRs or DHRs. The DMR/DHR edges were extended until no genomic window with an edgeR p-value less than 0.1 remained within 1000 bp of the DMR/DHR.

DMRs and DHRs were annotated using the biomaRt R package to access the Ensembl database. The genes that associated with DMRs/DHRs were then input into the KEGG pathway search to identify associated pathways. The DMR/DHR associated genes were then automatically sorted into functional groups using information provided by the DAVID and Panther databases incorporated into an internal curated database.

### Animal Model

Outbred Sprague Dawley gestating female rats (F0 generation) were administered an intraperitoneal dose of 25 mg/kg body weight of atrazine (4% of rat oral LD50 and 50% of NOAEL). These doses were administered at 90 days of age, during embryonic days 8-14 (E8-E14) of fetal gonadal sex determination. The F1 generation offspring was directly exposed as a fetus and F2 generation grand-offspring exposed as the germline in the F1 generation. These were each bred at 90 days of age within the lineage. The F3 generation great-grand-offspring is required to establish the transgenerational inheritance generation of ancestral exposure. This transgenerational generation was the focus of the current study. A control lineage was established that used F0 gestating rats exposed to the vehicle control dimethyl sulfoxide (DMSO). Disease pathology was evaluated in atrazine exposure and control lineages at 1 year of age. The atrazine exposure lineage transgenerational individuals with specific disease or pathology were grouped as representatives of the pathology exhibited. The remaining individuals were grouped as “no disease.” Comparisons between these two groups were made during analysis of sperm DNA methylation and histone retention. The differentially methylated regions and differential histone retention site allows the identification of specific disease associated epigenetic biomarkers.

### Pathology Analysis

Pathology analysis was assessed with histology sections of testis, kidney, prostate, and gonadal fat pads. The complete histological sections were analyzed by two different observers blinded to the exposure, unless they disagreed, and then an additional different third observer was used. Briefly, each counter records the incidence of abnormalities in each tissue. In testis, atrophy of a seminiferous tubule, the arrest of maturation of sperm (indicated by sloughed cells in the center of the tubule), and the presence of vacuoles were indicated disease pathologies. The abnormalities counted in kidney include a reduction in size of glomeruli, a thickening of the Bowman's capsule, and the presence of cysts. Prostate abnormalities counted include atrophy of the epithelial cells, hyperplasia in the epithelial layer, and the presence of vacuoles within the epithelial layer of the prostatic glands. Obese and lean phenotypes were assigned following assessment of adipocyte size (area), body mass index (BMI) and abdominal adiposity. Late puberty was noted during development. The individual animals are listed in Table 3. A (+) indicates presence of disease and (−) indicates absence of disease for the current F3 generation atrazine lineage male pathology. The control lineage males were analyzed in a similar manner to allow a comparison to assess atrazine induced disease in the atrazine lineage (Table 4). Only the individuals with a single disease for a specific pathology were used for that pathology molecular analysis. Animals exhibiting more than one disease are all listed under the category “Multiple Disease.” Due to low prevalence of disease in the control animal groups (Table 4), those animals were not used in the identification of epigenetic biomarkers.

### Sperm DNA Methylation

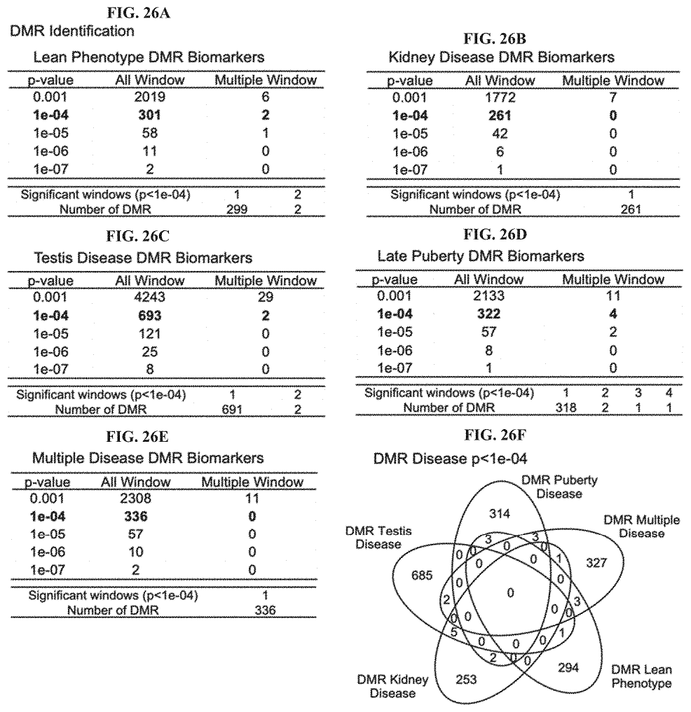

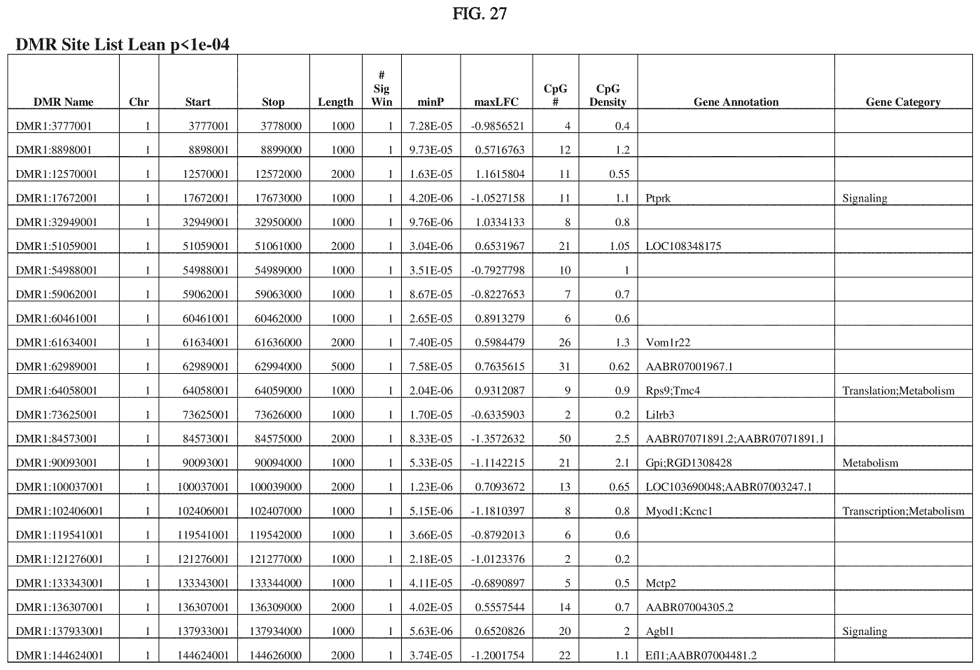

The experimental design was focused on the identification of transgenerational DMRs and DHRs in sperm. Sperm were collected from the atrazine lineage F3 generation males for epigenetic analysis. DNA from the sperm was isolated and fragmented with sonication. The methylated DNA immunoprecipitation (MeDIP) using a methyl-cytosine antibody was used to identify alterations in DNA methylation. The methylated DNA fragments were then sequenced for an MeDIP-Seq analysis. The differential DNA methylation regions (DMRs) were identified between the disease versus non-disease within the atrazine lineage animals (FIG. 26). The transgenerational sperm DMR numbers are presented in FIG. 26 for different edgeR statistical p-value cutoff thresholds, and p<1e−04 (diseased versus non-diseased) for the atrazine lineage were selected as the threshold for subsequent analyses. Disease-specific DMRs were then identified among the atrazine treated animals exhibiting disease phenotypes, including lean phenotype, kidney disease, testis disease, late puberty, and multiple disease, compared against atrazine lineage individuals exhibiting no disease (FIG. 26A-26E). The all windows represents all DMR windows, and multiple site are those with nearby 1 kb sites. Only the late puberty DMRs had multiple sites. In the current analysis 1000 bp windows were used in the identification of DMRs.

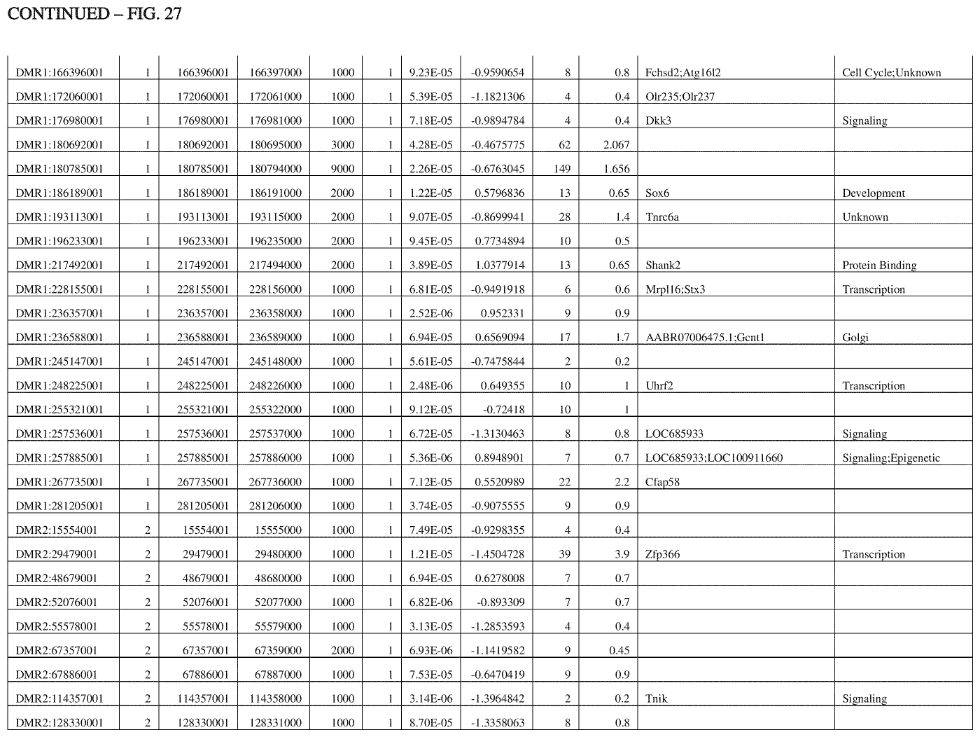

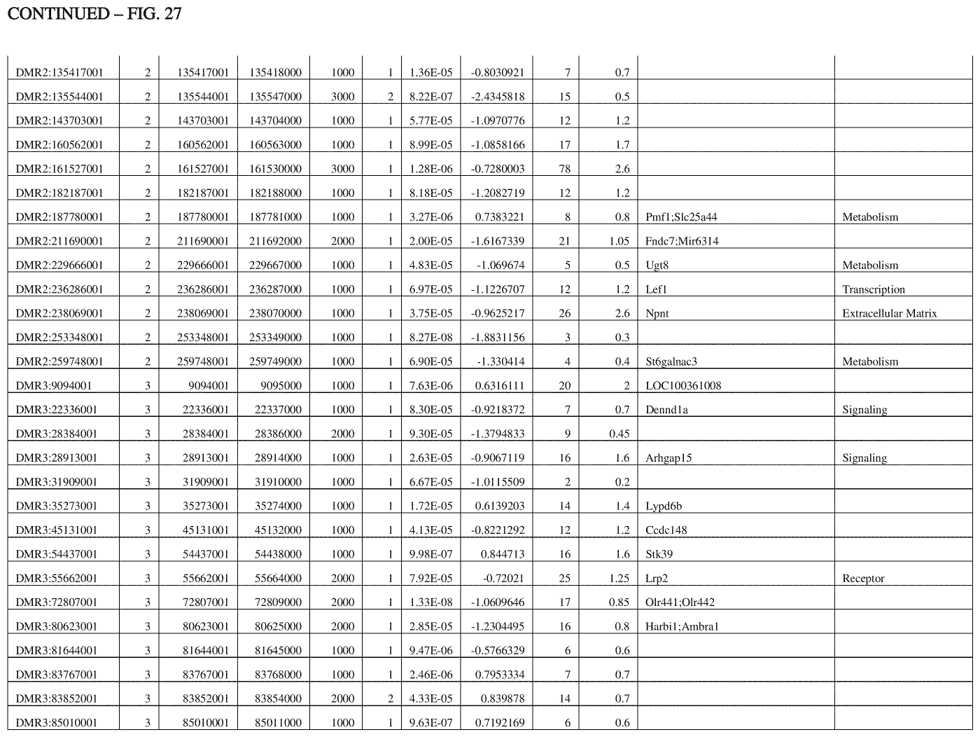

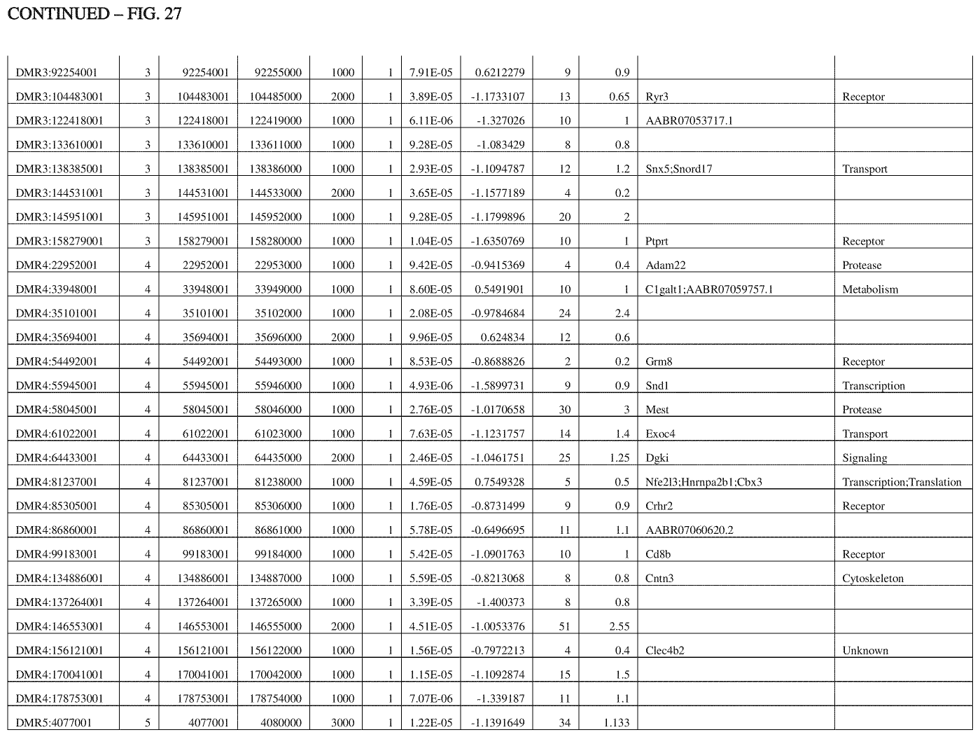

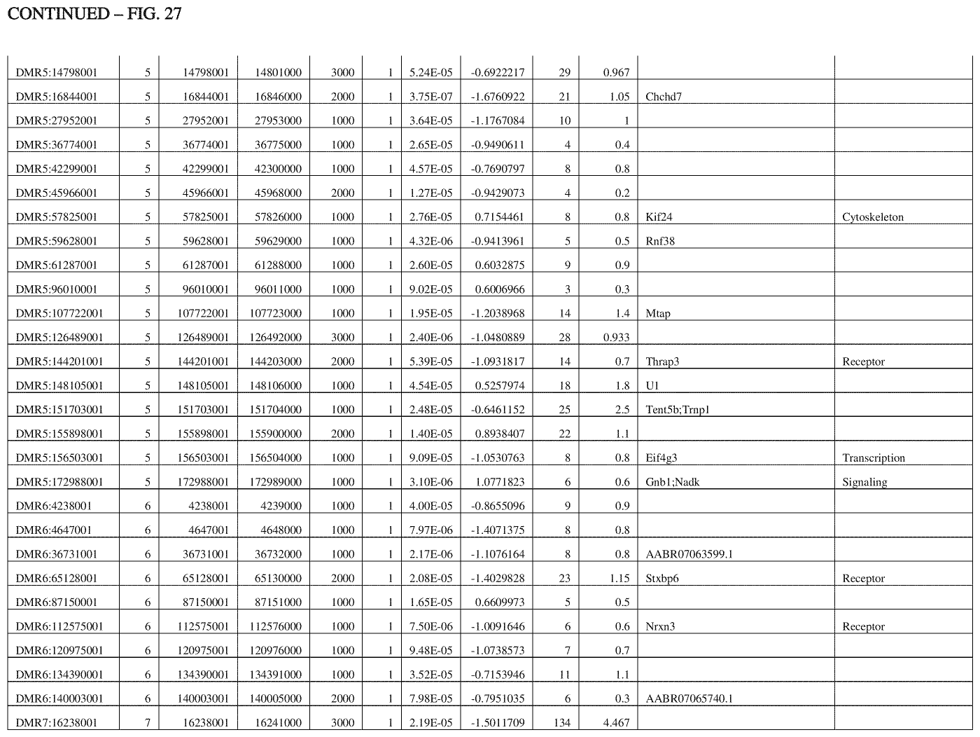

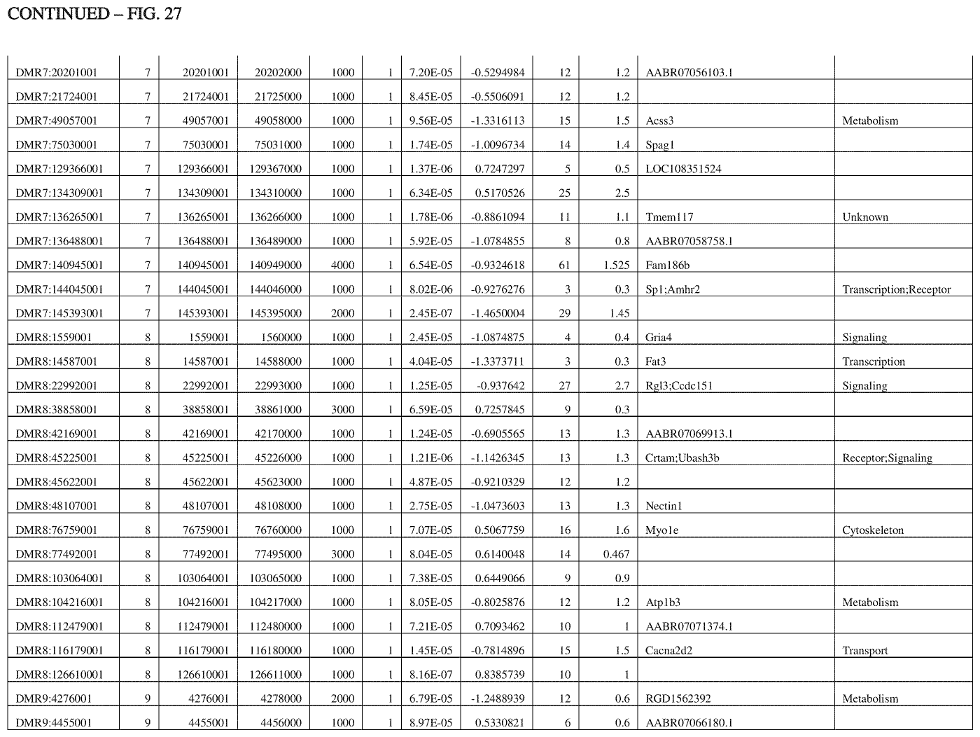

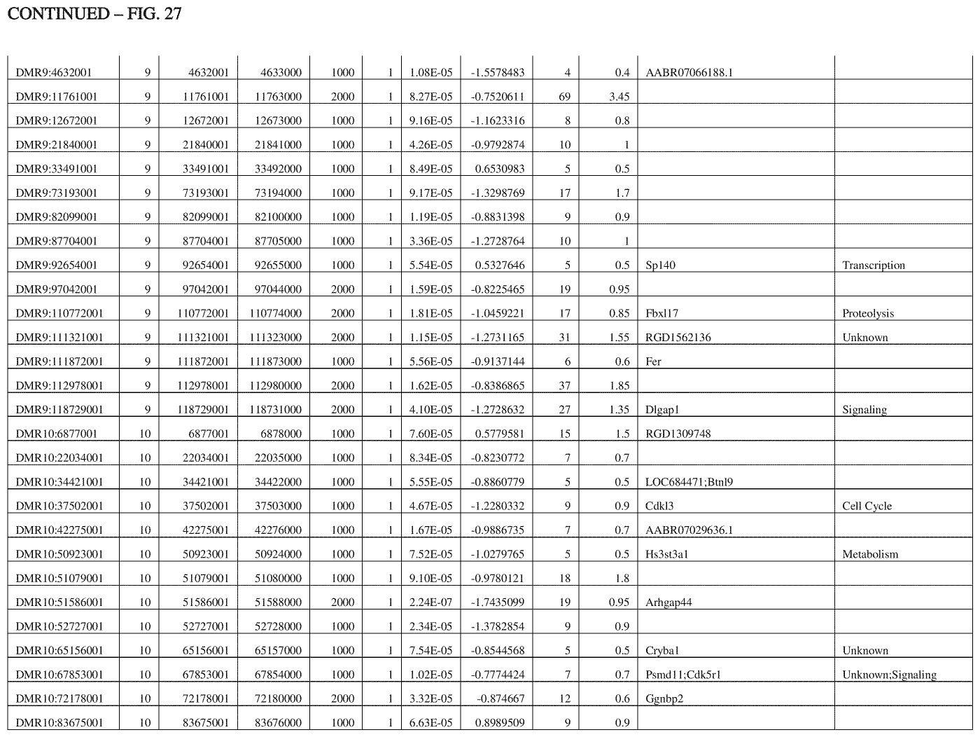

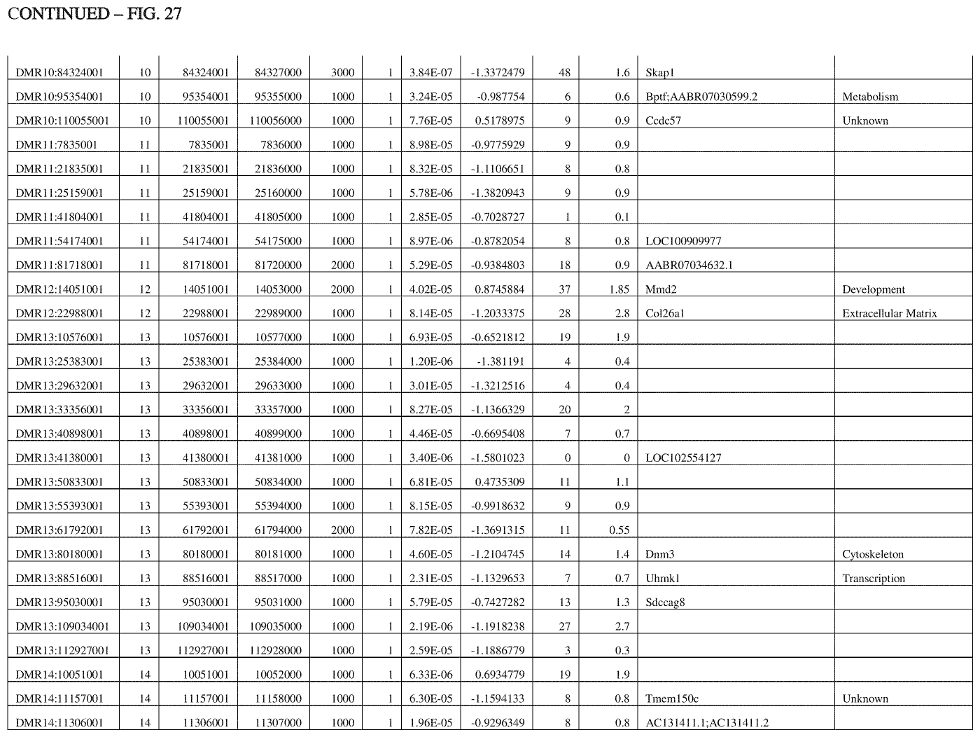

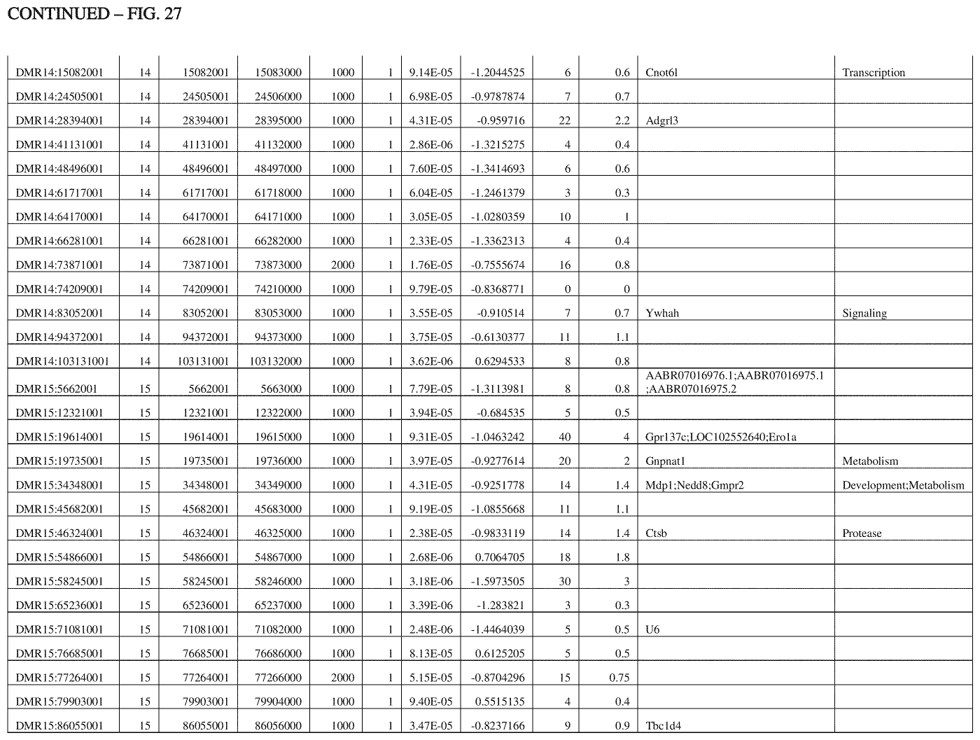

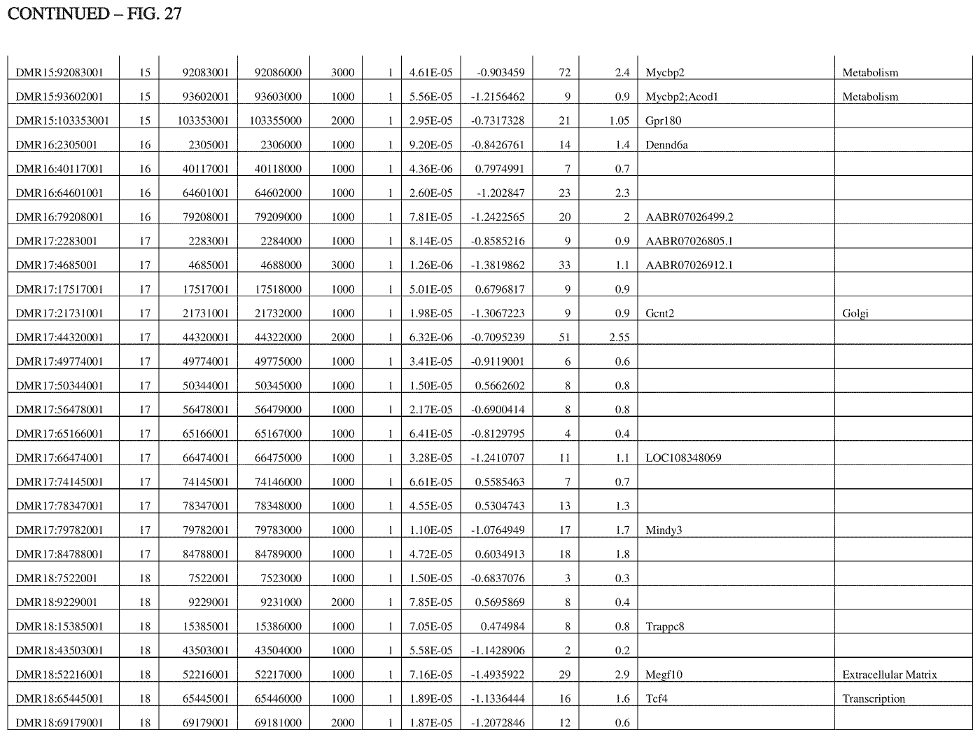

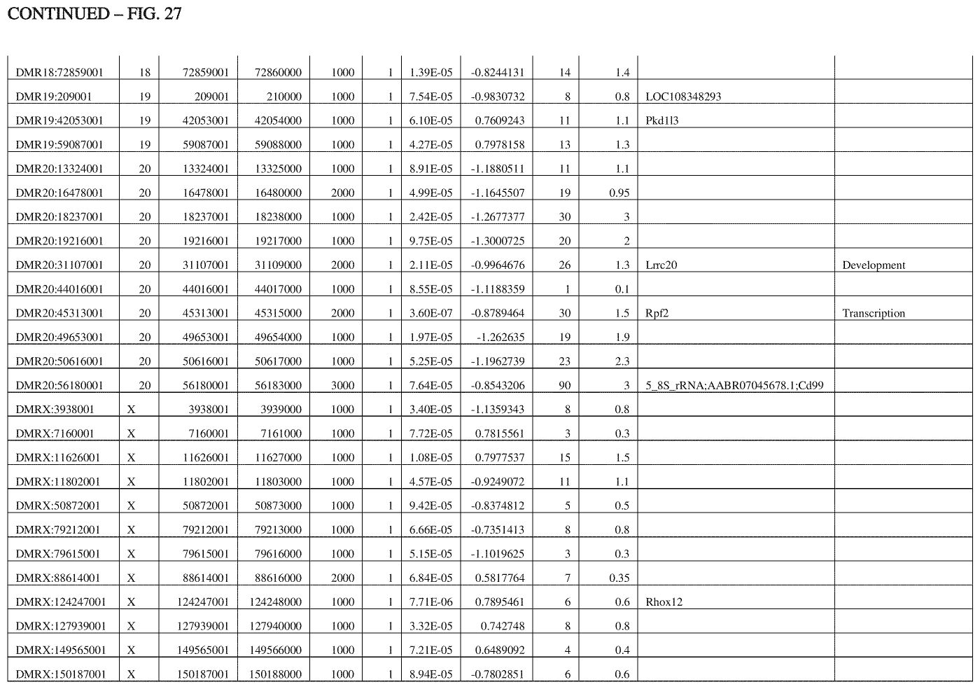

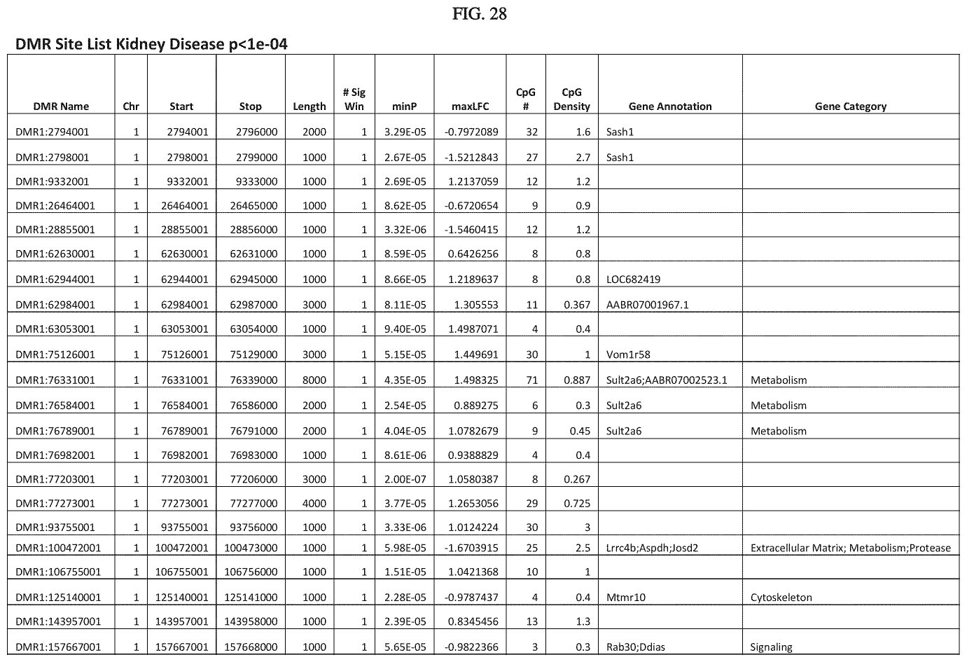

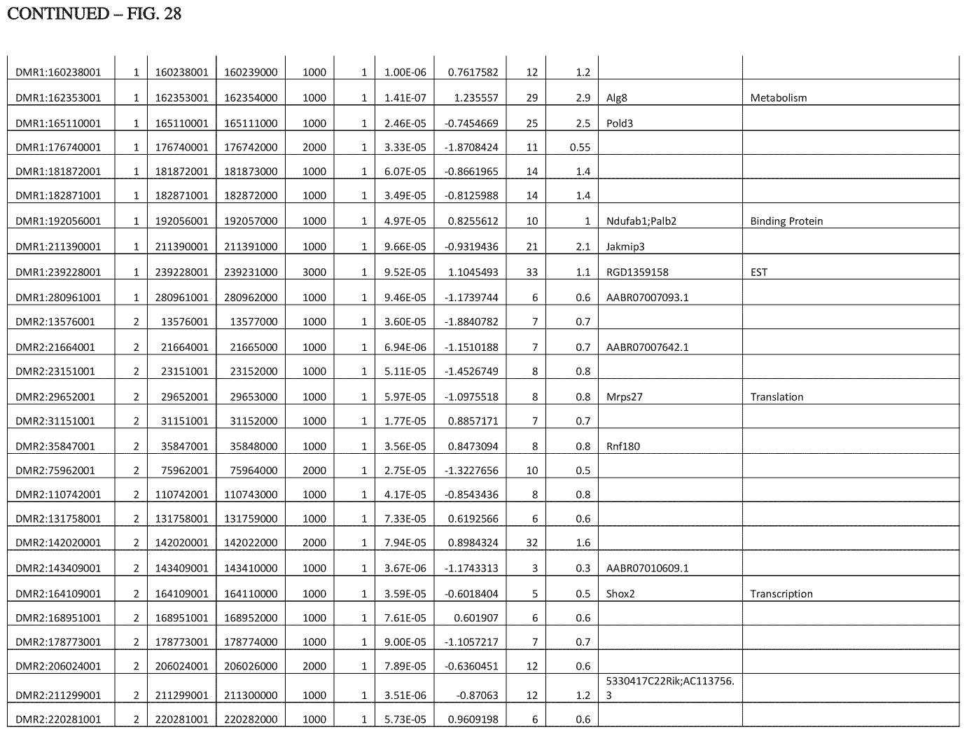

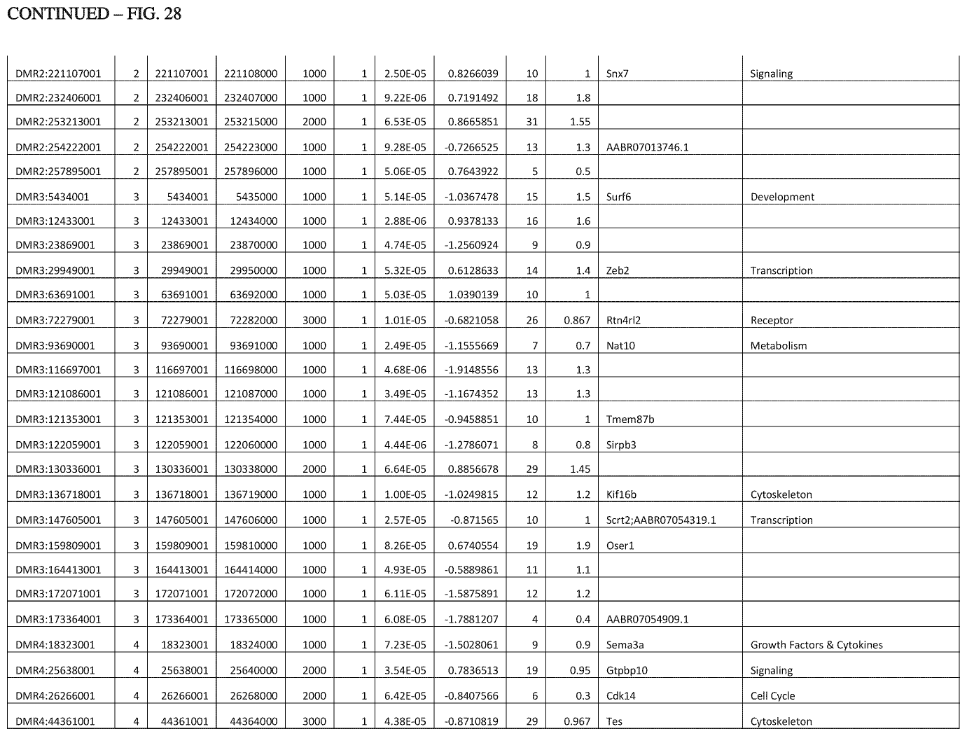

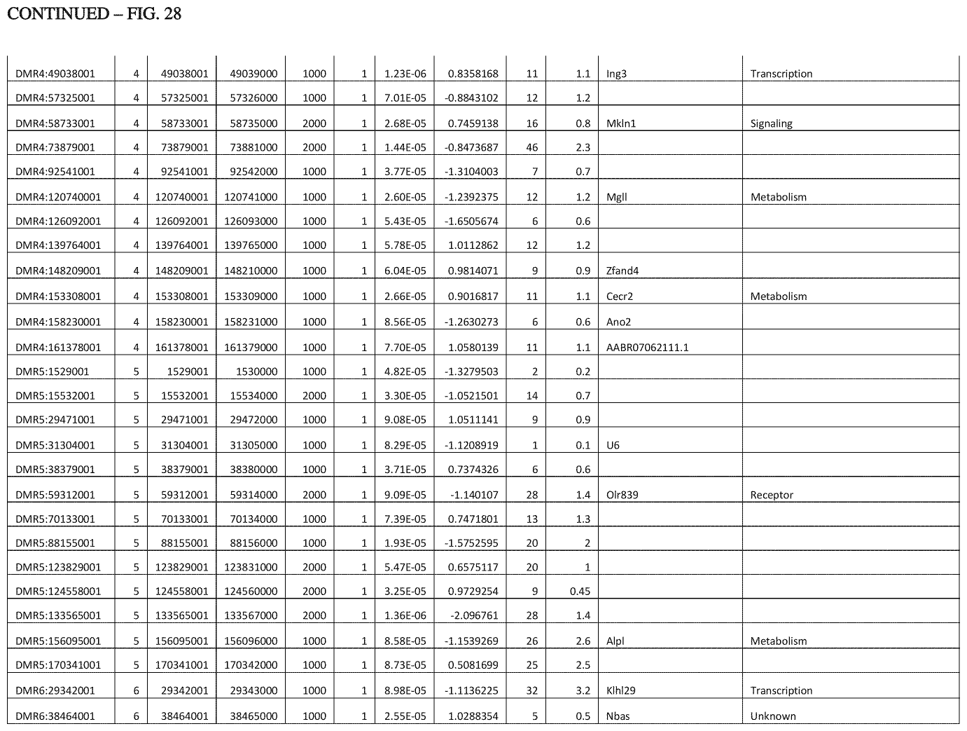

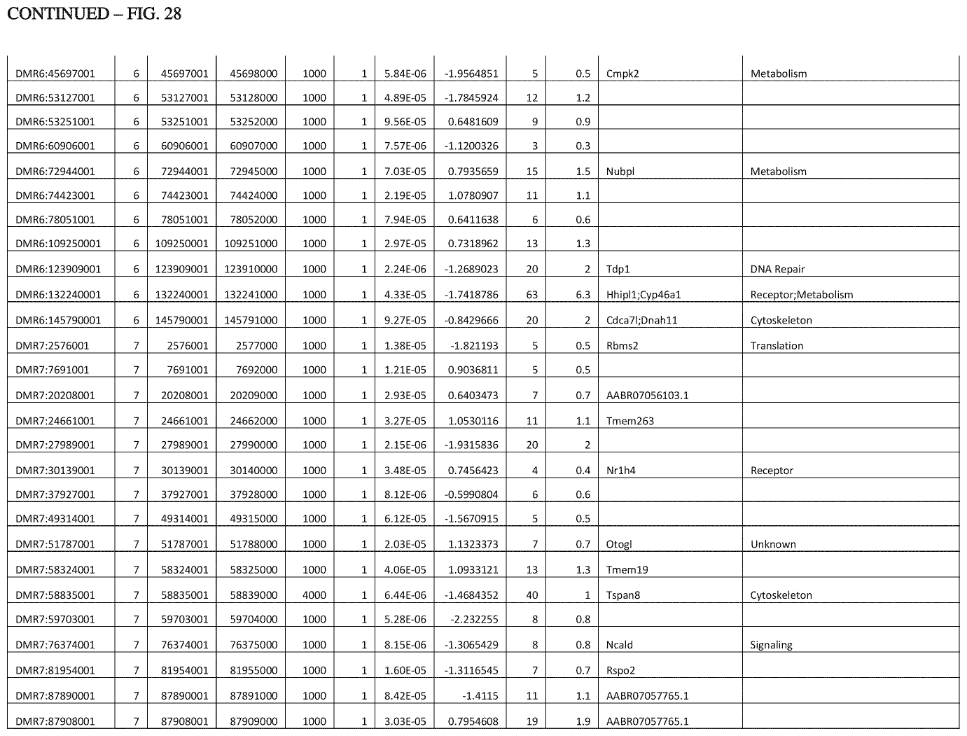

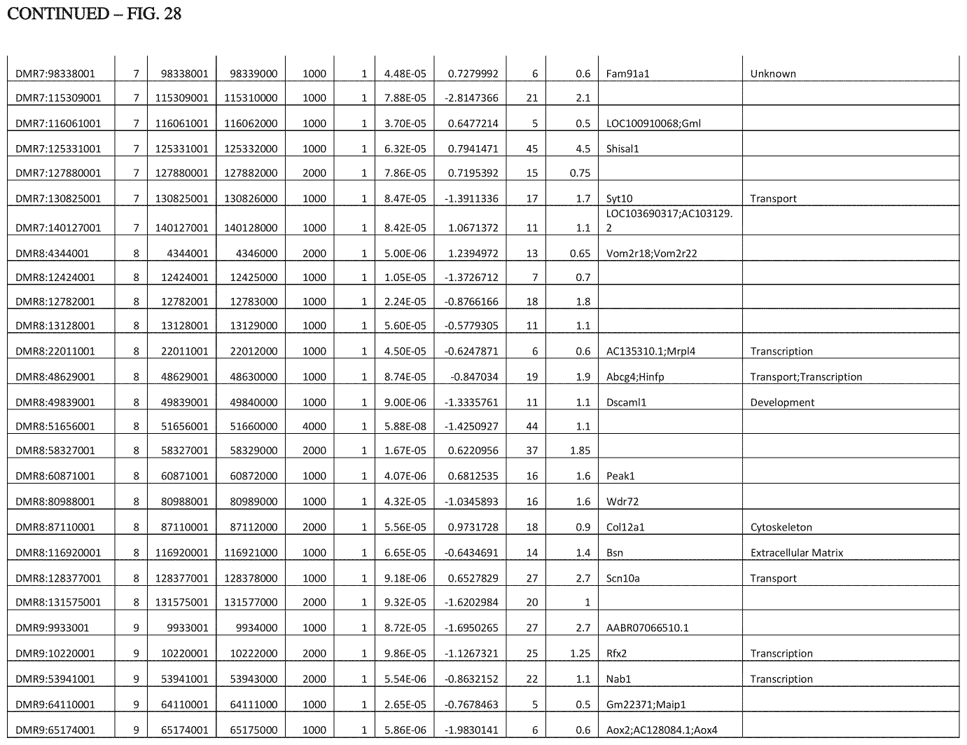

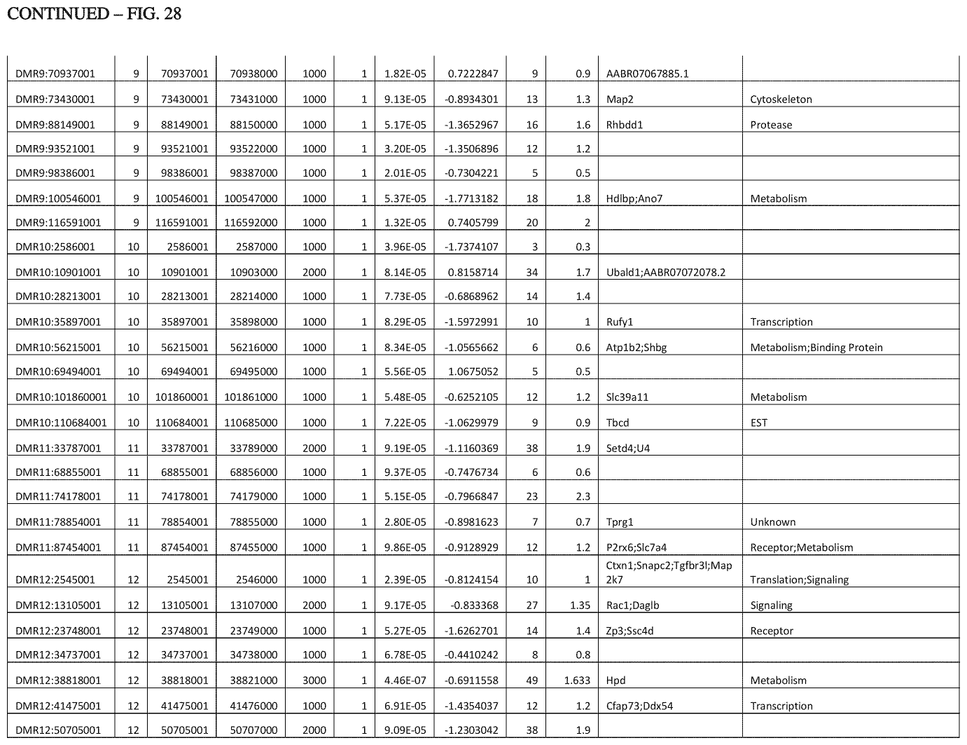

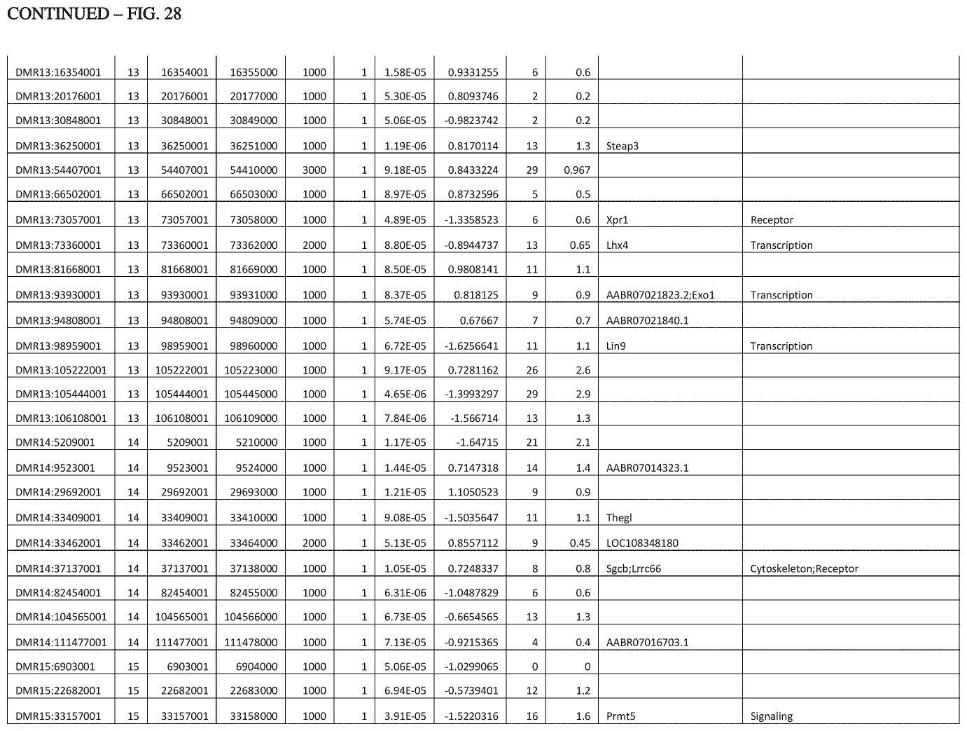

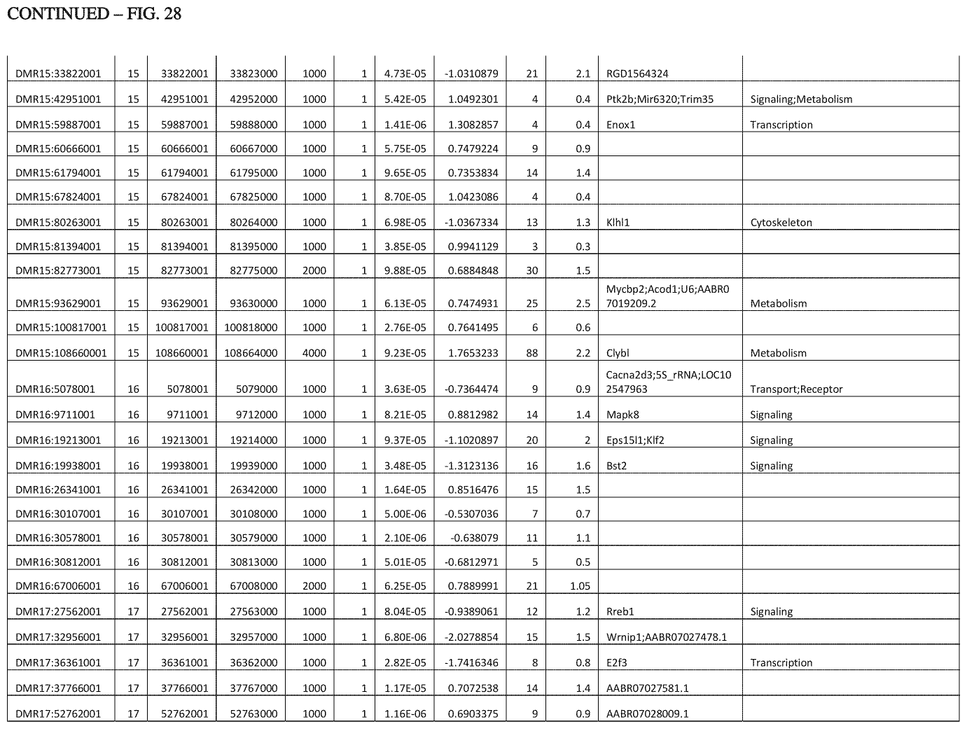

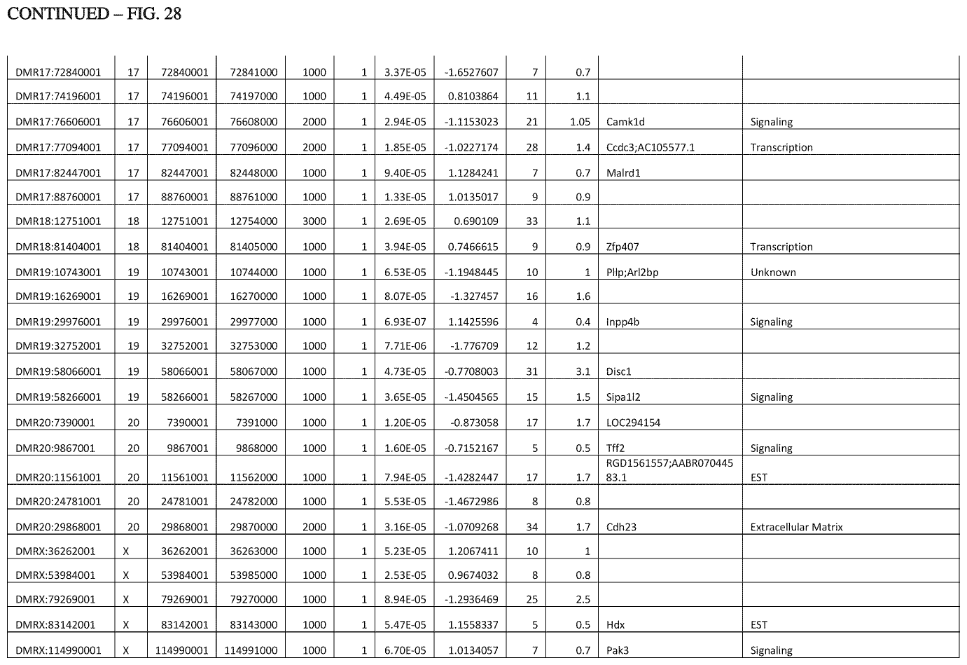

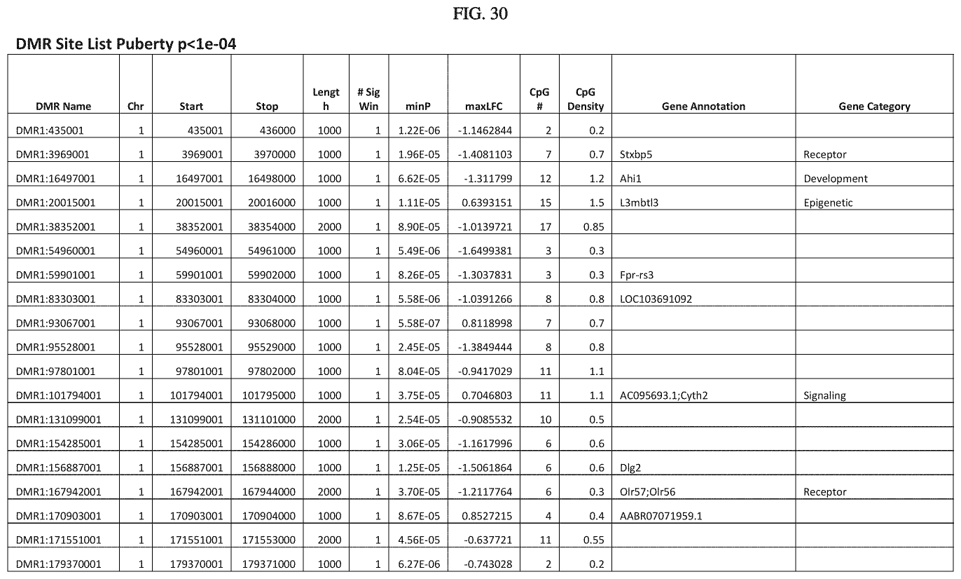

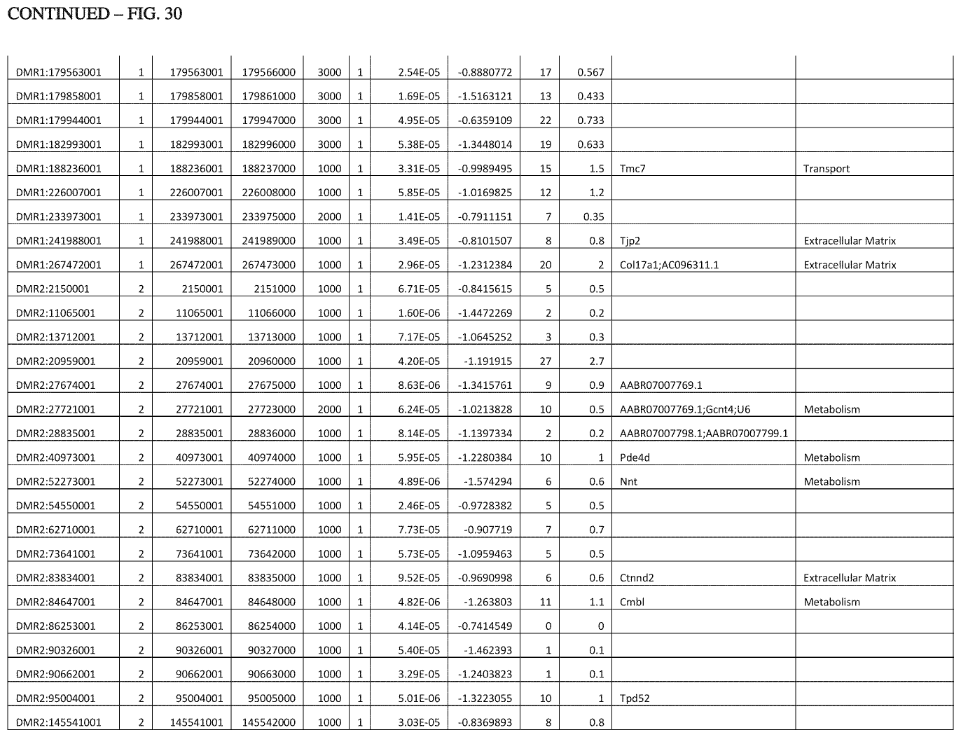

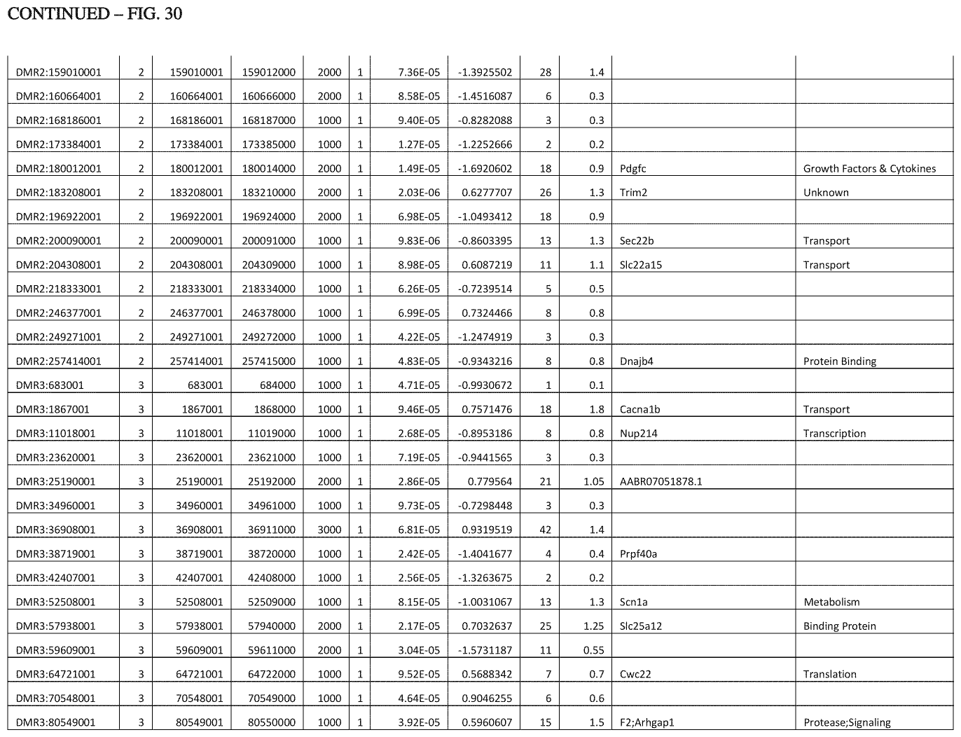

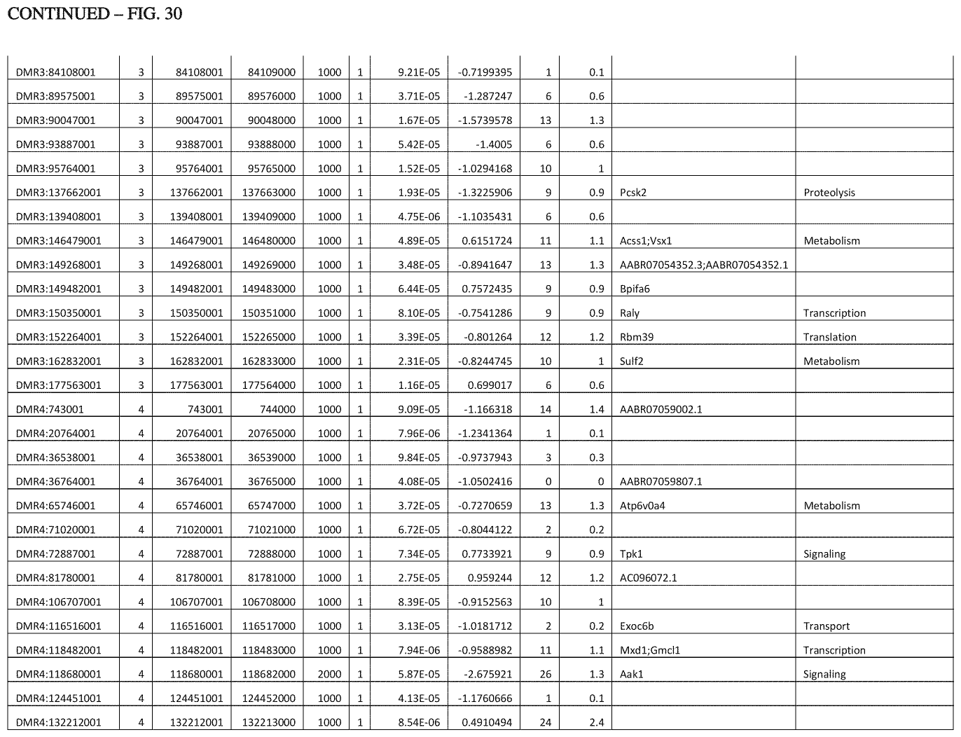

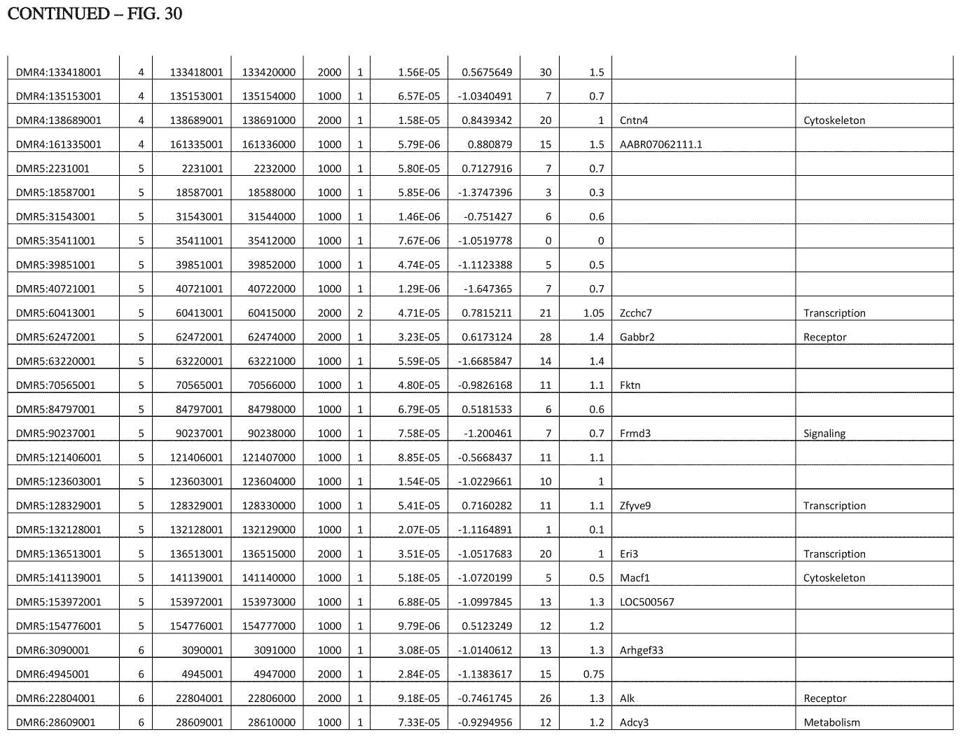

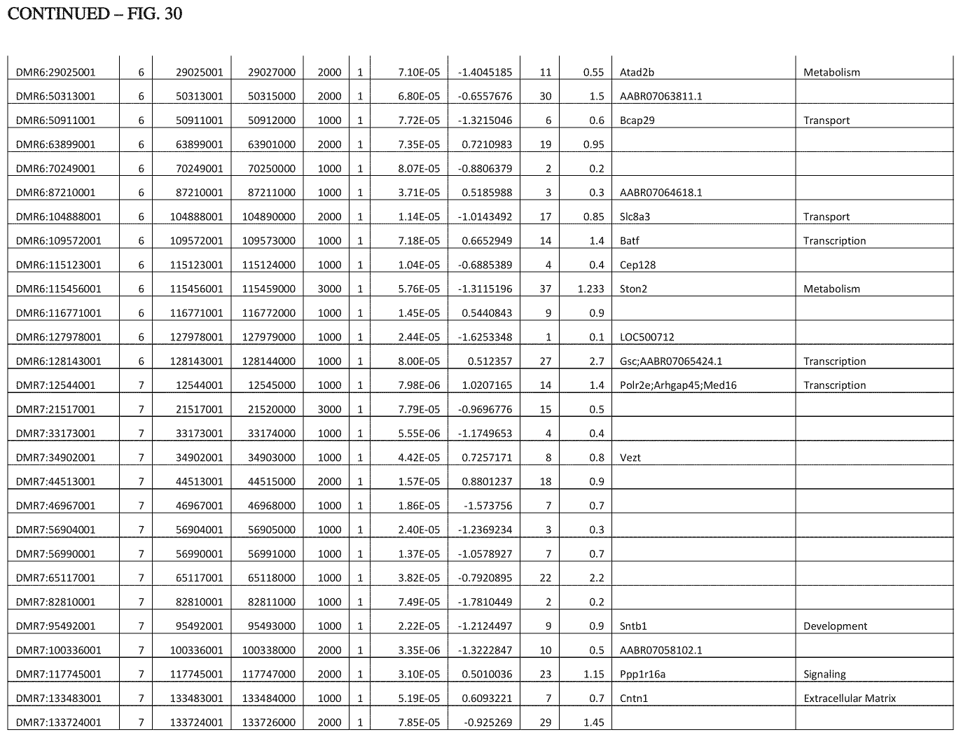

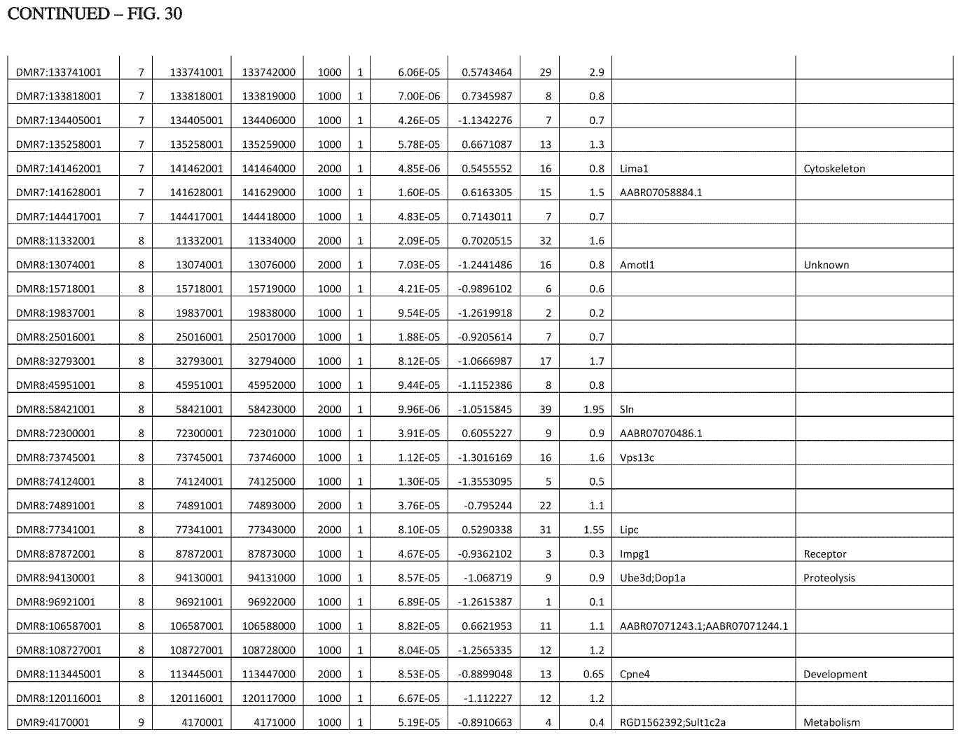

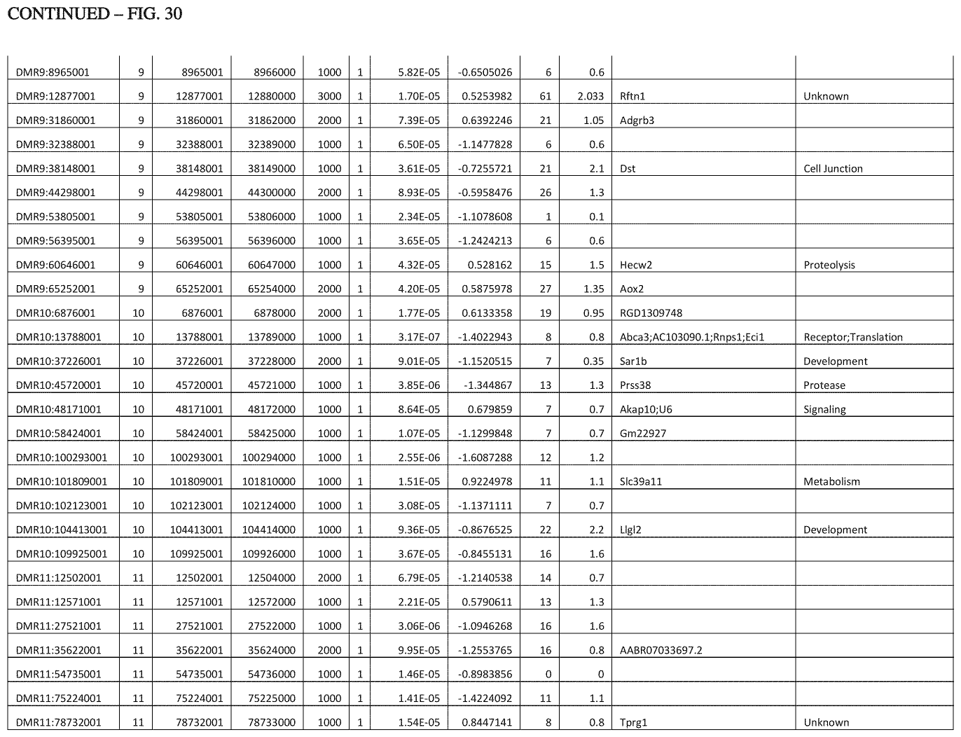

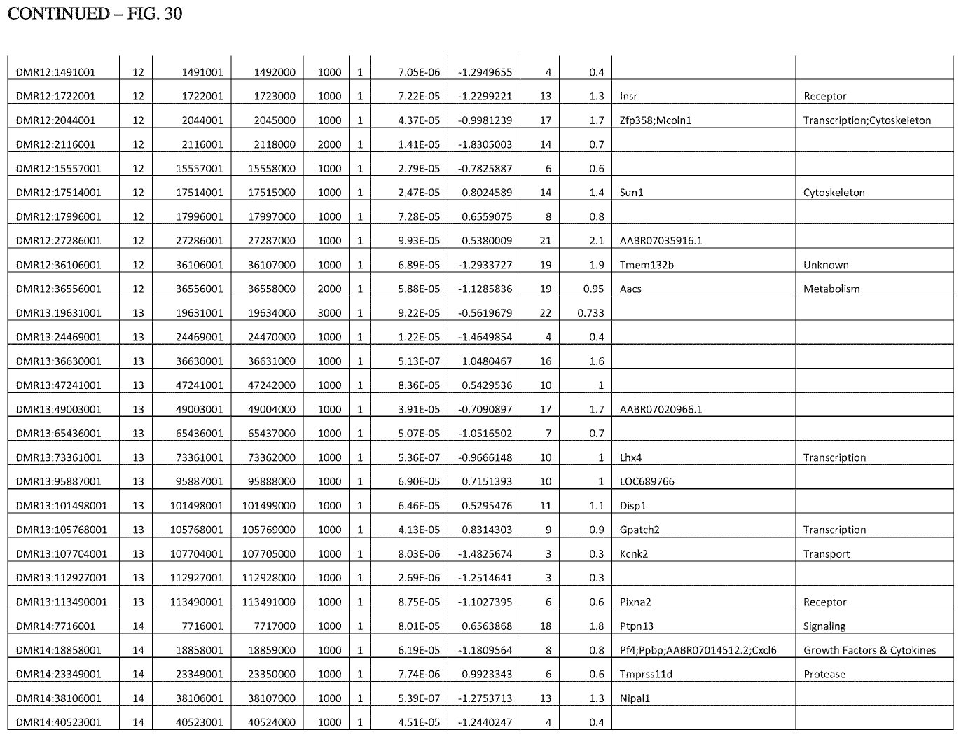

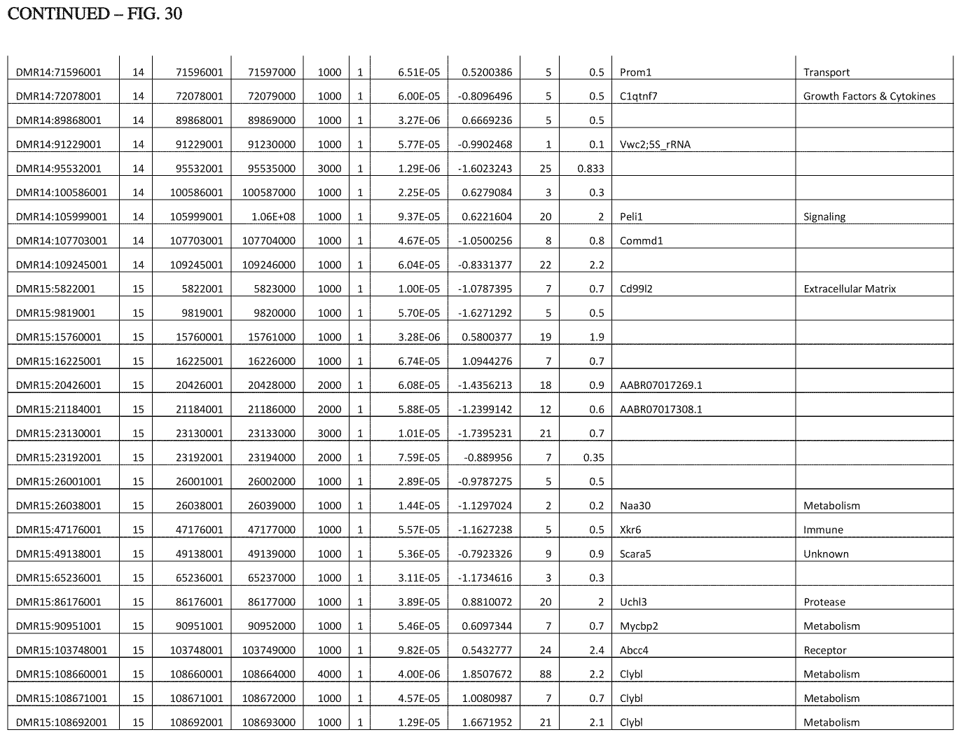

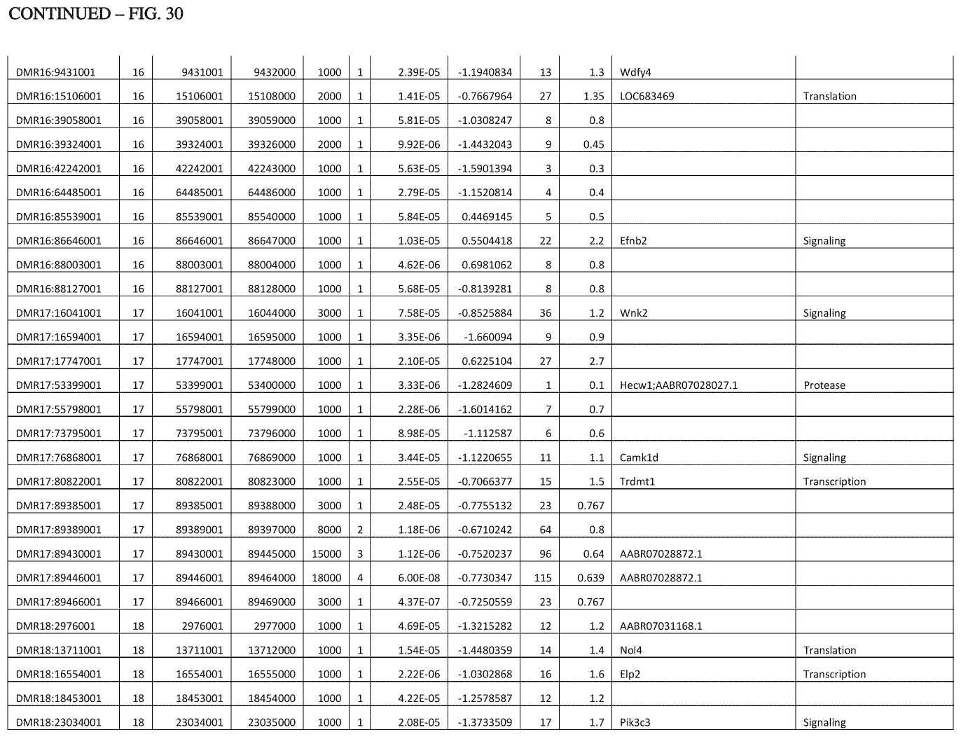

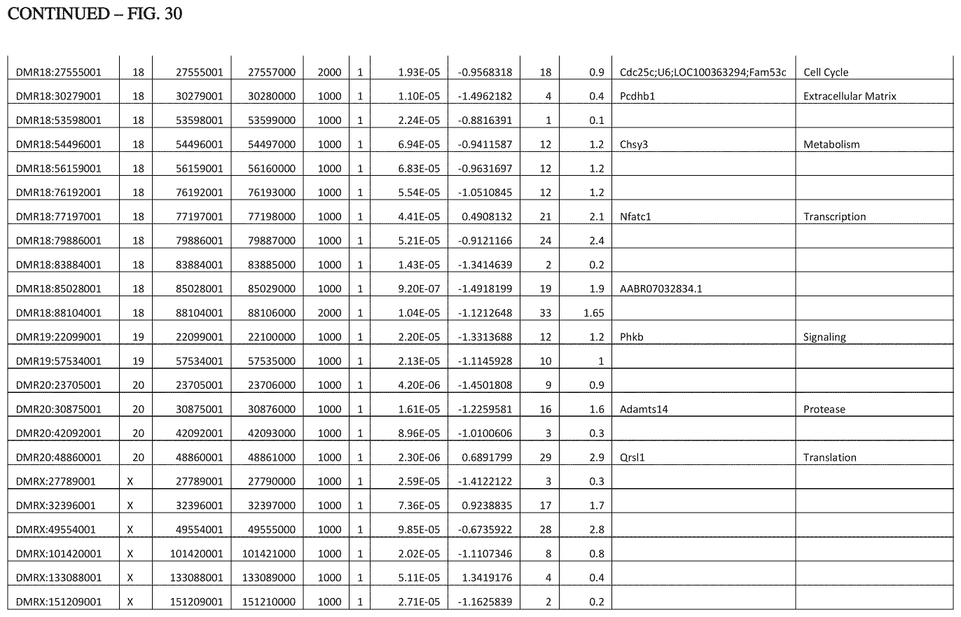

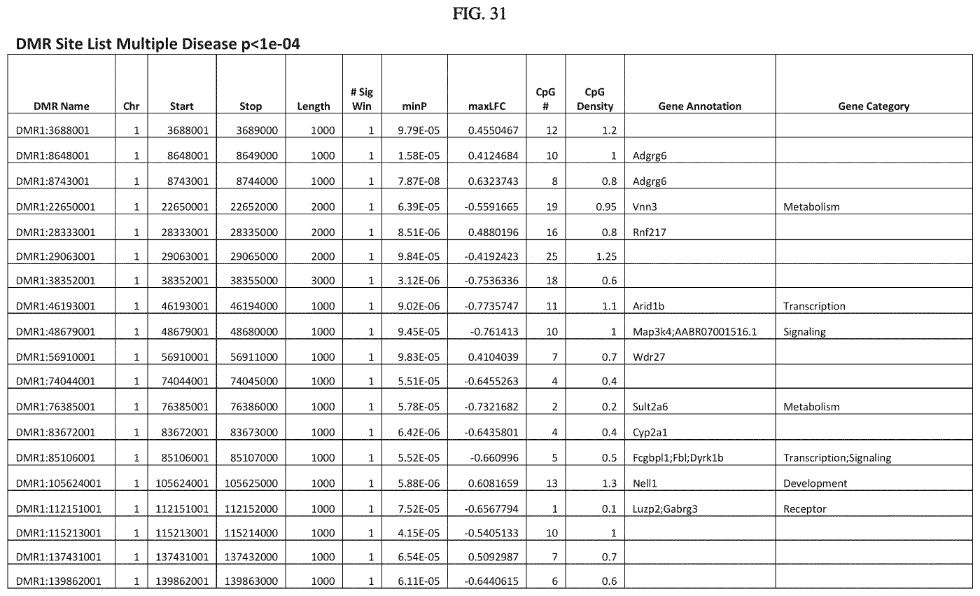

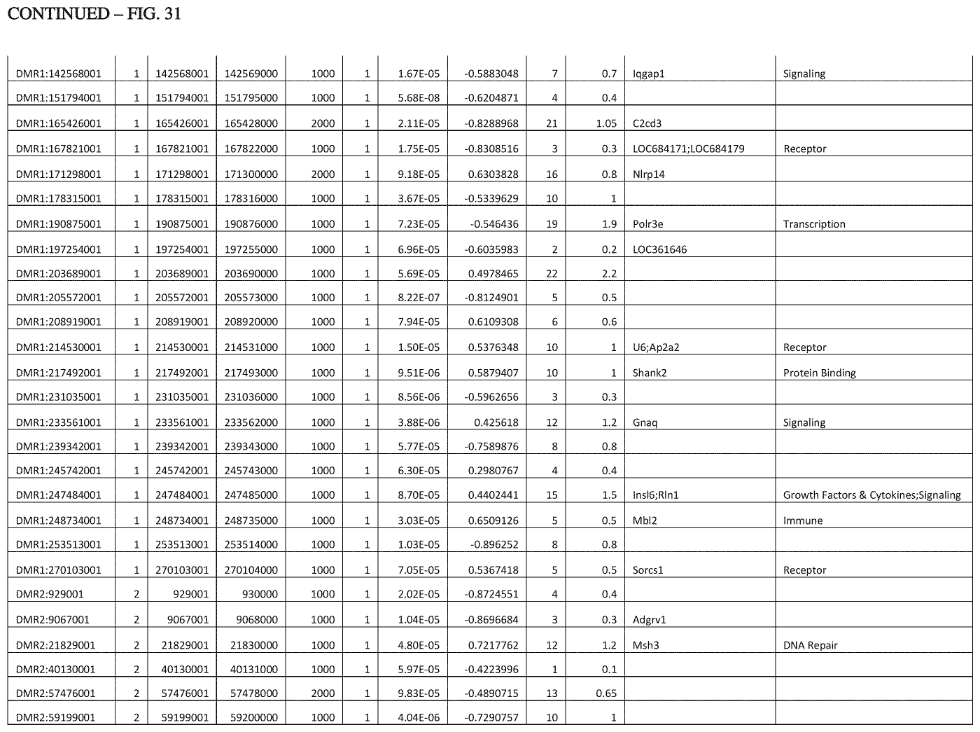

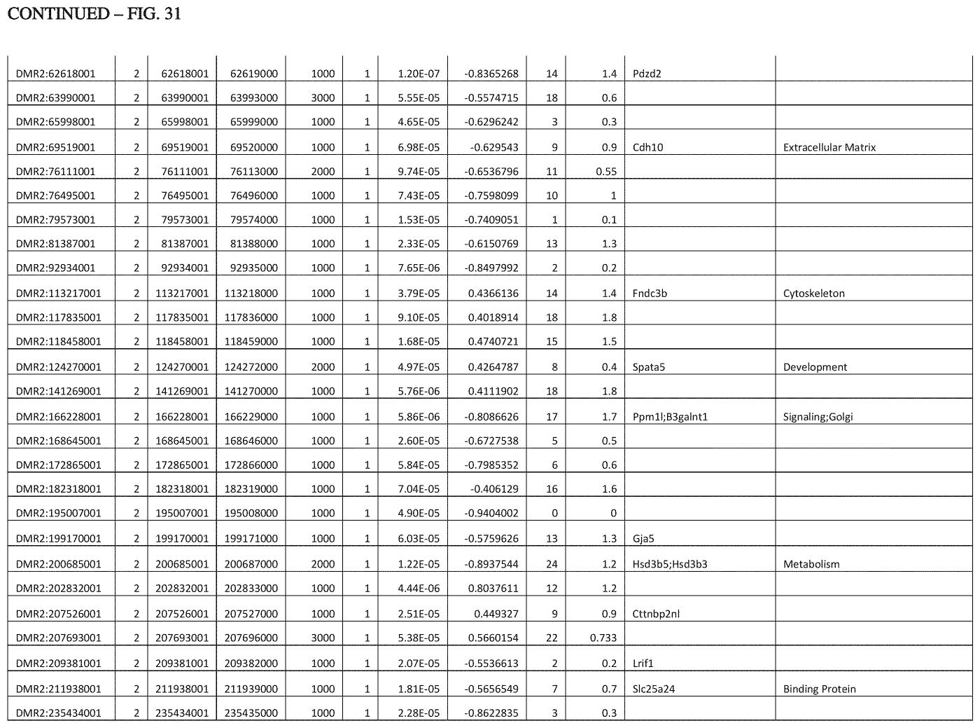

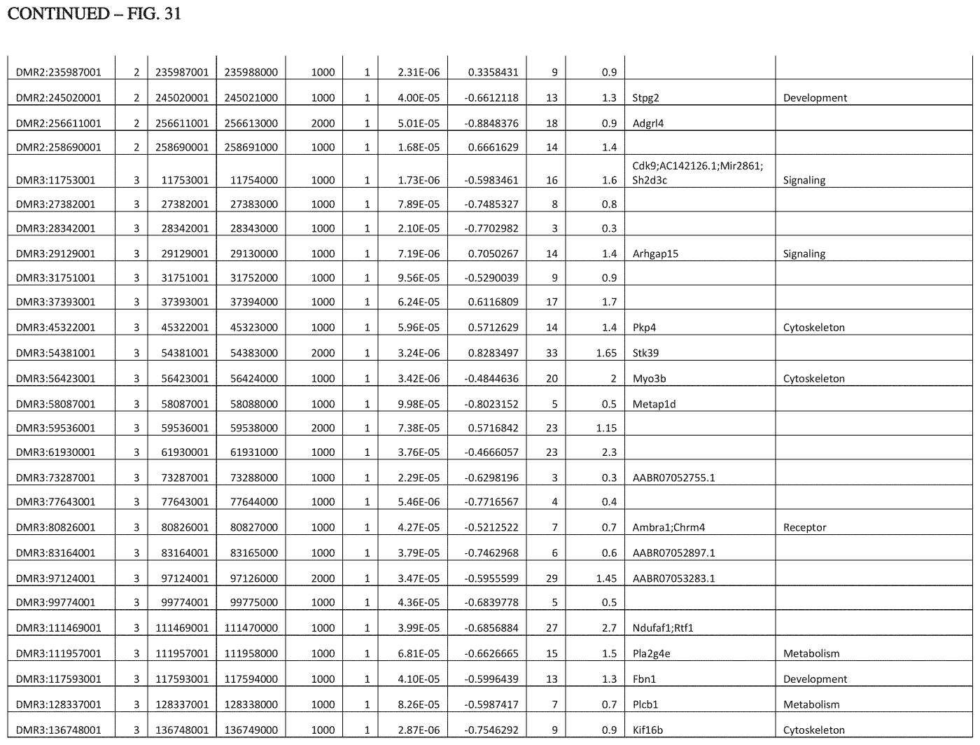

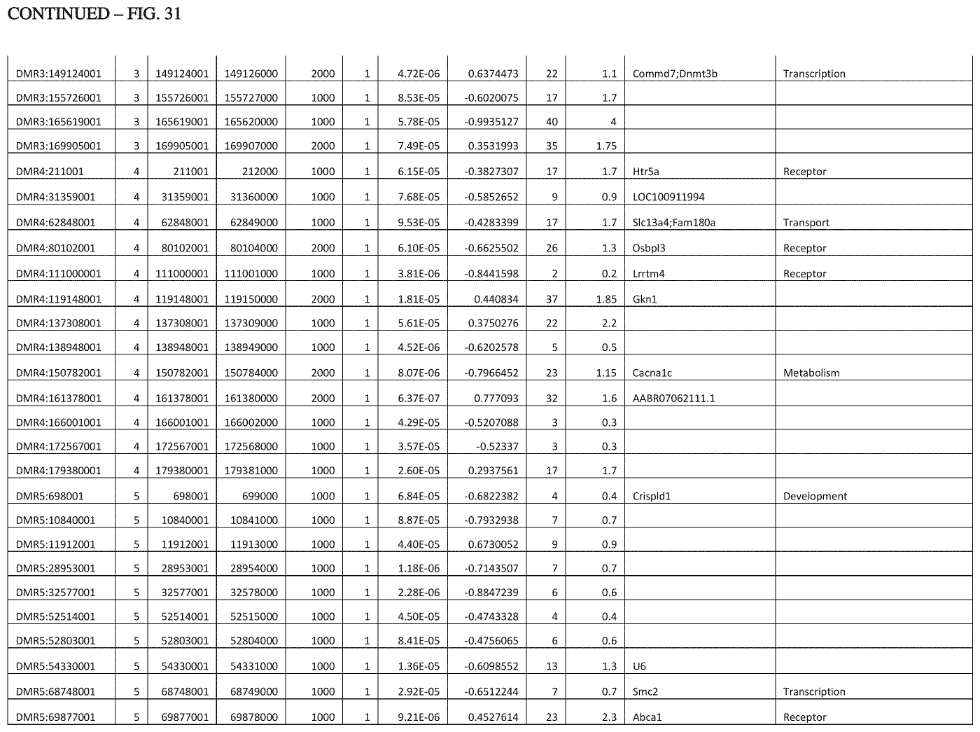

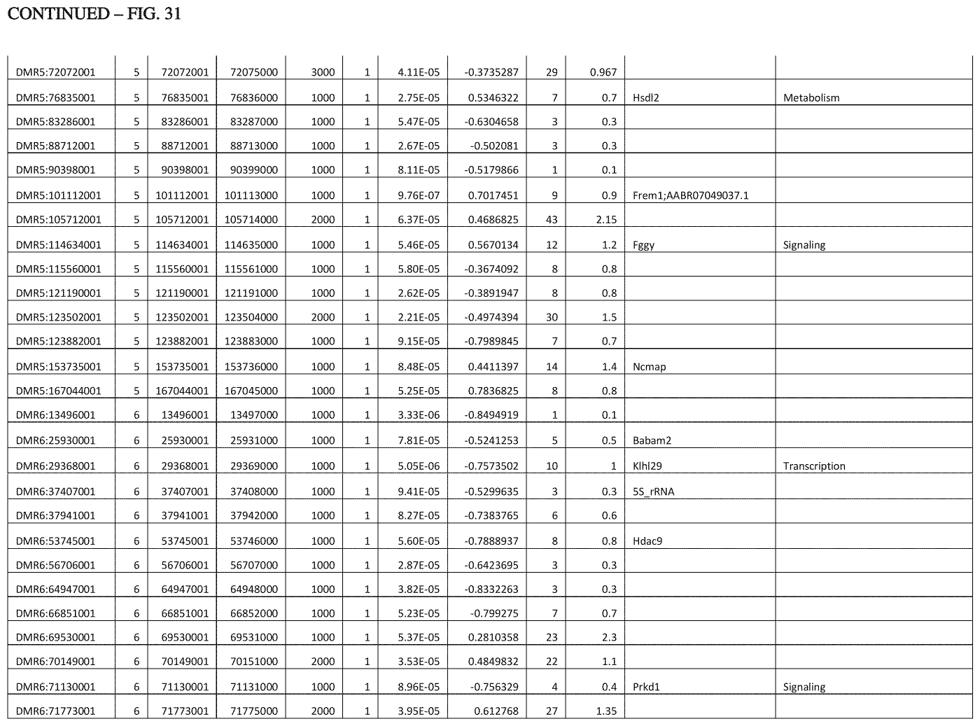

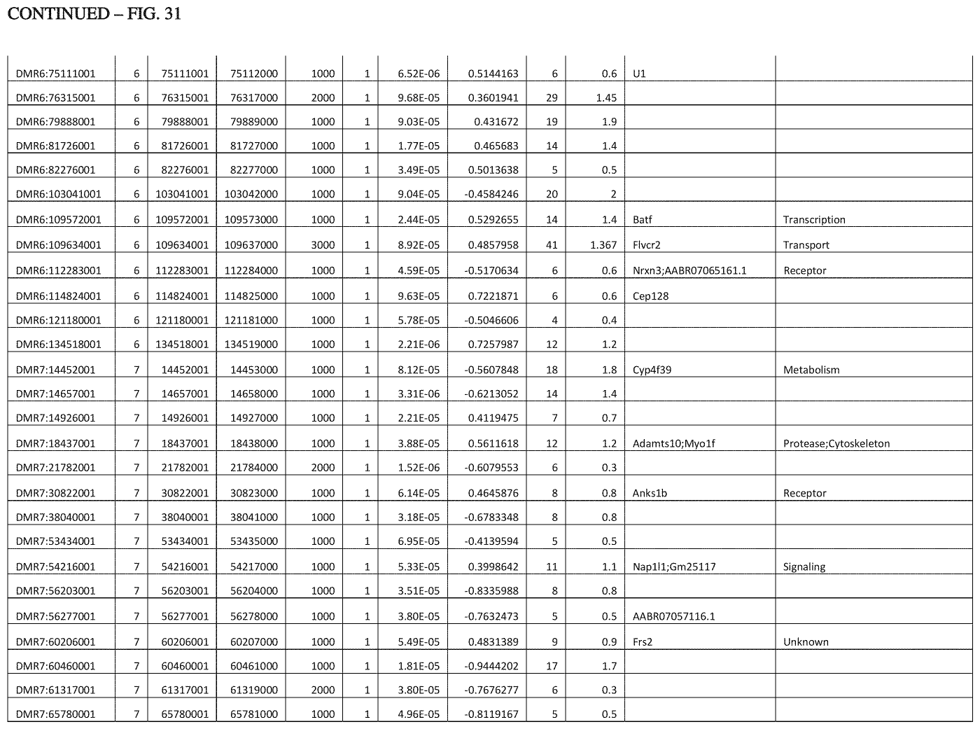

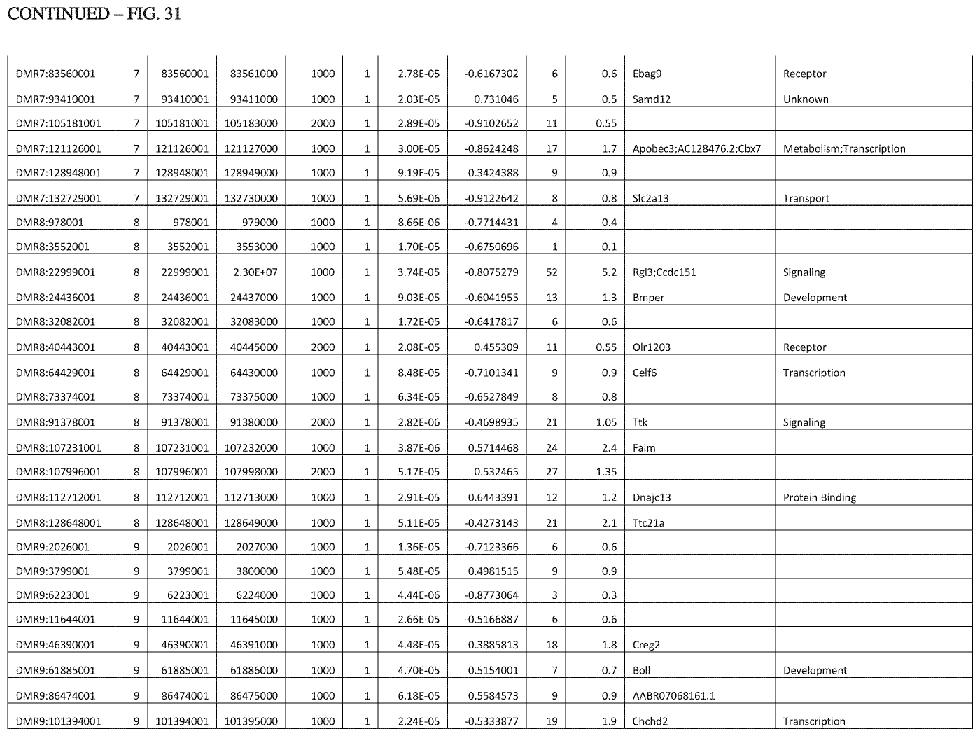

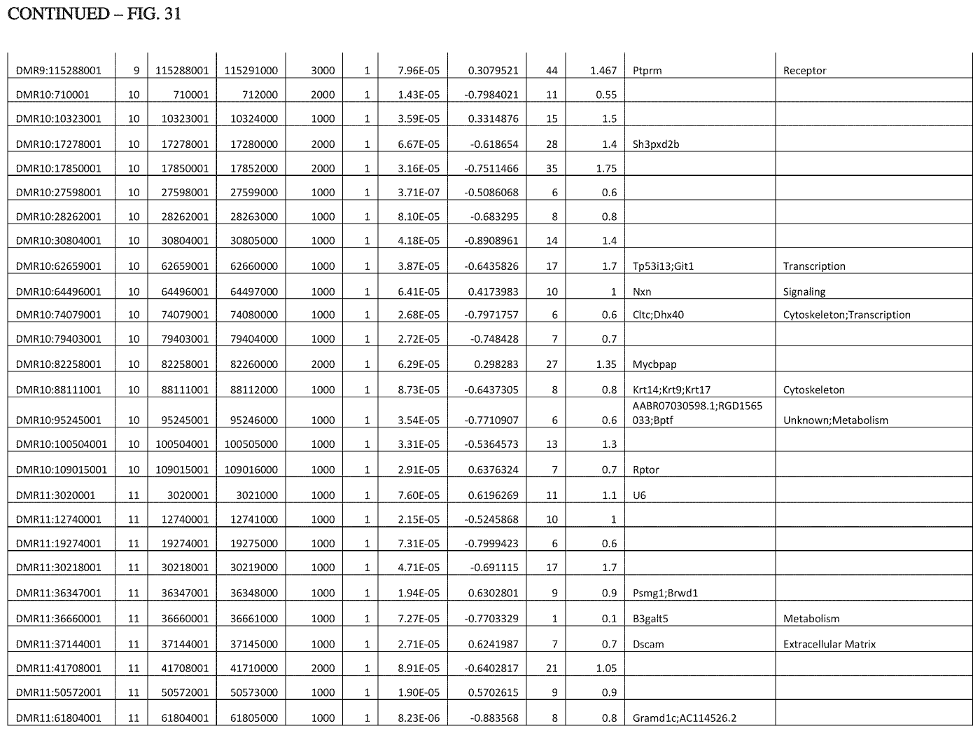

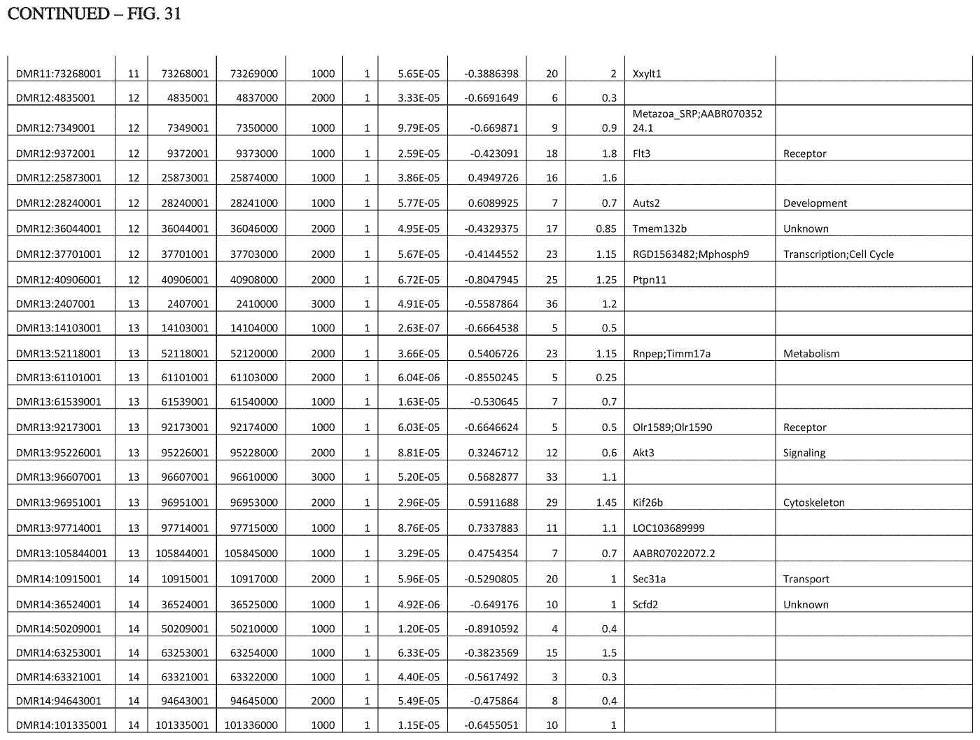

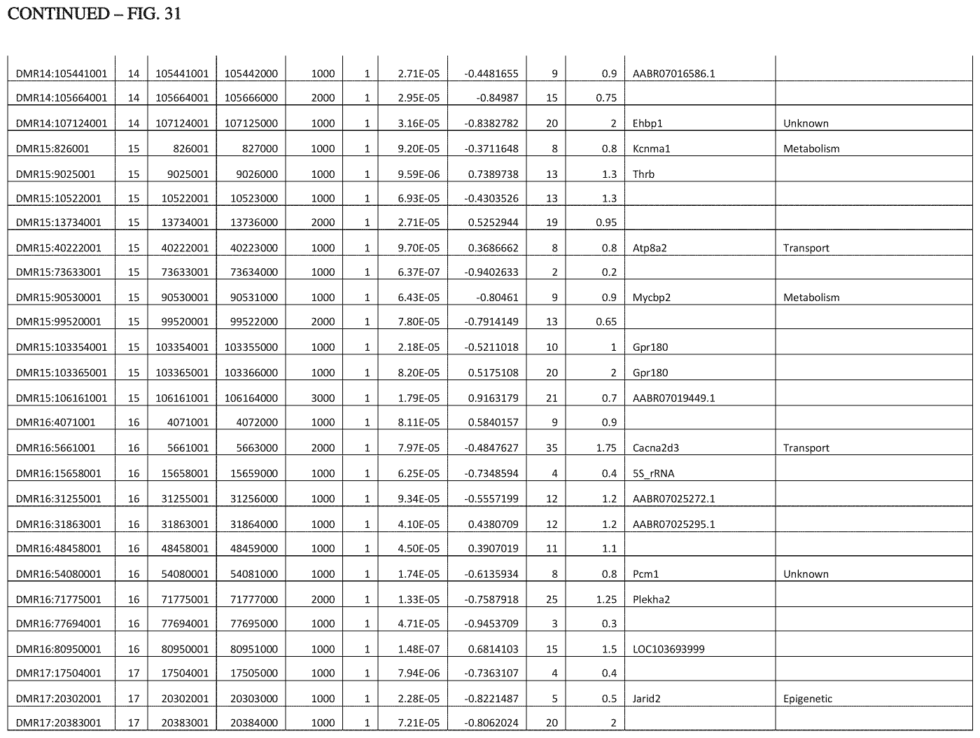

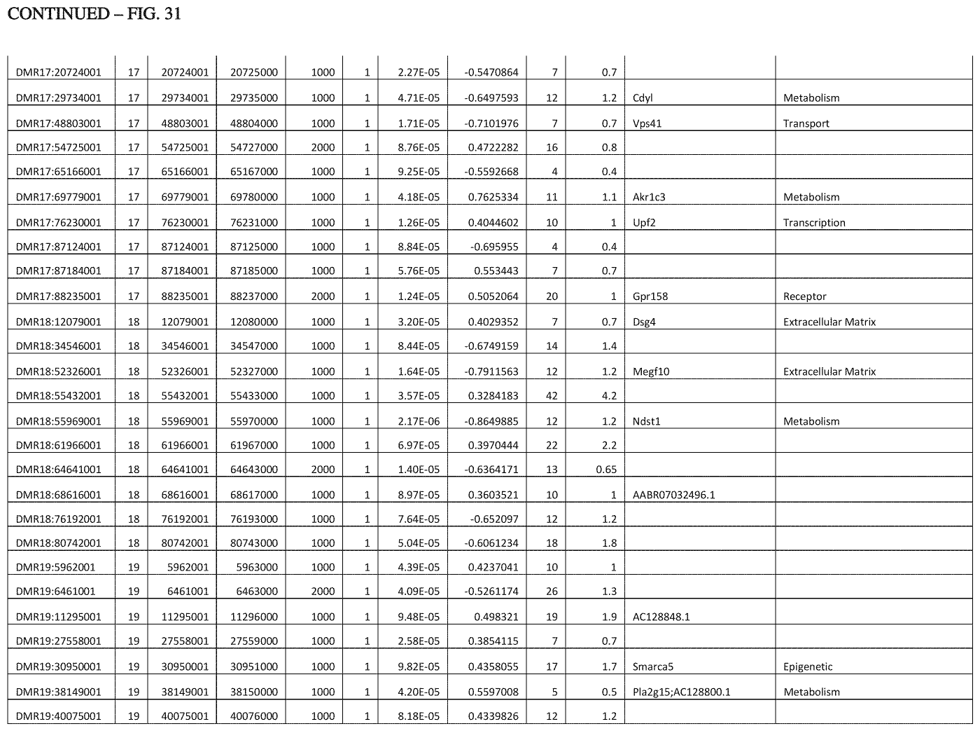

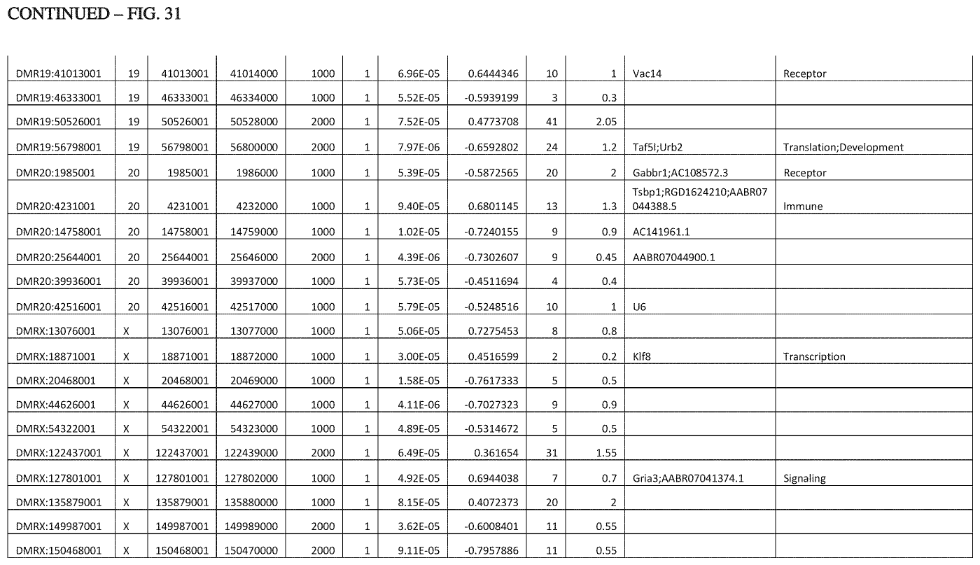

The previously reported transgenerational F3 generation sperm atrazine versus control lineage DMRs identified atrazine induced sperm differential DNA methylation. A bioinformatics reanalysis of these sperm samples used updated method parameters, including a wider 1000 bp window size and increased read depth required for each window. In the instant example, animals for each disease category were chosen only if they exhibit that single disease and no other. Any animals exhibiting multiple disease phenotypes were grouped in the multiple (≥2) disease category. A comparison of these two studies demonstrates a difference in the disease specific DMR sets, which were identified. For example, the lean phenotype was found to have 301 total DMRs at an edgeR p-value threshold of p<1e−4 with 2 of these having multiple neighboring windows (FIG. 26A) in the current study, and 467 total DMRs at an edgeR p-value threshold of p<1e−5 with 7 having multiple neighboring windows with the previous methodology. The overlap of these disease specific DMR sets was negligible with those of the previous disease DMRs. By removing the confounding inputs from multiple diseases in one disease category, the current methodology should identify more accurate epigenetic marks associated with each individual pathology at the risk of lowering analysis power due to smaller sample sizes. The current study identified disease-specific DMRs (301 lean, 693 testis, 261 kidney, and 322 late puberty, and 336 multiple disease) at p<1e−04 that are presented in FIG. 26 and shown in FIGS. 27-31. The log-fold change in DNA methylation is presented and an increase in methylation is associated with 27% pubertal abnormality DMRs, 56% testis disease DMRs, 43% kidney disease DMRs, 25% lean pathology DMRs, and 37% multiple disease DMRs. The others all had a decrease in DNA methylation. Observations indicate atrazine can promote germline epigenetic alterations in sperm DNA methylation that appear disease specific.

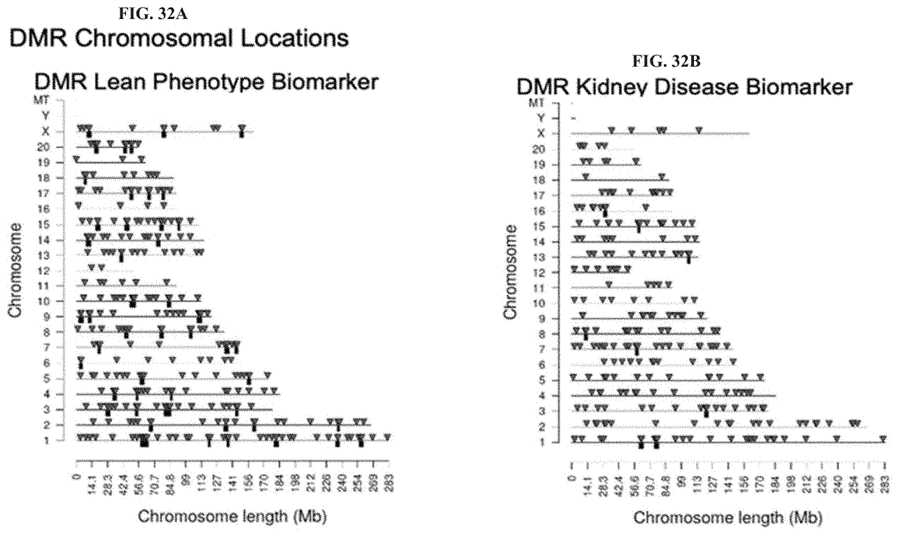

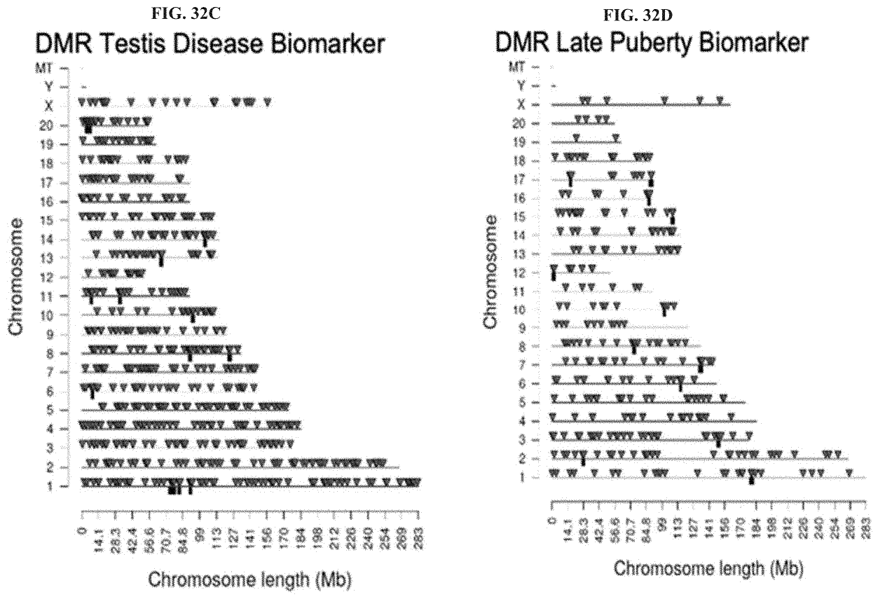

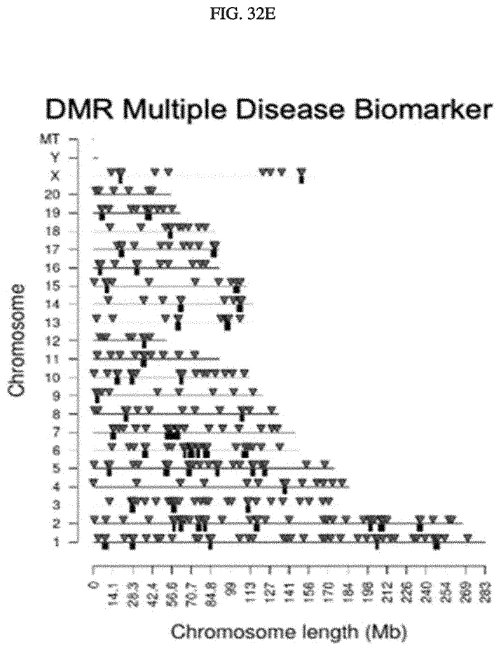

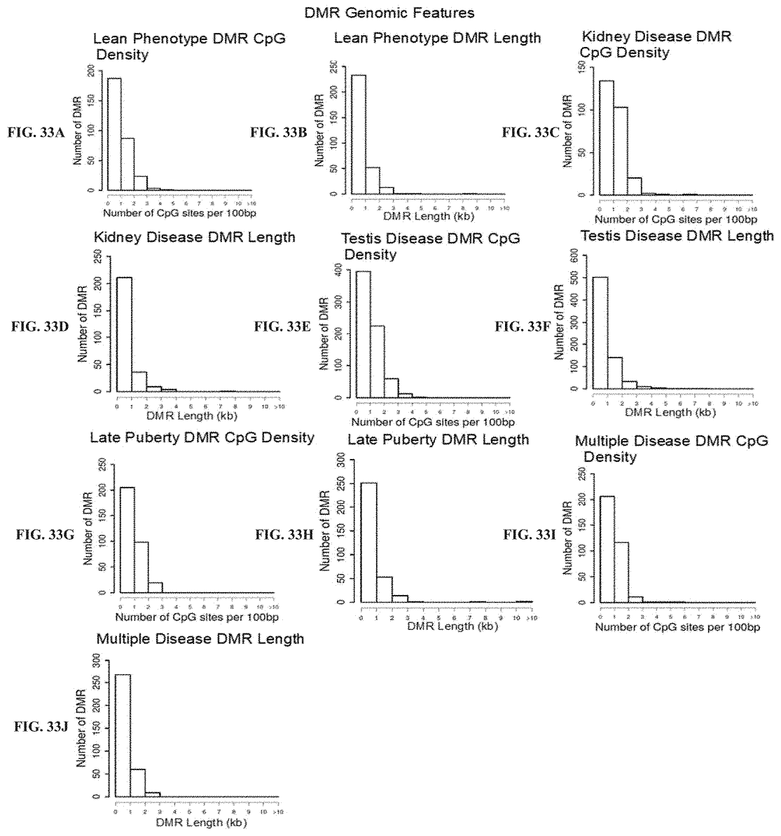

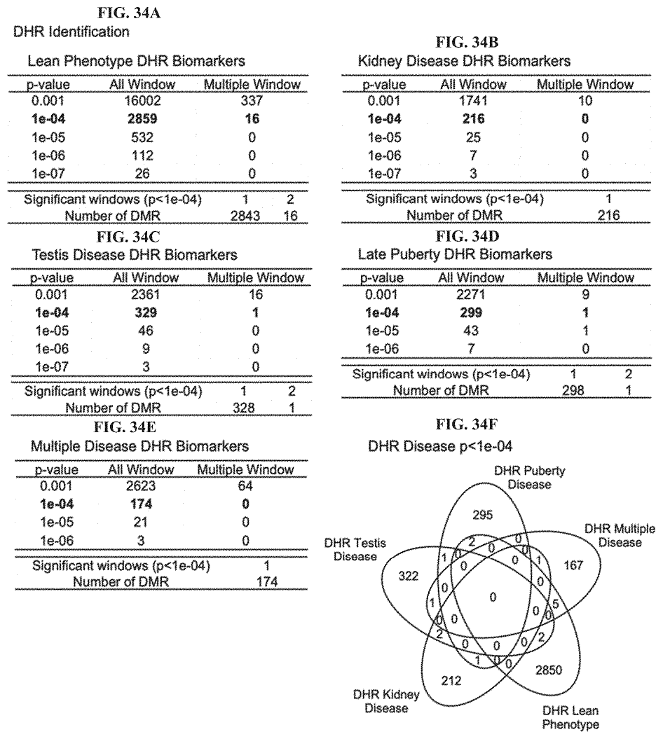

Chromosomal locations of the DMRs are presented in FIG. 32 with DMR represented as arrowheads and DMR clusters indicated by black boxes. DMRs are present on all chromosomes except the Y chromosome and mitochondrial DNA (MT). The wide distribution of DMRs across chromosomes provides evidence that the epigenetic effects of transgenerational atrazine exposure are genome-wide. The genomic features are detailed in FIG. 33. The CpG density of the DMRs is low with the majority of CpGs between 1 and 3 sites per 100 bp. The length of most DMRs is between 1,000 and 3,000 base pairs. The principal component analyses (PCA) of the RPKM adjusted read depths at differential DMR sites for each sample are shown in FIG. 25. The PCA plots shows how the DMR samples cluster according to disease compared to non-disease and indicate potential outliers in the data (none observed) when DMR sites are evaluated (see FIG. 25).

### Sperm Histone Retention

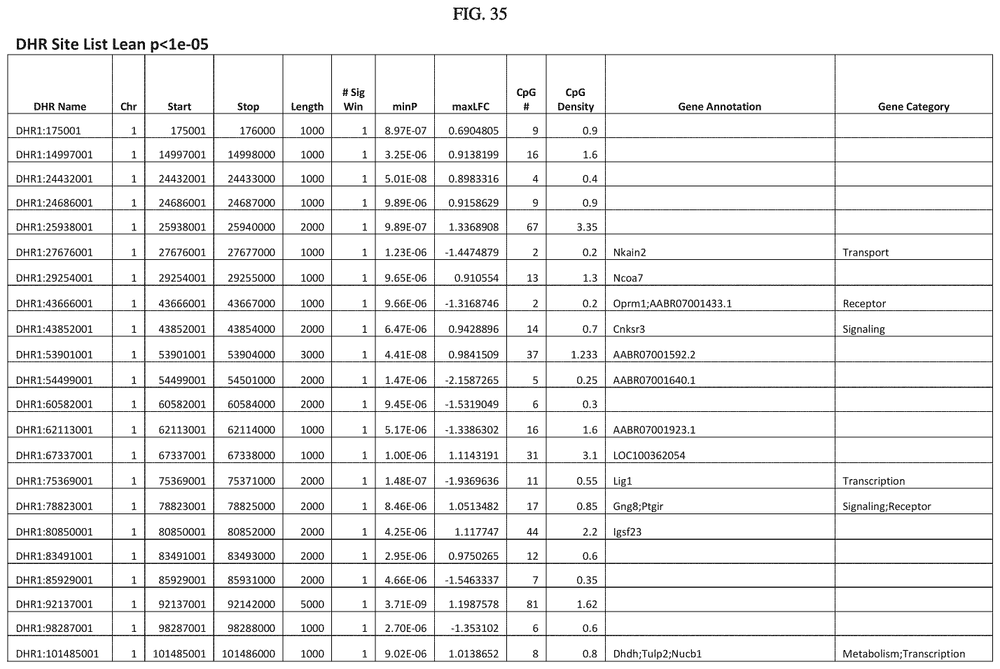

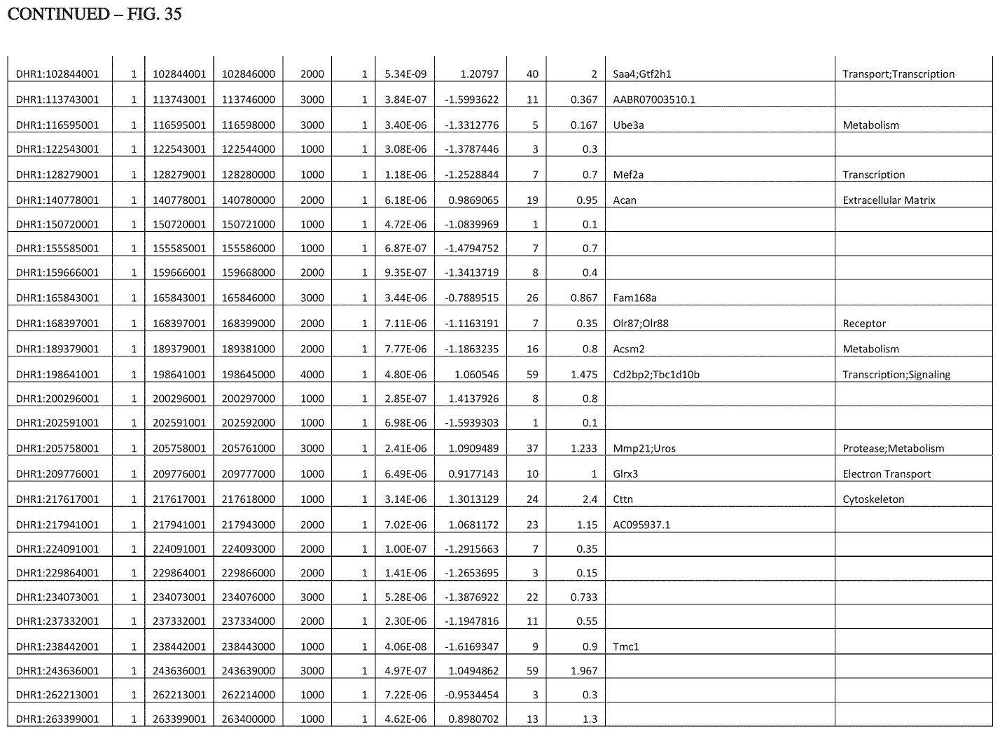

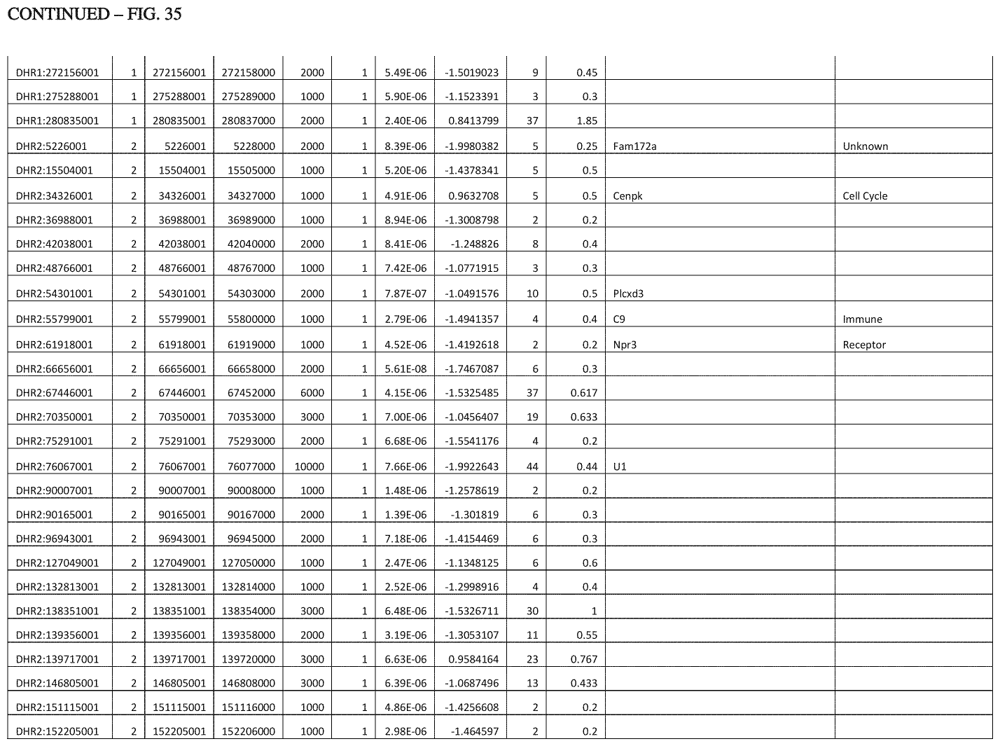

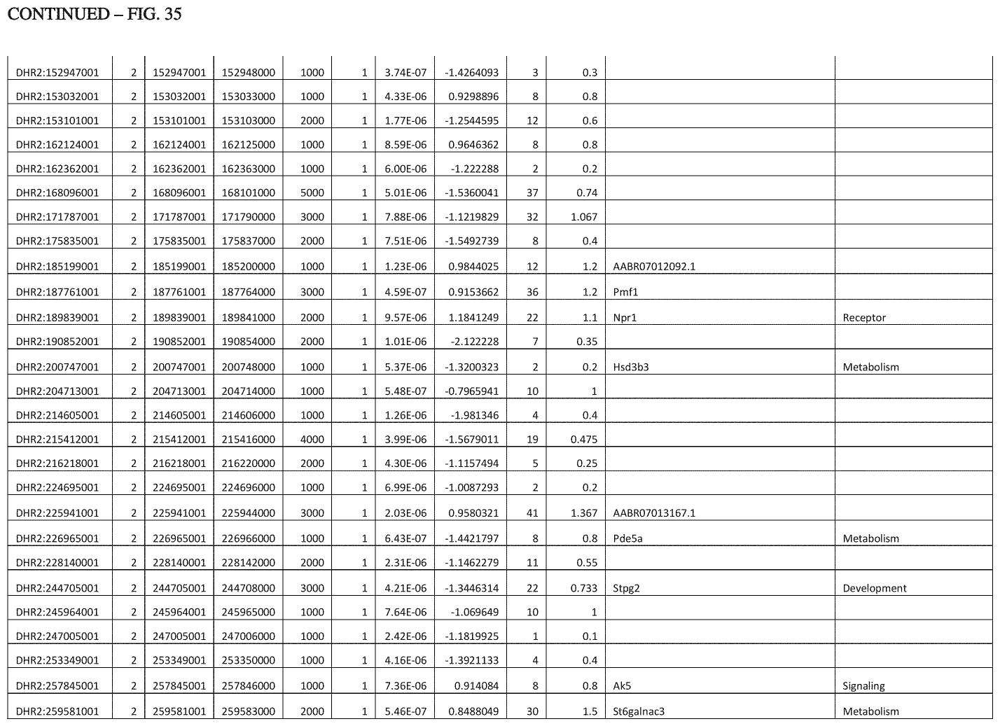

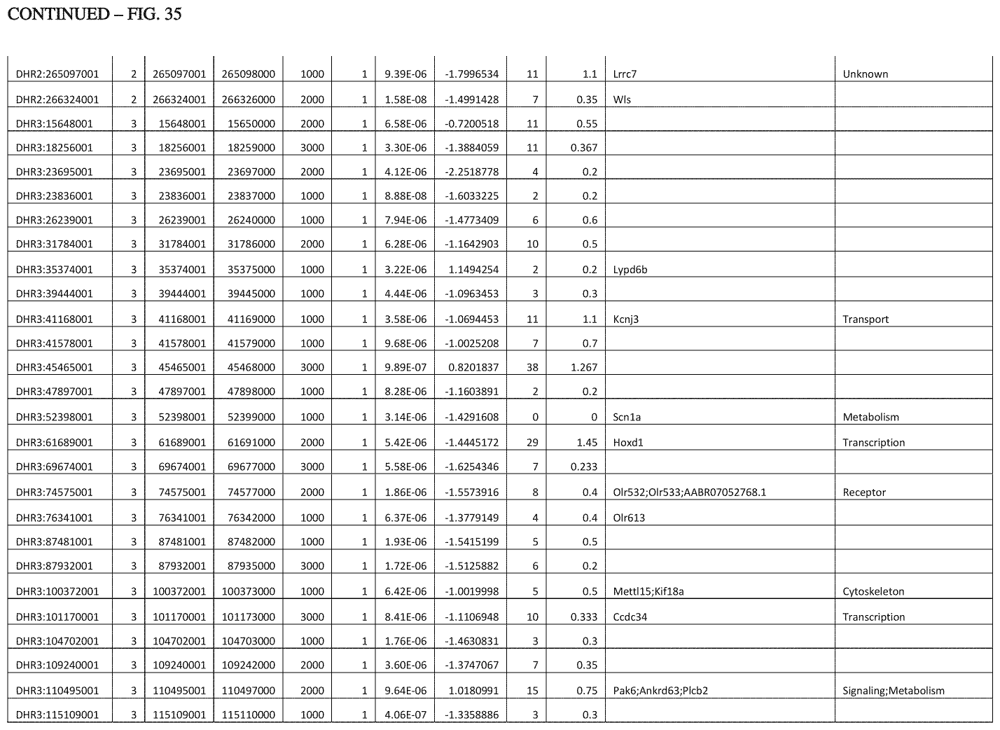

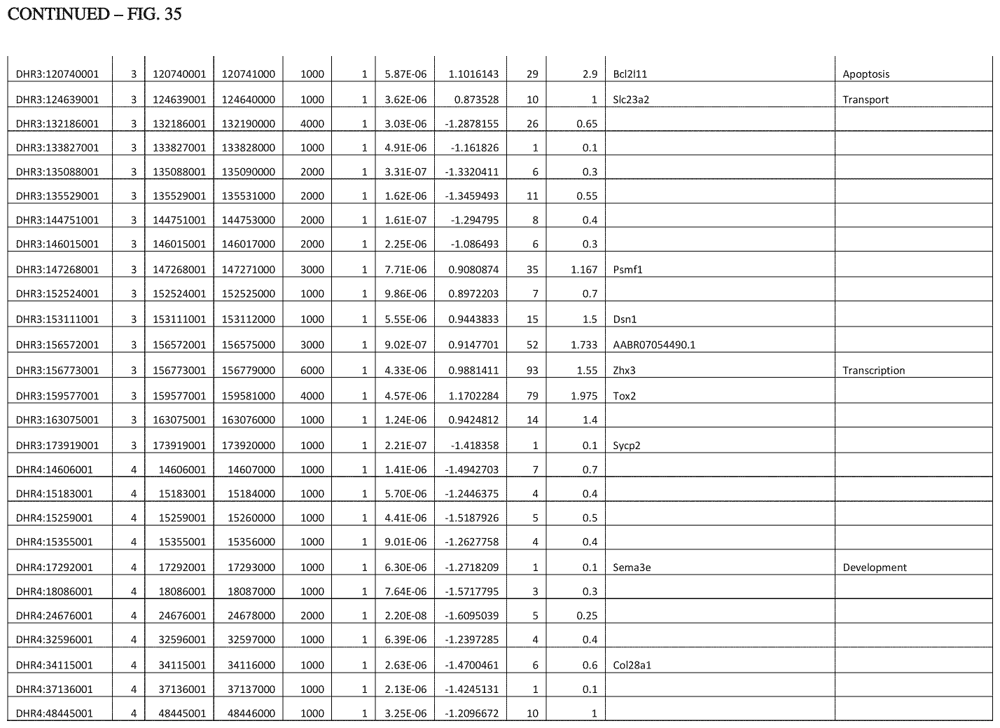

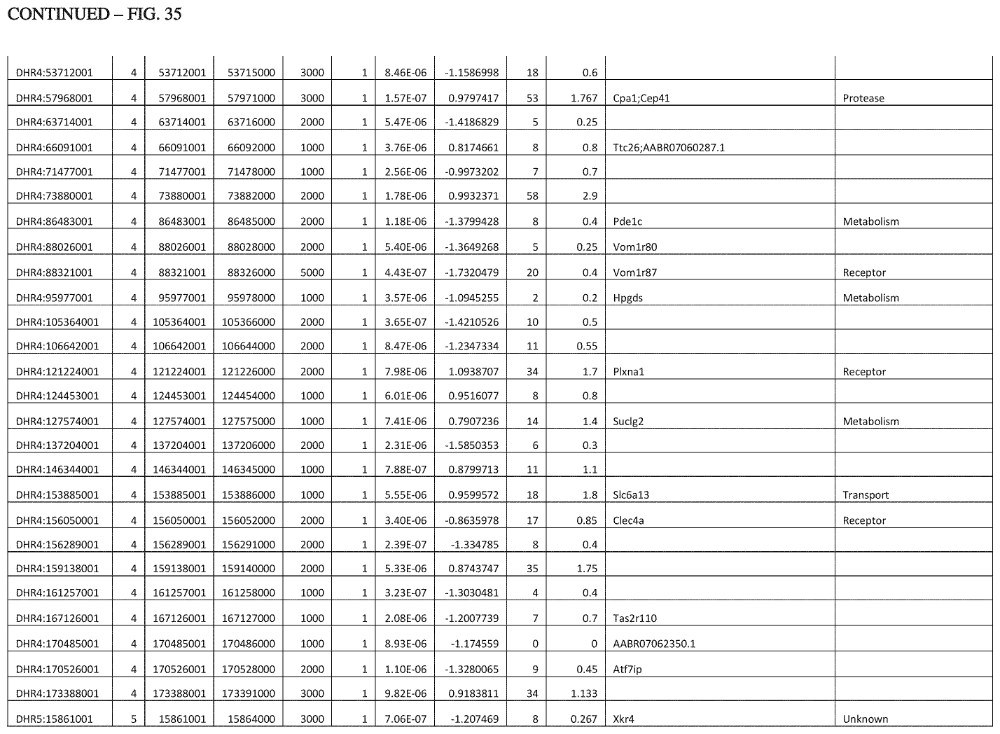

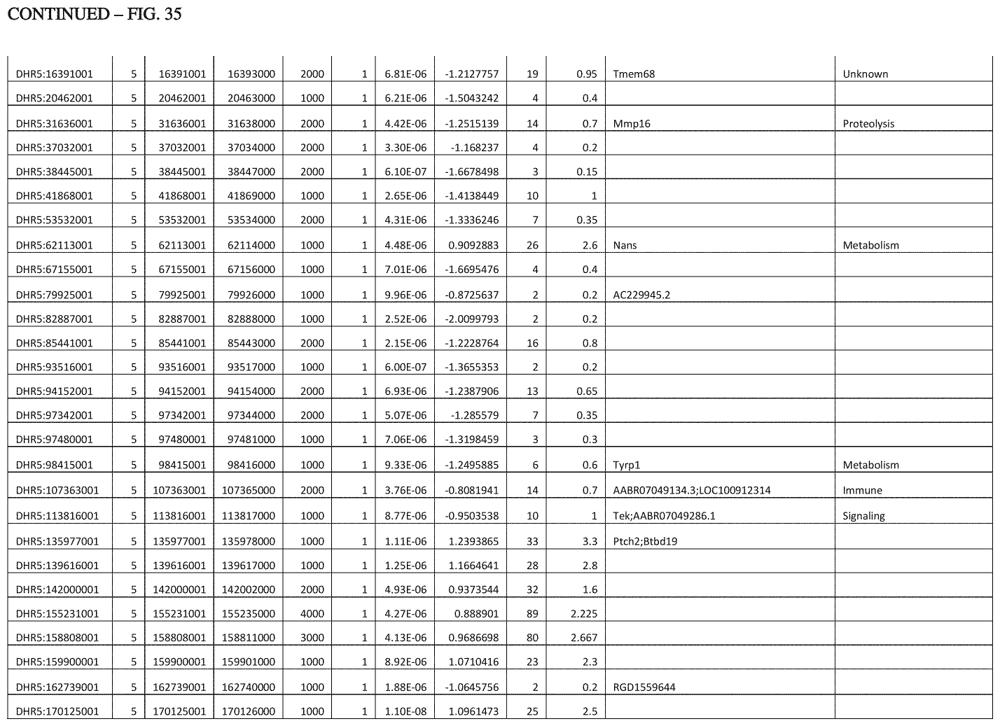

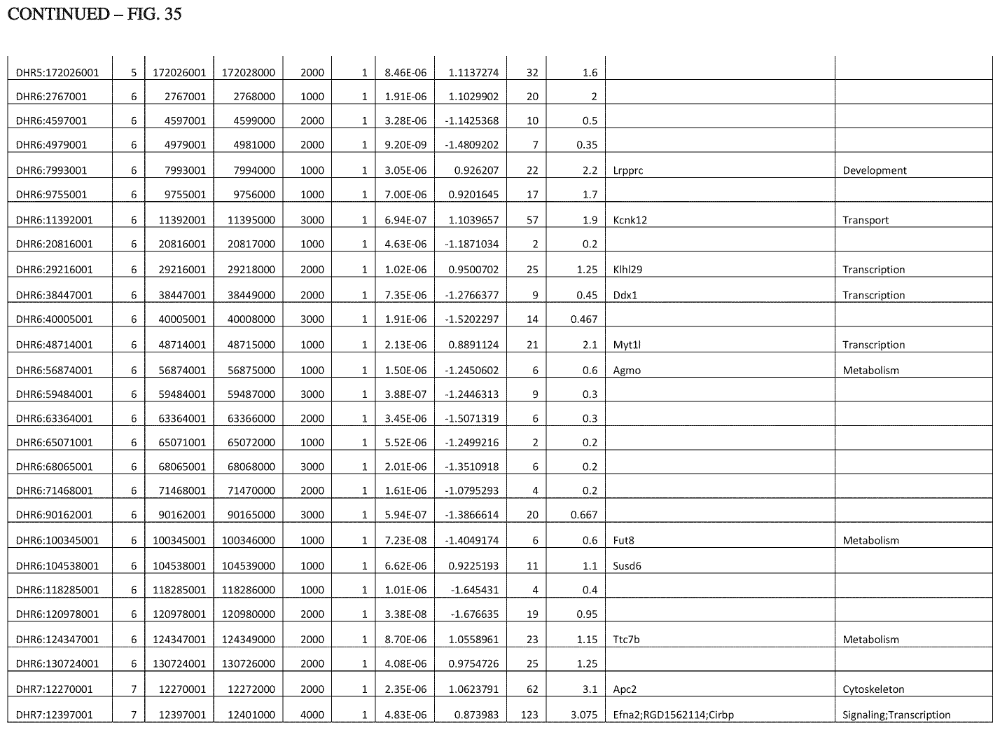

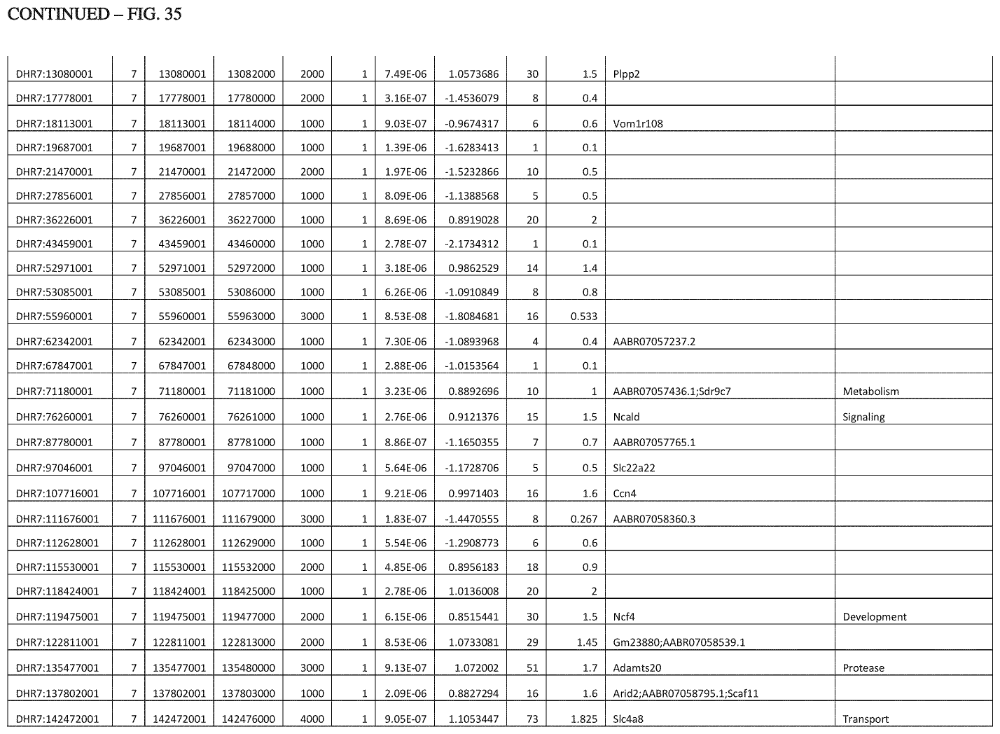

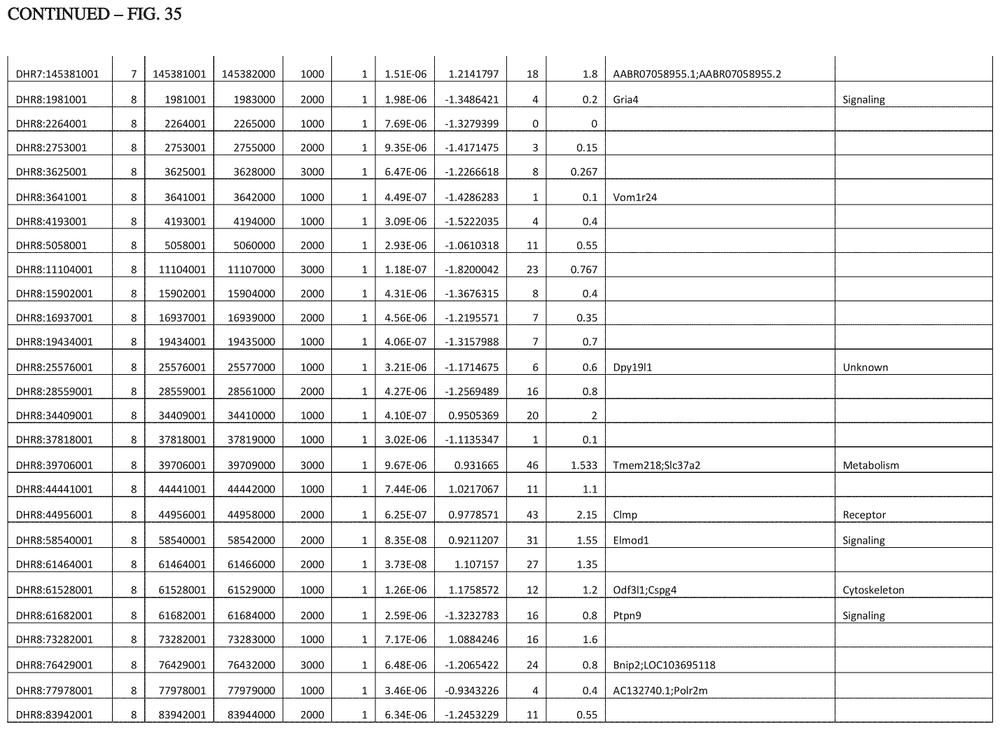

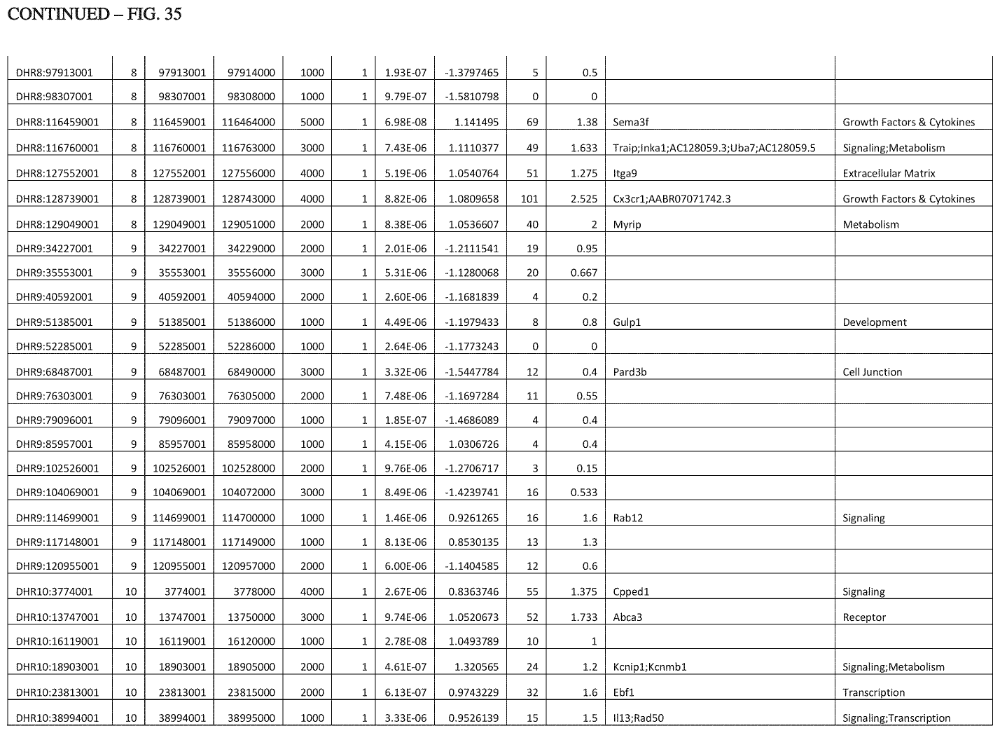

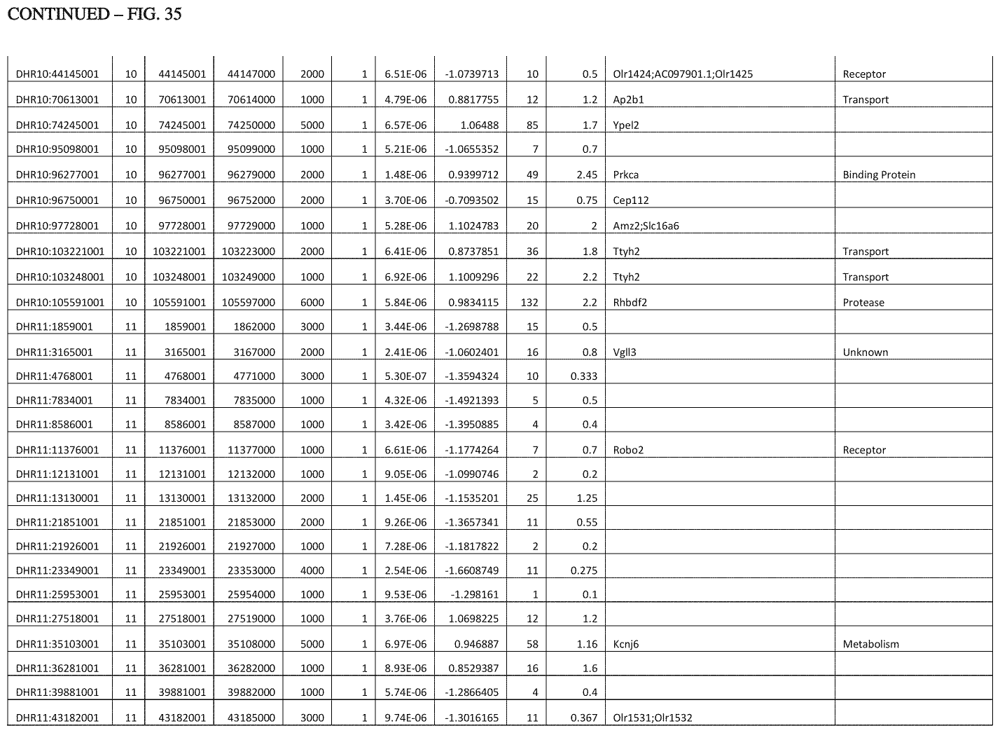

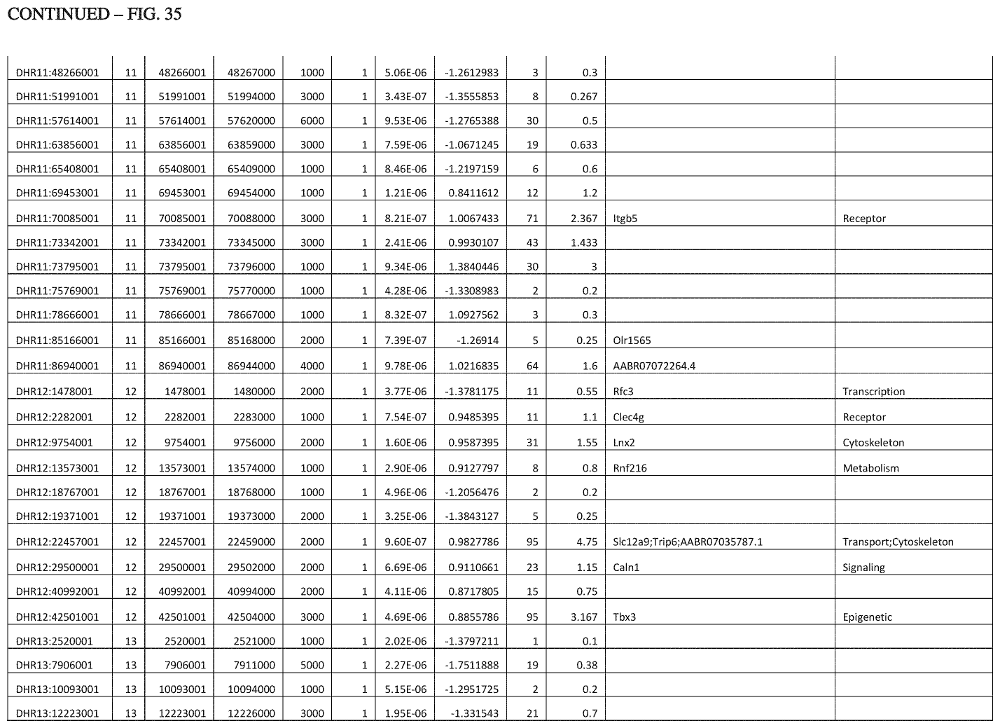

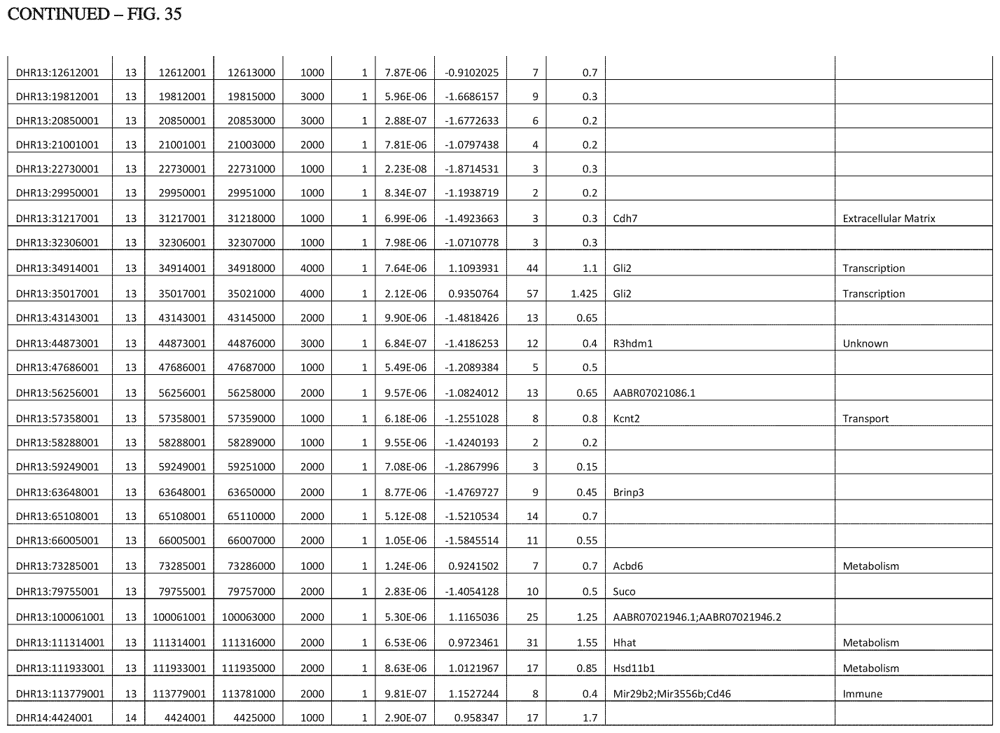

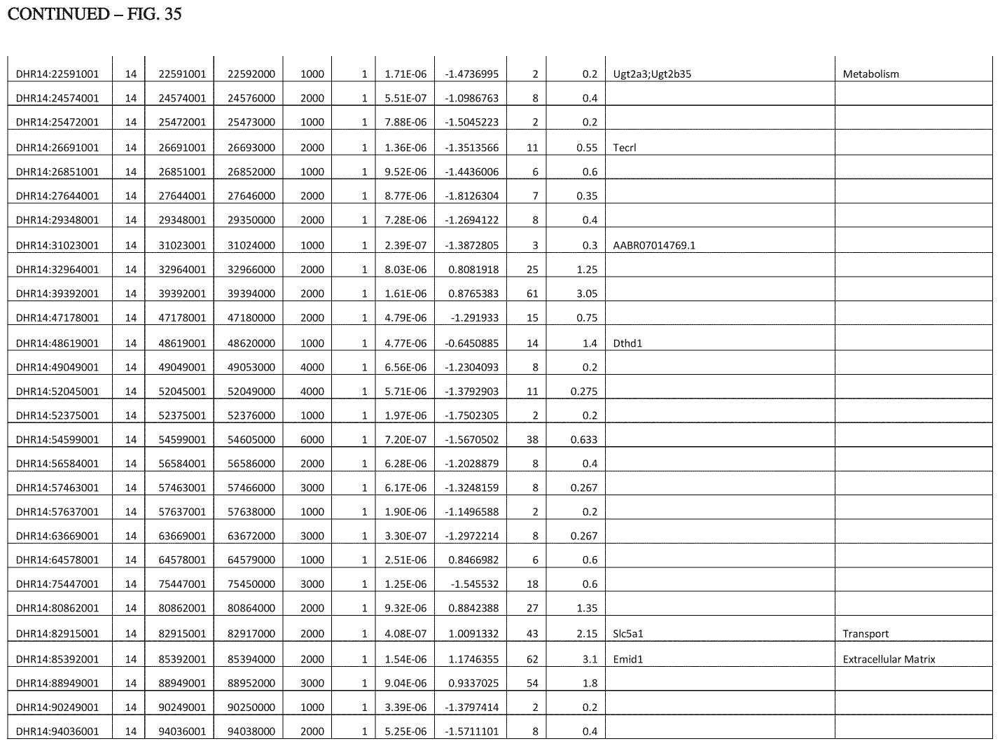

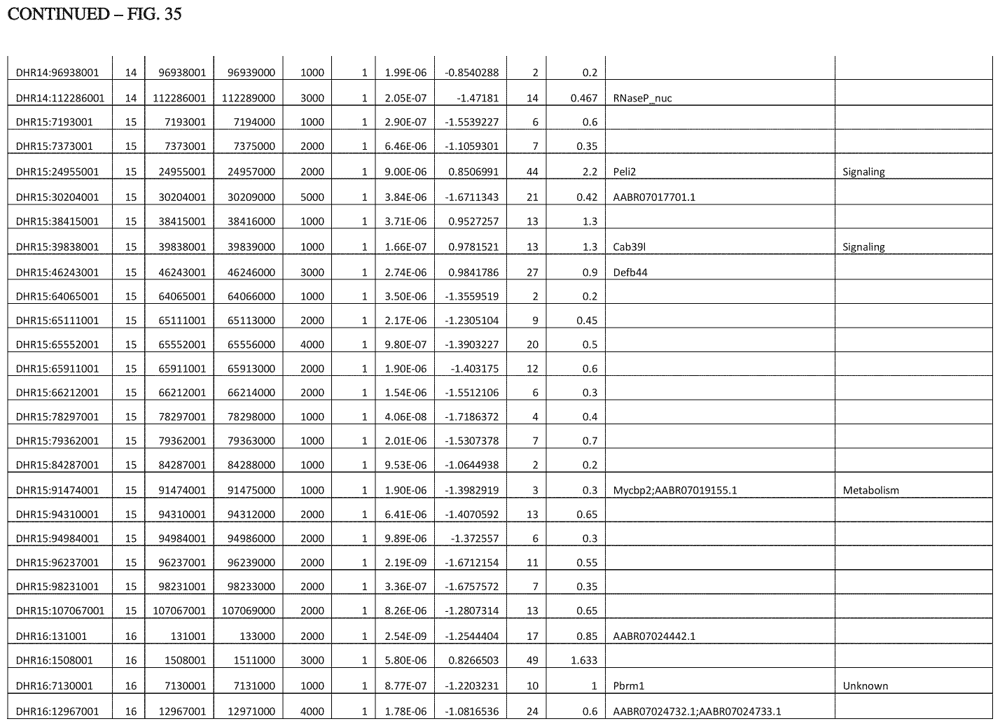

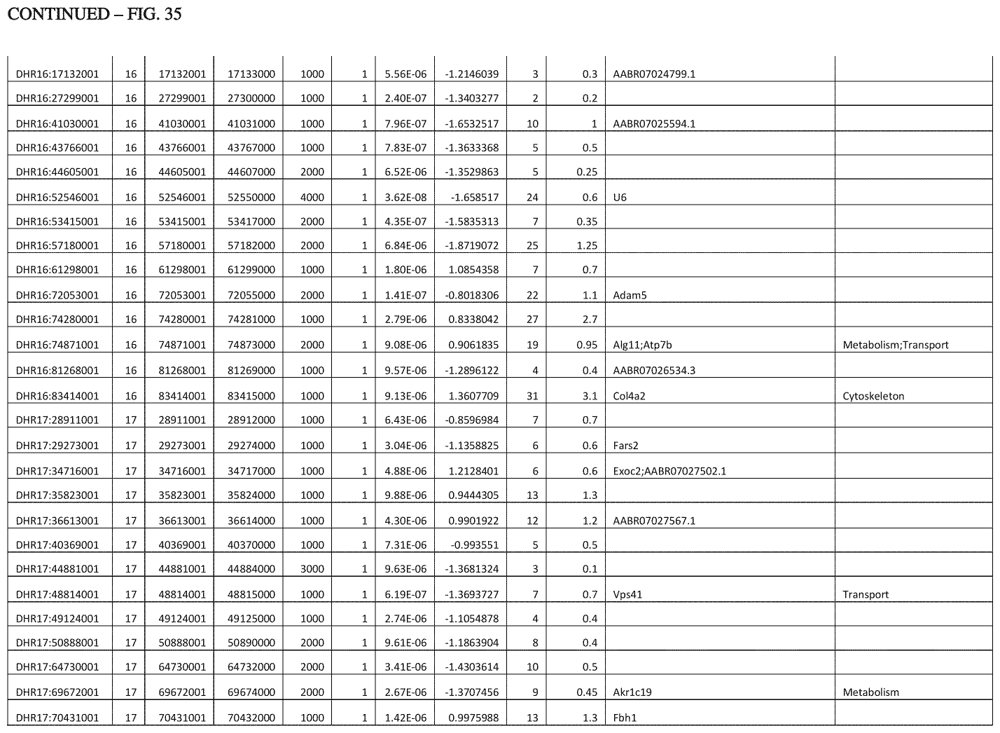

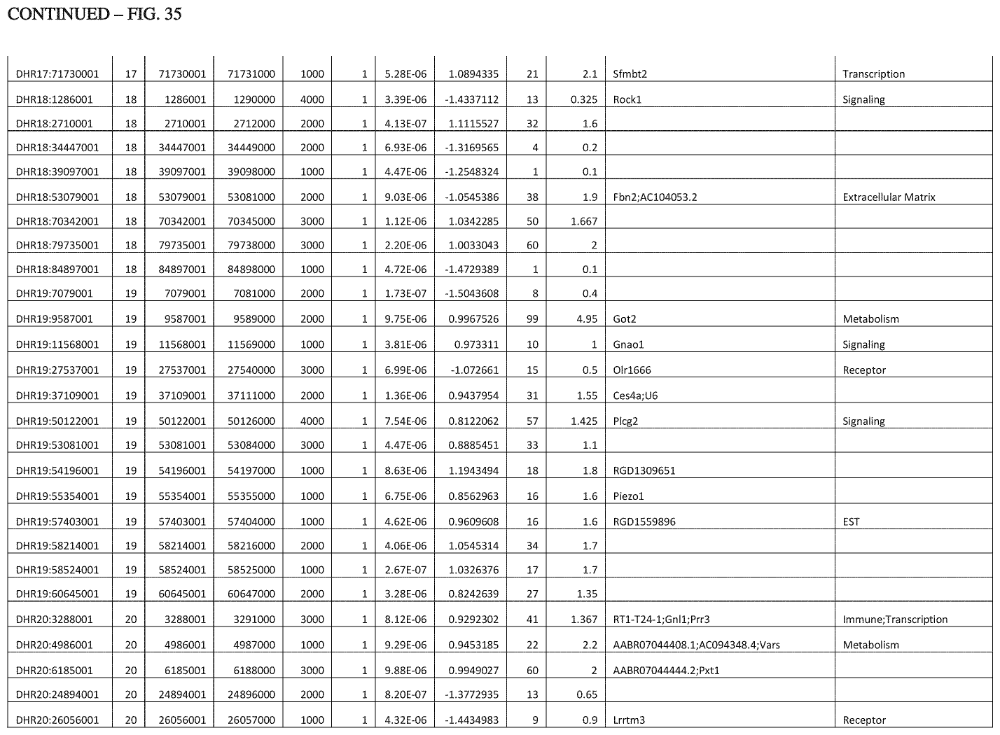

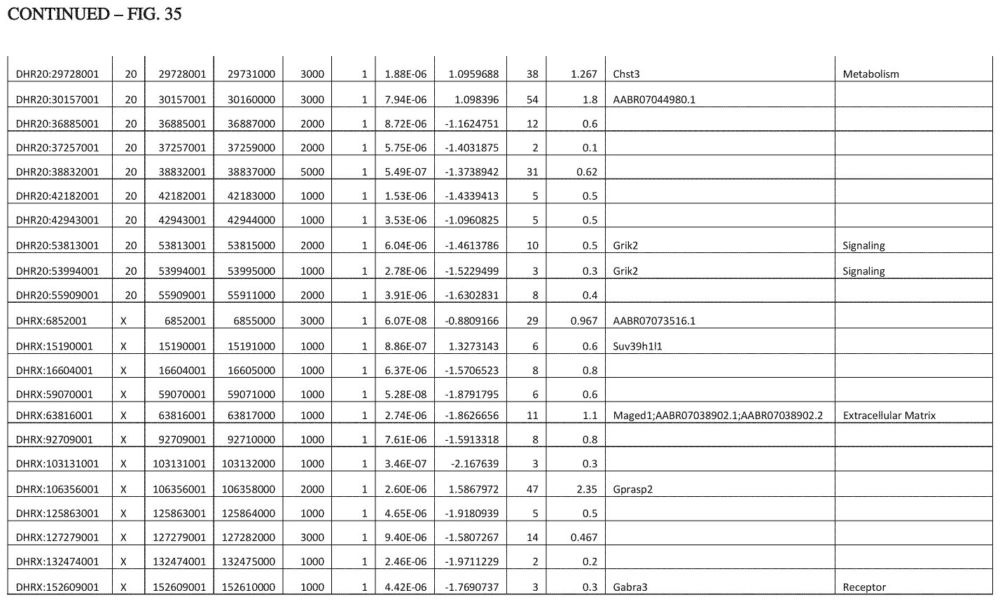

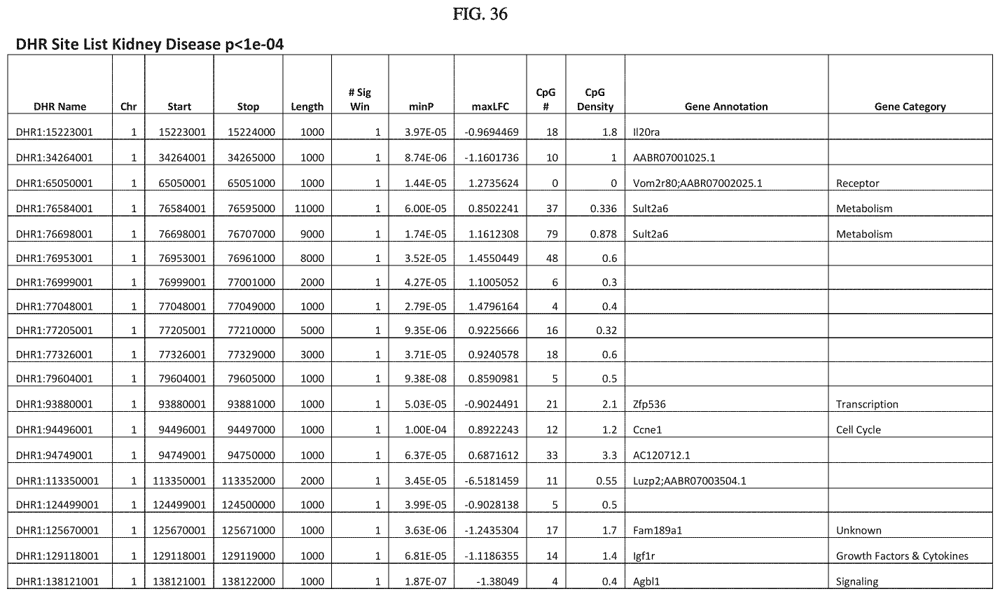

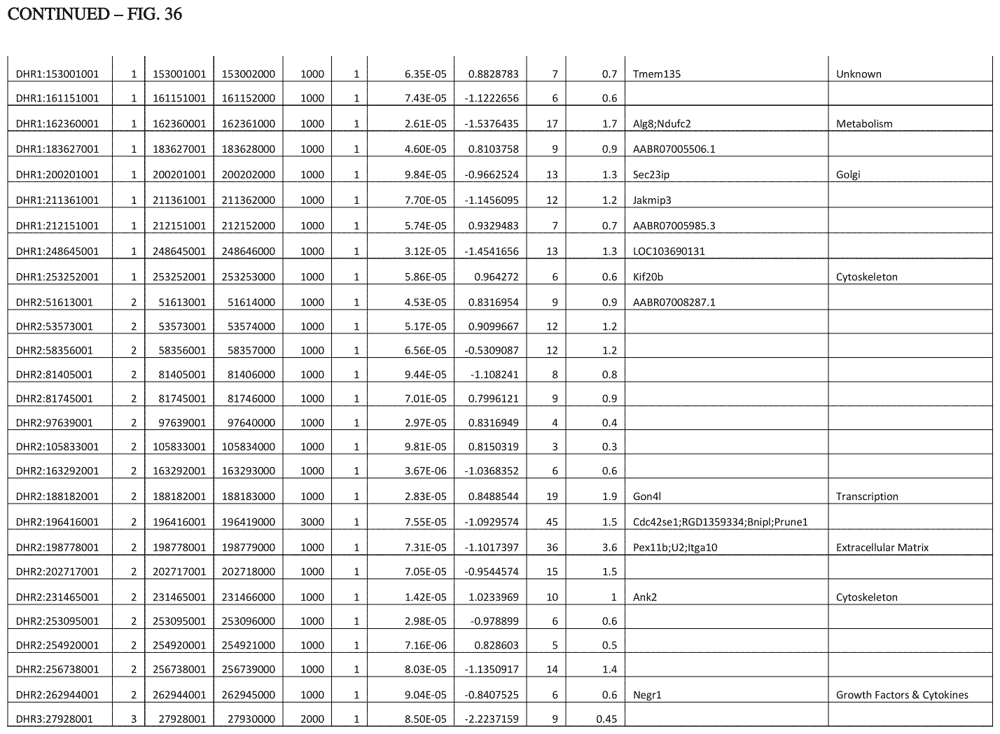

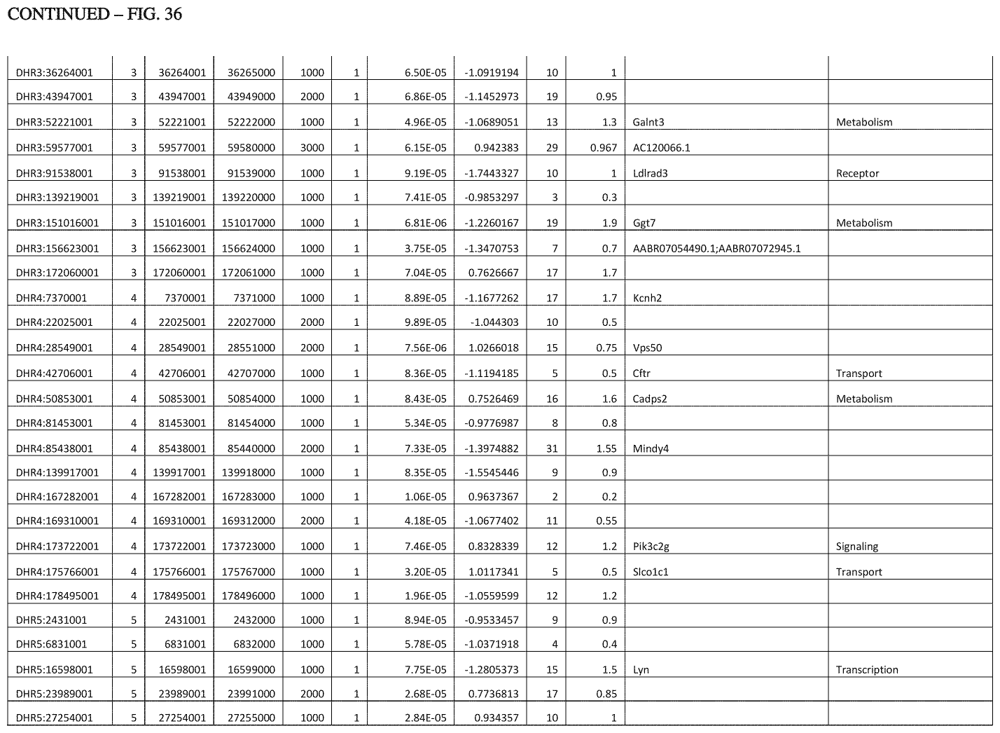

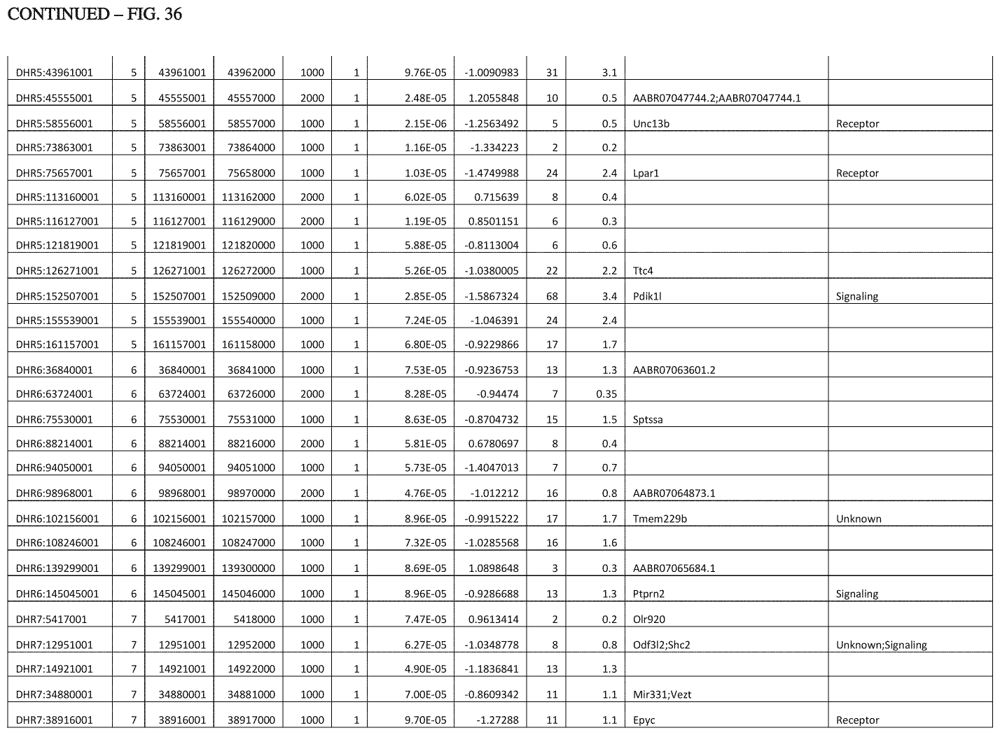

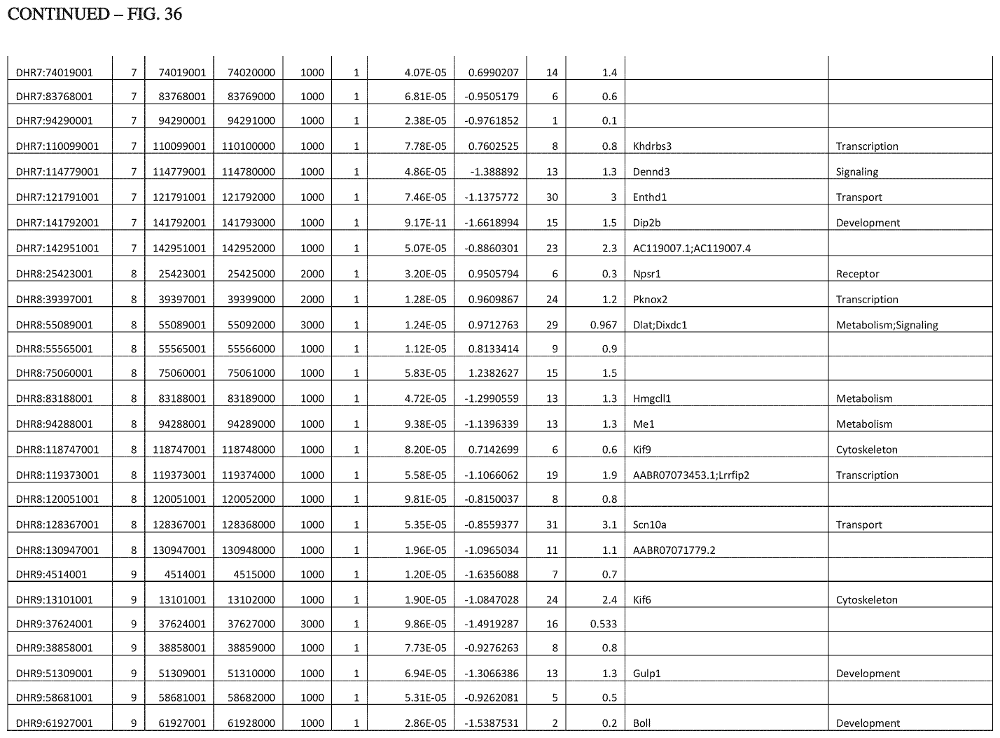

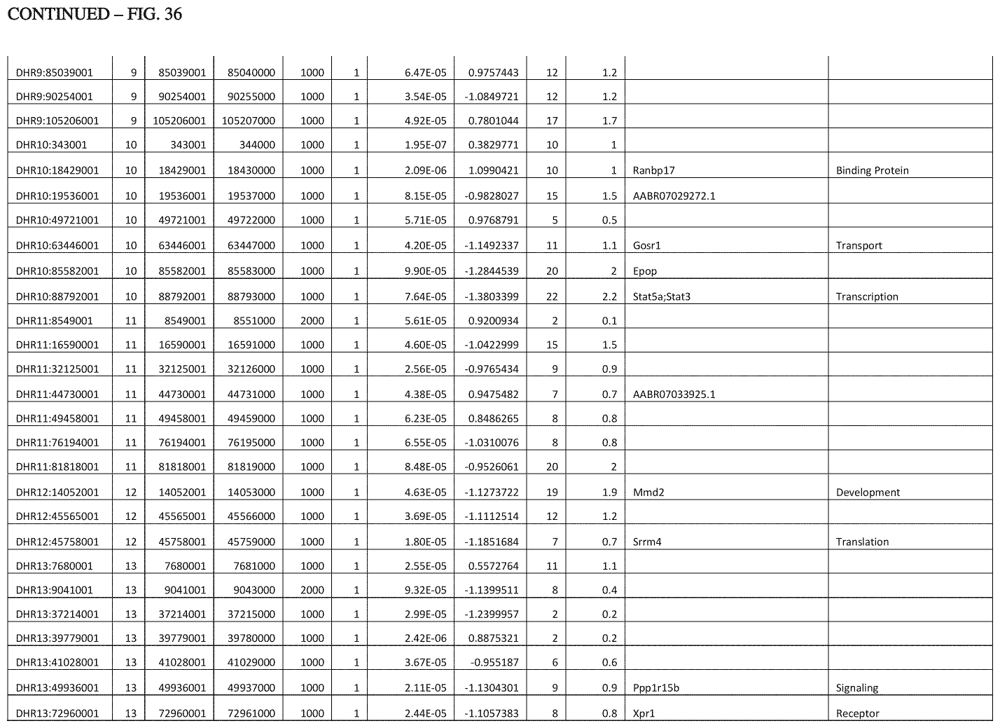

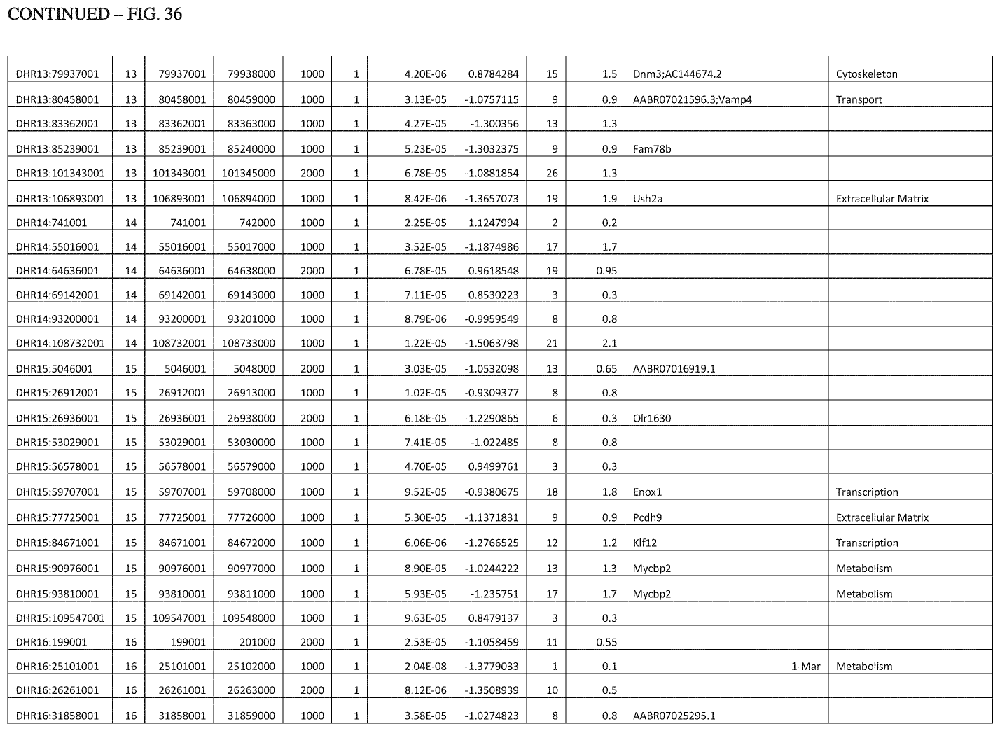

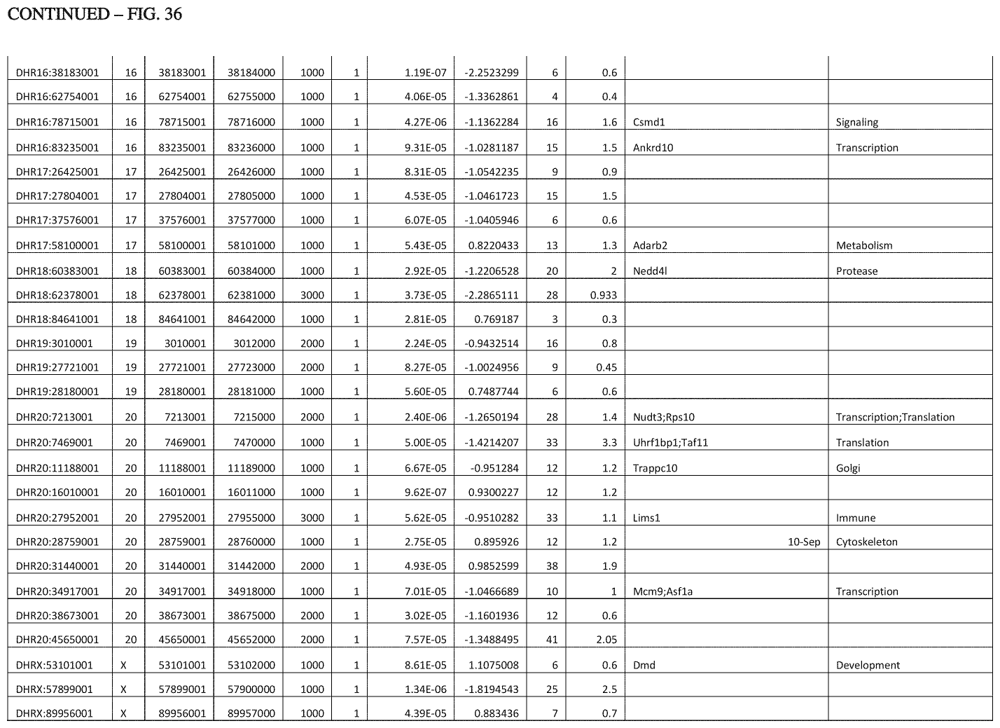

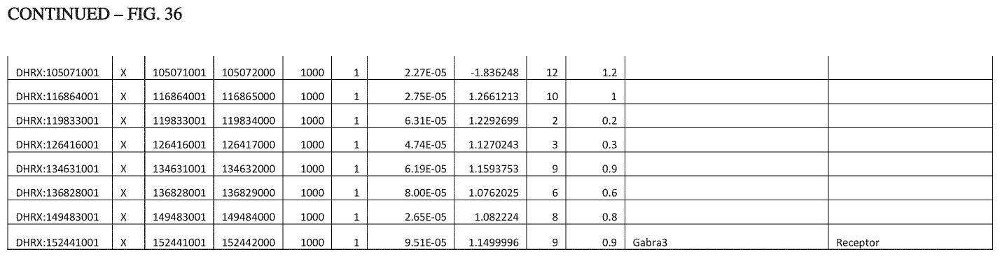

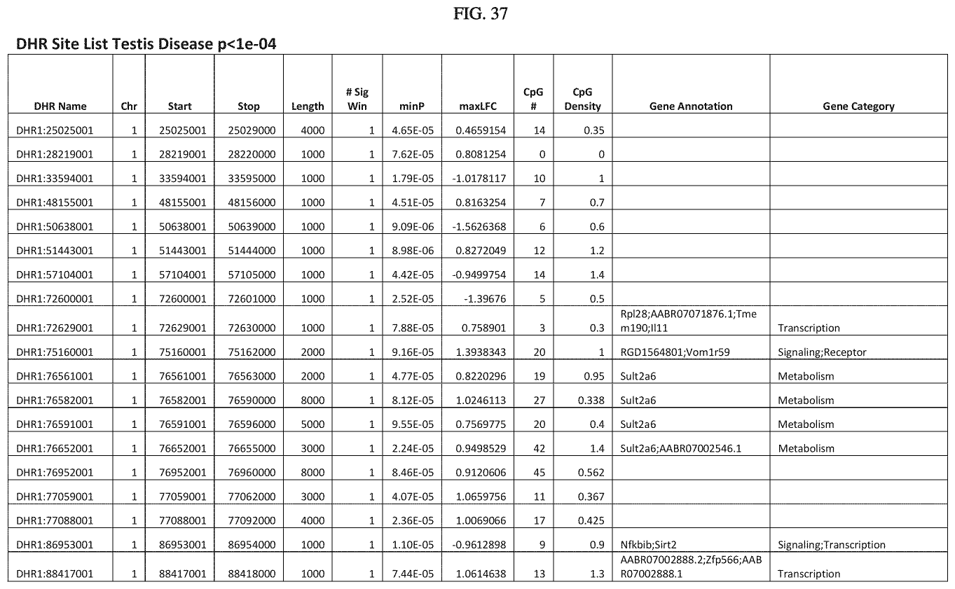

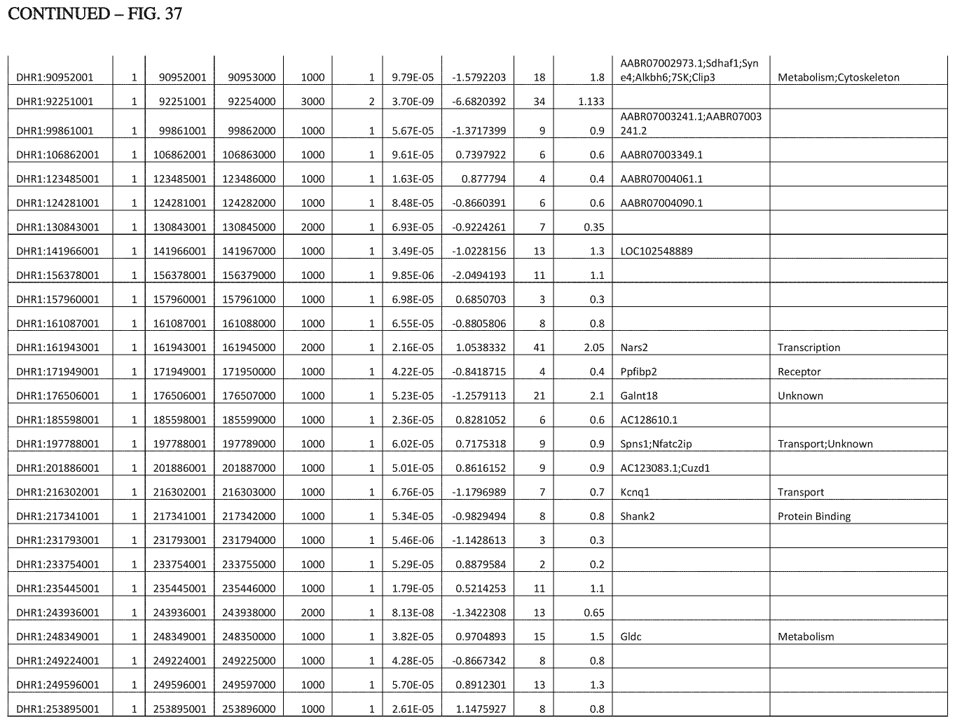

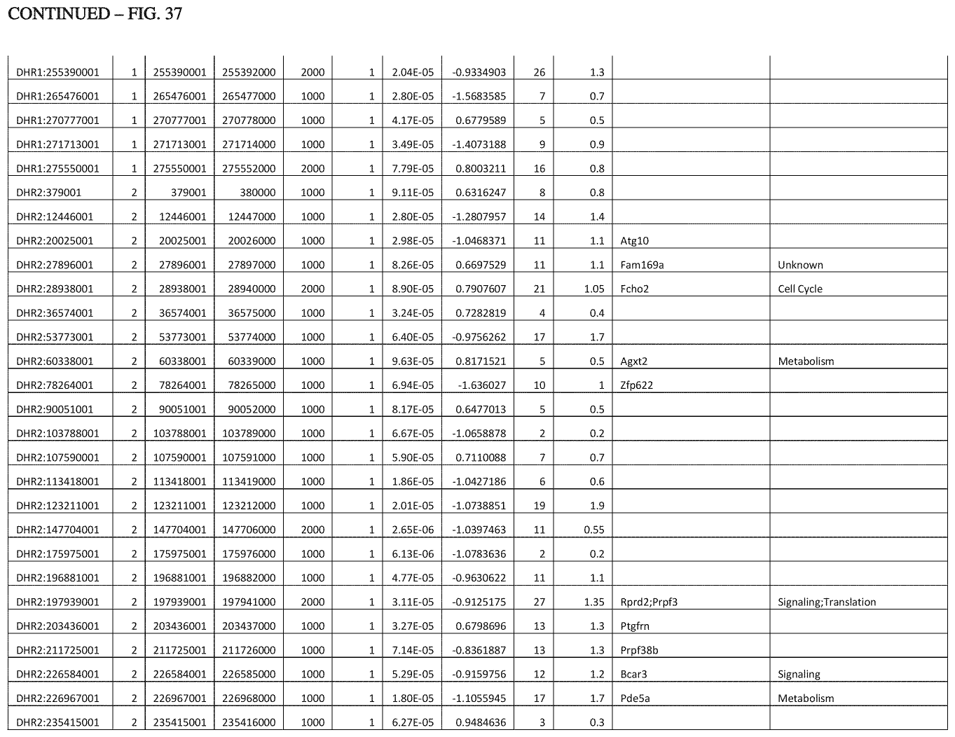

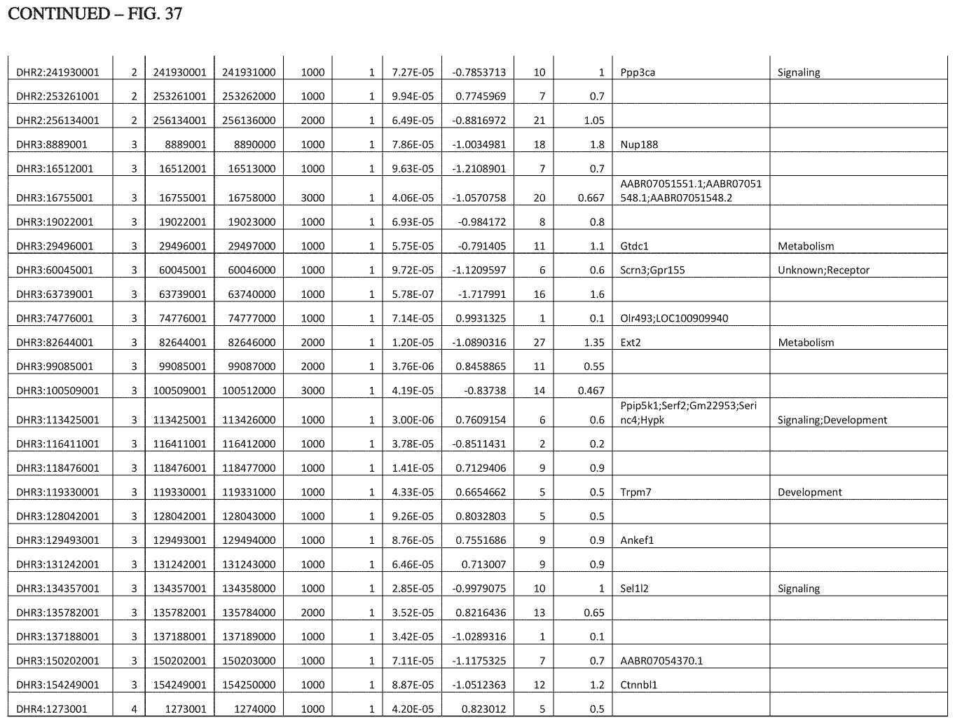

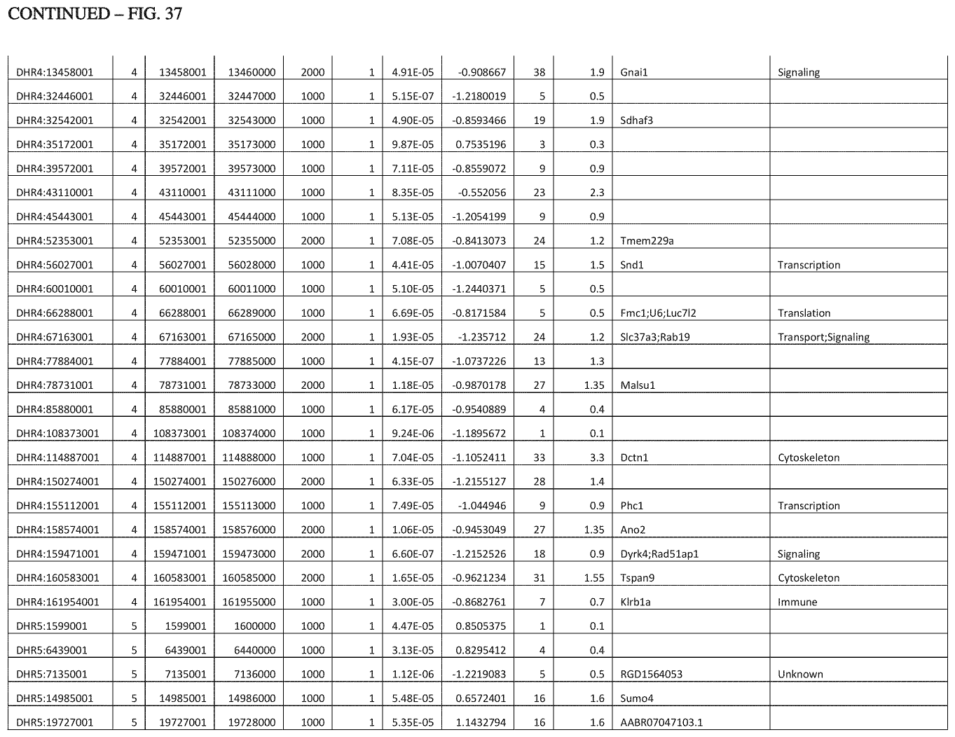

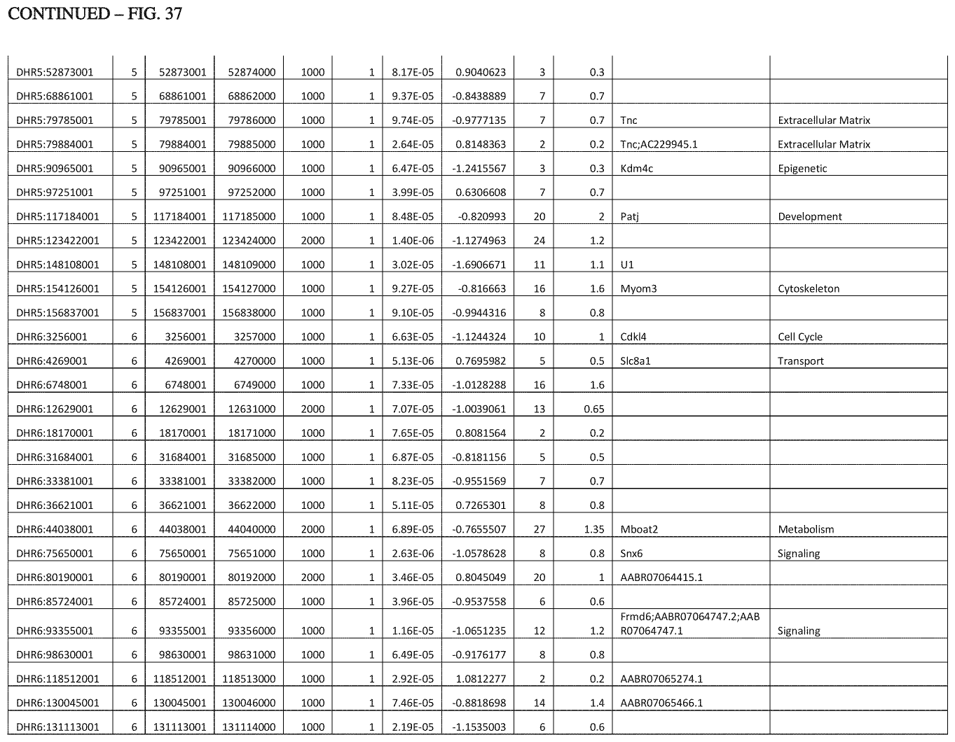

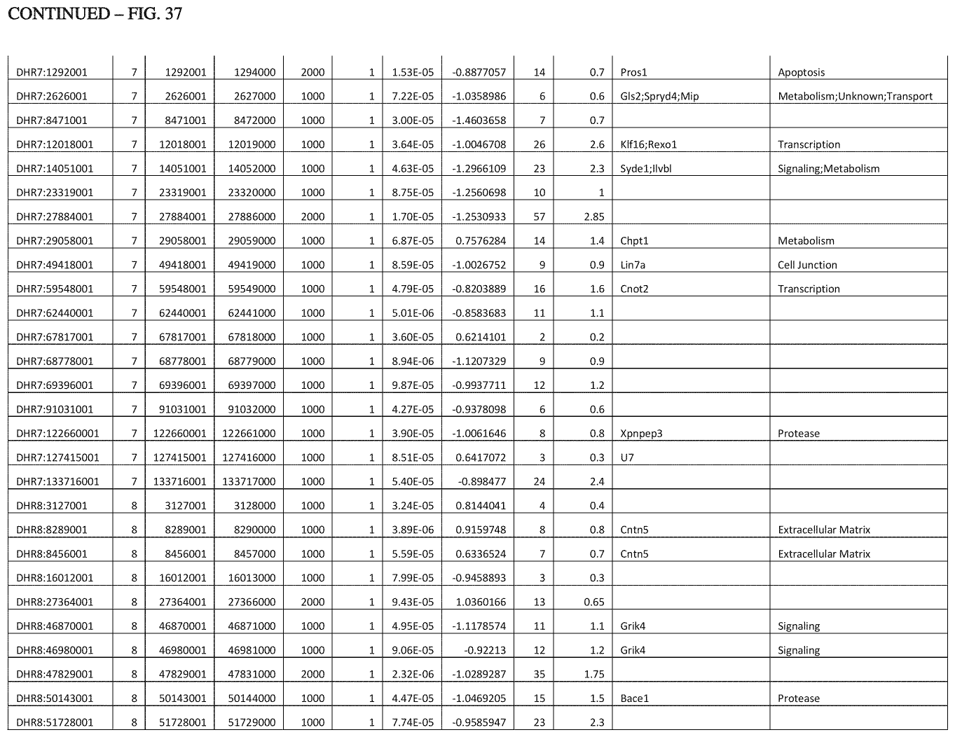

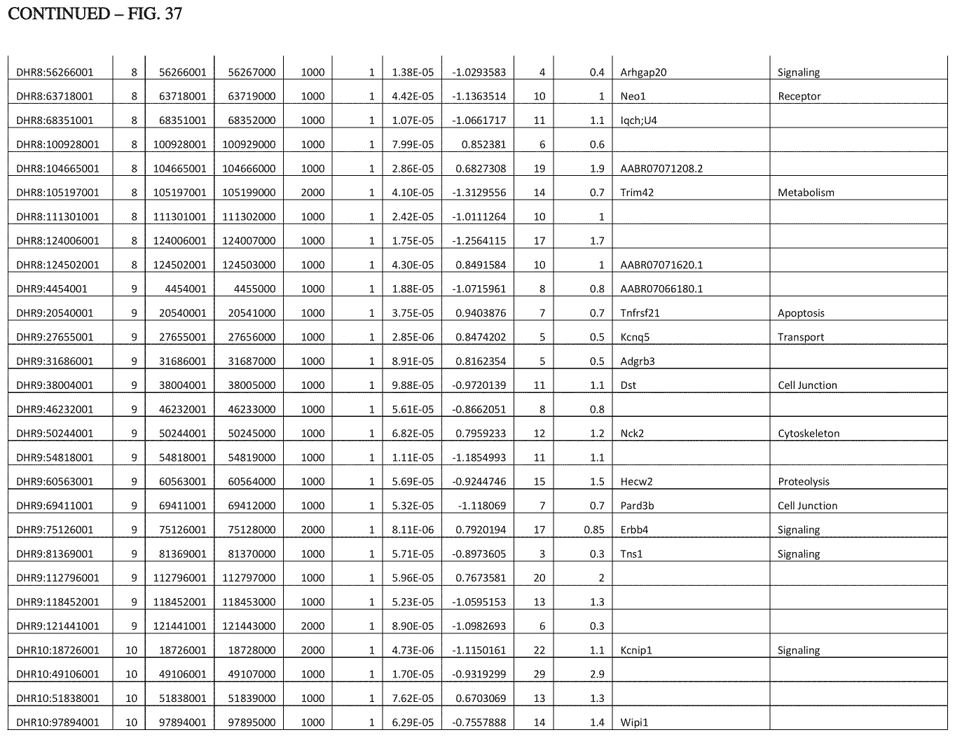

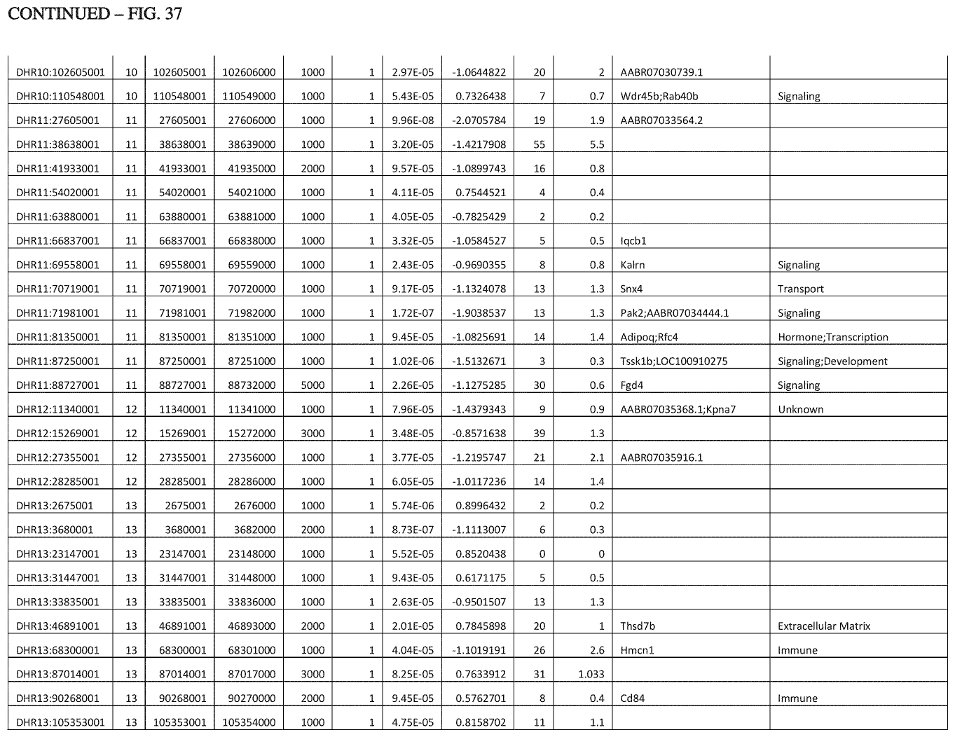

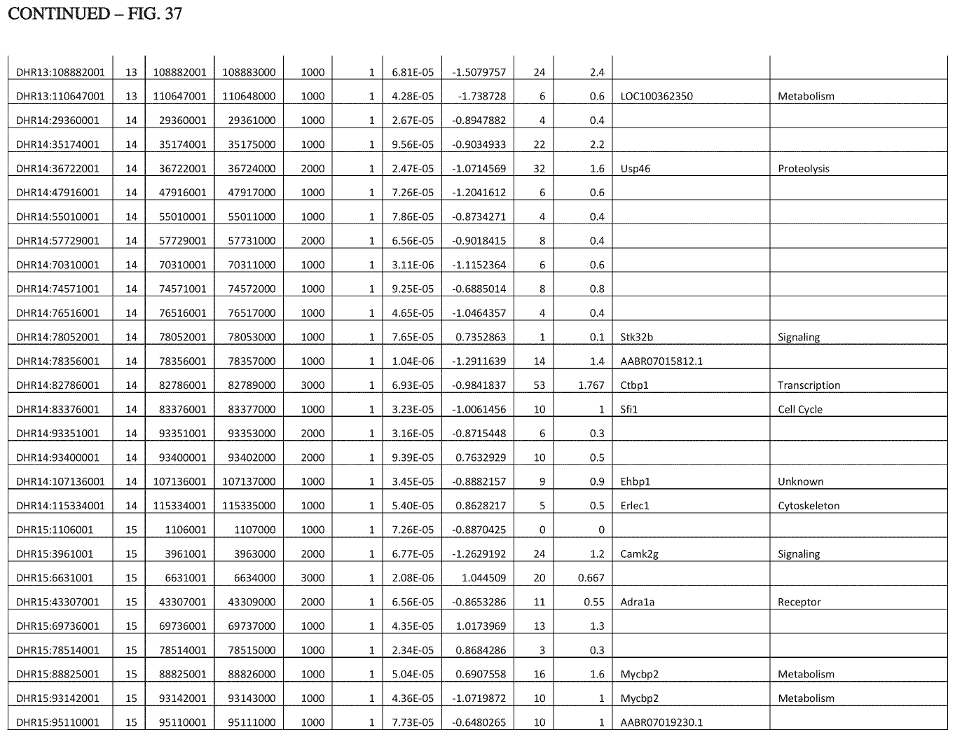

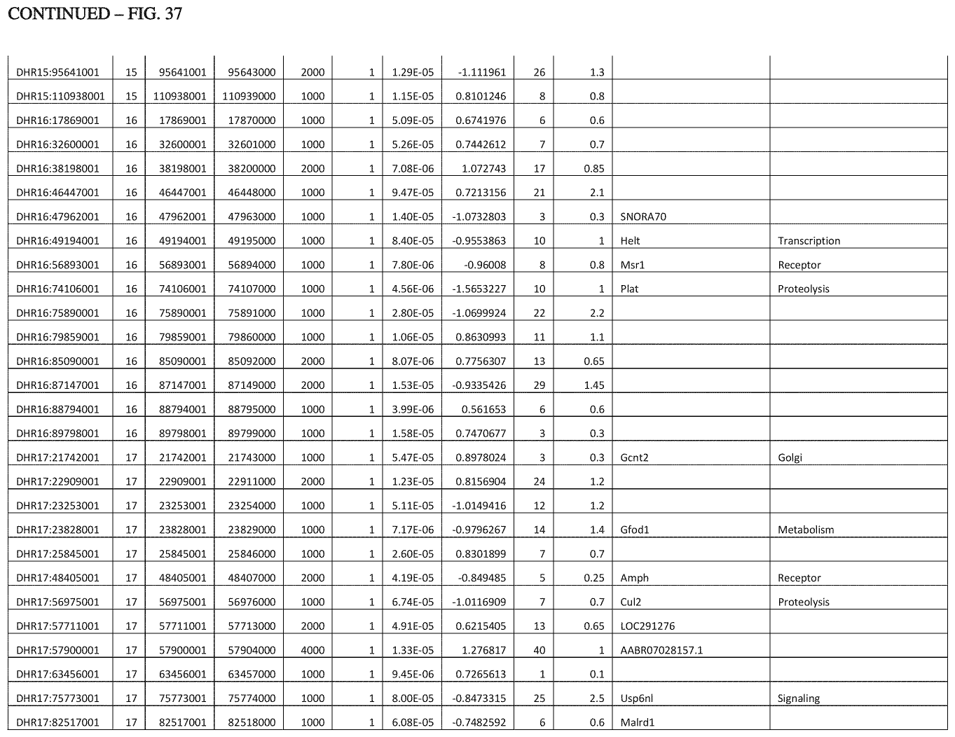

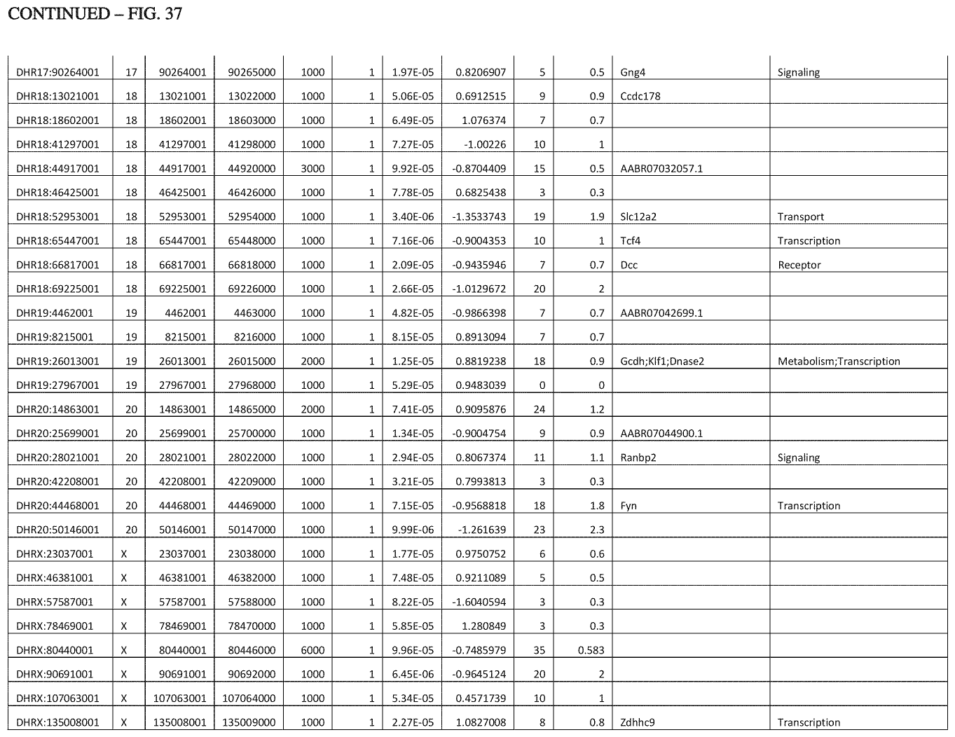

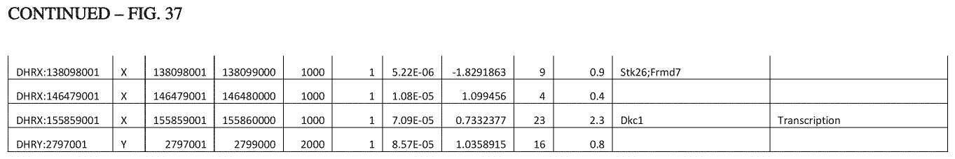

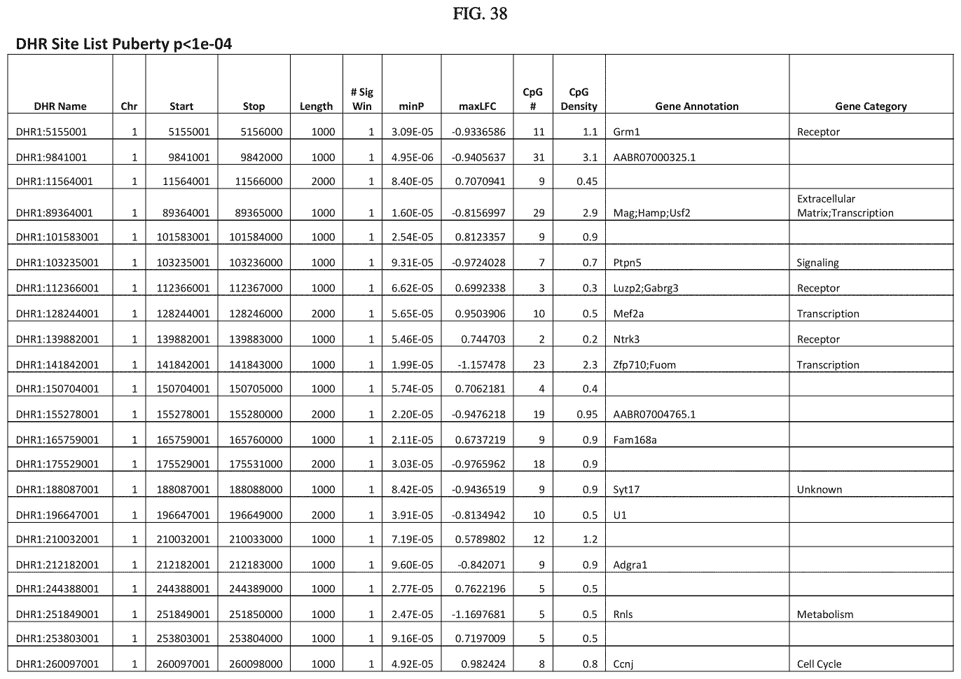

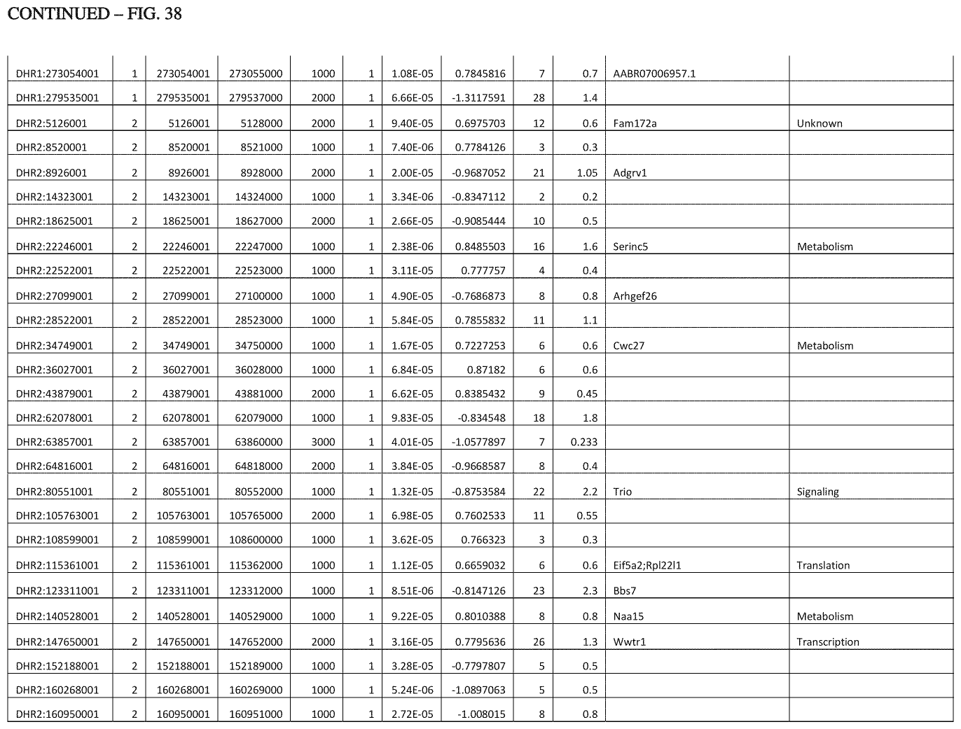

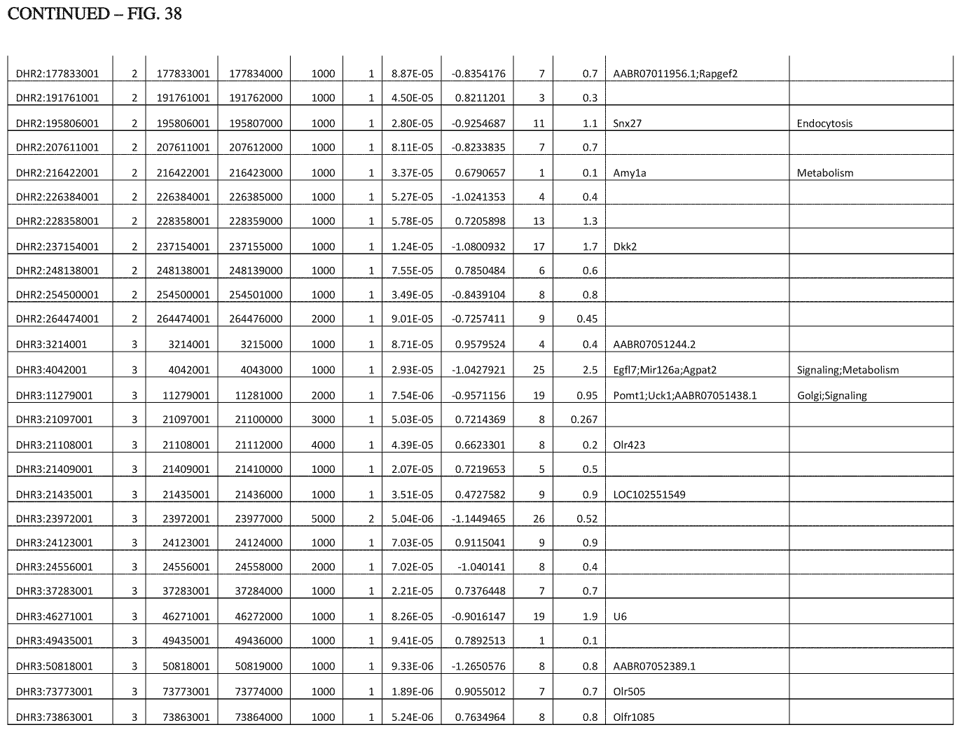

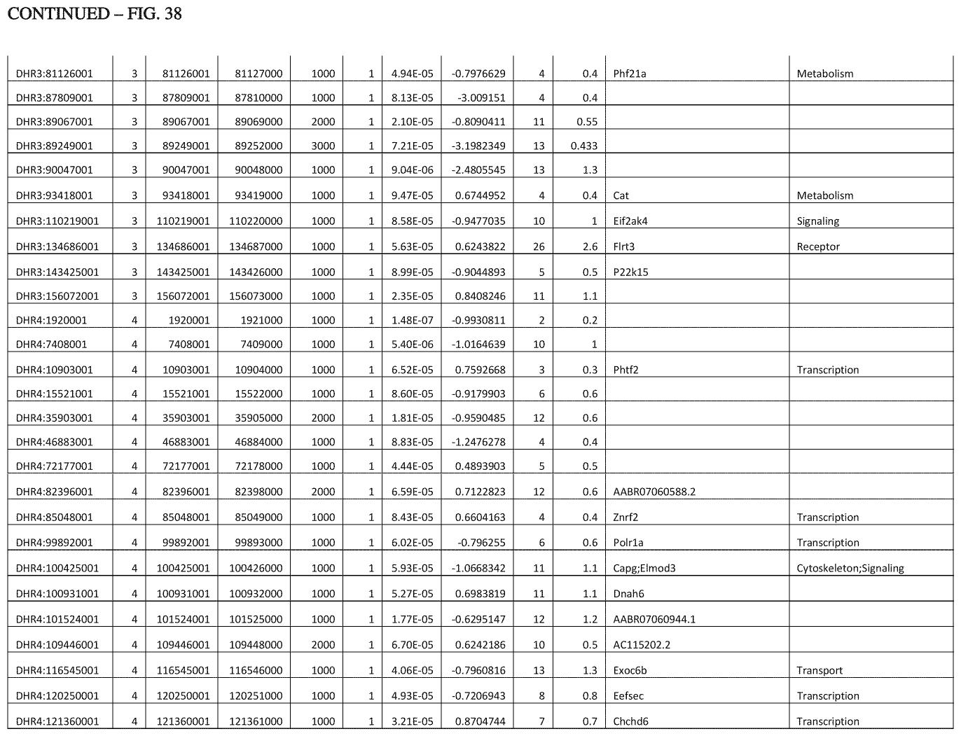

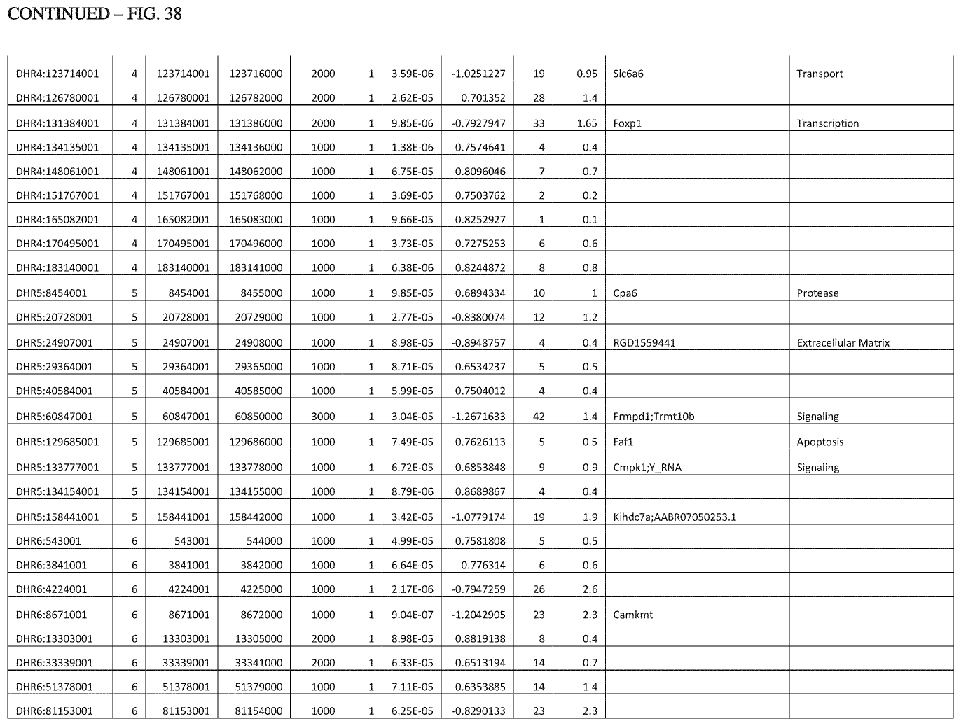

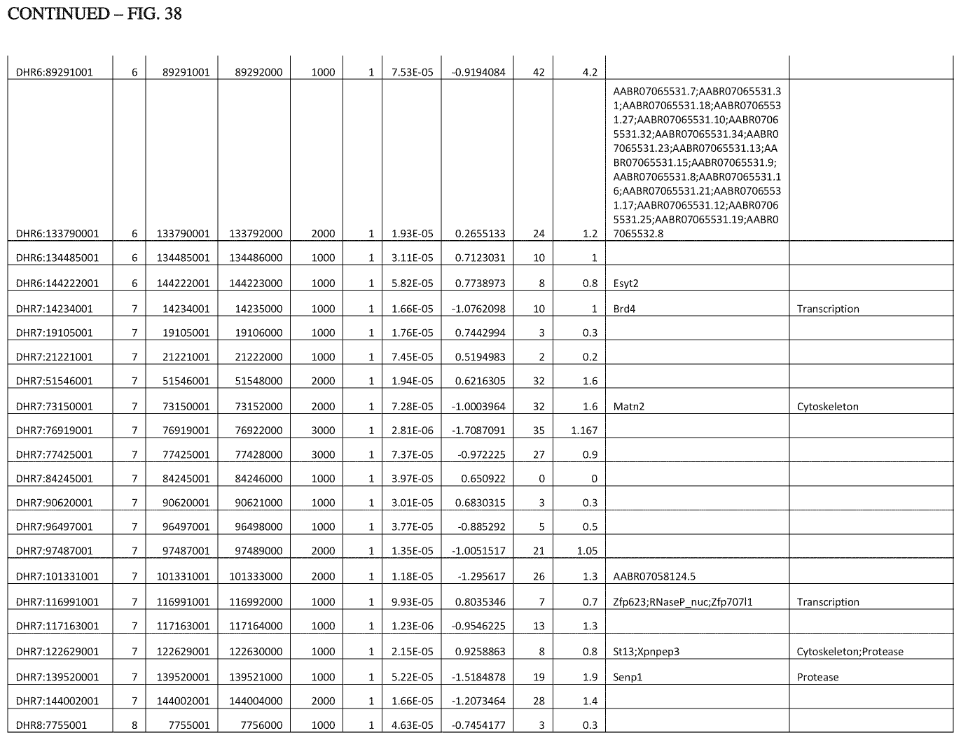

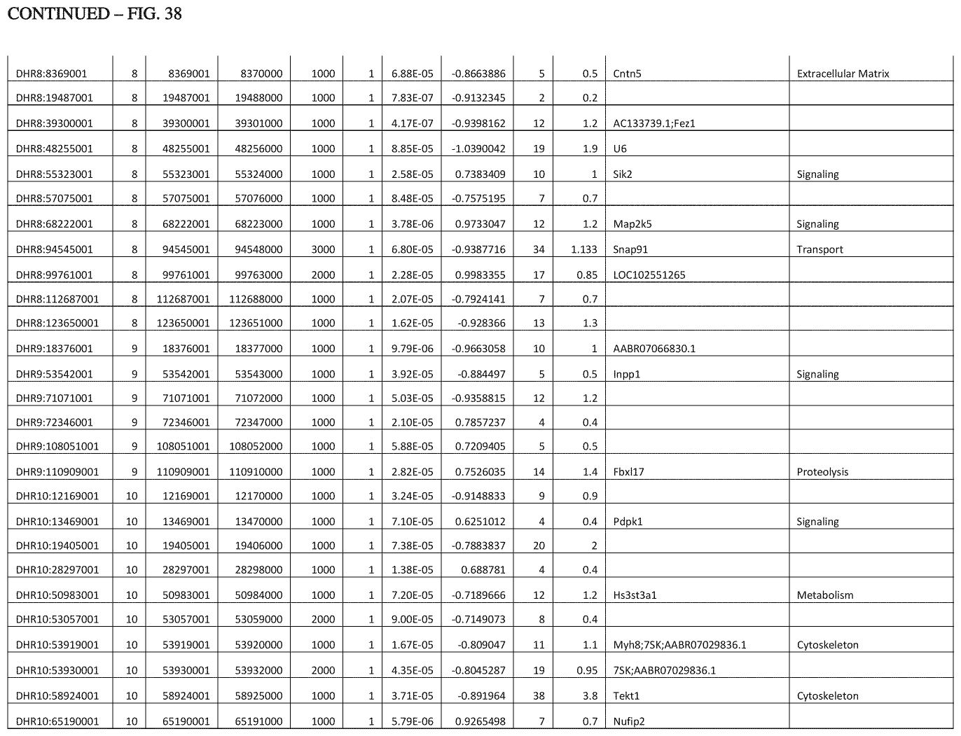

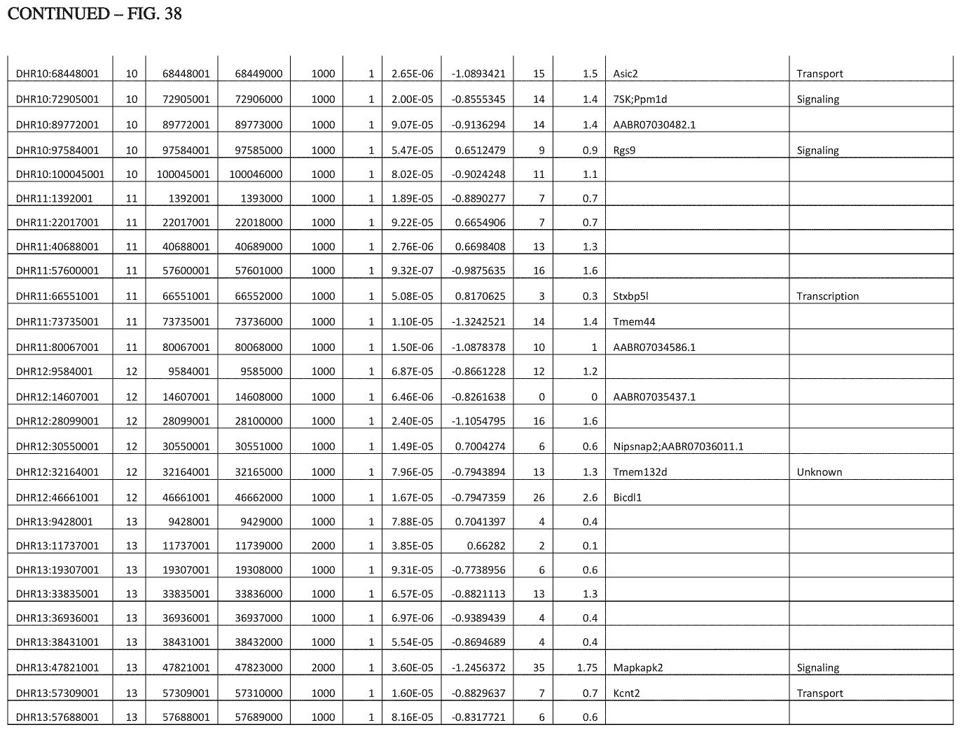

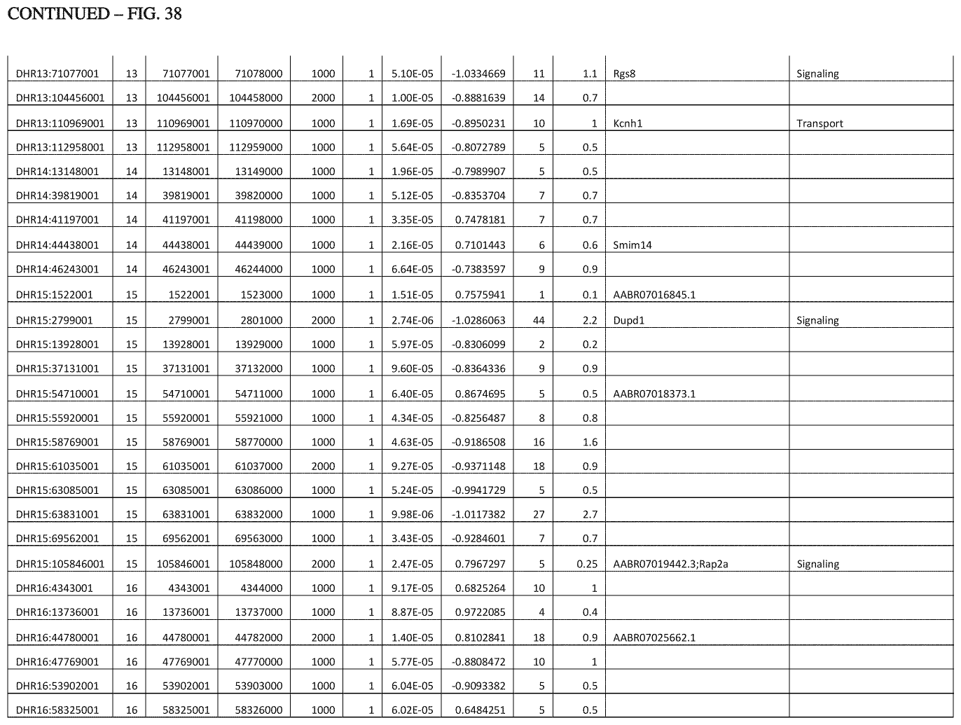

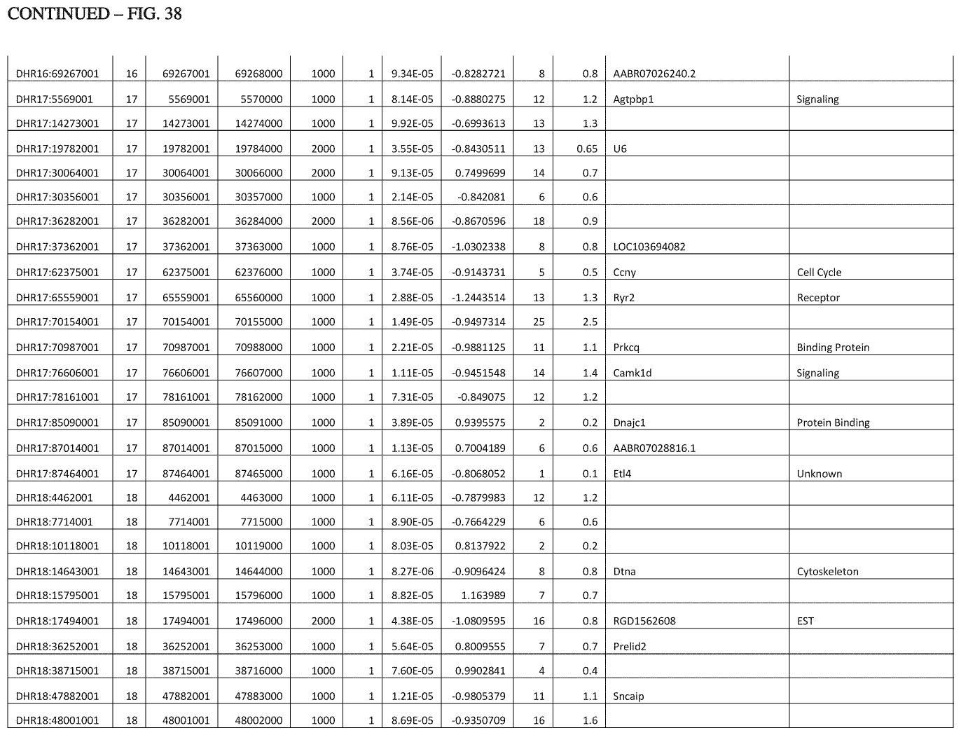

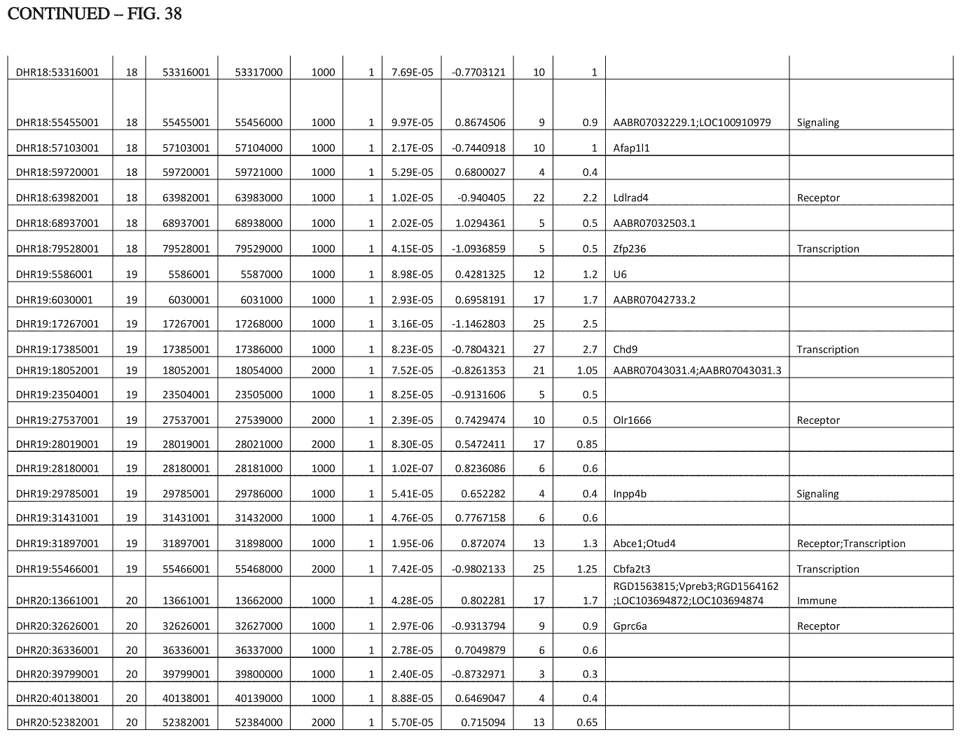

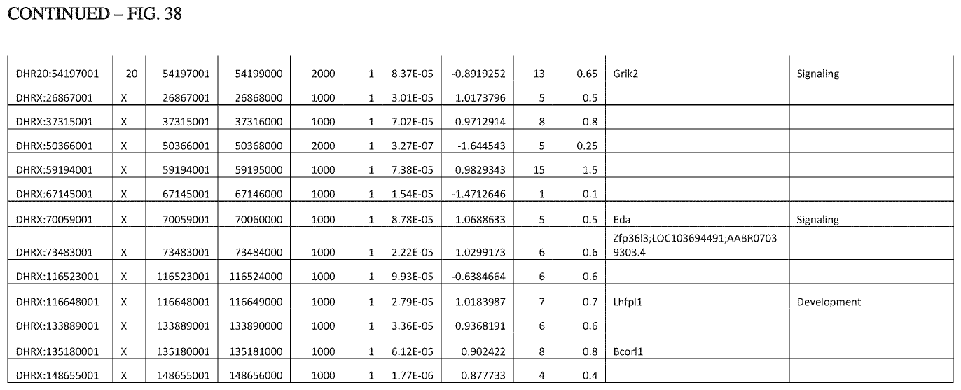

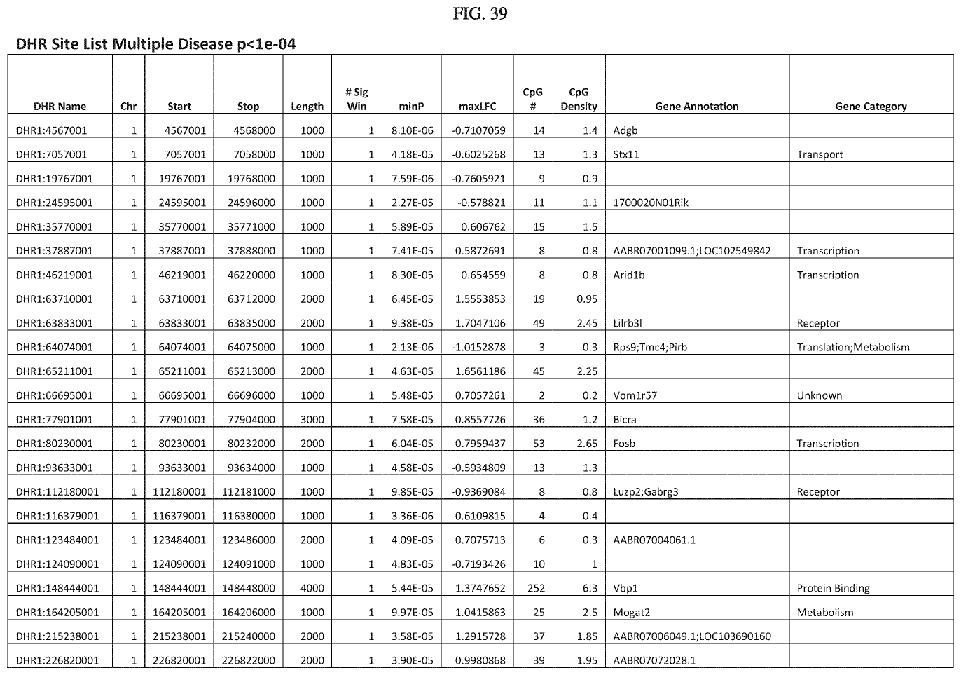

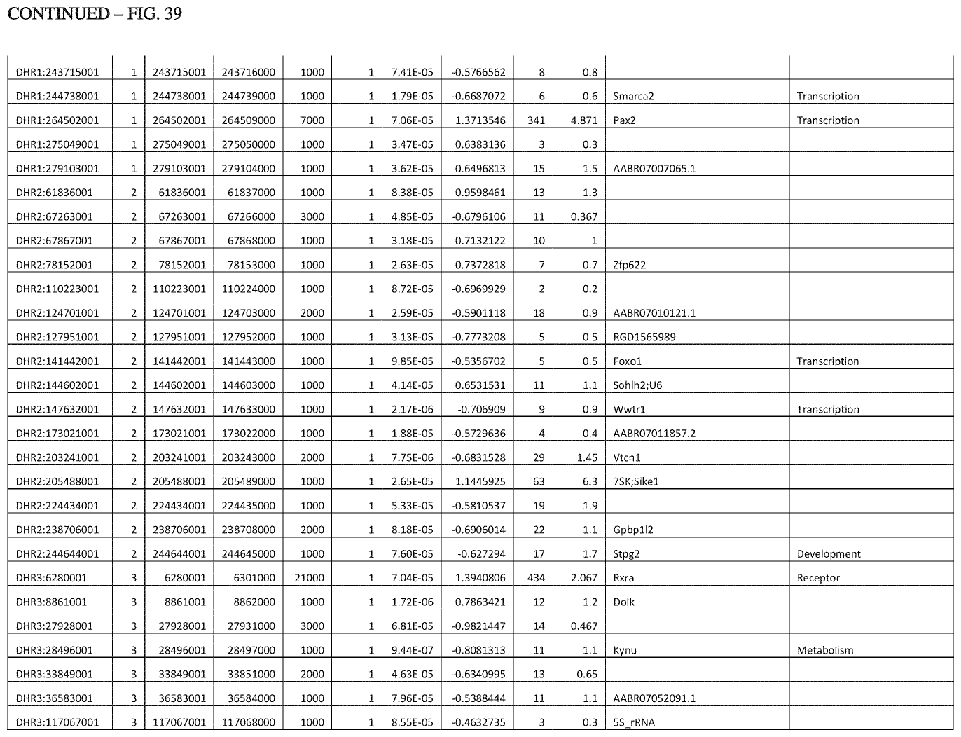

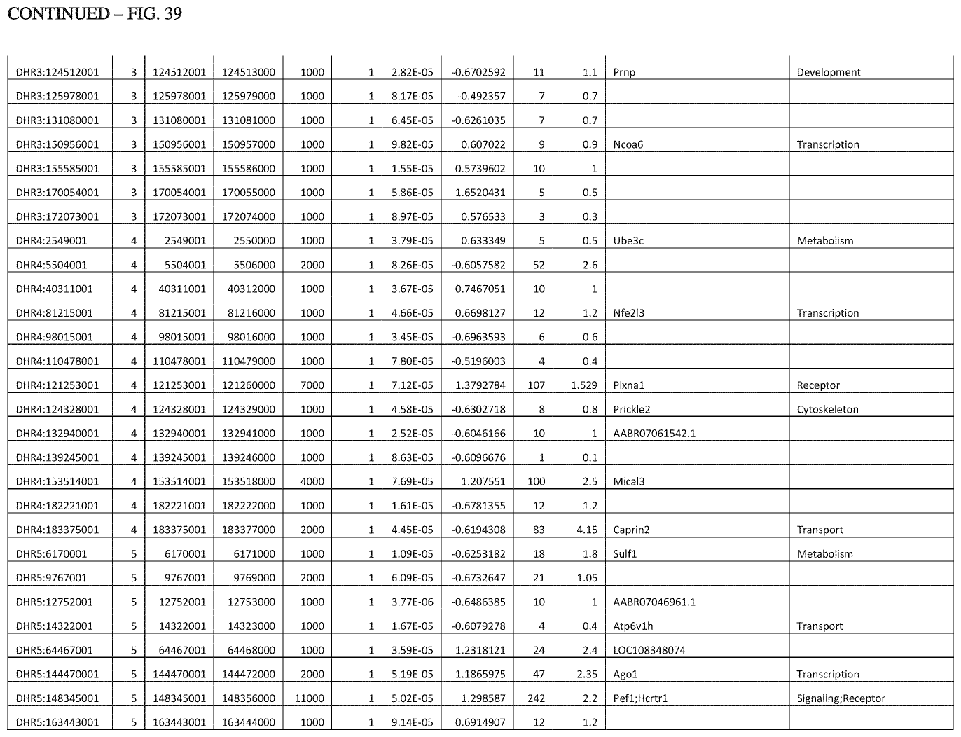

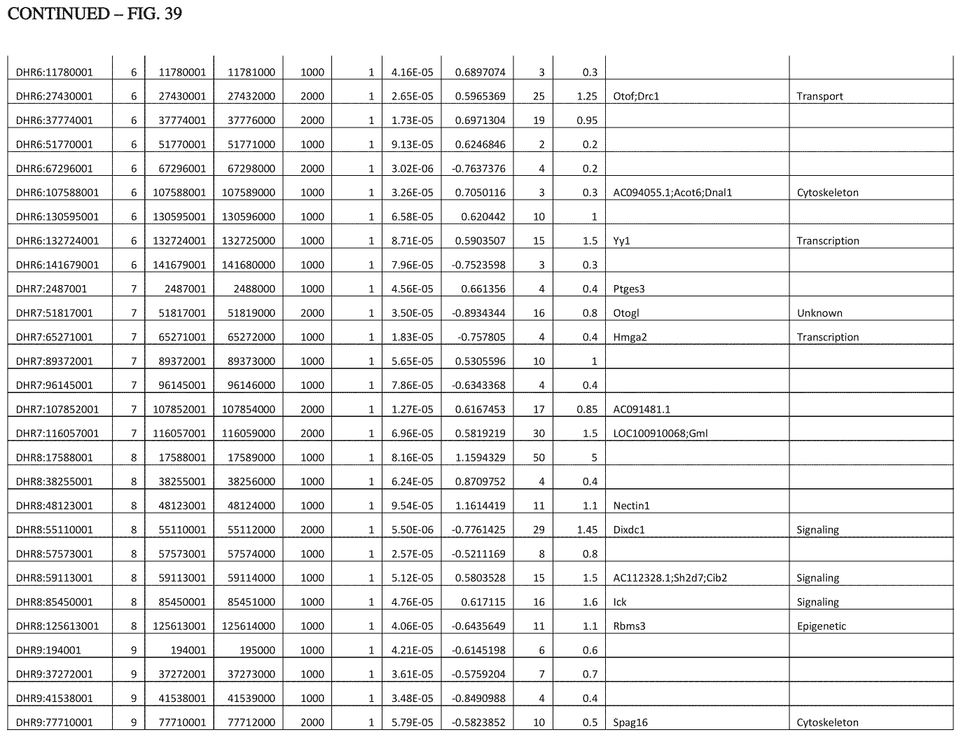

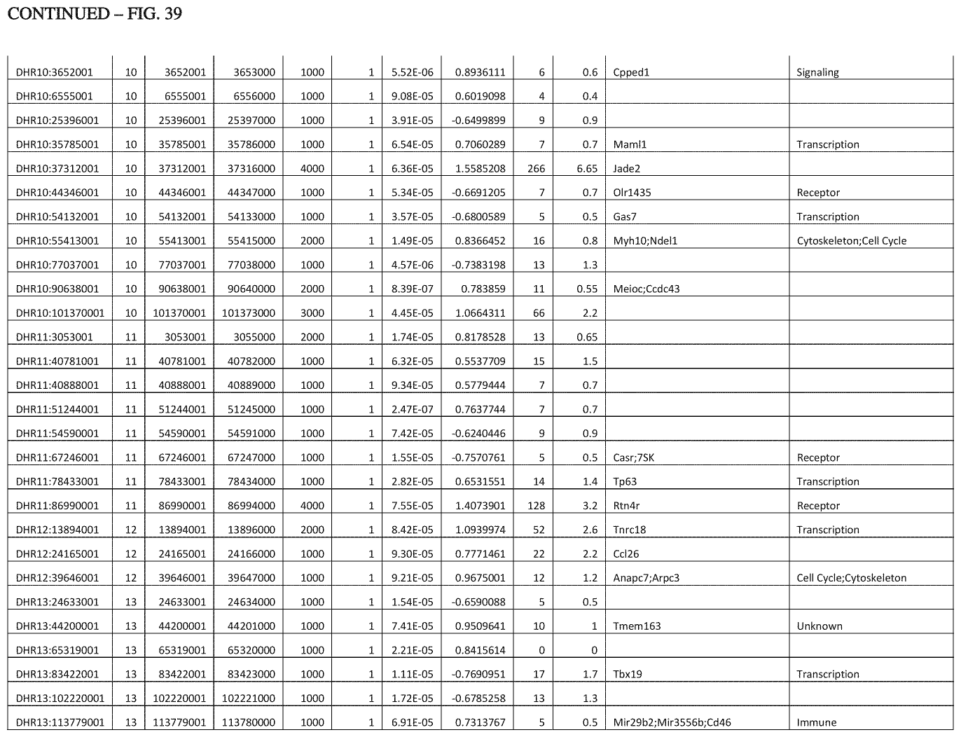

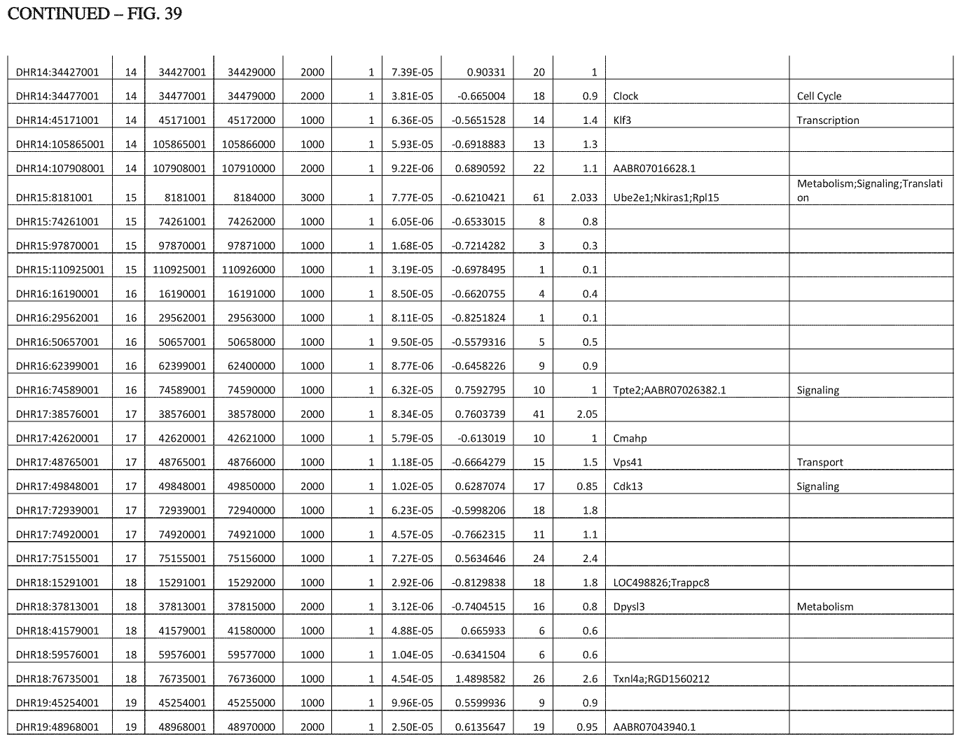

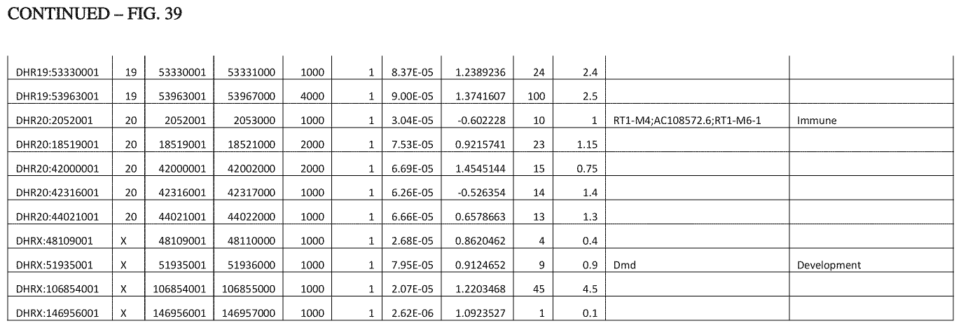

Sperm were collected from the atrazine lineage F3 generation males for analysis. Chromatin from the sperm was isolated and fragmented. A histone H3 antibody is used in a chromatin immunoprecipitation (ChIP) analysis. The retained fragments of DNA were then sequenced for a ChIP-Seq analysis, similar to the MeDIP-Seq analysis, as described in the Methods. This analysis yields the differential histone retention sites (DHRs), which were identified in the sperm using a comparison between the disease specific and non-disease atrazine exposure lineage males (FIG. 34 and FIGS. 35-39). The same sets of animals were used for the identification of DHRs associated with each disease as were used in the identification of DMRs associated with each disease. The lean phenotype exhibited the highest number of differentially retained histone sites, with 2859 found at edgeR p<1e−04, indicating the greatest amount of epigenetic shift was associated with individuals exhibiting a lean phenotype following transgenerational exposure to atrazine (FIG. 34A). The other diseases each yield several hundred transgenerational DHRs at an edgeR p-value threshold of p<1e−4 (FIG. 34B-34E and FIGS. 35-39). The log-fold change in DHRs is presented and an increase in histone retention is associated with 44% of pubertal abnormality DHRs, 39% of testis disease DHRs, 36% of kidney disease DHRs, 35% of lean pathology DHRs, and 53% of multiple disease DHRs.

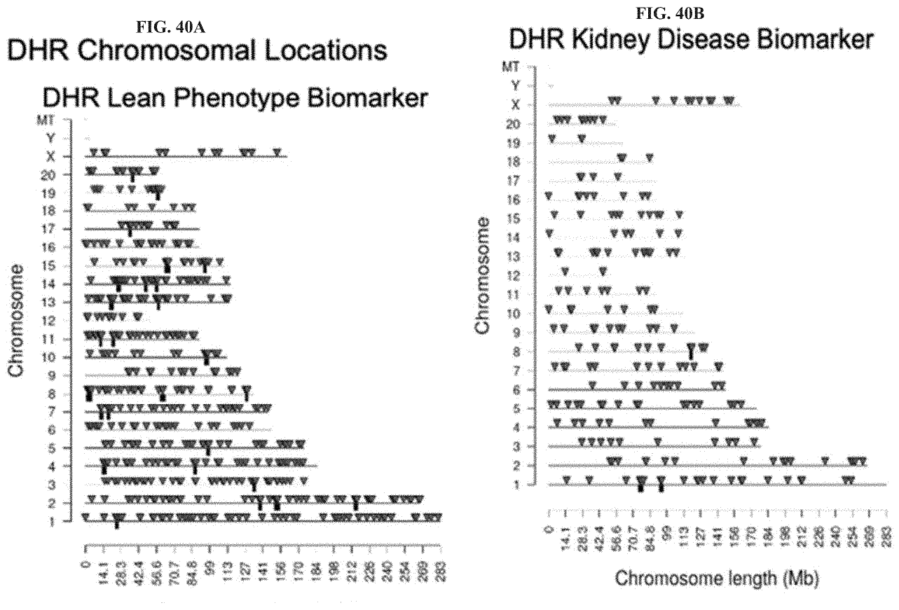

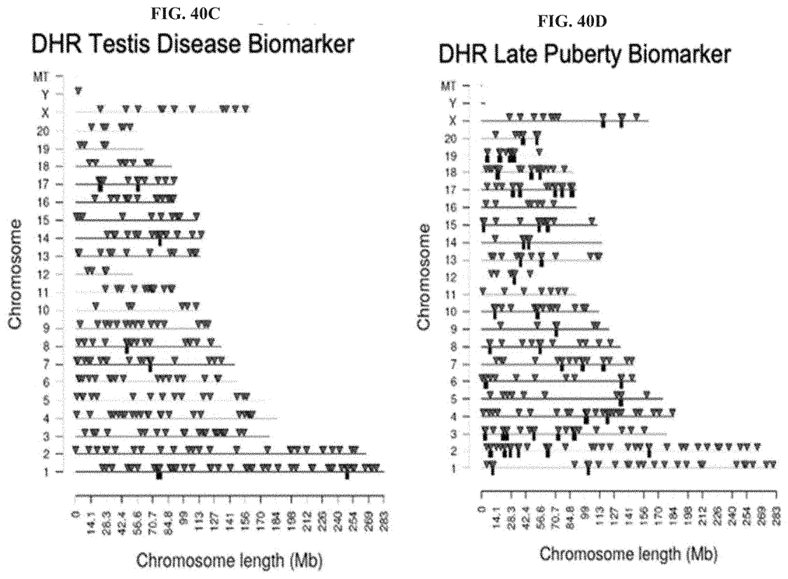

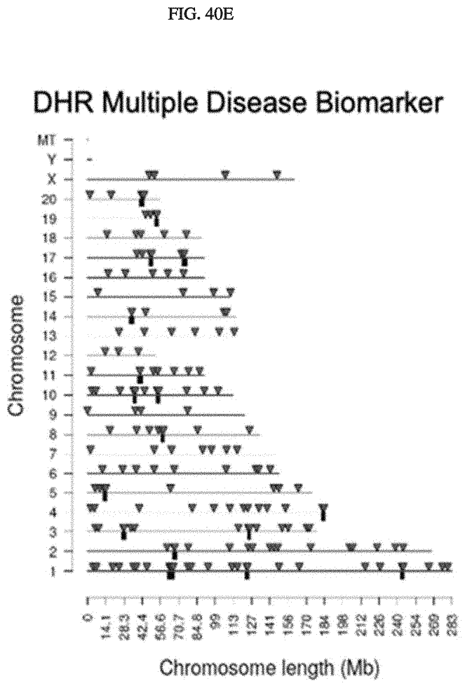

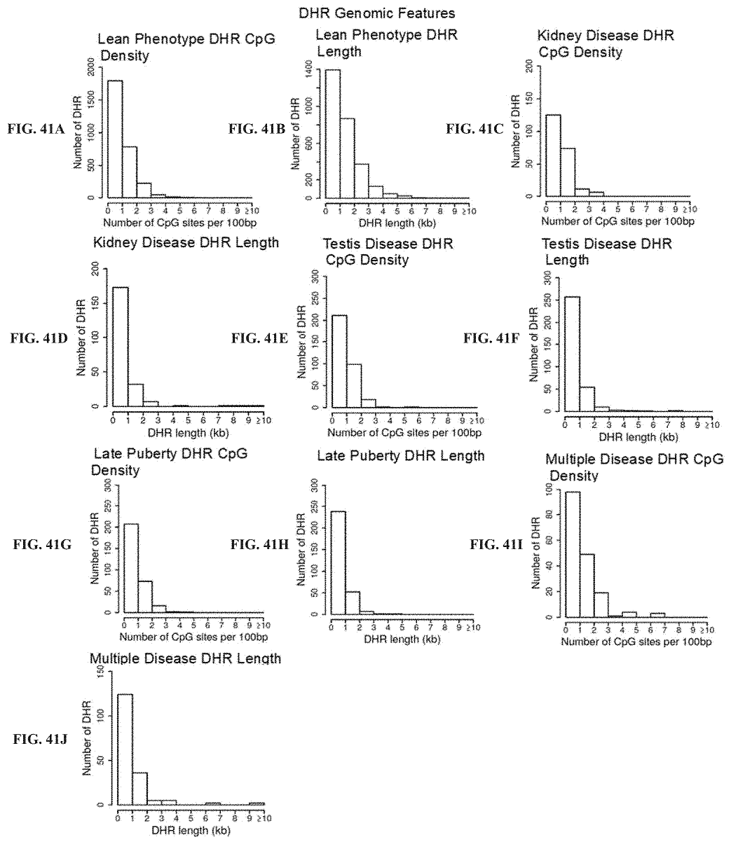

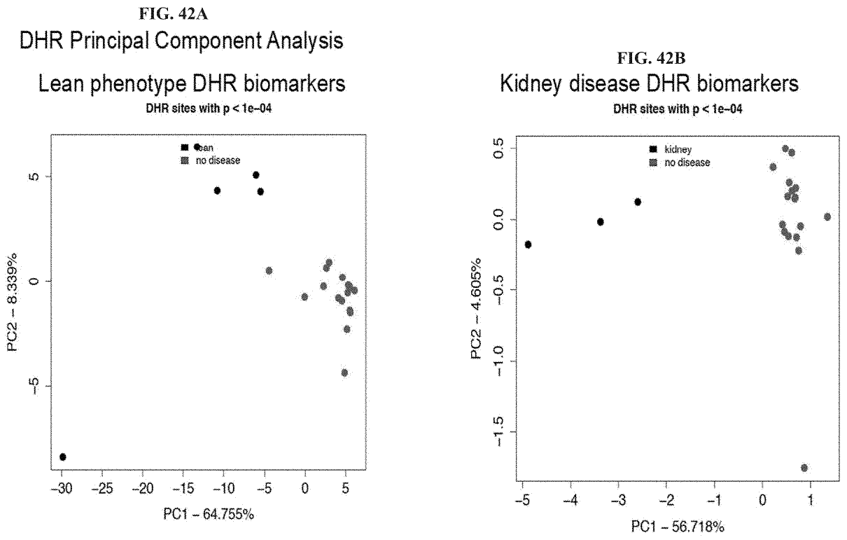

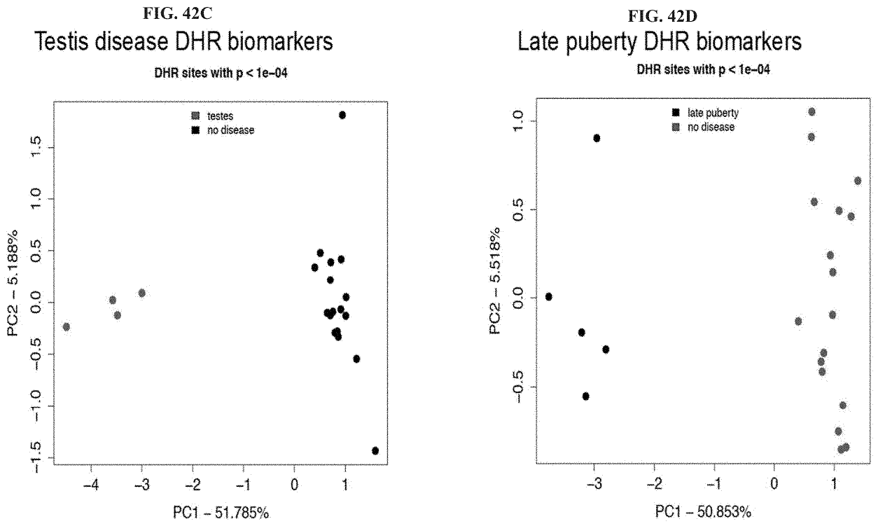

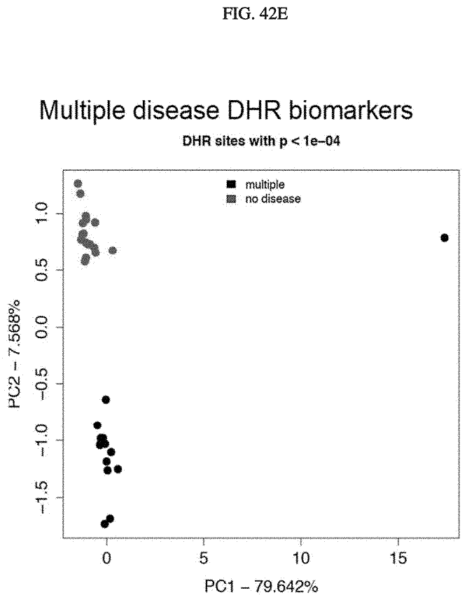

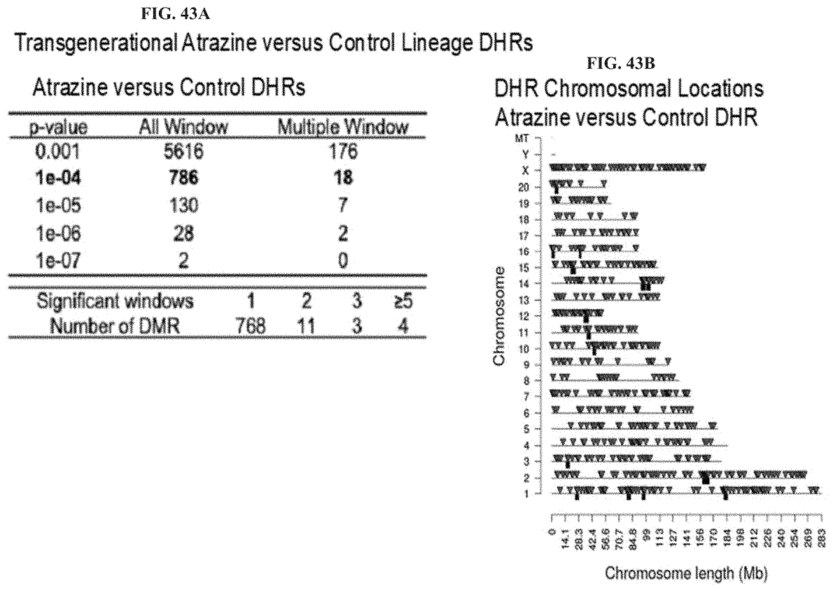

A similar genome wide response is seen in the widespread distribution of DHRs across chromosomes (FIG. 40) as was seen with the DMRs. Therefore, both epigenetic mechanisms examined, differential DNA methylation regions and differential histone retention regions, show this genome wide epigenetic response transgenerationally. The CpG density within differential histone retention sites is low with the majority of CpGs between 1 and 3 sites per 100 bp (FIG. 41). The length of most DHRs is between 1,000 and 3,000 base pairs are shown in FIG. 41. A principal component analysis (PCA) is presented for the disease specific DHRs (FIG. 42). There is distinct clustering and no outlier samples for each of the diseases analyzed compared to the non-disease when the RPKM read depth at DHR sites used in the analysis.

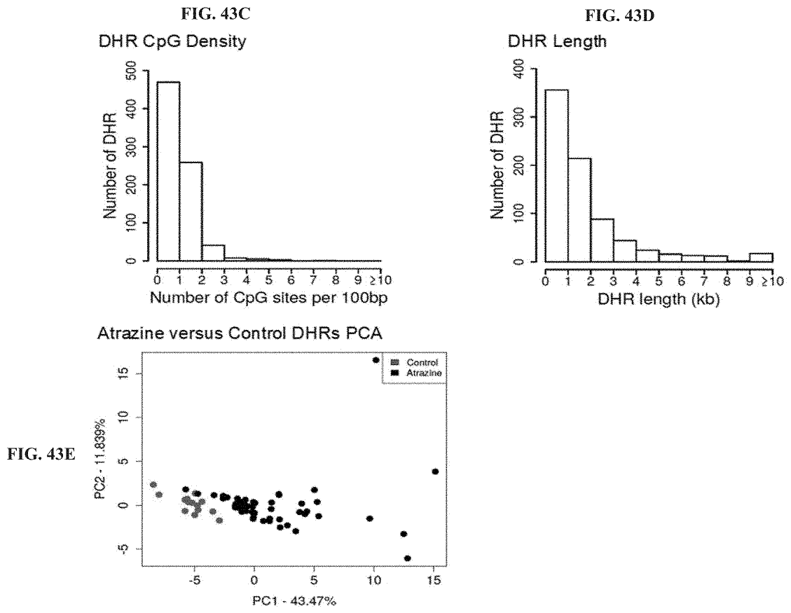

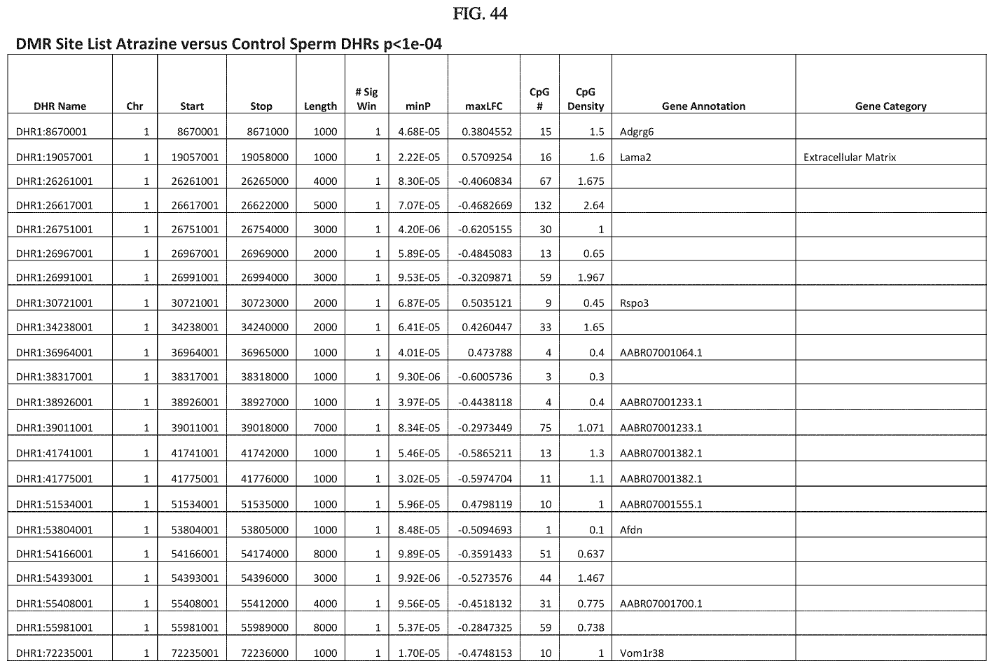

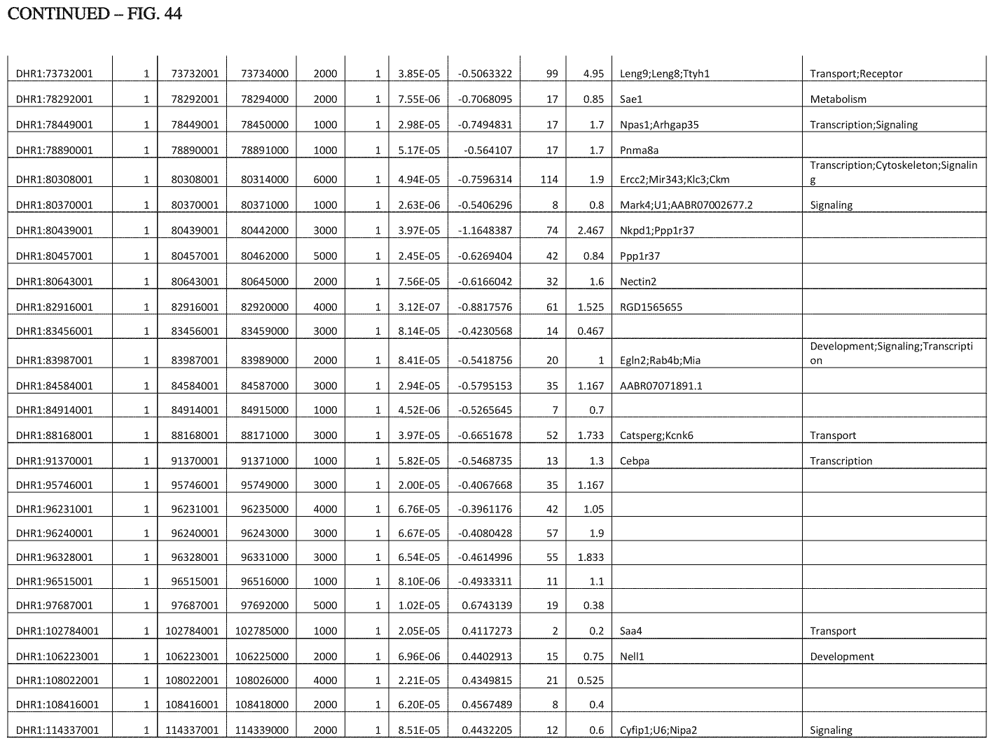

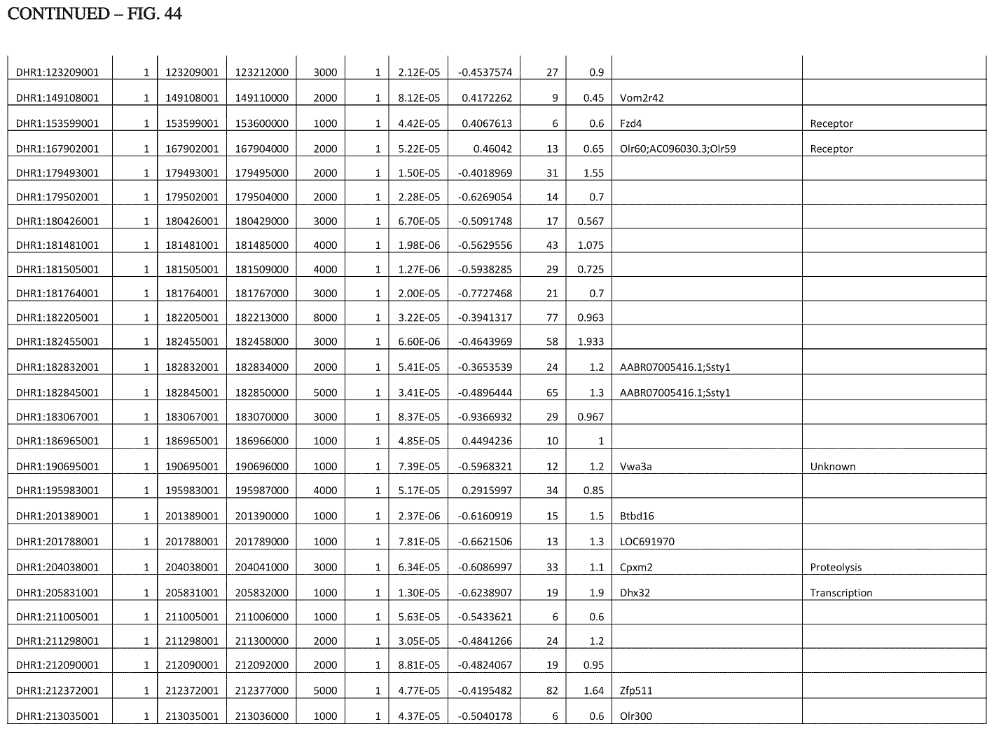

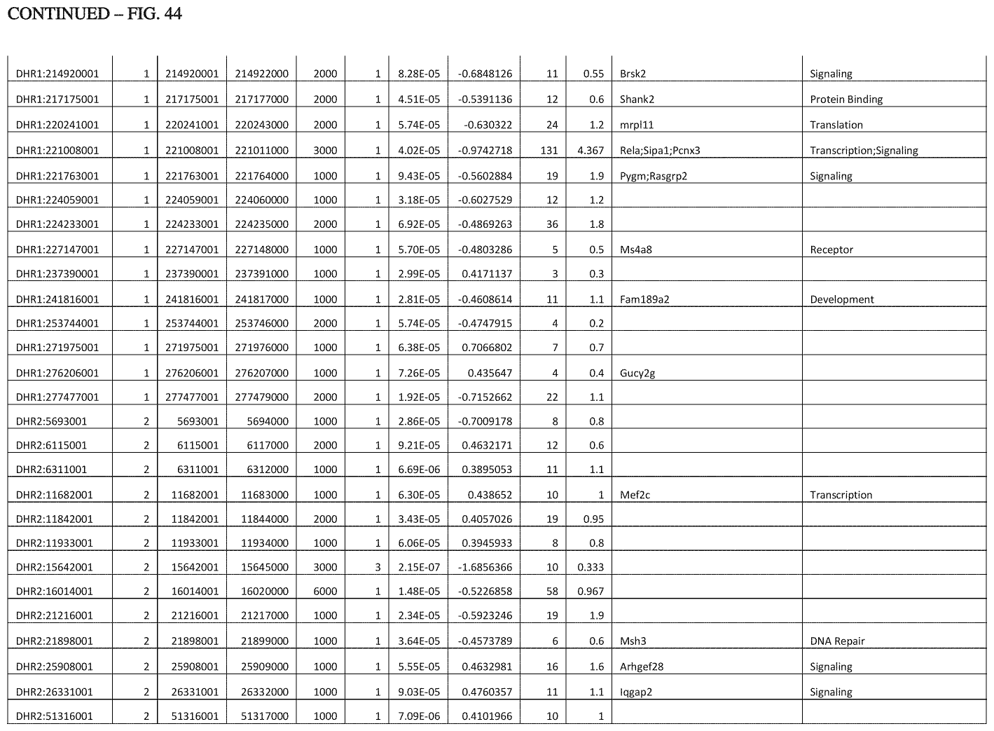

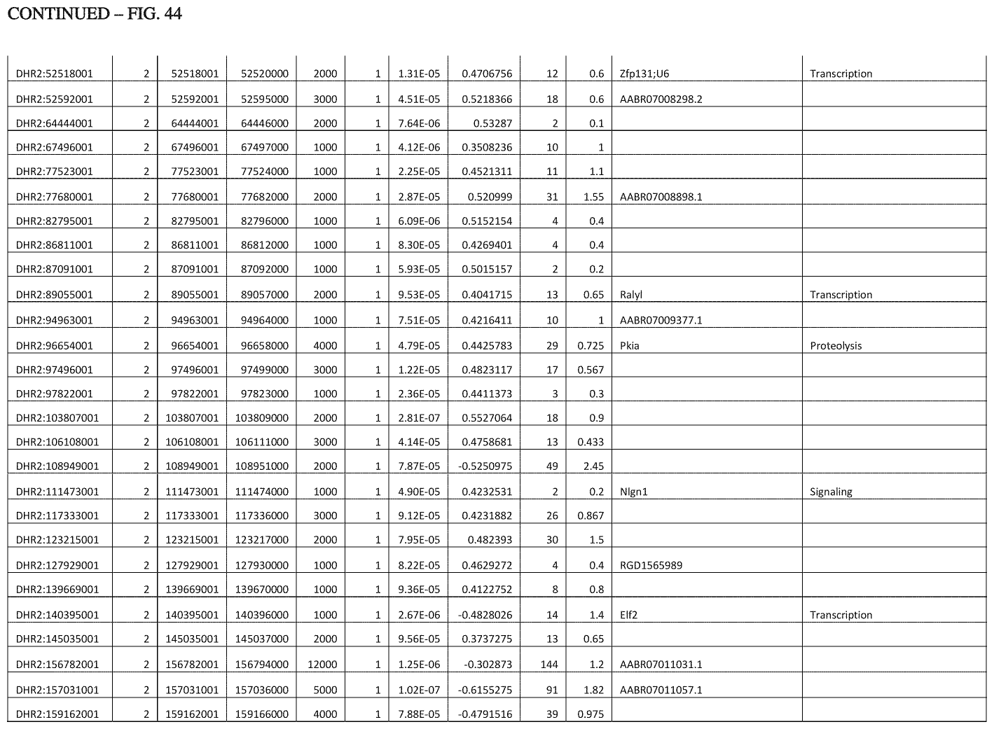

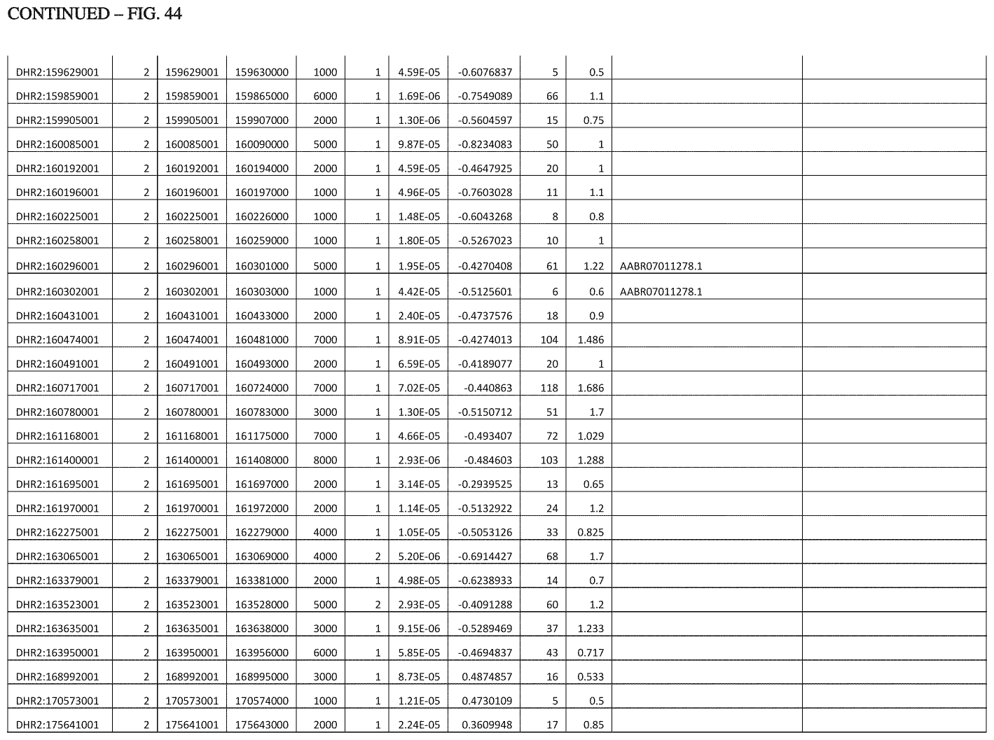

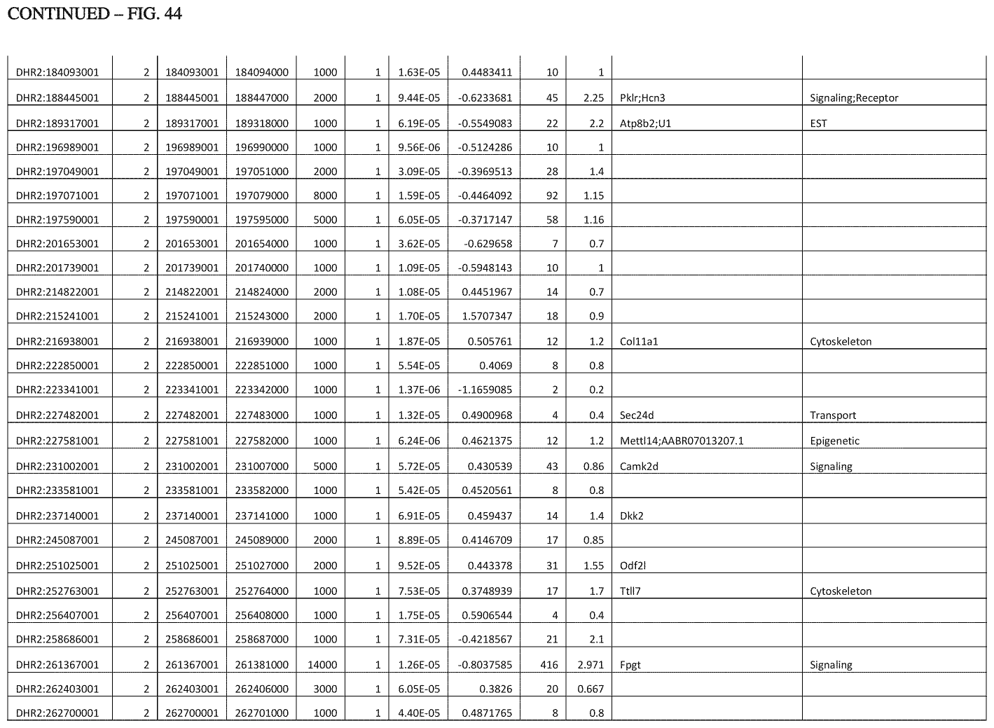

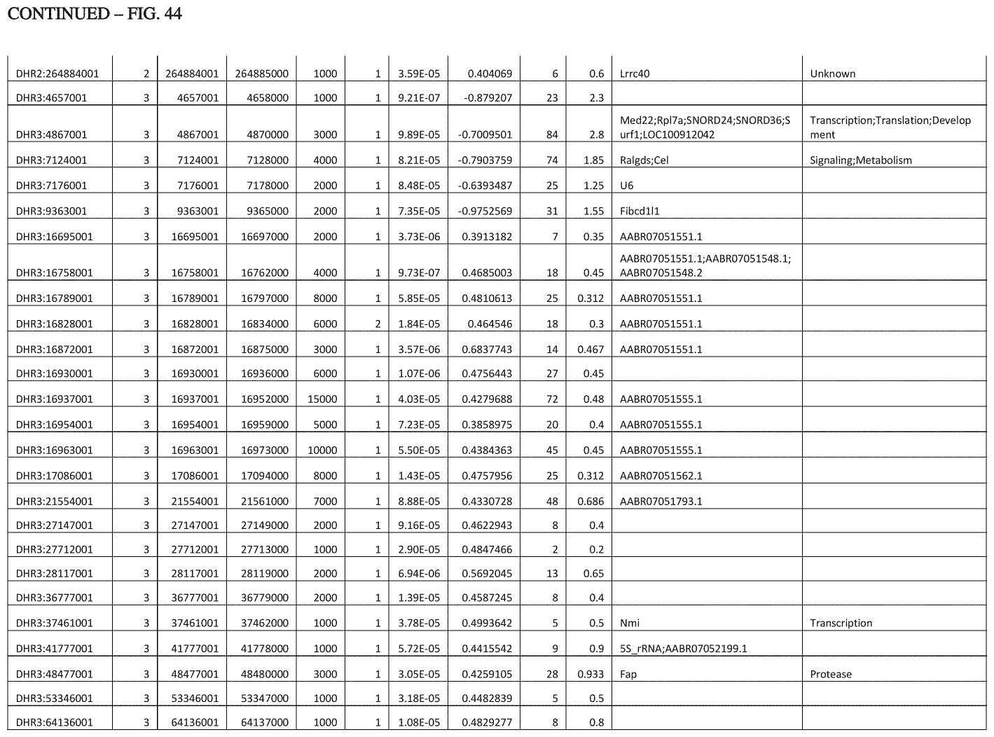

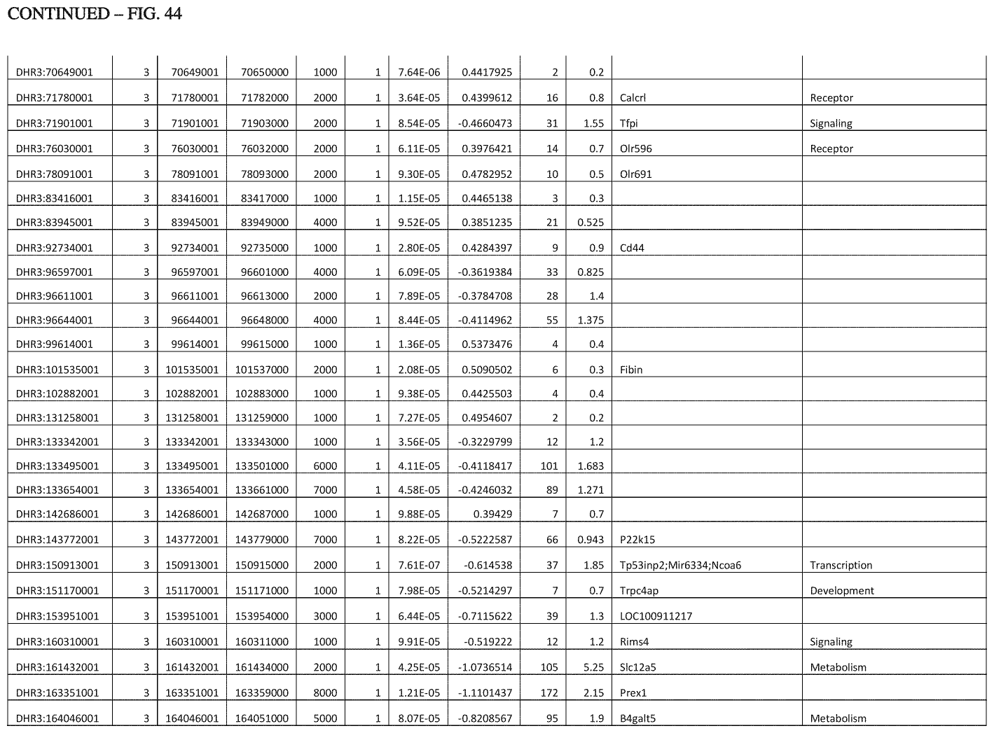

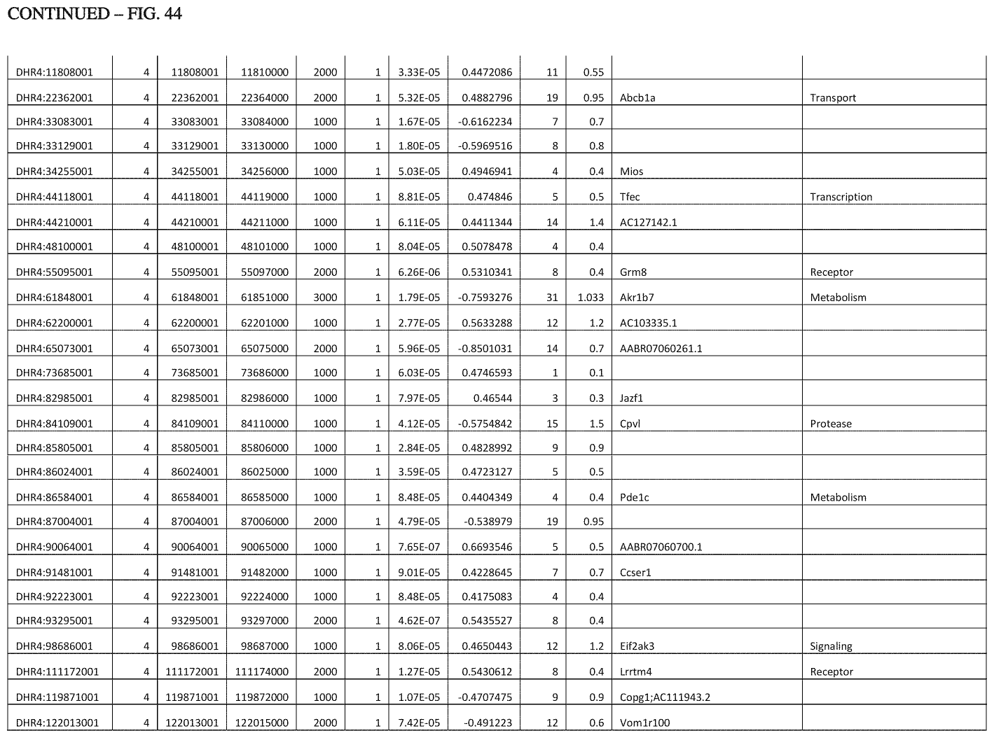

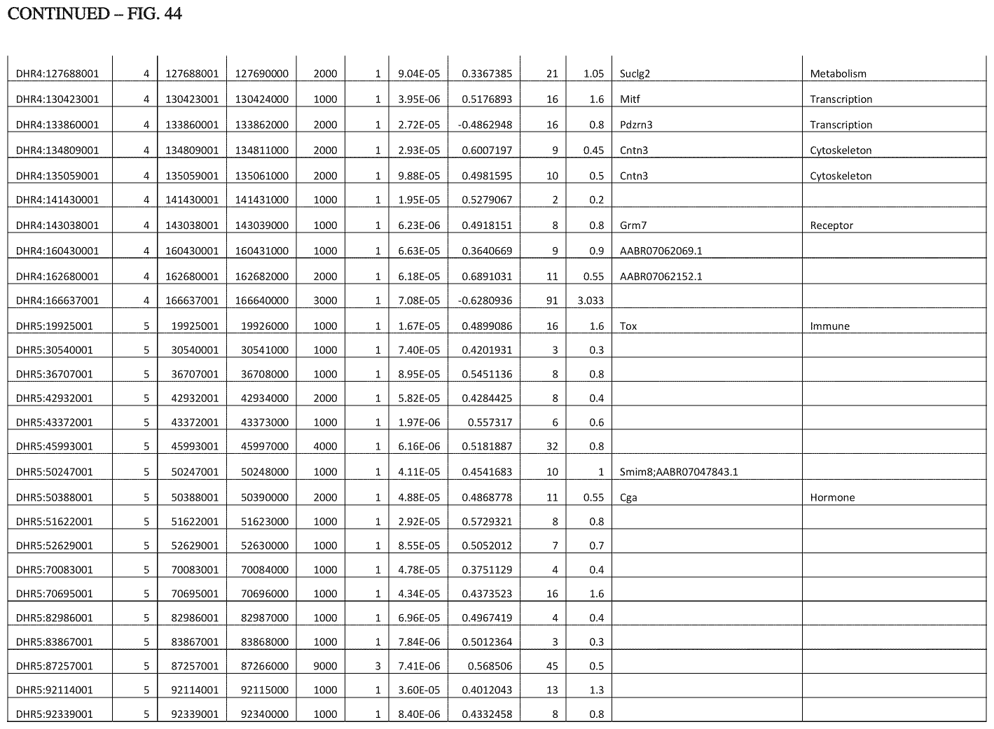

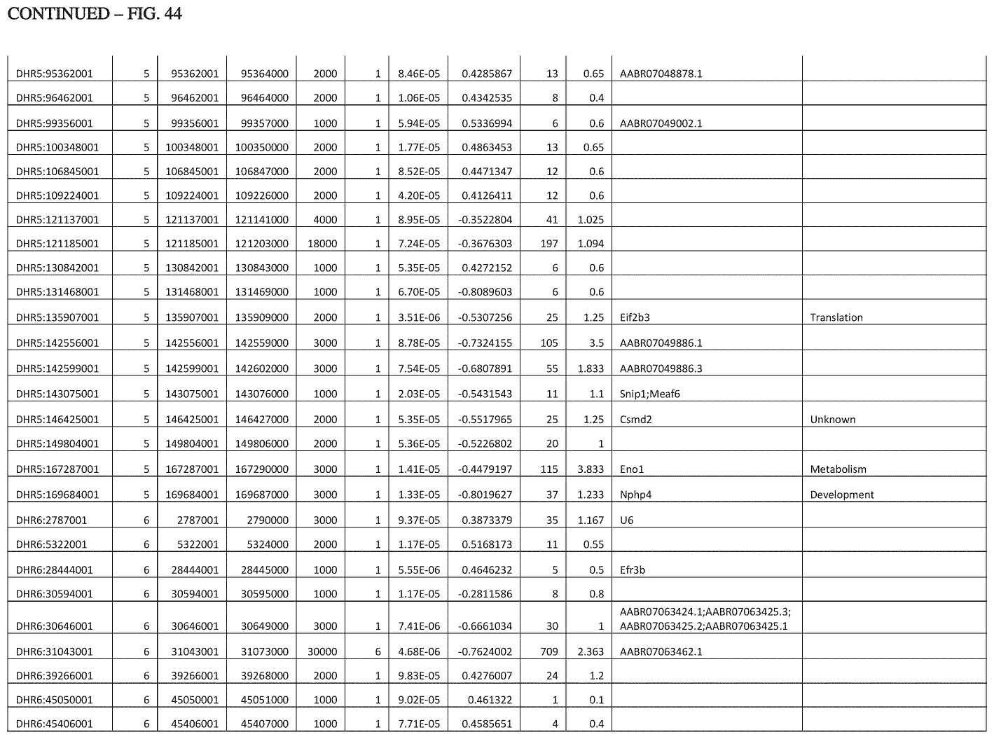

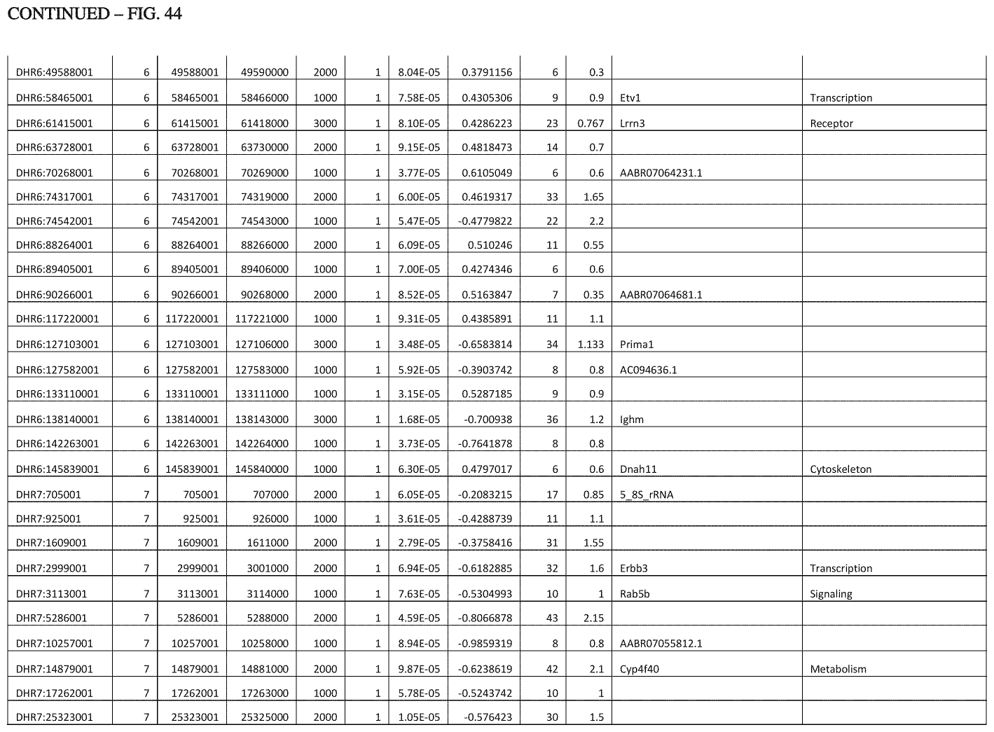

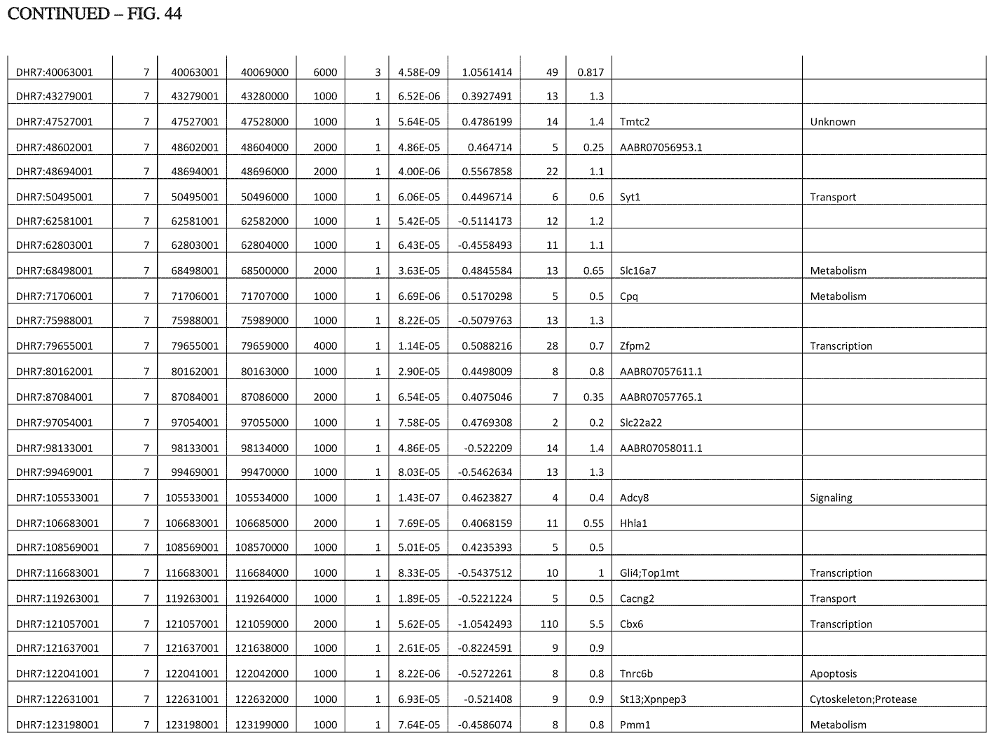

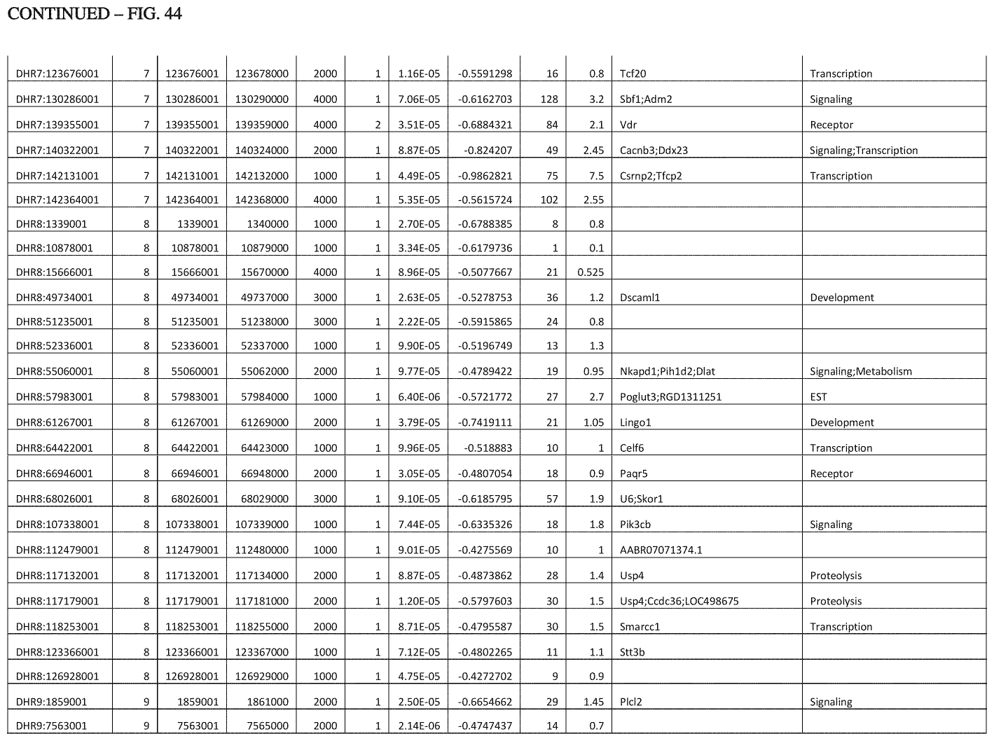

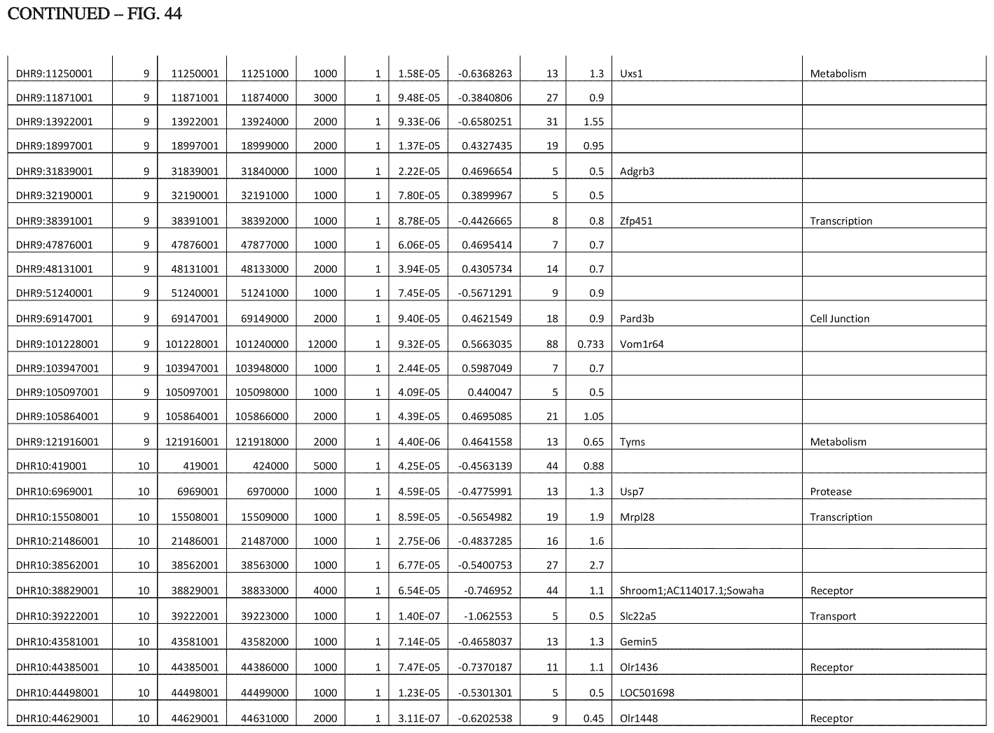

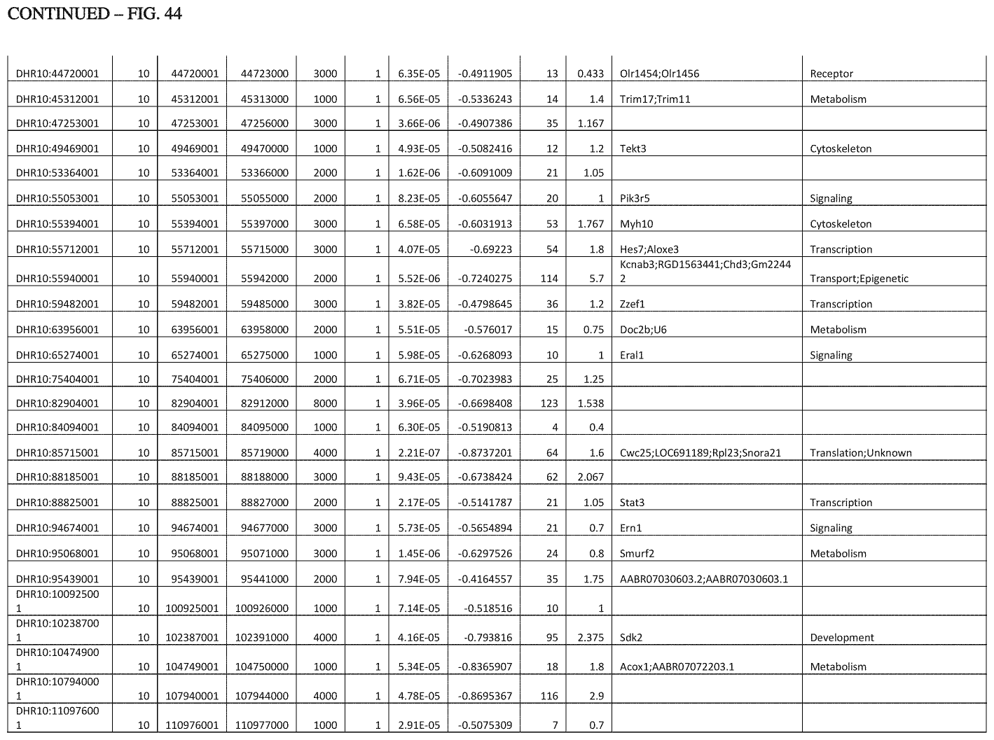

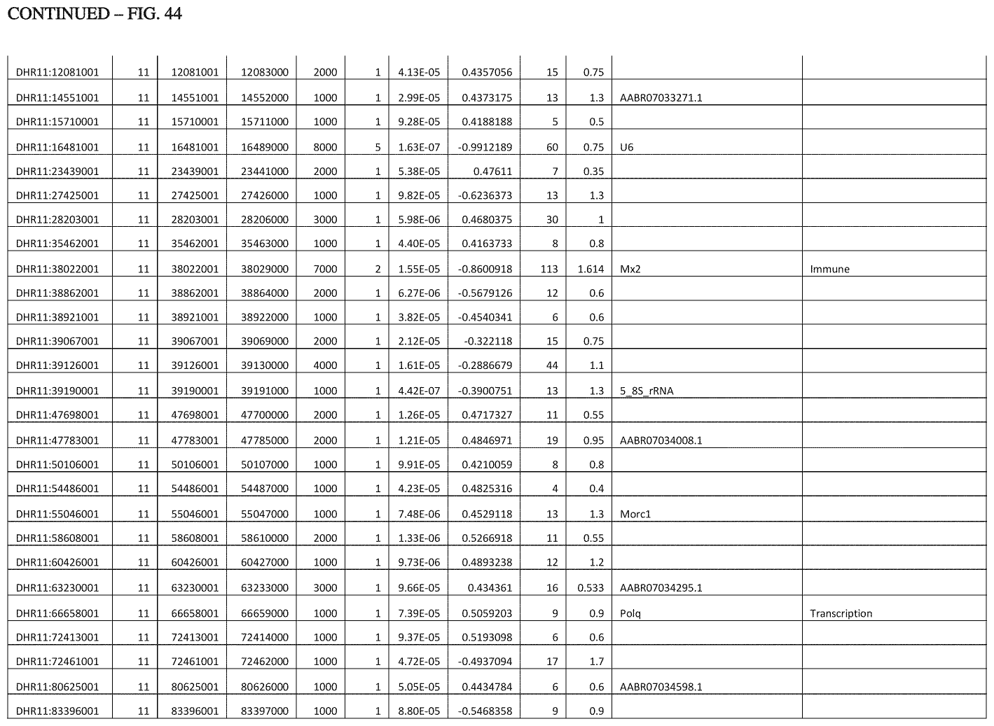

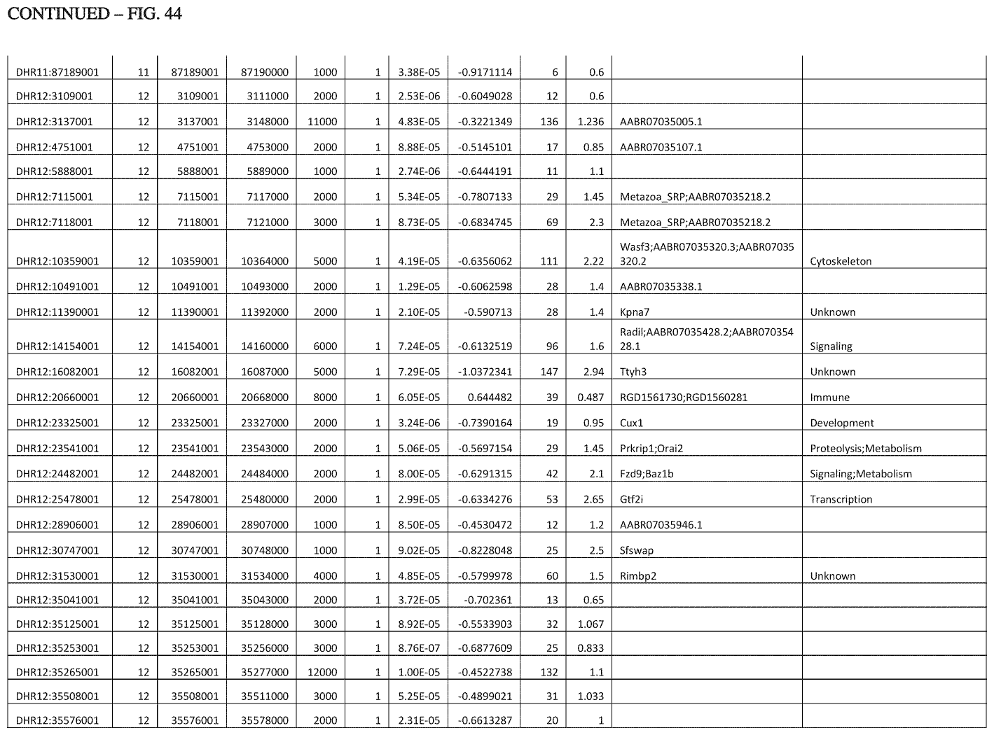

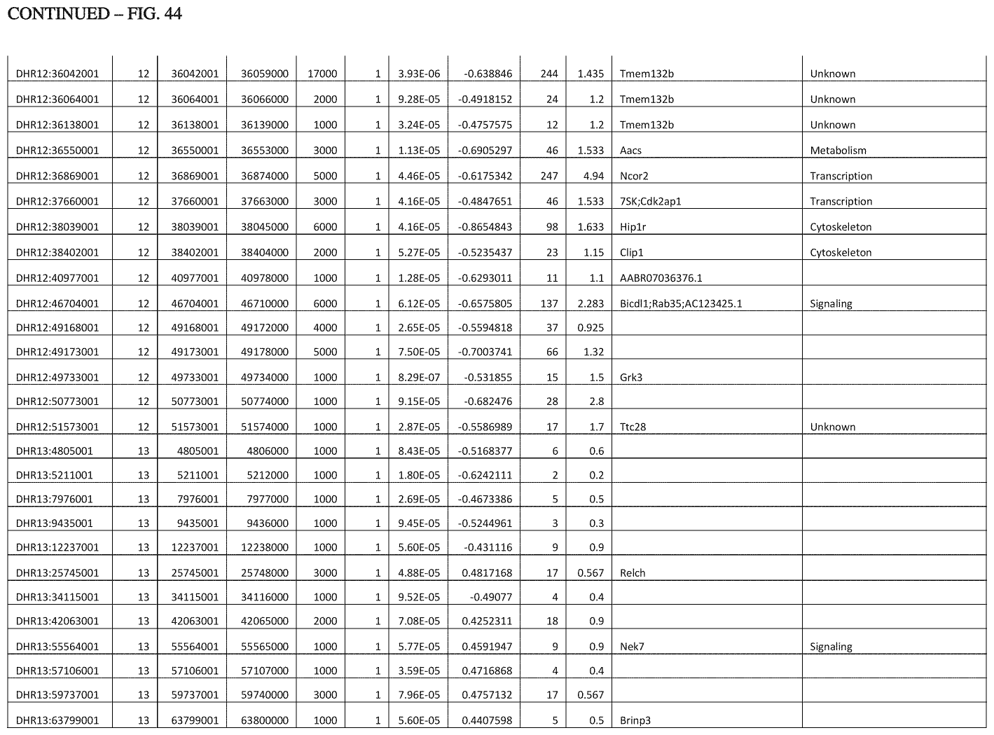

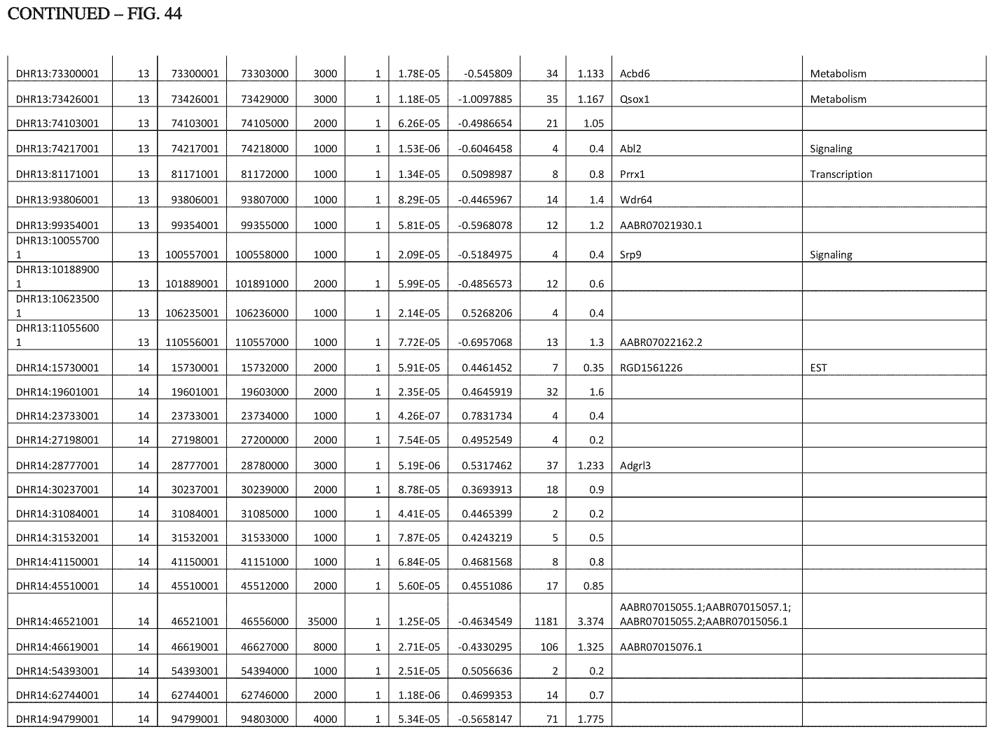

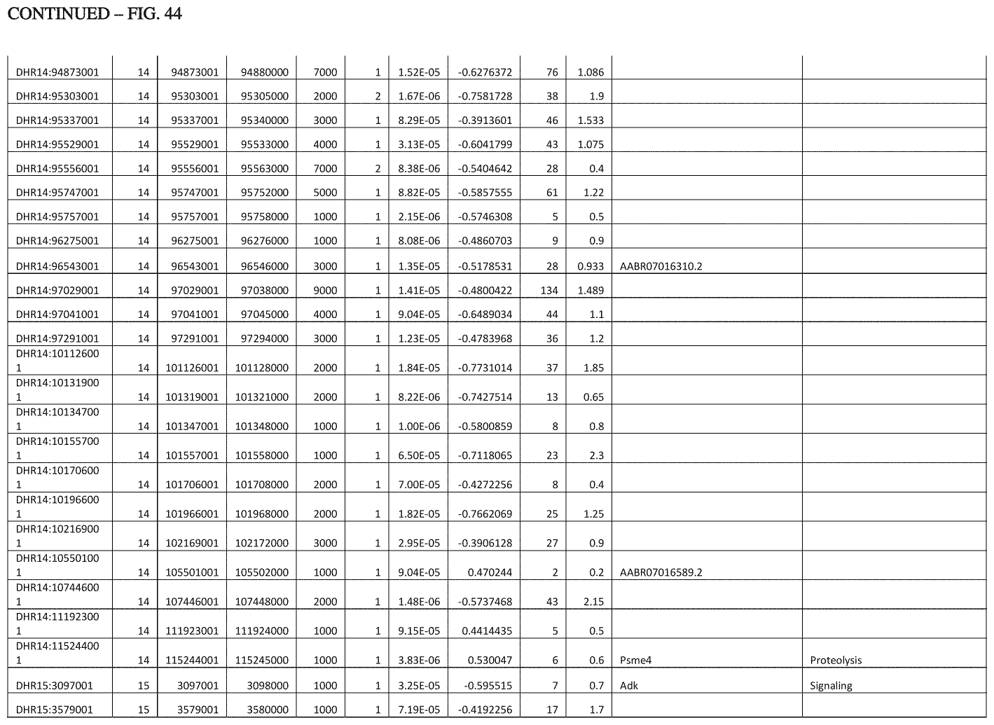

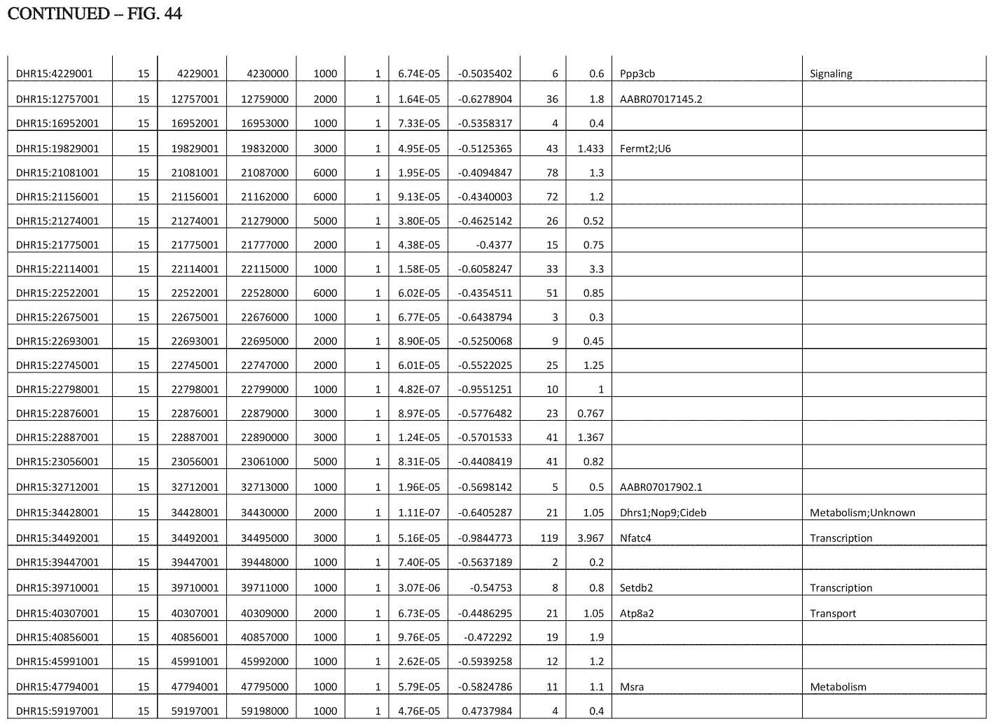

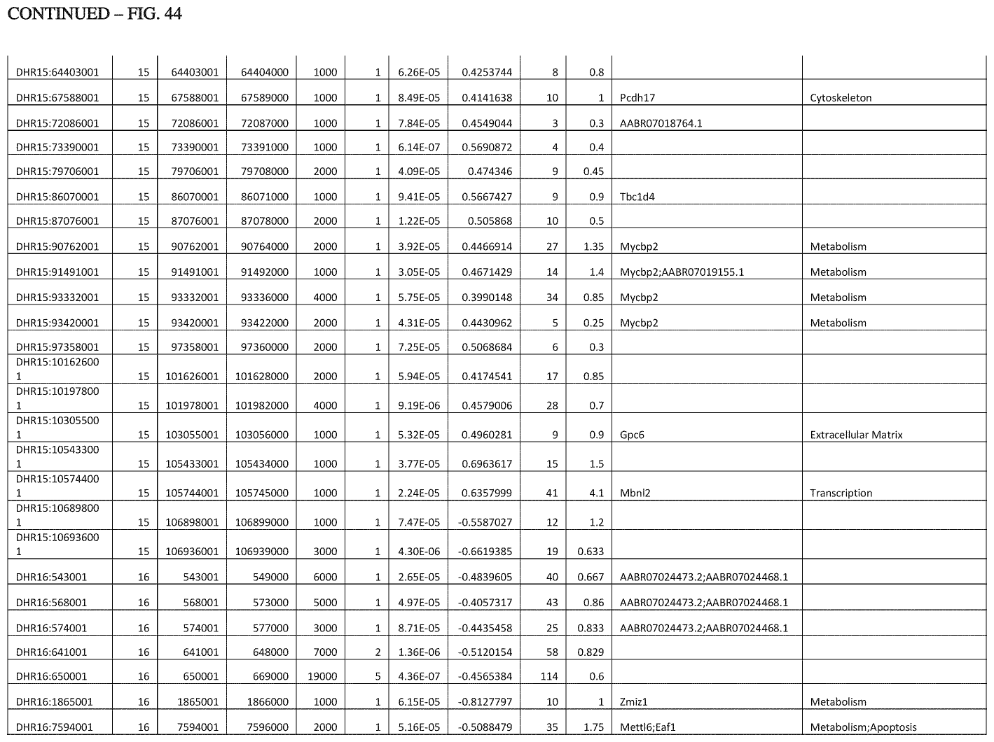

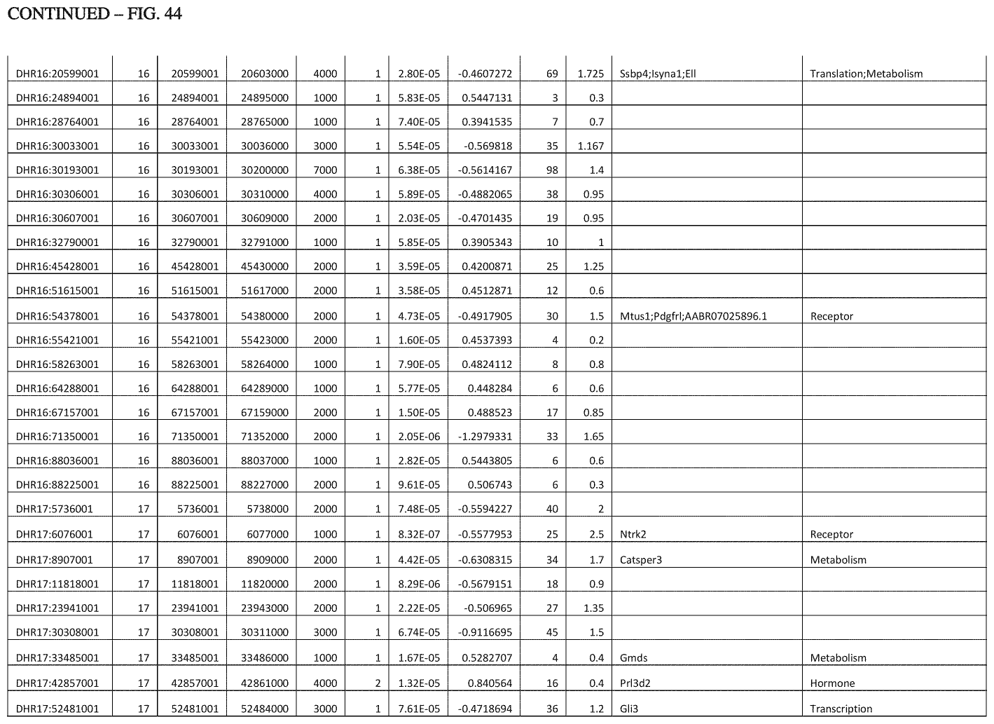

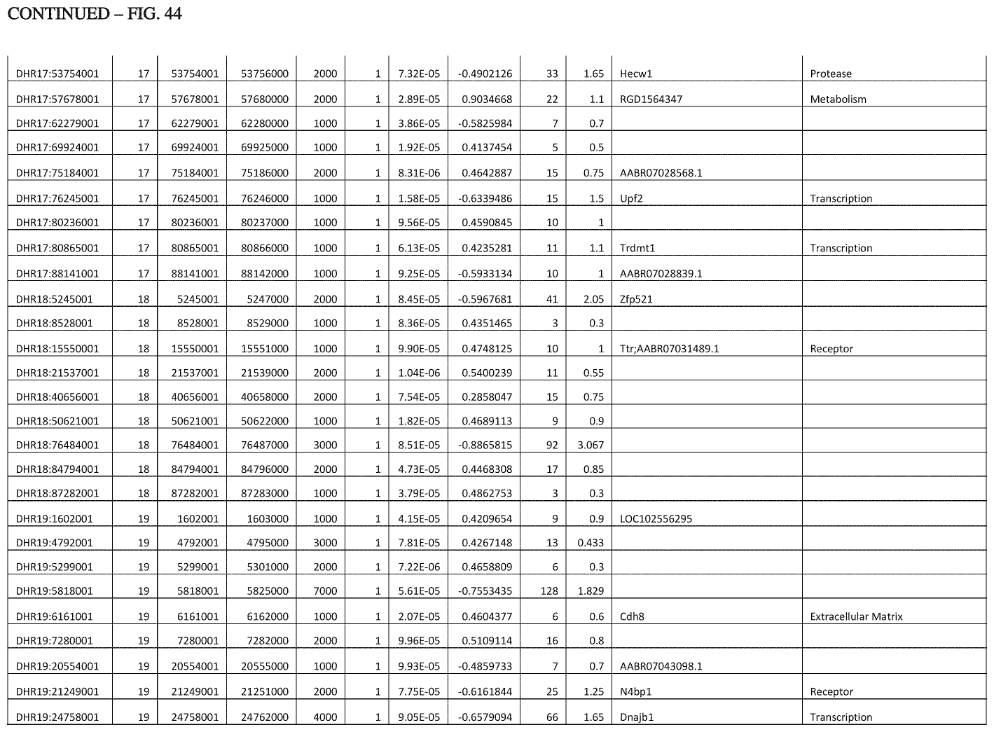

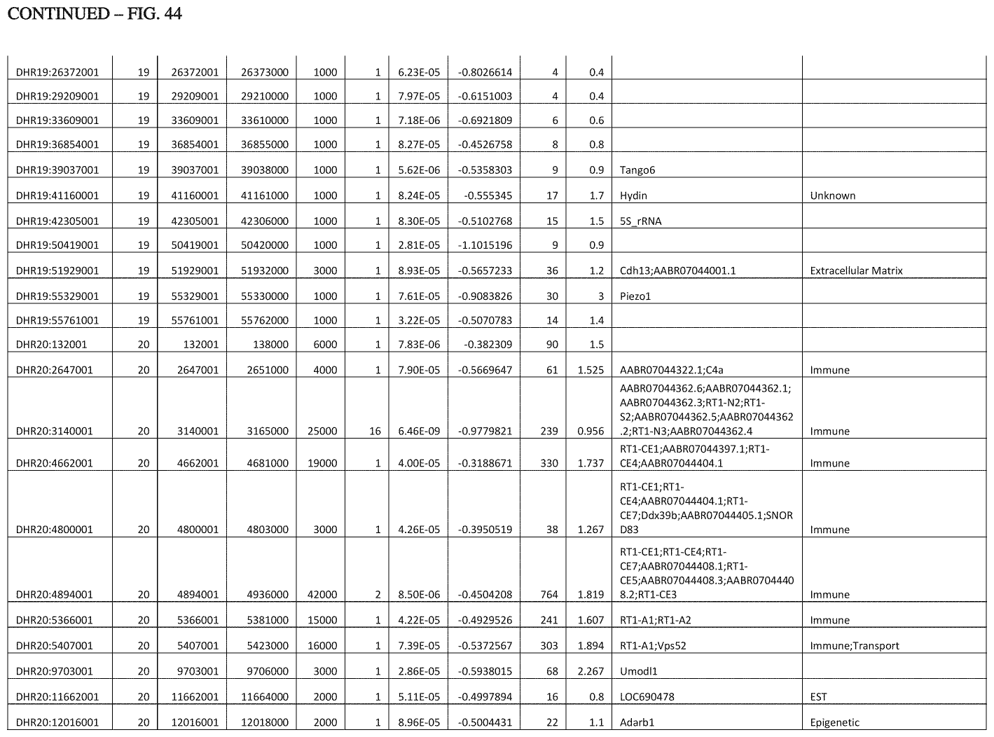

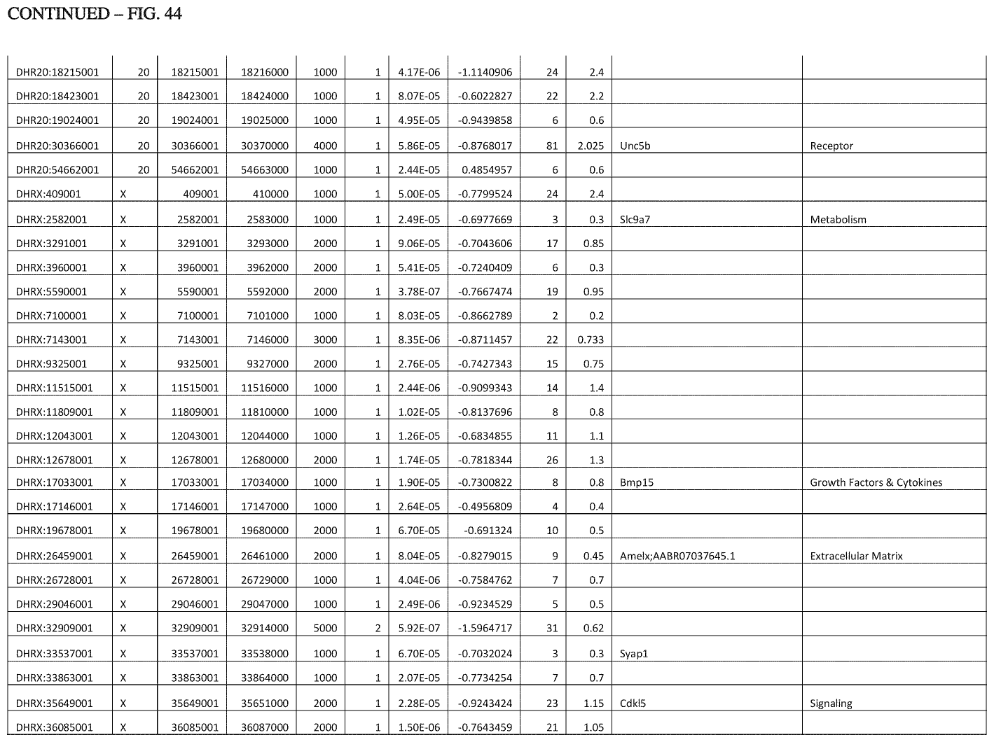

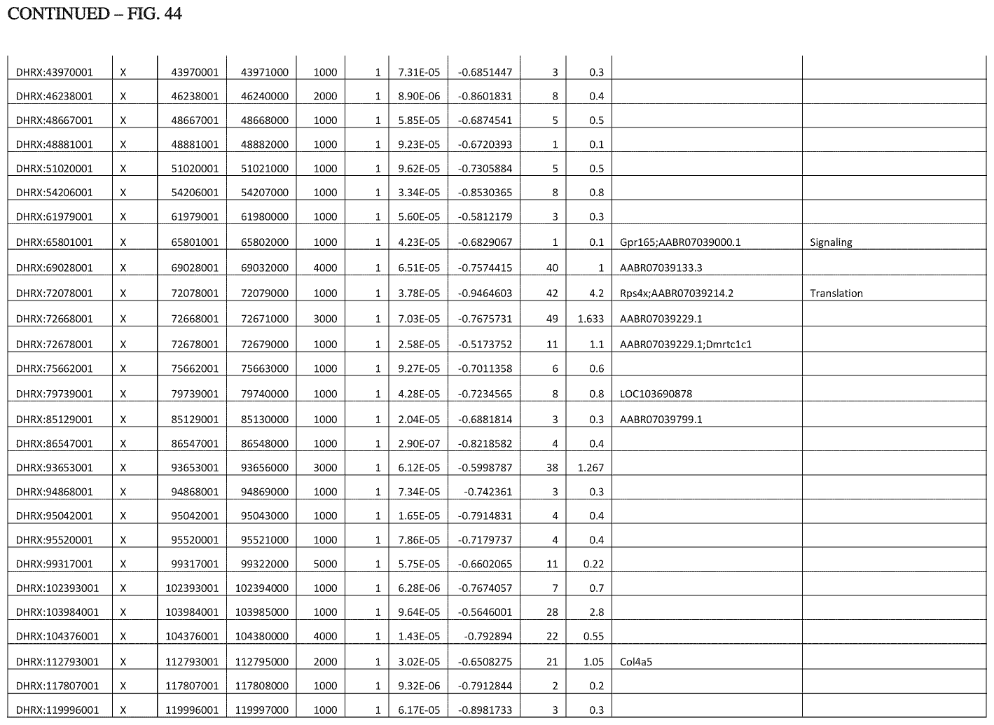

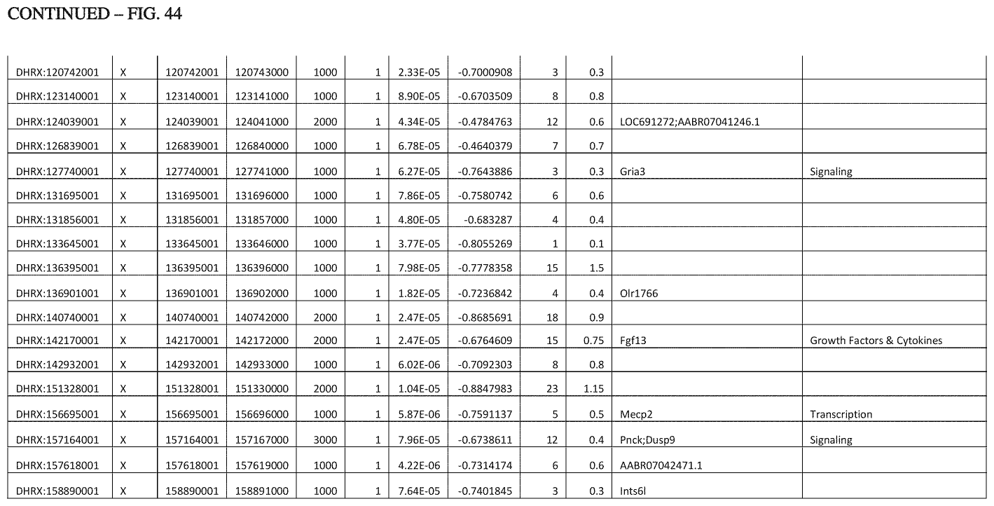

In addition to the atrazine lineage disease specific DHR analyses, control lineage F3 generation (Table 4) sperm was compared to the F3 generation atrazine lineage male sperm. The control versus atrazine lineage F3 generation sperm identified DHRs at a variety of statistical thresholds, with 786 DHRs at p<1e−04 (FIG. 43A and FIG. 44). The majority were single 1 kb sites, but some multiple nearby 1 kb sites were also observed. The chromosomal locations demonstrated a genome-wide distribution, FIG. 43B. The CpG density of the DHRs was predominantly 1-3 CpG/100 bp, FIG. 43C, and size of the DHRs was predominantly 1-4 CpG, FIG. 43D. A PCA demonstrated distinct clustering and no outliers of the control versus atrazine samples with read depths at DHR sites considered, FIG. 43E. Therefore, in addition to atrazine induced transgenerational DMRs, there is also an induction of DHRs in the sperm. This provides additional support for a role of sperm DHRs in the sperm mediated epigenetic transgenerational inheritance phenomenon.

### Epimutation Comparisons

A comparison of the different epigenetic data sets for each disease category among both DMRs and DHRs demonstrated only a handful of overlapping sites at the statistical threshold of p<1e−04 (see FIGS. 26F and 34F). Most of the epimutations associated with each disease category are unique to either a differentially methylated region or a differential histone retention site at this statistical threshold. To more rigorously compare the different datasets, an extended overlap was performed. A comparison with a reduced statistical threshold of edgeR p<0.05 was used to further evaluate the potential overlap of the DMR and DHR data sets at p<1e−04. By lowering the stringency to a p<0.05 for the comparison (herein called the extended overlap), this procedure allows for increased overlap with higher p-values. The extended overlaps between the atrazine lineage puberty, testis, kidney, lean, and multiple disease DMRs and DHRs are shown in Table 5. In Table 5, the p-value DMR/DHR set at p<1e−04 for specific diseases are compared to the p<0.05 DMR/DHR to identify potential overlap between the different pathologies with DMR or DHR number and percentage of the total presented. The boxes marked with an (*) shows 100% overlap.

TABLE 5
Extended Overlap Disease DMRs and DHRs

| p <0.05 | | |
|---|---|---|
| | Late Puberty | Multiple (2+) |

| p <1e-04 | Lean DMR | Kidney DMR | Testis DMR | DMR | DMR |
|---|---|---|---|---|---|
| Lean DMR | 301 (100.0%) * | 42 (14.0%) | 45 (15.0%) | 83 (27.6%) | 114 (37.9%) |
| Kidney DMR | 42 (16.1%) | 261 (100.0%) * | 62 (23.8%) | 50 (19.2%) | 48 (18.4%) |
| Testis DMR | 64 (9.2%) | 103 (14.9%) | 693 (100.0%) * | 63 (9.1%) | 122 (17.6%) |
| Late Puberty | 81(25.2%) | 44(13.7%) | 51(15.8%) | 322(100.0%)* | 113 (35.1%) |
| DMR | | | | | |
| Multiple (2+) | 124 (36.9%) | 68 (20.2%) | 79 (23.5%) | 125 (37.2%) | 336 (100.0%) * |
| DMR | | | | | |
| Lean DHR | 150 (5.2%) | 135 (4.7%) | 256 (9.0%) | 138 (4.8%) | 177 (6.2%) |
| Kidney DHR | 6(2.8%) | 14 (6.5%) | 24 (11.1%) | 5(2.3%) | 13 (6.0%) |
| Testis DHR | 14 (4.3%) | 17 (5.2%) | 20 (6.1%) | 14 (4.3%) | 12 (3.6%) |
| Late Puberty | 8 (2.7%) | 10 (3.3%) | 10 (3.3%) | 9 (3.0%) | 20 (6.7%) |
| DHR | | | | | |
| Multiple (2+) | 11(6.3%) | 10(5.7%) | 12(6.9%) | 9 (5.2%) | 9 (5.2%) |
| DHR | | | | | |
| Lean DMR | 35(11.6%) | 9(3.0%) | 14 (4.7%) | 10 (3.3%) | 25(8.3%) |
| Kidney DMR | 34 (13.0%) | 17 (6.5%) | 20 (7.7%) | 14 (5.4%) | 35 (13.4%) |
| Testis DMR | 111 (16.0%) | 54(7.8%) | 54(7.8%) | 35 (5.1%) | 93 (13.4%) |
| Late Puberty | 56 (17.4%) | 14 (4.3%) | 23 (7.1%) | 27 (8.4%) | 14 (4.3%) |
| DMR | | | | | |
| Multiple (2+) | 51(15.2%) | 10 (3.0%) | 16 (4.8%) | 14 (4.2%) | 20 (6.0%) |
| DMR | | | | | |
| Lean DHR | 2859 (100.0%) * | 241 (8.4%) | 256 (9.0%) | 297 (10.4%) | 739 (25.8%) |
| Kidney DHR | 39 (18.1%) | 216 (100.0%) * | 30 (13.9%) | 34 (15.7%) | 33 (15.3%) |
| Testis DHR | 40 (12.2%) | 48 (14.6%) | 329 (100.0%) * | 54 (16.4%) | 62 (18.8%) |
| Late Puberty | 48 (16.1%) | 36 (12.0%) | 38 (12.7%) | 299(100.0%)* | 40 (13.4%) |
| DHR | | | | | |
| Multiple (2+) | 63 (36.2%) | 23 (13.2%) | 34 (19.5%) | 29 (16.7%) | 174 (100.0%) * |
| DHR | | | | | |

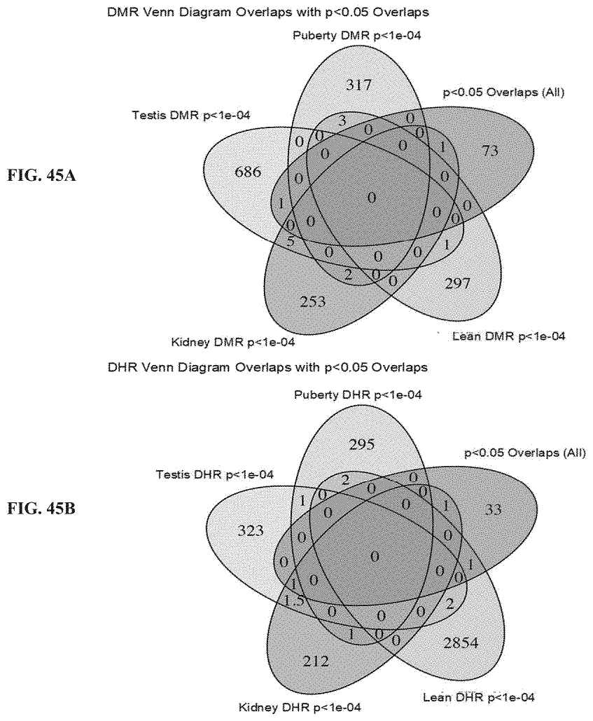

A comparison of the p<1e−04 for the DMRs and DHRs between the different data sets at p<0.05 demonstrates a much higher overlap between the various DMRs and DHRs identified than the Venn diagrams in FIGS. 26F and 34F. The extended overlap also shows any overlaps between DMRs and DHRs for the individual disease comparisons. The range of overlap between atrazine lineage disease DMR or DHR is 8-37%, Table 5. Comparison between the DMRs and DHRs for the specific diseases had a lower range of overlap with 2-17%, Table 5. The highest level of overlap was observed between the lean disease and multiple disease for both the DMRs and DHRs, Table 5. Therefore, the majority of the disease specific DMRs and DHRs at p<1e−04 are distinct, but an overlapping set of DMRs and DHRs are common between two diseases. An overlap of all the disease specific DMRs and DHRs was performed at p<0.05 and identified 75 DMRs and 36 DHRs that are common between puberty abnormalities, testis disease, kidney disease, lean pathology and multiple disease at p<0.05. A Venn diagram of this common set of DMRs and DHRs at p<0.05 with the specific diseases at p<1e−04 demonstrated no overlap in common with all pathologies, FIG. 45. Therefore, no common DMRs or DHRs were observed with all the different pathologies.

### Epimutation Gene Associations

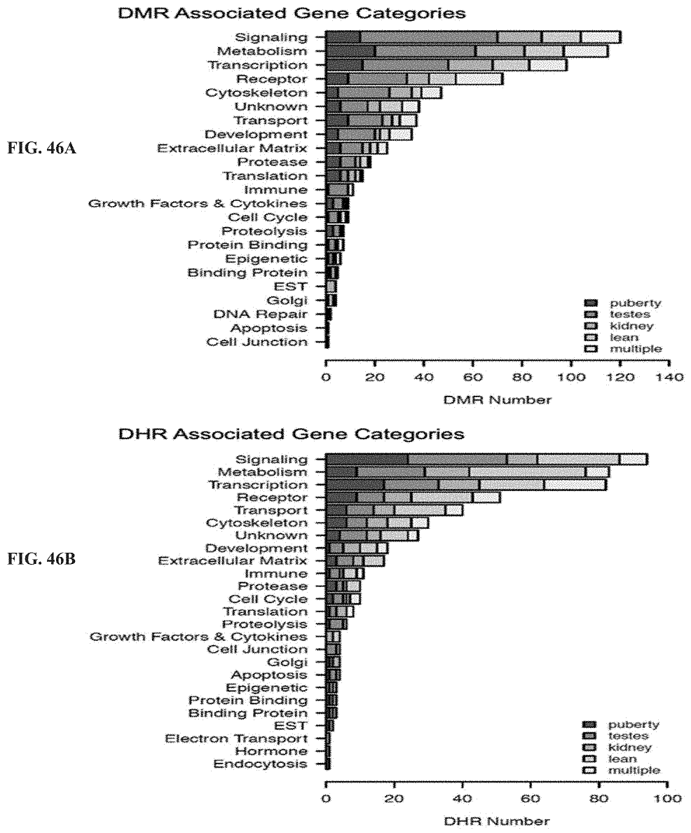

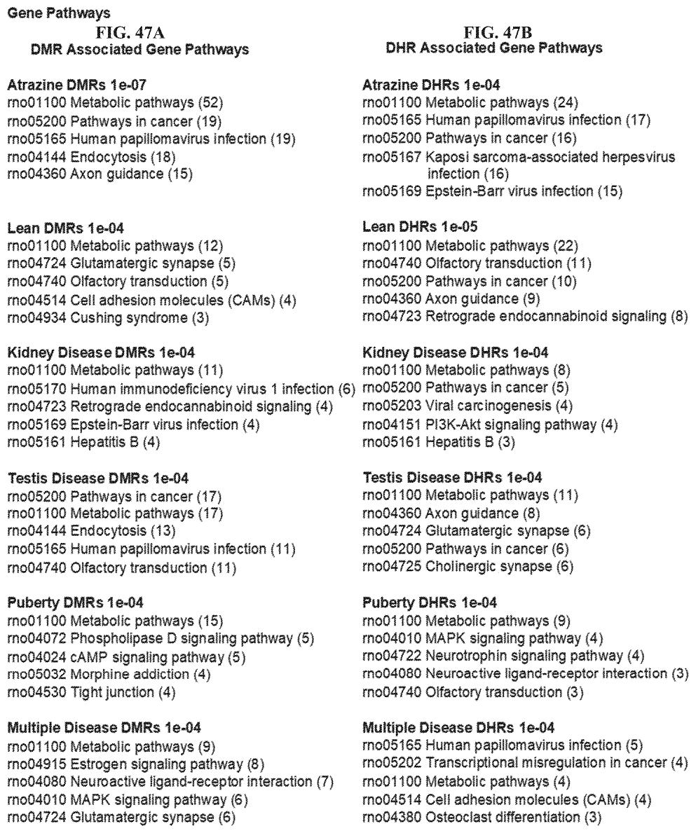

Between 49% and 55% of the DMRs and between 41% and 54% of the DHRs from the specific disease prevalence have epimutations associated with genes. These epimutation associated genes are presented in FIGS. 27-31 and 35-39. The gene associations were sorted into relevant functional categories for each specific disease biomarker dataset within the atrazine lineage (FIG. 46). Disease specific epimutation associated genes identified in the analysis of DMRs are shown in FIG. 46A, where the predominant categories identified were signaling, transcription and metabolism. The epimutation associated genes identified in the DHR analysis are shown in FIG. 46B, where the same categories are predominant. The DMRs and DHRs associated with gene pathways are presented in FIG. 47. Although some of the pathways were common for the specific disease and pathways, generally the DMR pathways and DHR pathways were distinct.

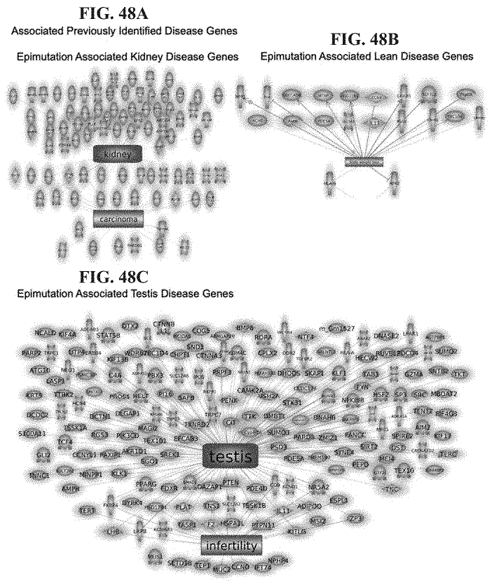

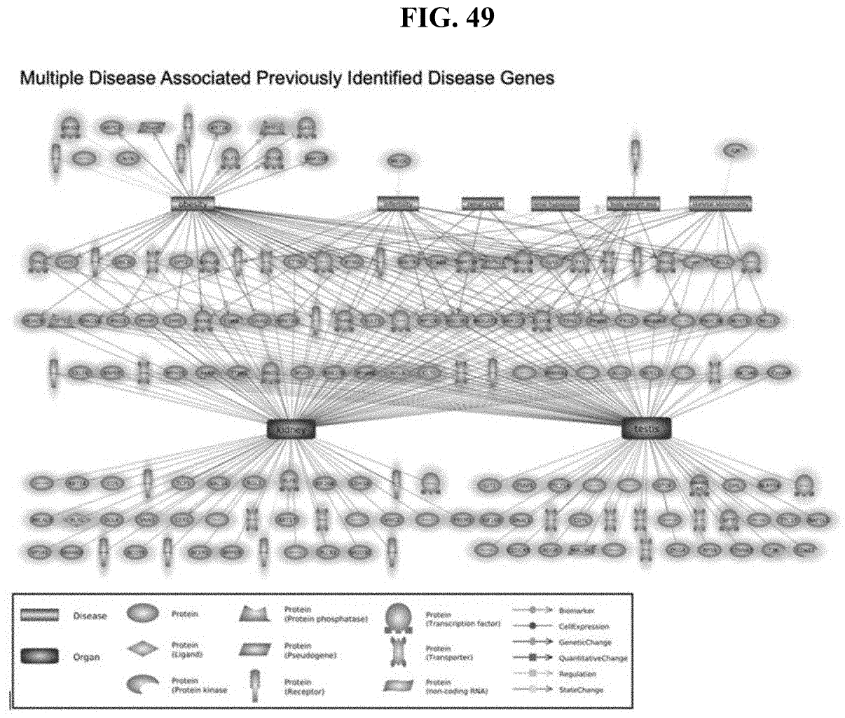

The final analysis used a Pathway Studio gene network approach to associate previously identified disease specific associated genes with the disease specific DMRs and DHRs identified. A large number of previously identified kidney disease linked genes were found to be within the DMR and DHR associated genes, FIG. 48A. A number of previously identified obesity and breast cancer-related genes were also associated with the DMR and DHR associated genes, FIG. 48B. The highest number of previously identified genes was associated with testis disease and male infertility associated genes, FIG. 48C. These observations help validate the DMR and DHR associated genes with the different specific diseases. Interestingly, the multiple disease associated DMRs and DHRs had disease genes previously identified for kidney disease, testis disease, obesity and male infertility, FIG. 49. The multiple disease DMR and DHR associated genes had a mixture of the various diseases identified involved.

### CONCLUSIONS

Surprisingly, the instant example demonstrates not only DNA methylation alterations but also provides one of the first observations of disease-specific alterations in histone retention sites. These transgenerational epigenetic shifts are associated with ancestral exposure to the toxicant atrazine, and potential disease specific biomarkers for pathologies were identified. The pathologies observed with sufficient numbers of animals include the lean phenotype, kidney disease, testis disease, late puberty, and multiple disease where individuals exhibited two or more different pathologies.

The frequency of epimutations tend to be much higher among individuals with disease. In the instant example, the number of differential DNA methylated regions (DMRs) occurring in the transgenerational males is between 300 and 600 at an edgeR p<1e−04 threshold (FIG. 26) for each pathology. This supports the prevalence of epigenetic alterations as important biomarkers of disease.

There is also a sub-population of DMRs and DHRs with overlap between the different individual disease pathologies, suggesting some of the epimutations are less disease specific and indicative of multiple pathologies. This suggests some common epimutations may have a role in promoting generational disease susceptibility. Therefore, the majority of epigenome-wide association study (EWAS) associated epimutations were disease specific. The DMR and DHR associated genes suggest the most affected gene categories were signaling, metabolism, and transcription. The analysis of previously identified disease associated genes yields a number of links with the diseases examined in the current study (see FIGS. 46 and 48). The association of these previously identified disease genes with the disease specific epigenetic marks in the current study, including DMRs and DHRs, provides further validation of the disease biomarkers identified herein. In addition to the altered methylation observed among the atrazine lineage males, there were also alterations in differential histone retention regions (DHRs). This represents an additional transgenerational epigenetic response to toxicant exposure. The DHRs also provide a potential epigenetic biomarker for disease. The altered histone retention signature was in the range of 200-300 DHRs at an edgeR p<1e−04 threshold (FIG. 34) for most disease pathologies. However, there were nearly 3,000 DHRs (FIG. 34B) associated with the lean phenotype, indicating the lean physiology is a particularly strong phenotypic response to transgenerational atrazine exposure. Unexpectedly, some DMRs and DHRs had overlap between the different individual diseases, suggesting some of the epimutations may act as biomarkers for susceptibility of multiple pathologies. Table 2 also shows the extended overlap between DMRs and DHRs. Late puberty associated DMR overlap 28% with lean phenotype DMR. A 24% overlap is found between testis and kidney disease associated DMR. Most of these overlaps including DMRs and DHRs are quite low. The gene categories for the DHRs are quite similar to the DMR categories, with the same three processes most relevant. DHR associated disease genes are also shown in FIGS. 48 and 49.

The atrazine induced transgenerational lean pathology was quite prevalent in the exposed population, with nearly a third of the animals presenting this pathology. The epigenetic signature was also strong, with 301 DMRs and 2,859 DHRs at an edgeR p<1e−04 threshold. A lean phenotype can be as significant an indicator of disease as an obese phenotype. More importantly, the early developmental effects of exposure to EDCs could have magnified effects in later development and this may be particularly important with regards to other metabolic disorders.

1. A method for preparing a DNA fraction from an animal useful for analyzing histone retention involved in an epigenetic alteration, comprising
(a) extracting DNA from a germline sample of an animal,
(b) producing a fraction of the DNA extracted in (a) by selecting DNA comprising histone retention, and
(c) analyzing the histone retention in the fraction of DNA produced in (b).

2. The method of claim 1, wherein the germline sample is sperm.

3. The method of claim 1, wherein analyzing the histone retention comprises histone antibody chromatin immunoprecipitation (ChIP) analysis.

4. The method of claim 1, wherein analyzing the histone retention comprises PCR.

5. The method of claim 1, wherein analyzing the histone retention comprises ChIP and PCR.

6. The method of claim 1, wherein the animal is a male progeny animal born from a female parental animal, wherein the female parental animal is administered one or more toxicants.

7. The method of claim 6, wherein the epigenetic alteration is associated with in reduced fertility of the male progeny animal or sterility of the male progeny animal.

8. The method of claim 7, wherein the reduced fertility or sterility of the male progeny animal is associated with abnormal testicular development, abnormal spermatogenesis, decreased sperm motility, decreased forward sperm movement, or any combination thereof.

9. The method of claim 1, wherein the histone retention comprises an altered histone retention DNA profile associated with a disease.

10. The method of claim 9, wherein the disease is selected from the group consisting of kidney disease, prostate disease, male infertility, immune cell activation, cancer, and any combination thereof.

11. The method of claim 9, wherein the method further comprises identification of one or more genes associated with the altered histone retention DNA profile.

12. The method of claim 1, wherein the histone retention analysis is combined with a differential DNA methylation region analysis.

13. The method of claim 1, wherein the animal is a human.

14. A method of identifying a disease or a disease propensity in a male progeny animal, comprising: identifying a profile of histone retention DNA in the male progeny animal, wherein the profile is associated with the disease or the disease propensity in the male progeny animal, and wherein the disease or the disease propensity is associated with an epigenetic alteration in germline DNA resulting from contact of the germline DNA of the male progeny animal with one or more toxicants during gestation of the male progeny animal.

15. The method of claim 15, wherein the male progeny animal is administered a pharmaceutical composition to treat the identified disease or the disease propensity.

16. The method of claim 14, wherein the disease is selected from the group consisting of kidney disease, prostate disease, male infertility, immune cell activation, cancer, and any combination thereof.

17. The method of claim 17, wherein the profile of histone retention DNA is identified according to a method for preparing a DNA fraction from the male progeny animal comprising
(a) extracting DNA from a germline sample of the male progeny animal,
(b) producing a fraction of the DNA extracted in (a) by selecting DNA comprising histone retention, and
(c) analyzing the histone retention in the fraction of DNA produced in (b).

18. The method of claim 14, wherein the histone retention analysis is combined with a differential DNA methylation region analysis.

19. The method of claim 14, wherein the animal is a human.

20. The method of claim 14, wherein the animal is a companion animal.
